SEQUENCE LISTING (306 sequences)

SEQ ID NO: 1 - protein, 157 aa, Ajellomyces capsulatus
PHVNKATLALIKEFEGFVPRPEPDPIGLPTVGYGHLCKTKGCKEVKFPLSKGTATTLLKKDLRSFQQAITLSTKTAVKLNANQYGALVSWAYNVGPNAARSSSLISRLNQGEDPNQVIAQELPKWRLAGGKVFEGLVRRRKAEVKLAKTPTKSKALP

SEQ ID NO: 2 - protein, 159 aa, Coprinopsis cinerea
INKKTLDLLKEFEGWAASPYKDVAGYPTVGYGHKCSKNDCSELGYKFPMTKAQGEELLAKDVKGFEKCISDYINDTIKLNDNQYGALVSWSFNVGCGAAKDSTLISRLNKGDSPNTVAGEELPRWNKAGGKVVDGLTNRRKKEVELFKTSSTVIAHPPC

SEQ ID NO: 3 - protein, 154 aa, Aspergillus terreus
GKLLRRGISDAAVELIGSLEGFRPDFYYINGHKTVGYGHDCVAKQDCDSIDTPLTKEEGAALLKKDLAGYENCVCEMDNAKYLNANQYGALVSFAYNSGCGGVQSWWHGAMEQKNFKGICSALPNTNTLGGELSSRRAKEGAFCAKPTNQTSGC

SEQ ID NO: 4 - protein, 166 aa, Aspergillus terreus
ACSGPNVNEATIKLMKGYESWEADVYDDGYGNPTVGYGHLCDDWSCSDVSYDIPLSESDGEKLFAEDIVAYQNGVVAALSDDVTLNDNQYGALVSWCFNVGTGAVAESTLAKRLNNGEDPDTVAEEELPKWVYANGAPSEGLKNRRAAELKLFTTSSDTEALPVDC

SEQ ID NO: 5 - protein, 154 aa, Aspergillus oryzae
ATVVRRGASAATVSLIGEVEGFRADFYDMMGHKTIGYGHDCVAKQDCDSIKAPISNAQGDEILQKDLAGFEQCVCALPNAKALNANQYGALVSYAFNTGCGGLQQAWTAAMTSKNFDSICADLPHTNTLNGVLDNRRKKEAALCSTPTTQMCGC

SEQ ID NO: 6 - protein, 163 aa, Aspergillus oryzae
GPPVNQNGLNLIKSFESFQPSVYDDGFGNPTIGYGHLCGDATCSEVTYPKPLSEADASRLLADDLVSYQDALTNALADPVTLNDNQYAALVSWTFNIGNGNMQKSDLVARMNKGENVATVAHDELPQWNKANGQVVNGLTRRRKAELDLFDAPAIYGALPVPC

SEQ ID NO: 7 - protein, 162 aa, Emericella nidulans
PDVNTATTDLMKAFESWEPDVYDDGYGNPTIGYGHLCSDWSCSDVAYDIPLSEEDGVKLFAEDIAVYQDGVVSALDSSVTLNDNQYGALVSWCYNVGAGAVAESTLAARLNAGEDPNTVAEEELIKWVYANGEVSEGLKRRRNAEIELFQTSSDGEALPVSC

SEQ ID NO: 8 - protein, 159 aa, Laccaria bicolor
ISSATVNLIKGSESLVPIPSPDPIGLLTVGYGHKCLKPQCSEVTFPFPLSSSTASQLFAQDMTQYINCLHRSISKSVVLNDNQFGALVSWTYNAGCEGMGTSTLVKRLNNGEDPNTVVAQELPKWNIAKKKISKGLVNRRNREISFFQTPSNVVAHPLC

SEQ ID NO: 9 - protein, 160 aa, Laccaria bicolor
PPANADTINLIERFEGFVPSPRPDPIGLPTVGYGHLCKTKGCSEVPFKFPVTKANAVTLLHSDLTTFQNCVNSDIKRSVHLNDNQYGALVSWAYNVGCGNIKTSSLVRRLNAGEDPNTVAAQELPQWNKGGGKVLPGLVRRRAEEVKLFKTPSSVSAHPC

SEQ ID NO: 10 - protein, 162 aa, Laccaria bicolor
PAVNAATIALIKKFEGFVASPSPDPIGLPTVGYGHLCQTKNCAEVPFSFPLTEAEASTLLNSDLKTYEACITKDIVSSVRLNDNQYGALCSWAFNEGCGAAGSSTLIARLNAGQDPDAVAAQELPKWDIAGGKVLQGLVNRRAAEVALFKTPSSVIAHPPHC

SEQ ID NO: 11 - protein, 163 aa, Aspergillus flavus; identical to SEQ ID NO: 6

SEQ ID NO: 12 - protein, 154 aa, Aspergillus flavus
ATVVRRGASAATVSLIGEVEGFRADFYDMMGHKTIGYGHDCVAKQDCDSIKAPISNAQGDEILQKDLAGFEQCVCALPNAKALNANQYGALVSYAFNTGCGGLQQAWTAAMTSKNFDSICADLPHTNTLNGVLDNRRKKEAALCATPTTQMCGC

SEQ ID NO: 13 - protein, 157 aa, Ajellomyces capsulatus
PNVNKATLDLIKEFEGFVPRPEPDPIGLPTVGYGHLCKTKGCKEVKFPLSKETATTLLKKDLRSFQQAITLSTKTAVKLNANQYGALVSWAYNVGPNAARSSSLISRLNKGEDPNKVIAQELPKWRLAGGKVFKGLVRRRKAEVKLAKTPTKSKALP

SEQ ID NO: 14 - protein, 162 aa, Arthroderma otae
PDVNDETIALIKHFEGFVPRPAPDPIGLPTVGYGHLCRTKGCGEVPFPFPLTEESATELLHQDVKSPQQSITLSTADSVVLNANQYGALVSWAFNVGGGAAKKSSLIKRLNQGQDVDTVIREELPLWNKAGGHVLPGLVRRRKAEVELAMEHTDDGALPVDC

SEQ ID NO: 15 - protein, 162 aa, Arthroderma otae
PDVNAATISLVKEFERFVPSPSPDPIGLPTVGYGHLCQSKNCGEVGFPFPLTEDTATQLLAQDIKAPQQTITLKTVNGVHLNENQYGALVSWTFNVGPGNVATSSLLKRLNALEDVNTVLREELPKWKYAGGKVLPGLVRRRAAEVALGETPSNVGALPVDC

SEQ ID NO: 16 - protein, 160 aa, Ajellomyces dermatitidis
PNVNKATLALIKEFEGFVPRPAPDPIGLPTVGYGHLCKTKGCKEVKFPLTKKTATALLKKDLRSFQQAITLSTKKAVKLNANQYGALVSWAYNVGPNAARSSSLIRRLNRGENPNKVIAQELPKWRLAGGKVFKGLVRRRKAEVKLAKTPTKKKALPVKC

SEQ ID NO: 17 - protein, 157 aa, Ajellomyces capsulatus
PNVNKATLALIKEFEGFVPRPEPDPIGLPTVGYGHLCKTKGCKEVKFPLSKGTATTLLKKDLRSFQQAITLSTKTAVKLNANQYGALVSWAYNVGPNAARSSSLISRLNKGEDPNKVIAQELPKWRLAGGKVFKGLVRRRKAEVKLAKTPTKSKALP

SEQ ID NO: 18 - protein, 163 aa, Hypocrea jecorina
KTINGAGVDLIAKWEGFVASPKPDPIGLPTVGYGHLCQQKNCAEVKYKFPLTKTTAKELLLDDLPKYTKCLADVLNSKPKLNANQWAALSSWVFNVGCGNAKTSTLVKRLNNGEAPNTVAAQELPKWRMAGGKVMPGLEARRKDEVKLFKTASSKEAYPKCQA

SEQ ID NO: 19 - protein, 162 aa, Arthroderma benhamiae
PDVNPATIALIKEFEGFVPAPAPDPVGLPTVGYGHLCQSKNCGEVGFPFPLTEDTATQLLIQDVKAPQQTITLKTADGVHLNENQYGALVSWTFNVGPGNVATSSLLQRLNALEDVNTVLREELPQWKYGGGKVLPGLVRRRAAEVALGETASDVPALPVAC

SEQ ID NO: 20 - protein, 162 aa, Trichophyton verrucosum; identical to SEQ ID NO: 19

SEQ ID NO: 21 - protein, 162 aa, Trichophyton verrucosum
PDVNDETIALIKHFEGFVPRPAPDPIGLPTVGYGHLCRTNGCSEVPFSFPLTEETATELLMQDVKSPQQSITLSTTDQVVLNANQYGALVSWAYNVGGDAAKKSSLISRLNQGQDVDVVIREELPLWNKAGGHVLPGLVRRRAAEVELASENTDQPALPVDC

SEQ ID NO: 22 - protein, 161 aa, Coprinopsis cinerea
NTATVNLIQEFEGFVASPEPDPIGLPTVGFGHLCRQPNCAEVTAQGLSFPLSRAQAEQLLQSDVQTFTNCLARFIDDSVVLNENQFGALTSWAFNVGCGNVQRSTLRRRLNAGQDPNTVAAQELPRFNRAGGRVLNGLTRRRNAEVALFQTPSNTVALPVC

SEQ ID NO: 23 - protein, 162 aa, Arthroderma gypseum
PDVNAATIALVKEFEGFVPSPAPDPIGLPTVGYGHLCQSKNCGEVGFPFPLTEDTATQLLSQDIKAPQQTITLKTADGVHLNENQYGALVSWTFNVGPGNVATSSLLKRLNALEDVNTVLREELPKWKYAGGKVLPGLVRRRAAEVALGETPSDVPALPVDC

SEQ ID NO: 24 - protein, 162 aa, Arthroderma gypseum
PDVNEATISLIKHFEGFVPRPAPDPIGLPTVGYGHACRTKGCAEVPFPFPLTEDTATELLMQDVKSFQQSITLSTTDEVVLNANEYGALVSWAFNIGGGAAKKSSLIRRLNQGQDVNTVLREELPLWNKAGGKVLPGLVRRRAAEVELASEHTDEPALPVDC

SEQ ID NO: 25 - protein, 162 aa, Hypocrea virens
KTINGAGVDLIAKWEGFVASPKPDPIGLPTVGYGHLCQQKNCREVKYKFPLTKTTAKELLLDDLPKYTKCLADYLNTKPKLNDNQWAALTSWVFNVGCGNAKTSTLVKRLNNGEAANTVAAEELPKWRMAGGKVLPGLEARRKDEVKLFKTASSKQAYPKCQ

SEQ ID NO: 26 - protein, 162 aa, Agaricus bisporus
PRVNSATISLIKEFEGFVKSPSPDPIGLPTVGFGHLCKSKGCAEVPYKFPLTEANAGKLLQTDIKSFTKCVSDNIKDAVKLNANQFGALSSWAFNVGCGNVKASALVARLNRGEKPNTVAAEELPKWRLAGGKVLKGLVRRRAAEVKLFKTASKAIAHPPKC

SEQ ID NO: 27 - protein, 163 aa, Metarhizium robertsii
KTLNKAGTDLITRWEGFVDRPKPDPIGLPTVGYGHLCQKKSCAEVKYTFPLTKATALQLLNDDLPSYTKCLGKVLDAGKVKLNENQWAALTSWVFNVGCGAAQSSSLVKRLNRGENANTVASEELPKWKMGGGRVLPGLVKRRADEVALFKIASSRSAFPQCQ

SEQ ID NO: 28 - protein, 157 aa, Ajellomyces capsulatus
PNVNKATLALIKEFEGFVPRPEPDPIGLPTVGYGHLCKTKGCKEVKFPLSKGTATTLLKKDLRSFQQAITLSTKTAVKLNANQYGALVSWAYNVGPNAARSSSLISRLNKGEDPNKVIAQELPKWRLASGKVFKGLVRRRKAEVKLAKTPTKSKALP

SEQ ID NO: 29 - protein, 162 aa, Trichophyton tonsurans
PDVNDETIALIKHFEGFVPRPAPDPIGLPTVGYGHLCRTKGCSEVPFPFPLTEETATELLMQDVKSPQQSITLSTTDQVVLNANQYGALVSWAYNVGGSAAKKSSLISRLNQGQDVDAVIREELPLWNKAGGHVLSGLVRRRAAEVELASEHTDQPALPVDC

SEQ ID NO: 30 - protein, 159 aa, Trichophyton equinum
PDVNPATIALIKEFEGFVPSPAPDPVGLPTVGYGHLCQSKNCGEVGFPFPLTEDTATQLLLQDVKAPQQTITLKTADGVHLNENQYGALVSWTFNVGPGNVATSSLLQRLNALEDVNTVLREELPQWKYGGGKVLPGLVRRRAAEVALGETPSNVAALP

SEQ ID NO: 31 - protein, 162 aa, Trichophyton rubrum; identical to SEQ ID NO: 19

SEQ ID NO: 32 - protein, 161 aa, Trichophyton rubrum
NVNDETIGLIKHFEGFVLRPAPDPIGLPTVGYGHLCRTKGCSEVSFPFFLTEETATELLIQDVKSSQQSITLSTTDQVVFNANQSGALVSWAYTVGGATAKKSSLISRLNREQDVDAVIREELPLWNKAGRHVLPGQVRRRAAEVELASGHTDQPPLLVDC

SEQ ID NO: 33 - protein, 150 aa, Pleurotus ostreatus
MIKSFESFVPSPAPDPVGKLTVGYGHKCIQPGCSEAGPFPLTEAGAVALMHRDLRIATTCLSPAISSTVRLNDNQFGALADWAFNVGCGAMRRSTLVSRLNSGENPNTVAAQELPKWNVAEGHVSNGLIRRRAAEVALFQTASSVIAHPC

SEQ ID NO: 34 - protein, 150 aa, Pleurotus ostreatus
LIKEFEGFVARPAPDPIGLPTVGYGHLCQTTSCSEAGAFPLTEARATTLLLSDSRVATSCLNTAISRNVRLNANQFGALTSWTFNVGCGNMRSSSLLSRLNAGEAPNTVAAQELPKWNKAGGQVLAGLTRRRAAEVVLFQTASSVIAHPC

SEQ ID NO: 35 - protein, 150 aa, Pleurotus ostreatus
LIKSSEGFVPSPAPDPIGLPTVGYGHLCQTSSCSEAGPFPLTEARATTLLLADSRTATSCLNTAISSSVRLNDNQFGALTSWTFNVGCANMRSSTLVSRLNAGETPNTVAAQELPRWNLAGGVVQPGLVTRRANEVKLFQTASSVVAHPC

SEQ ID NO: 36 - protein, 146 aa, Serratia marcescens
ELQTSEAGLRLIADLEGCRLSPYQCSAGVWTQGIGHTAGVIPGKAIDEHKAAMDLVDDVRRTERGMAACLPDTLSQQTYDAAIAFAFNVGVSAACHSTLVALLQQRQWRQACDQLPRWVYVNGKKNKGLEQRRAMERALCLQGIAS

SEQ ID NO: 37 - protein, 162 aa, Galerina marginata
PEVNAATISLIKKFEGFVSKPAPDPIGLPTVGYGHLCKTTGCAEVPFSFPLTEAEATTLLNTDLKTFTACITKDISSKIKLNDNQYGALSSWAFNEGCGNVGSSTLIARLNAGDNPNTVAAQELPKWDIAGGMVLQGLVNRRVAEVKLFQTPSSVIAHPPQC

SEQ ID NO: 38 - protein, 161 aa, Galerina marginata
TPPNAATVSLIKSFEGFVASPKPDPIGLPTAGFGHKCVKANCAEVPFSFPLSQDTATQLLQSDAQKFVTCLHGLISKKVTLNDNQFGALTSFAFNLGCGAVQSSTLLKRLNNGEDPNTVAAAEIPRFNKAGGKVSSGLARRRAAEVQLFQTPSSTTAQPLC

SEQ ID NO: 39 - protein, 163 aa, Agaricus bisporus
PRVNSATISLIKEFEGFVKSPSPDPIGLPTVGFGHLCKSKGCAEVPYKFPLTEANAGKLLQTDIKSFTKCVSDNIKDAVKLNANQFGALSSWAFNVGCGNVKASALVARLNRGEKPNTVAAEELPKWRLAGGKVLKGLVRRRAAEVKLFKTASSAIAHPPKCS

SEQ ID NO: 40 - protein, 167 aa, Trichosporon asahii
VNADGIALIQEFESFQPRPYKDPKGLWTVGYGHLCANGGKDSTCSGTGFKYPLTLATAGELLKKDIPRYTKCLRDNLNEDKVKLNKNQWAALSSFVFNLGCGNFQRSDLMARLNKGEDVNTVIAAEFPRWNKADGAVLTGLVRRRAAEVALAKKTSHGTSKAFPTCT

SEQ ID NO: 41 - protein, 164 aa, Trichosporon asahii
CPSINDAGIALIKEFEGFFKNPYKDPVGLWTVGYGHLCANGGKDSSCSGTGFKYPLTEATATELLKKDLPSYTSCFPKYLTSGAKLNKNQYAALTSFTFNLGCGVLKDFAGRLNKGEDASTVFAQEFPKYVHAGGNVLQGLVRRRNAEVALAKKAGSTEAWPKC

SEQ ID NO: 42 - protein, 160 aa, Trichosporon asahii
INDAGVELIKSFEGCVANPYQDAGGKWTVGYGHLCSGGDDSTCSDTGFSYPLTEETATELFKRDLPNYVSCMPQYLTDGAKLNKNQWAALTSFCYNLGCGILSDFAGRLNAGEDANVVIGEEFPKYTLAEGVELEGTGAGGDEEVALAQSPGSTEAWPNC

SEQ ID NO: 43 - protein, 168 aa, Trichosporon asahii
VNADGIALIQEFESFQPRPYKDPKGLWTVGYGHLCANGGKDSTCSGTGFKYPLTLATAGELLKKDIPRYTKCLRDNLNEDKVKLNKNQWAALSSFVFNLGCGNFQRSDLMARLNKGEDVNTVIAAEFPRWNKADGAVLTGLVRRRAAEVALAKKTSHGTSKAFPTAPK

SEQ ID NO: 44 - protein, 131 aa, Rhizophagus irregularis
MSLFKENFYDDPVGITTIGYGHNCKANKDSDKIKAPISIKEAEELLRKDLVKFEDYVNKQVPELNSNQFSAVVSFAYNLGCDKLRTSILLKKLKAGDTQGASEEFGRWVHAKRKRLPGLVRRREDERQLFL

SEQ ID NO: 45 - protein, 141 aa, Rhizophagus irregularis
VNKEGLDLIEGFEGFYPNFYKDPVGIKTIGYGHACHVHDCSKIQAPISLAEGETLLKSDLAVFESCVESLTRTNLNPDQFSALVSFTFNLGCGAYQKSTLRRKLNAGDTKGASLEFAKWVYGGGKKLPGLVRRRNAERDLF

SEQ ID NO: 46 - protein, 105 aa, Rhizophagus irregularis
INKAGLKLIKKFEGFRSRFYVDKGIITIGYGHACHVYDCSKIHPPISRAKGEALLKKDLVVFEKCVDSLTQIKLNSNQFSALVSFTYNLCCEAYRRSTLRRKLNA

SEQ ID NO: 47 - protein, 155 aa, Coniosporium apollinis
PTVNDGTIIMLKELEGFSATPYLDVNKAAIGYGHQCNEPECAGLSPPITEVEATELMLQDLRTYRDCLTAKLGSVTLNENQYGALASWTFNVGCGNMRGSTLVIRLLAGEDPNTVAADELPKWRLVNGQVSQALVRRREKEIALFKTESGVQVLP

SEQ ID NO: 48 - protein, 114 aa, Rhizophagus irregularis
TIGYGHNCEANNDGDKINAPISIAQAEDLLRRDLSMYENYVNSQVPGLNSNQFSALVSFTFNVGCGNLGSSTLLKKLKAGDTQGAANEFGRWVYANKKKLPGLIRRREAEKRLF

SEQ ID NO: 49 - protein, 163 aa, Penicillium roqueforti
GPAVNTNGLDLIKSFESFQANVYDDGYGNPTIGYGHLCSDSTCSEVTFSKPLTEDTASQLLAKDLVTYQNGVTNALATAVTLNDNQYAALVSWTFNVGVGNMQSSSLVSRMNAGENVETVASEELPKWNKANGAVVAGLTRRRADEVKLFEEASQTKALPVGC

SEQ ID NO: 50 - protein, 163 aa, Aspergillus ruber
GPPANIPTLDLLKGFETMQPDPYDDGFGNPTIGYGHLCTNKACSDVPFSKPLSEDSATRLLQGDLTSAQDAVTNALADPVTLNDNQYGALISWTFNVGNGNMKSSDLVRLMNAGEEIVAVANNELPLWNKANGKVVNGLVRRRKAEVDLFNTPSNFGALPVPC

SEQ ID NO: 51 - protein, 162 aa, Pyronema omphalodes
INAATISLIKSFEGFVPSPKPDPIGLPTVGYGHLCKKKGCAEVKYKFPLTEATAAQLLQDDAYTFRKCVAAAIKDSVTLTDNQYGALVSWAFNVGCGGVQTSTLVKRLNRGENKNIVASQELIKWNKAGNPPKPMNGLTRRRNAEIALFKKASSVQAHPPKC

SEQ ID NO: 52 - protein, 161 aa, Pyronema omphalodes
PPVNTATVNLIKEFEGFVPSPAPDPIGLPTVGYGHLCKQTNCAEVPYPFPLTTTTAAALLQTDLKTYTKCISDYIVDSVRLNDNQYGALSSWAFNVGCGNAKTSTLISRLNAGQNPNTVASEELPKWNKAGGSVLPGLTRRRAAEVTLFKTASTKIAHPAC

SEQ ID NO: 53 - protein, 144 aa, Trichophyton interdigitale
PDVNDETIALIKHFEGFVPRPAPDPIGLPTVGYGHLCRTKGCSEVPFPFPLTEETATELLMQDVKSPQQSITLSTTDQVVLNANQYGALVSWAYNVGGSAAKKSSLISRLNQGQDVDAVIREELPLWNKAGGHVLPGLVRRRAA

SEQ ID NO: 54 - protein, 134 aa, Rhizophagus irregularis
INKEGLALIERFEGFSPNFYKDPVGIKTIGYGHAYHVNDCSKIRPPISRAEGEALLKKDLARFEKCVESLTRVKLNSNQFSALCSFTFNIGCGAYQKSTLRRKLNAGDTKGASLEYRKWVYGGGKPLPGLVKRR

SEQ ID NO: 55 - protein, 141 aa, Rhizophagus irregularis; identical to SEQ ID NO: 45

SEQ ID NO: 56 - protein, 163 aa, Metarhizium anisopliae
KTLNKAGTDLITRWEGFVDRPQPDPIGLPTVGYGHLCQKKSCAEVKYTFPLTKATALQLLNDDLPSYTKCLGKVLDAGKVKLNENQWAALTSWVFNVGCGAAQSSSLVKRLNRGENANTVASEELPKWKMGGGRVLPGLVKRRADEVALFKMASSRSAFPQCQ

SEQ ID NO: 57 - protein, 132 aa, Beauveria bassiana
MLTDLEGVRYTPYYDVAGVLTVCYGHTGADIIKTKTYSATECQAMLDKDLVPFARSVERSVKVPTTEYQKAALISFSYNVGVTAFERSSLLRQLNAGNYQAACDGLRQWTYAGGKQWKGLMNRRDIEREVCM

SEQ ID NO: 58 - protein, 137 aa, Beauveria bassiana
GVDLIKSFEGLRLAKYKDAVGKWTIGYGHLILATENYDRPLTEQEAENLLRSDLALTERGVYRQVSMALNQNQFDALVAFAFNVGLGNLQNSTLLRLLNQGNYSAAADEFLRWNKAGGNVLAGLTRRREAERKLFLS

SEQ ID NO: 59 - protein, 138 aa, Beauveria bassiana
LLPELEGVSYTPYYDVAGVLTVCYGHTGQDIIPDKTYTLSECRRWLDQDLRPAAQVVSRAVTVPVSEYQRAALISFTYNVGTAAFLRSSVLRTLNAGDYEQACAGLKKWIWAGGKPWQGLINRREVEYQLCTWPEPEK

SEQ ID NO: 60 - protein, 163 aa, Metarhizium guizhouense
KTLNKAGTDLVTRWEGFVDRPKPDPIGLPTVGYGHLCQKKSCAEVKYTFPLTKTTALQLLNDDLPSYTKCLGEVLDASKVKLNENQWAALTSWVFNVGCGAAQSSSLVKRLNHGENANTVASEELPKWKMGGGRVLPGLVKRRADEVALFKIASSRSAFPQCQ

SEQ ID NO: 61 - protein, 163 aa, Metarhizium majus
KTLNKAGTDLVTRWEGFVDRPKPDPIGLPTVGYGHLCQKKSCAEVKYTFPLTKATALQLLNDDLPNYTKCLGEVLDASKVKLNENQWAALTSWVFNVGCGAAKSSSLVKRLNHGENANTVASEELPKWRMGGGKVLPGLVKRRADEVALFKIASSRSAFPQCQ

SEQ ID NO: 62 - protein, 163 aa, Metarhizium brunneum
KTLNKAGTDLITRWEGFVDRPKPDPIGLPTVGYGHLCQKKGCAEVKYTFPLTKATALQLLNDDLPSYTKCLGRALDAGKVKLNENQWAALTSWVFNVGCGAAQSSSLVKRLNRGENANTVASEELPKWKMGGGRVLPGLVKRRADEVGLFKIASSRSAFPQCQ

SEQ ID NO: 63 - protein, 163 aa, Magnaporthe oryzae
PKLNQASLDLVKEFEGWFPDIYLDPVGLPTVGYGHLCSNPTCSEVPYPIPLSVANGEALLQSDLGIARRCLSADLVDSVVLNPNQYGALVSWVFNMGCGAQKSSTLTARLNAGEDKSVVARQELPRWVYAGGQVLNGLVRRRAAEVALFDTPAEGVAHPPRPC

SEQ ID NO: 64 - protein, 162 aa, Coprinopsis cinerea
TTVNARTVREIKSSEGFVKSPAPDPIGLPTVGYGHLCKTKGCAEVPYKFPLTDAQATSLLKSDLKTFQNCISKDLRDTVRLNENQYGALVSWAFNVGCRATGSSTLIARLNRGDNPAKVAEEELPKWNKANGKVLQGLVNRRKREIAMFKTPSKVIHHPPKC

SEQ ID NO: 65 - protein, 162 aa, Coprinopsis cinerea
TNVNSKTVEHIKQWEGFVKSPAPDPIGLPTVGYGHLCKTKGCSEVPYKFPLTEAQATSLLKTDLKTFQNCISSQLKDSVRLNANQYGALVSWAFNVGCGNTSGSALISRLNKGESPNTVASQELPKWNKAGGKVLQGLVNRRKAEVTLFKTSSSVIHHPPTC

SEQ ID NO: 66 - protein, 162 aa, Coprinopsis cinerea
SPVNSRTVQEIKNSEGFVRSPAPDPIGLPTVGYGHLCKNKGCSEVPYSFPLTEAQATSLLMTDLKTFQKCISDQINDSIRLNENQYGALVSWAFNVGCGNTASSALISRLNKGESPNKVAEEELPRWKYAGGQVLPGLVARRNREIALFKTASSVVGHPPRC

SEQ ID NO: 67 - protein, 162 aa, Chaetomium globosum
PKSNSATVDLIAKSEGFRANVYNDPAGHPTVGYGHLCTKAKCAEIKYKIPLSTTDGKKLLADDMKKFEKCITAMLNSKAKLNLNQYGALVSWSFNVGCGAAQGSQLVKRLNKGENVNTVLSNELPKWVNAGGKKLPGLVTRRNNEIALAKKSGSGAALPVKC

SEQ ID NO: 68 - protein, 162 aa, Nectria haematococca
PKSNQATVNLIASFEGFRANIYKDAAGYPTVGYGHLCSNSKCTDVKYSIPLSQANGKKLLASDMAKFEKCITAMVKSNVKLNKNQYGALVSWSFNNGCGAAKTSTLIKRLNKGEAPNTVISQELPKWVYAGGKKLNGLVRRRKAEVALAKKATTEKALPKSC

SEQ ID NO: 69 - protein, 162 aa, Nectria haematococca
PPVNQATLSLVEEFEGFRADVYIDATGNPTVGYGHLCKQSGCSEIPYPIPLSQADGQKLLQDDIKVAQQCITLDTTSAVVLNANQYGALVSWAFNVGCGASGDSTLIRRLNNGEDANTVASEELPKWNKGNGQPIAGLTRRRAAEVELFKAPTDVGALPVGC

SEQ ID NO: 70 - protein, 162 aa, Nectria haematococca
PSSNQATVDLIGEFEGFVPHIYKDAAGYPTVGYGHLCSNSKCTDVKYPIPLSKTNGKKLLADDMRKFEKCIAKMVSSKVTLNKNEFGALVSWSFNVGCGAAEGSQLIKRLNKGEKPNTVISGELPKWVYAGKRKLPGLVRRRNAEIALAKKATSEKALPVKC

SEQ ID NO: 71 - protein, 155 aa, Verticillium alfalfae
PNVNAATISLITEFEGWYPNIYIDPVGLPTVGYGHLCADSSCSDVRYPIPLSRANGEQLLRDDIAGFQNCITLQTASSVVLNANQYGALVSWAFNVGCGATKTSTLIQRLNAGGNPNTVAAEELPKWNRGGGQVLPGLTRRRAAEVALHRTATSA

SEQ ID NO: 72 - protein, 162 aa, Thielavia terrestris
PKSNQATVNLIAEYEGFVDHVYTDATGHPTVGYGHLCSNSKCSDVPYHIPLSQADGKKLLADDMKKYEKCITAMLTSKAVVNLNQYGALVSWAFNMGCGAAESSTLVKRLNAGENVNTVLSQELPKWVHGDGKVLPGLVRRRNAEIALAKTPTSDKALPVKC

SEQ ID NO: 73 - protein, 162 aa, Thielavia heterothallica
PKSNSATVDLIAKSEGFRSKIYTDATGHATVGYGHMCTKPKCAEVKYKIPLSTADGKKLLAEDMRKFEKCITNMLNSKAVLNYNQFGALVSWSFNVGCGAAQSSQLVKRLNKGENVNKVLSEELPKWVHGGGKVLPGLVTRRKNEVALAKKPGSSKALPVKC

SEQ ID NO: 74 - protein, 162 aa, Coprinopsis cinerea
PNVNTRTLDLIKLSEGFVASPEPDPIGLPTVGYGHLCQRTGCTEVPYSFPLTQAQAHALLISDLRTYQNCIARDIVDSVRLNDNQYGALVSWAFNVGCTNTASSTLIRRLNAGENPNTVAEQELPRWNMAGGQVLPGLVTRRAREVTLFKTASSVIAHPPPC

SEQ ID NO: 75 - protein, 162 aa, Fusarium oxysporum
PSSNQATVDLIGEFEGFVPHIYKDAAGYPTVGYGHLCSNSKCTDVKYAIPLSKANGKKLLADDMRKFEKCIAKMVSSKVTLNKNQFGALVSWSFNLGCGAAEGSQLLKRLNKGEKPNTVISQELPKWVYAGGRKLPGLVRRRNAEVALAKKATSEKALPVKC

SEQ ID NO: 76 - protein, 162 aa, Metarhizium acridum
PKANEDTVKFISGFEGWSDHVYRDPGPQHLETLGYGHLCKKPNCAEVKYPFPPLSKADGLKLLSDDMSVAENCIYKDVNPKVALNANQYGALVSWAFNVGCGNVASSRLIRRLNAGEDPNTVAAQELPQWNKAGGKVLPGLTRRRNAEVELFKTPTNDPALP

SEQ ID NO: 77 - protein, 162 aa, Metarhizium robertsii
PKANEATVKFISTFEGWYDHVYPDPGPQHLETLGYGHLCKKPNCAEVKYPFPPLSKADGLKLLSDDMSVAENCIYQDTSAKVTLNANQYGALVSWAFNVGCGNVASSRLIRRLNAGEDPNTVAAQELPQWNRAGGKVLPGLTRRRNAEVELFKTPTNDPALP

SEQ ID NO: 78 - protein, 162 aa, Fusarium oxysporum
PSSNQATVDLIGEFEGFVPHIYKDAAGYPTVGYGHLCSNSKCTDVKYPIPLSKANGKKLLADDMRKFEKCIAKMVSSKVTLNKNQFGALVSWSFNLGCGAAEGSQLLKRLNKGEKPNTVISQELPKWVYAGGRKLPGLVRRRNAEVALAKKATSEKALPVKC

SEQ ID NO: 79 - protein, 161 aa, Fusarium oxysporum
PKSNAATVKLISSFEGFRPDVYNDPTGNPTVGYGHLCDAPQCSEVKYPVPLSVANGKKLLADDMKEFEVCITAMLNSKARLNRNQYGALISWAFNMGCGNAESTLVGRLNNGEDPNTVISQELPQWVYASGQRLPGLVHRRNAEIELAQKPTRRSALPKRC

SEQ ID NO: 80 - protein, 162 aa, Chaetomium thermophilum
PKSNQATVDLIAEFEGFRANIYTDAAGYATVGYGHKCQKAKCAEVKYKIPLSKADGKKLLADDMRSFEVCITNMLNSKAKLNYNQFGALVSWSFNVGCGAAKSSTLIKRLNNGENVNKVLSEELPKWNKAGGKVLQGLVRRRAAEVALAKKSGSSQALPVKC

SEQ ID NO: 81 - protein, 154 aa, Verticillium dahliae
PNVNAATISLITEFEGWYPNIYIDPVGLPTVGYGHLCADSSCSDVRYPIPLSRANGEQLLRDDIAGFQNCITLQTASSVVLNANQYGALVSWAFNVGCGATRTSTLIQRLNAGGDPNTVAAEELPKWNRGGGQVLPGLTRRRAAEVALHRTATS

SEQ ID NO: 82 - protein, 162 aa, Fusarium oxysporum
PSSNQATVDLIGEFEGFVPHIYKDAAGYPTVGYGHLCSNSKCTDVKYPIPLSKANGKKLLADDMRKFEKCIAKMISSKVTLNKNQFGALVSWSFNLGCGAAEGSQLLKRLNKGEKPNTVISQELPKWVYAGGRKLPGLVRRRNAEVALAKKATSEKALPVKC

SEQ ID NO: 83 - protein, 162 aa, Fusarium oxysporum
PSSNKATVDLIGEFEGFVPHIYKDAAGYPTVGYGHLCSNSKCTDVKYAIPLSKANGKKLLADDMRKFEKCIAKMVSSKVTLNKNQFGALVSWSFNLGCGAAEGSQLLKRLNKGEKPNTVISQELPKWVYAGGRKLPGLVRRRNAEVALAKKATSEKALPVKC

SEQ ID NO: 84 - protein, 162 aa, Fusarium oxysporum
PKSNAATVNLIASFKGFRPDVYNDPTGNPTVGYGHLCDAPQCSEAKYPIPLSIVNGKKLLADGMKEFEICITAMLNSKANLNRNQYGALISWAFNMGCGNADSSTLVGRLNNGEDPNTVISQELPQWVNVSGQRLPGLVRRRNAEIELAQKPTRRRALPKRC

SEQ ID NO: 85 - protein, 162 aa, Fusarium oxysporum; identical to SEQ ID NO: 75

SEQ ID NO: 86 - protein, 162 aa, Fusarium oxysporum
PKSNAATVNLIASFEGFRPDVYNDPTGNPTVGYGHLCDAPQCSEVKYPVPLSVANGKKLLADDMKEFEVCITAMLNSKARLNRNQYGALISWAFNMGCGNAESSILVGRLNNGEDPNSVISQELPQWVYASGQRLPGLVLRRNAEIELAQKPTRRRALPKRC

SEQ ID NO: 87 - protein, 162 aa, Fusarium oxysporum; identical to SEQ ID NO: 75

SEQ ID NO: 88 - protein, 162 aa, Fusarium oxysporum
PSSNQATVDLIGEFEGFVPHIYKDAAGYPTVGYGHLCSNSKCTDVKYAIPLSKANGKKLLADDMRKFEKCIAQMVSSKVTLNKNQFGALVSWSFNLGCGAAEGSQLLKRLNKGEKPNTVISQELPKWVYAGGRKLPGLVRRRNAEVALAKKATSEKALPVKC

SEQ ID NO: 89 - protein, 126 aa, Fusarium oxysporum
PKSNAATINLIASFEGFRPDVYNDPTGNPTVGYGHLCDAPQCSEVKYPIPLSIVNGKKLLADGMKEFEICITAMLNSKANLNRNQYGALISWAFNMGCGNADSSTLVGRLNNGEDPNIVISQELPQ

SEQ ID NO: 90 - protein, 162 aa, Gibberella moniliformis
PKSNAATVNLIASFEGFRPDVYNDPTGNPTVGYGHLCDAPQCSEVKYPIPLSVVNGKKLLADDMKEFEVCITAMLNSKAKLNRNQYGALISWAFNMGCGNAESSTLIGRLNNCEDPNTVISQELPQWEYASGQRLPGLLRRRNAEIELAQKPTRRRALPKRC

SEQ ID NO: 91 - protein, 162 aa, Fusarium oxysporum; identical to SEQ ID NO: 78

SEQ ID NO: 92 - protein, 162 aa, Fusarium oxysporum
PKSNAATVNLIASFEGFRPDVYNDPTGNPTVGYGHQCDAPQCSEVKYPVPLSVANGKKLLADDMKEFEVCITAMLNSKARLNRNQYGALISWAFNMGCGNGESSTLVGRLKNGEDPNTVISQELPQWVYASGQRLPGLVHRRNAEIELAQKPTRRRALPKRC

SEQ ID NO: 93 - protein, 161 aa, Fusarium oxysporum; identical to SEQ ID NO: 79

SEQ ID NO: 94 - protein, 139 aa, Fusarium oxysporum
PKSNVATINLIASFEGFRPDFYNDPTGNPTVGYGHLCDAPQCSEVKYPIPLSIVNGKKLLADGMKEFEICITAMLNSKANLNRNQYGALISWAFNMGCGNANSSTLVGRLNNEEDPNTVISQELPQWVNVSGQRLPGLV

SEQ ID NO: 95 - protein, 162 aa, Fusarium oxysporum
PKSNAATVNLVASFEGFKPDVYNDPTGNPTVGYGHLCDAPQCSEVRYPVPLSVANGKKLLADDMKGFAVCISAMLNSKARLNRNQYGALISWAFNMGCGNAESSTLIGRLNNWEDPNTVISQELPRWVYASGKRLPGLVRRRNAEIELAQKPTRRRALPKRC

SEQ ID NO: 96 - protein, 162 aa, Gibberella fujikuroi
PKSNAATVNLIASFEGFRPDVYSDPTGNPTVGYGHLCDAPQCSEVKYPIPLSVANGKKLLVDDMKDFEVCLTAMLNSKAKLNRNQYGALISWAFNMGCGNAQSSTLVGRLNNGEDPNTVISQELPHWVYASGQRLPGLVRRRNAEIELAQKPTRRRALPKRC

SEQ ID NO: 97 - protein, 145 aa, Glarea lozoyensis
PKVNTATLKLIEGFEGWSATAYKDPDGNPTIGYGHLCSSASCKEIKYSIPLSRAEGDSLIQDDLAVARKCIAKQIKDSIKLNANQFGALVSWAFNVGCGNSGDSTLIRRLNAGEAPNTVAGQELPKWNRGKNGVLPGLTRRRNAE

SEQ ID NO: 98 - protein, 160 aa, Coniosporium apollinis
PDVNAATVALIKEFEGFVRSPAPDPIGLPTVGFGHLCKTAGCAEVPYKFPLTEAQAAALLQTDLKTYEKCLADKVADSVRLNDNQYGALVSWTFNVGCGNMGSSTLVRRLNAGEAPNTVAAQELPKWNKAGGNVLAGLTRRRAAEVRLFQTASGTQALPS

SEQ ID NO: 99 - protein, 162 aa, Endocarpon pusillum
PDVNAATVALIKEFEGFVKSPAPDPIGLPTVGYGHLCKTNGCSEVPYPFPLSEAQAAALLQTDLKSYEKCLADKVSDSVKLSDNQYGALVSWTFNVGCGNMGGSTLVTRLNNGESPNTVAAQELPKWNKAGGSALAGLTRRRAAEVQLFQTASGTQALPAKC

SEQ ID NO: 100 - protein, 105 aa, Fusarium oxysporum
DPTGNPTVGYGHLCDAAQCSEVKYPIPLSIVNGKKLLADGMKEFEICNPAMLNSKANLNRNQYGALISWAFNMGRGNADSSTLVGRLNNGEDPNTVISQELPQWV

SEQ ID NO: 101 - protein, 150 aa, Stachybotrys chartarum
PTTNSATVSLIQEFEGWSPTIYLDPSGYPTVGYGHLCANSRCTDVPYPIPLSTANGNLLLQSDMLVARRCIAQDVNVRLNANQYGALVSWAFNVGCGASGSSTLVRRLNAGENPNTVAAEELPRWVNSGGVQLPGLVRRRNAEVALFRTA

SEQ ID NO: 102 - protein, 160 aa, Acremonium chrysogenum
PKSNKATVDLISEFEGFRADIYIDATGHPTVGYGHLCSDKKCSDVSYPIPLSKANGKKLLAKDMRRFEKCITNMVDAKVNKNQYGALVSWSFNVGCGAAQGSQLVKRLNKGQKVNKVLSEELPKWVHGGGQVLPGLVRRRKAEIALAKKKTDAKALPPTC

SEQ ID NO: 103 - protein, 150 aa, Acremonium chrysogenum
PEVNQPALNLVQQFEDFRATIYKDPAGHPAVGYGHLCSDSACSDVPYTIPLSYTDAERLLRDDLADCITNDTGNNVVLNANQYGALVSWAFNVGCENSGFSALIERLNMGEDPTSVATDELPRWNMIGDEVVPAFERRRAAEVDLFLAPS

SEQ ID NO: 104 - protein, 155 aa, Acremonium chrysogenum
PAANAATVSLVAEFEGFSPDIYDDPSGYPTVGYGHLCGDPSCSEVPYPIPLSEEDGRKLLASDMAVAQNCITMQTAESVVLNDNQYGALVSWAFNVGCGNSGSSTLISRLNAGEDPNTVAAEELPKWKYSGGVELPGLVRRRAAEVDLHKTPSDV

SEQ ID NO: 105 - protein, 157 aa, Metarhizium anisopliae
PKANEATVKFISTFEGWYDHVYPDPGPQHLETLGYGHLCKKPNCAEVKYPFPPLSKADGLKLLSDDMSVAENCIYQDTSAKVTLNANQYGALVSWAFNVGCGNVASSRLIRRLNAGEDPDTVAAQELPQWNRAGGKVLPGLTRRRNAEVELFKTPTN

SEQ ID NO: 106 - protein, 150 aa, Stachybotrys chlorohalonata
PTTNSATVSLIQEFEGWSSTIYLDPSGYPTVGYGHLCANSRCTDVPYPIPLSTANGNLLLQSDMLVARRCIAQDVNVRLNANQYGALVSWAFNVGCGASGSSTLVRRLNAGENPNTVAAEELPRWVNSGGVQLPGLVRRRNAEVALFRTA

SEQ ID NO: 107 - protein, 158 aa, Pseudogymnoascus pannorum
PKSNGATVDLIAEFEGFVPNVYTDATGHATVGYGHLCQKSKCSEVPYHIPLTKANGKKLLASDIGVYEKCVTAMLNSKAKVNLNQYGALVSLTFNMGCGAIKSSAIVTRLNKGEKATTVISSEFPKWVHGGGKVLPGLVRRRKAEVALAKKTAGKALP

SEQ ID NO: 108 - protein, 158 aa, Pseudogymnoascus pannorum
PKSNAATVNLIAEFEGFVPHVYTDATGHPTVGYGHLCSNSKCSDAGYPIPISKANGKKLLAKDMGKAEKCITAMVNSKVTLNANQYGALVSLAFNVGCGAMKSSTLVKRLNNGEKASVVYPKEFPKWVHGNGKVLPGLVRRRKAEVALSKKAAGKALP

SEQ ID NO: 109 - protein, 158 aa, Pseudogymnoascus pannorum
PKSNAATVDLIAEFEGFVPKVYTDATGHPTVGYGHLCSNSKCSDAGYPIPLSKANGKKLLAKDMGKAEKCVTAMVNSKVTLNANEYGALVSLAFNVGCGAMQSSSLVKRLNNGEKASVVYPVEFPKWVHGNGKVLPGLVRRRKAEVALSKKAAGKALP

SEQ ID NO: 110 - protein, 158 aa, Pseudogymnoascus pannorum
PHSNTATVNLIAEFEGFVPNVYTDATGHPTVGYGHLCSNSKCSGIGYPIPLSVANGKKLLAKDMGVAERCITAMTNSKAVLNANQYGALVSLTFNMGCGAIQSSAIIKRINNGEKASVVFPNEFPKWVHGGGVVLPGLVRRRNAEIALSKKAADKVLP

SEQ ID NO: 111 - protein, 158 aa, Pseudogymnoascus pannorum
PHSNTATVNLIAEFEGFVPNVYTDATGHPTVGYGHLCSNSKCSGIGYPIPLSVANGKKLLAKDMGVAEKCITAMTNSKAVLNANQYGALVSLTFNMGCGAIQSSDIIKRINKGEKASVVFPSEFPKWVHGGGVVLPGLVRRRNAEIALSKKAADKVLP

SEQ ID NO: 112 - protein, 162 aa, Torrubiella hemipterigena
PAVNQATVNLVAEFEGFRASEYTDATGHPTIGYGHLCTAKGCADVAYPKPLSEADGKKQLAKDLAVAQNCITTMTANPVTLNANQYGALVSWAFNVGCGAAKSSTLIKELNAGKDARTVIDTELPKWNKGNGKPIAGLTRRRKAEVDLSHVATSQGALPAKC

SEQ ID NO: 113 - protein, 164 aa, Torrubiella hemipterigena
PVANAATVEFVSGFEGWRDYVYRDPGNGFQTLGYGHLCKKPNCAEVPYKFPPLSKADGLKLLSSDMAIAENCVTKDIKDKIVLNANQYGALVSWAFNVGCGNVASSSLIRRLNAGENPNVVAASELPKWNKDAKGHVLPGLTRRRAAEVKLFQTPTSEHALPAC

SEQ ID NO: 114 - protein, 158 aa, Pseudogymnoascus pannorum
PHSNTATVNLIAEFEGFVPNVYTDATGHPTVGYGHLCSNSKCSGIGYPIPLSVANGKKLLAKDMGVAERCITAMTNSKAVLNANQYGALVSLTFNMGCGAIQSSDIIKRINNGEKASVVFPKEFPRWVHGGGVVLPGLVRRRNAEIALSKKAADKVLP

SEQ ID NO: 115 - protein, 158 aa, Pseudogymnoascus pannorum; identical to SEQ ID NO: 109

SEQ ID NO: 116 - protein, 158 aa, Pseudogymnoascus pannorum
PKSNAATVDLIAEFEGFVAKVYIDATGHPTVGYGHLCTKSKCAEVGYPIPLSKANGKKLLATDMAKAEKCVTAMLNSKAVLNANQYGALVSLAFNVGCGAVQSSSIVTRLNKGEKAATVYPQEFPKWVHGGGKVLPGLVRRRNAEVALAKKAAGKALP

SEQ ID NO: 117 - protein, 158 aa, Pseudogymnoascus pannorum; identical to SEQ ID NO: 116

SEQ ID NO: 118 - protein, 158 aa, Pseudogymnoascus pannorum
PKSNAATVDLIAEFEGFVPNVYTDATGHATVGYGHLCQKSKCSEVPYHIPLTKANGKKLLASDIGVYEKCVTAMLNSKAKLNLNQYGALVSLTFNMGCGAIKSSAIVTRLNKGEKATTVISGEFPKWVHGGGKVLPGLVRRRKAEVALAKKTAGKALP

SEQ ID NO: 119 - protein, 158 aa, Pseudogymnoascus pannorum
PKSNAATVDLIAEFEGFVPNVYIDATGHPTVGYGHLCTKSKCAEIGYPIPLSKANGKKLLAKDIAKFEKCVTAMLKSKAVLNLNQYGALVSLAFNVGCGAVQSSSIVTRLNKGEKAATVFPQEFPKWVHGNGKVLPGLVRRRKAEVALAKKAAGKALP

SEQ ID NO: 120 - protein, 162 aa, Pseudogymnoascus pannorum
PASNQATIDLIAGSEGFSGDVYTDPTGNPTVGYGHLCQDSTCADTGFPIPLSEEDGKKLLAGDMKQFESCITTMTGDAVTLNLNQFGALVSWSFNNGCGAAQSSTLISRLNNGEDPNTVIEQELPQWVYGNGQILDGLVKRRQAEIDLAKTPTSDGALPAPC

SEQ ID NO: 121 - protein, 162 aa, Metarhizium majus
PKANEATVKFISTFEGWYDHVYPDPGPQHLETLGYGHLCKKPNCAEVKYPFPPLSKADGLKLLSDDMSVAENCIYQDTNAKVTLNANQYGALVSWAFNVGCGNVASSRLIRRLNAGEDPNMVAAQELPQWNRADGKVLPGLTRRRNAEVELFKTPTNDPALP

SEQ ID NO: 122 - protein, 162 aa, Metarhizium guizhouense
PKANEATVKFISTFEGWYDHVYPDPGPQHLETLGYGHLCKKPNCAEVKYPFPPLSKADGLKLLSDDMSVAENCIYQDTNAKVTLNANQYGALVSWAFNVGCGNVASSRLIRRLNAGEDPNTVAAQELPQWNRADGKVLPGLTRRRNAEVELFKTPTNDPALP

SEQ ID NO: 123 - protein, 74 aa, Podospora anserina
YPITGDVVNCRSGPGTSYSVVKQYTQGQDVTITCQTEGTNVNGVTIWDKTADGNCYVSDYYVQTGVNGYVTERC

SEQ ID NO: 124 - protein, 73 aa, Penicillium chrysogenum
YPITGDGVNCRSGPGTSYSVVKSYKQGADVAITCQTAGTSVNGNEIWDKTEDGCYITDYYIRTGSSSYVTKKC

SEQ ID NO: 125 - protein, 73 aa, Nectria haematococca
YPVTSDNLNCRSGPGTAFAIKKSYKNGQDVTITCQTEGDNIEGNSIWDKTSDGCYVADKYVKTGKDGYVKGKC

SEQ ID NO: 126 - protein, 61 aa, Metarhizium acridum
YPITGTTVNCRAGPATDPAIVRAYKKDDKVSIARQTQGPDINGGTIWDKTADGCYVSDYFV

SEQ ID NO: 127 - protein, 73 aa, Metarhizium robertsii
FPVTADSLNCRAEPNTSSAVKKTYKKTDDVKISCQTEGPSINGNSIWDKTQDGCYVADYYIKTGSSGYVTGKC

SEQ ID NO: 128 - protein, 73 aa, Metarhizium robertsii
YAIEADGVNCRSGPSTSDKVVRTYNKGNDVKLECQTAGQAIHGDSLWDKTTDGCYVADYYVKTGTTNMVTGQC

SEQ ID NO: 129 - protein, 73 aa, Fusarium oxysporum
YPVTSDNLNCRSGPGTAFAIKKSYKKGQDVTITCQTQGDNVEGNSIWDKTSDGCYVADKYVKTGKDGYVKGKC

SEQ ID NO: 130 - protein, 69 aa, Fusarium oxysporum
DEGVRCRSGPTTSHAIQRQFTKGTDVTITCQIEGTNIEGNALWDKTTFGCYVSDYYVATGSSGYVTSKC

SEQ ID NO: 131 - protein, 73 aa, Thielavia terrestris
YPVKADTLNCRSGPGTSYKVIKTYKKGTDLKITCQTPGTSVNGDNLWDKTSDGCYVADYYVKTGTSGYVTAHC

SEQ ID NO: 132 - protein, 73 aa, Thielavia terrestris
YPIKGDGVNCRTGPGTSYKVVKSYAKGVDVKITCQTHGESINGDTLWDKTSDGCYVADYYVKTGTTNMVTGQC

SEQ ID NO: 133 - protein, 73 aa, Trichosporon asahii
YPIKTDGVRCRSGPGTSYDIKKTYSAGDKVTLSCYKTGTSVEGNTYWDKTGDGCYVSDYYVKTGSTTPVVSKC

SEQ ID NO: 134 - protein, 72 aa, Trichosporon asahii
YPVKETLHCRSSPSTSGKIVKDYPKGTKIKLSCYSRGQSIGGNTIWDKTTDGCFVADYYVTTGTTNPVVAAC

SEQ ID NO: 135 - protein, 73 aa, Fusarium oxysporum
YPITGNNVNCREGPSTGYEVVKTYHKGDDVKLTCQTSGEGVLGNSLWDKTSDGCYVADYYVKTGTSGMVTKDC

SEQ ID NO: 136 - protein, 73 aa, Fusarium oxysporum
YPVTSDNLNCRSGPGTAFAIKKSYKKGQDVTITCQTQGDKVEGNSIWDKTSDGCYVADKYVKTGKDGYVKGKC

SEQ ID NO: 137 - protein, 72 aa, Trichosporon asahii
YPVKEGLNCRSEPNTGGGIVTSYAAGTQVTITCATHGEAVNGHDVWDKTTDGCFVSDWYVSTGTAEFVASEC

SEQ ID NO: 138 - protein, 73 aa, Trichosporon asahii; identical to SEQ ID NO: 133

SEQ ID NO: 139 - protein, 73 aa, Fusarium oxysporum
YPVTSDNLNCRSGPGTAFAIKKSYKKGQDVTINCQTQGDNVEGNSIWDKTSDGCYVADKYVKTGKDGYVKGKC

SEQ ID NO: 140 - protein, 69 aa, Fusarium oxysporum
DEGVRCRSGPTTSNAIQRQFAKGTDVAITCQTEGTHIKGNALWDKTTFGCYVSDCYVATGSSGYVTSKC

SEQ ID NO: 141 - protein, 73 aa, Fusarium oxysporum; identical to SEQ ID NO: 129

SEQ ID NO: 142 - protein, 69 aa, Fusarium oxysporum; identical to SEQ ID NO: 130

SEQ ID NO: 143 - protein, 73 aa, Fusarium oxysporum; identical to SEQ ID NO: 129

SEQ ID NO: 144 - protein, 73 aa, Fusarium oxysporum; identical to SEQ ID NO: 136

SEQ ID NO: 145 - protein, 69 aa, Fusarium oxysporum
DEGVRCRSGPTTSNAIQRQFAKGTDVAITCQTEGTHIKGNVPWDKTTFGCYVSDYYVATGSSGYVTSKC

SEQ ID NO: 146 - protein, 69 aa, Gibberella moniliformis
DEGVRCRSGPTTSHAIQRQFTKGTDGTITYQTEGTNIEGNTLWDKTTFGCFVSDYYVATGSSGYVTSKC

SEQ ID NO: 147 - protein, 73 aa, Fusarium oxysporum
YPVTSDNLNCRSGPGTAFAIKKSYKKGQDVTITCQTQGDNVEGNSIWDKTSDGCYVADKYVKTGKDGYVKDKC

SEQ ID NO: 148 - protein, 69 aa, Fusarium oxysporum; identical to SEQ ID NO: 130

SEQ ID NO: 149 - protein, 73 aa, Fusarium oxysporum; identical to SEQ ID NO: 129

SEQ ID NO: 150 - protein, 69 aa, Fusarium oxysporum; identical to SEQ ID NO: 130

SEQ ID NO: 151 - protein, 73 aa, Penicillium oxalicum
YPVKTDGLHCRSGPGTSYSIVKTYNTGTDLTITCQTPGPVINGDELWDKTSDGCYVSDYYVKTGTSGYVAPHC

SEQ ID NO: 152 - protein, 73 aa, Penicillium oxalicum
YPVKTDDLHCRSGPGTNYAVVKSYKIGTDLTITCQAPGTVVSGDELWDKTSDGCYVSDYYVKTGTSGYVTKQC

SEQ ID NO: 153 - protein, 69 aa, Fusarium oxysporum
DEGVRCRSGPTTSNAIQRQFAKGTDVAITCQTEGTHIKGNVPWDKTTFGCYISDYYVAKGSSGYVTSKC

SEQ ID NO: 154 - protein, 69 aa, Fusarium oxysporum
DEGVRCRSGPTTSHAIQRQFTKGTDVTITCQIEGTNIEGNALWDKTTFGCYLSDYYVATGSSGYVTSKC

SEQ ID NO: 155 - protein, 73 aa, Fusarium oxysporum
YPVTSDNLNCRSGPGTGFAIKKSYKKGQDITITCQTQGDNVEGNSIWDKTSNGCYVADKYVKTGKDGYVKGKC

SEQ ID NO: 156 - protein, 69 aa, Gibberella fujikuroi
DEGVRCRSGPTTSHAIQTHFVKGTDVTITCQTEGTYIGGSTLWDKTTFGCYVSDYYVATGSSGYVTSKC

SEQ ID NO: 157 - protein, 59 aa, Penicillium roqueforti
YPVTGSVIDCHSGPGASHSVVKTYEERADIEIVCQATGTTVDGSDIWHQTVDDCYVSDF

SEQ ID NO: 158 - protein, 74 aa, Pyronema omphalodes
YPLVHTDTLNCRSSPSTSSSITKTYKKSDDIKITCQTYGDTIKGNNIWDKTPDGCYVSDYYVKTGKSGFVVGKC

SEQ ID NO: 159 - protein, 72 aa, Pyronema omphalodes
YPAKETLRCRTSPSTSASIHKTYPAGADIKITCQTTGTKVLTSNVWDKTSDGCYVSDYYVSTGHSGIFLSKC

SEQ ID NO: 160 - protein, 73 aa, Pseudogymnoascus pannorum
FPITGNTVNCRSGPGTDFSIKQTYAKGEAVAITCQTSGTKINGNDIWDLTTDGCYVTDFYVKTGSISYVLPKC

SEQ ID NO: 161 - protein, 68 aa, Fusarium oxysporum
DKGVRCRSGPTTSHAIQRQFTKGTDVTITCQIEGTNIEGNALWDKTTFGCYVSDYYVATGSSGYVTLK

SEQ ID NO: 162 - protein, 68 aa, Fusarium oxysporum; identical to SEQ ID NO: 161

SEQ ID NO: 163 - protein, 73 aa, Acremonium chrysogenum
YPITGNEVNCRSGPSTSSDIVTSYKKGDEVQVTCQIDGEDIFGNTIWDQTEDGCYVADFYVKTGSNAFVTEAC

SEQ ID NO: 164 - protein, 73 aa, Acremonium chrysogenum
YPITADDVKCRAGPSTSHDIVTAFAEGHEVELECQIVGENIFGNSIWDKTTDGCYVSDYYVRTGSDGMVVDNC

SEQ ID NO: 165 - protein, 73 aa, Acremonium chrysogenum
YPVTADSLNCREGAGTDTAVVTTYTAGTDVEVVCQAEGEVIEGSSIWDQTQDGCYVSDVYVDTGSDGYVADKC

SEQ ID NO: 166 - protein, 73 aa, Metarhizium anisopliae; identical to SEQ ID NO: 128

SEQ ID NO: 167 - protein, 73 aa, Metarhizium anisopliae; identical to SEQ ID NO: 127

SEQ ID NO: 168 - protein, 73 aa, Pseudogymnoascus pannorum
YPITGTYVNCRTGPSTSFDIVRSYELGDEVDLTCQIAGETVTGDNLWDFTTDGCYVADYFVKTGTFGMVVDEC

SEQ ID NO: 169 - protein, 73 aa, Pseudogymnoascus pannorum
YPITGTYVNCRSGPSTSFDIVRSYELGDEVSLTCQIAGETVEGDYLWDLTTDGCYVADYFVKTGTVGMVAEEC

SEQ ID NO: 170 - protein, 74 aa, Pseudogymnoascus pannorum
YPISGDSVNCRSGPATSYKVIKTYAKGHDVKVTCQTVGETVKGDNLWDKTADGCYVADYYVKTGTTGRVVKTEC

SEQ ID NO: 171 - protein, 73 aa, Pseudogymnoascus pannorum
VTSPTTVKCRSGPGTQYKIVKTYPASGRECYSCYESGTCINGNCSWDYNYMDNCYISGYYTGSACTTAALGKC

SEQ ID NO: 172 - protein, 74 aa, Pseudogymnoascus pannorum
YPISGTSVNCRSGPATSYKVIKTYKKGQDVKITCQTVGETVSGDNLWDKTSDGCYVADYYVKTGTTGRVVKTEC

SEQ ID NO: 173 - protein, 73 aa, Pseudogymnoascus pannorum
YPIKGSVVNCRAGPGTNFPIVKTFKKGDTVDITCQTPGTSISGNSIWDLIPDNCFITDYYVKTGTGKYIKPRC

SEQ ID NO: 174 - protein, 64 aa, Pseudogymnoascus pannorum
VTNPTTVKCRSGPGTQYRIVKTYRAGDRECYSCYESGTCINGNCSWDYNYMDNCYISGYYTDSG

SEQ ID NO: 175 - protein, 73 aa, Pseudogymnoascus pannorum; identical to SEQ ID NO: 171

SEQ ID NO: 176 - protein, 73 aa, Pseudogymnoascus pannorum
YPITGNTVNCRSGPGTDFPIKKTFAKGSIVSITCQTPGTKINGNEIWDLTSDGCFVSDFYVKTGSITYVKPKC

SEQ ID NO: 177 - protein, 73 aa, Pseudogymnoascus pannorum
YPITGTSVNCRSGPSTKFDVVRSYVLGDEVTLTCQIAGETVTGDYLWDLTTDGCYVADYFVKTGTVGMVTEAC

SEQ ID NO: 178 - protein, 73 aa, Pseudogymnoascus pannorum
YPITGTYVNCRSGPSTSYDIIRSYELGDEVDLTCQIAGETVTGDNLWDFTTDGCYVADYFVKTGTFGMVVDEC

SEQ ID NO: 179 - protein, 74 aa, Pseudogymnoascus pannorum; identical to SEQ ID NO: 170

SEQ ID NO: 180 - protein, 73 aa, Pseudogymnoascus pannorum; identical to SEQ ID NO: 168

SEQ ID NO: 181 - protein, 73 aa, Pseudogymnoascus pannorum; identical to SEQ ID NO: 169

SEQ ID NO: 182 - protein, 72 aa, Pseudogymnoascus pannorum
YPISGDSVNCRSGPATSYKVIKTYLKGHDVDLTCQTVGETVKGDSLWDKTTDGCYVADYYVKTGTTGRVVKK

SEQ ID NO: 183 - protein, 73 aa, Pseudogymnoascus pannorum
YPIKGSVVNCRSGPGTNFAIVKTFKKGDTVDITCQTPGTSISGNSIWDLTPDNCFITDYYVKTGTGKYIKPRC

SEQ ID NO: 184 - protein, 67 aa, Pseudogymnoascus pannorum
CRSGPGTGYSVIATVKKGSYYSFGCYKTGTCVSGNCTWDRIFWDGKSCYVSGYYTDSACSASALGKC

SEQ ID NO: 185 - protein, 73 aa, Pseudogymnoascus pannorum
YPITGTFVNCRSGPSTKFDVVRSYVLGDEVTLTCQIAGETVTGDYLWDLTTDGCYVADYFVKTGTVGMVTEAC

SEQ ID NO: 186 - protein, 67 aa, Pseudogymnoascus pannorum; identical to SEQ ID NO: 184

SEQ ID NO: 187 - protein, 73 aa, Pseudogymnoascus pannorum
YPITGTKVNCRTGPSTSFEIIRSYKLGDEVSLTCQIAGETVQGNYLWDLTTDGCYVADYFVKTGSDGMVTEGC

SEQ ID NO: 188 - protein, 73 aa, Aspergillus clavatus
YPITGDGVNCRSGPGTSYKVVKSYPKGHQVSIVCQATGTDVKGDSLWDKTSDGCYVADYYVKTGTTGYVTKHC

SEQ ID NO: 189 - protein, 73 aa, Aspergillus clavatus
YPITGDTVNCRSGPGTSHAVVKSYKKGEDVKIVCQAPGTDVKGESIWDKTSDGCYVADYYVKTGTTGYVTKKC

SEQ ID NO: 190 - protein, 73 aa, Neosartorya fischeri
YPITGNEVNCRAGPSTNDKVVKSYHKGDDVKLSCQTYGENVQGNSIWDKTTDGCYVSDFYVKTGSNSMVTKEC

SEQ ID NO: 191 - protein, 73 aa, Neosartorya fischeri
YPITGDGVNCRSGPGTNHPVVKSYPKGHDVSIVCQAPGTDVKGDKLWDKTSDGCYVADYYVKTGSTDYVTKHC

SEQ ID NO: 192 - protein, 73 aa, Neosartorya fischeri
YPITGDGVNCRSGPGTNHPVVKSYPKGHDVSIVCQAPGTDVKGDKLWDKTSDGCYVADYYVKTGSSDYVTKHC

SEQ ID NO: 193 - protein, 73 aa, Aspergillus terreus
YPITGDGVNCRSGPGTSHAVVKSYPKGHEISIVCQAAGTDVKGDNLWDKTSDGCYVADYYVKTGSTGYVTKHC

SEQ ID NO: 194 - protein, 64 aa, Aspergillus terreus
CRTGPSTNDGITKTYKKGDDVKLSCQTYGESIQGSTIWDKTTDGCYVADYYVKTGTSGMVTGEC

SEQ ID NO: 195 - protein, 73 aa, Aspergillus terreus
YTITADGVNCRSGPGTSNKSVKTYAKGTDVKISCQQAGESIFGNSLWDKTSDGCYVSDYYVKTGSTGYVTDKC

SEQ ID NO: 196 - protein, 73 aa, Chaetomium globosum
YPITGSVVNCRSGPGTSYAVKKSYAKGADVKISCQTSGTSVNGNNIWDKTQDGCYVADYYVKTGTNGYVTKKC

SEQ ID NO: 197 - protein, 73 aa, Chaetomium globosum
YPITGSVVNCRSGPGTSYAVKKSYNKGADVTISCQTTGTSVNGNSIWDKTQDGCYVADYYVKTGTNGYVTKKC

SEQ ID NO: 198 - protein, 73 aa, Chaetomium globosum
YPITGNDVNCRSGPDTSYKSVKTYKKGADVKLTCQTYGESINGNAIWDKTSDGCYVSDYYVKTGSNSMVTKEC

SEQ ID NO: 199 - protein, 73 aa, Neosartorya fumigata
YPITGNGVNCRAGPSTNDKVIKSYAKGTDVKLSCQTYGENINGNSIWDKTTDGCYVADYYVKTGSNSMVTKEC

SEQ ID NO: 200 - protein, 73 aa, Neosartorya fumigata
YPITGNGVNCRSGPGTNYPVVKSYPKGHEVSIVCQAPGTDIKGDKLWDKTSDGCYVADYYVKTGTTGYVTKHC

SEQ ID NO: 201 - protein, 73 aa, Neosartorya fumigata
YPITGDGVNCRSGPGTNYPVVKSYPKGHEVSIVCQAPGTDIKGDKLWDKTSDGCYVADYYVKTGTTNYVAKHC

SEQ ID NO: 202 - protein, 73 aa, Penicillium chrysogenum
YPITGDSVNCRSGPGTTHAVVKSYKKAQDVTVTCQTAGESIFGDNLWDKTSDGCYVADYYVQTGTSNYVTTKC

SEQ ID NO: 203 - protein, 73 aa, Penicillium chrysogenum
YPITSDQLNCRSGPSTSDSVVKTYKSGADVKVSCQTYGESINGNTIWDKTSDNCYVADYYVKTGSDSMVTESC

SEQ ID NO: 204 - protein, 73 aa, Nectria haematococca
YPITGDDVRCRSGPGTSYAIKKTFKKGTNVKITCQTTGTNIKGNNIWDKVSEGCYVSDYYVKTGSSGFVTKKC

SEQ ID NO: 205 - protein, 73 aa, Nectria haematococca
YPITGDDVKCRSGPGTSYAVKKVLKKGTDVTITCQTEGTNISGNTIWDKISDGCYVSDYYVKTGSNGYVKPKC

SEQ ID NO: 206 - protein, 73 aa, Hypocrea jecorina
YPITGDVVNCRTGPGTSYAIKTSYKKSHDISISCQTTGTSVNGNNIWDKTADGCYVADYYVKTGSSGFVTKKC

SEQ ID NO: 207 - protein, 73 aa, Thielavia terrestris
YAITGDTVNCRSGPGTNYAVKKTYAKGHDVTLSCQTSGTTVNGNSIWDKTSDGCYVSDYYVKTGSNSYVTKKC

SEQ ID NO: 208 - protein, 74 aa, Thielavia heterothallica
YPVTANGGLSCRSGPGTSYPVKKTYKKGFDIKISCQTTGTSVNGYNIWDKTQDGCYVSDYYVKTGKSGFVTTKC

SEQ ID NO: 209 - protein, 73 aa, Hypocrea jecorina
YPITGDTVNCRSGPGTSFAIKKTYKKSQDVSVTCQTSGTSVNGNSIWDKTSDGCYVADYYVKTGSSSYVTKKC

SEQ ID NO: 210 - protein, 73 aa, Chaetomium thermophilum
YPITGEGVNCRSGPGTSYKVIKSYKKGTDVKISCQIKGESINGNNLWDKTQDGCYVSDYYVKTGSNSMVTKQC

SEQ ID NO: 211 - protein, 73 aa, Chaetomium thermophilum
YKITGNNVNCRSGPGTSYSVKKTYAKGTDVKITCQTTGTNINGNNIWDKTSDGCYVSDYYVKTGTNGYVTTKC

SEQ ID NO: 212 - protein, 73 aa, Fusarium oxysporum
YPITGNDVKCRSGPGTSYAVKKVLKKGTDVKITCQTEGTNISGNTIWDKISDGCYVSDYYVKTGSSGYIKPKC

SEQ ID NO: 213 - protein, 73 aa, Thielavia heterothallica
YPITGDDVNCRTGPGTSFKSVKTYPKGTDVKLSCQTYGEVIFGNSIWDKTQDGCYVSDYYVKTGSNNMVTGEC

SEQ ID NO: 214 - protein, 74 aa, Thielavia heterothallica
YPVTANGGLSCRSGPGTSYAVKKTYKKGFDVKISCQTTGTSVNGNNIWDKTQDGCYVADYYVKTGKNGFVTSKC

SEQ ID NO: 215 - protein, 73 aa, Hypocrea virens
YPITGEAVNCRTGPGTSFAIKKTYKKSQDVSVTCQTSGTSVNGNSIWDKTSDGCYVADYYVKTGSSSYVTKKC

SEQ ID NO: 216 - protein, 73 aa, Metarhizium robertsii
YPITGTTVNCRSGPSTHDKVIKTYNKGNDIKISCQVAGETVSGNNLWDKTQDGCFVSDYYVKTGSNGMVTGQC

SEQ ID NO: 217 - protein, 73 aa, Fusarium oxysporum; identical to SEQ ID NO: 212

SEQ ID NO: 218 - protein, 73 aa, Chaetomium thermophilum
YAITGDNVNCRSGPGTSYAVKKVYKKGTDVKISCQTTGTNINGNNLWDKTSDGCYVSDYYVKTGSNGYVTSKC

SEQ ID NO: 219 - protein, 73 aa, Thielavia terrestris
YPITGNGVNCRSGPGTSHAVKKVYAKGTDIKVTCQTEGTSINGNSIWDKTSDGCYVADYYVKTGSNSYVTKKC

SEQ ID NO: 220 - protein, 73 aa, Fusarium oxysporum
YPITGNDVKCRSGPGTSYAVKKVLKKGTDVKITCQTEGTNISGNTIWDKTSDGCYVSDYYVKTGSSGYIKPKC

SEQ ID NO: 221 - protein, 73 aa, Fusarium oxysporum
YPITGNDVKCRSGPGTSYAVKEVLKKGTDVKITCQTEGTNISGNTIWDKISDGCYVSDYYVKTGSSGYIKPKC

SEQ ID NO: 222 - protein, 73 aa, Fusarium oxysporum
FPITGNTVNCRSGPGTSYSVKRTYKKGQDVSITCQTYGTNVNGNSIWDKTSDGCYVADYYVKTGSDEFVTKKC

SEQ ID NO: 223 - protein, 73 aa, Fusarium oxysporum
YPITGNDVKCRSGPGTSYAVKKVLKKGTDVKITCQTEGTNISGNTIWDKISDGCYVSDYYVKTGSNGYIKPKC

SEQ ID NO: 224 - protein, 73 aa, Fusarium oxysporum; identical to SEQ ID NO: 212

SEQ ID NO: 225 - protein, 73 aa, Fusarium oxysporum; identical to SEQ ID NO: 212

SEQ ID NO: 226 - protein, 73 aa, Fusarium oxysporum; identical to SEQ ID NO: 212

SEQ ID NO: 227 - protein, 73 aa, Penicillium roqueforti
YPITGDTVNCRSGPGTSHTVVKTYKKAHDVKITCQTTGDSISGNNIWDKTSDGCYVADYYVKTGSNSYVTAKC

SEQ ID NO: 228 - protein, 73 aa, Penicillium roqueforti
YAITASVANCRTGPSTSNAVVTTYKKGADVKITCQTYGENIQGNSIWDKTSDGCYVADYYVKTGSNSMVTKDC

SEQ ID NO: 229 - protein, 73 aa, Trichoderma reesei; identical to SEQ ID NO: 209

SEQ ID NO: 230 - protein, 73 aa, Metarhizium anisopliae
YPITGTTVNCRSGPSTHDKVIKTYNKGNDIKISCQVAGENVSGNNLWDKTRDGCYVSDYYVKTGSNGMVTGQC

SEQ ID NO: 231 - protein, 73 aa, Pseudogymnoascus pannorum
YPITGSTVNCRSGPGTSYAVKKTYKKGDAVTITCQKEGPVVSGNSIWDKTSDGCYVADYYVKTGSNGYVKPKC

SEQ ID NO: 232 - protein, 72 aa, Pseudogymnoascus pannorum
YPISGDSVNCRSGPATSYKVIKTYKKGHDVKITCQTVGETIKGGNLWDKTSDGCYVTDYYVKTGTTGRVVKK

SEQ ID NO: 233 - protein, 72 aa, Pseudogymnoascus pannorum
FPVTATVNCRSGPGTGFAVKKSYTKGHAVTISCQTGGTSVQGNSIWDKTSDGCYVADYYVKTGSSGYVKPKC

SEQ ID NO: 234 - protein, 73 aa, Pseudogymnoascus pannorum
FPITGDTVNCRSGPGTSYAVKKSYNKGHSVTITCQTGGTSVKGNSIWDKTSDGCYVADYYVKTGSSGYVKPKC

SEQ ID NO: 235 - protein, 73 aa, Pseudogymnoascus pannorum
FPITGDSVNCRSGPGTSYAVKKSYAKGHSVTITCQTGGTSVNGNSIWDKTSDGCYVADYYVKTGSSGYVKPKC

SEQ ID NO: 236 - protein, 73 aa, Pseudogymnoascus pannorum
YPINAAGVNCRSGPGTSYAVKKSYAKDHAVTVTCQTAGTSVNGNSIWDKTSDGCYVADYYVNTGSSGYVKPKC

SEQ ID NO: 237 - protein, 73 aa, Pseudogymnoascus pannorum
YPINAAGVNCRSGPGTGYAVKKSYAKDHAVTVTCQTAGTTVNGNSIWDKTSDGCYVADYYVNTGSSGYVKPKC

SEQ ID NO: 238 - protein, 73 aa, Pseudogymnoascus pannorum
YPITGTTVNCRSGPGTSYAVKKTYKKGDAVTITCQKEGPVISGNSIWDKTSDGCYVADYYVKTGSNGYVKPKC

SEQ ID NO: 239 - protein, 73 aa, Pseudogymnoascus pannorum
YPITGKTVNCRAGPGTSYPVKKTYSKGDTVTITCQTSGTKVNGNAIWDLTSDGCYLTDYYVKTGTSKYIKPQC

SEQ ID NO: 240 - protein, 69 aa, Pseudogymnoascus pannorum
YPISGTDVNCRSGPATSYKVVKTYKKGHDVKVTCQTVGETIKGDNLWDKTSDGCYVADYYVKTGTTGRV

SEQ ID NO: 241 - protein, 73 aa, Pseudogymnoascus pannorum
YPINTAGVNCRSGPGTSYAVKKSYAKDHAVTVTCQTAGTSVNGNAIWDKTSDGCYVADYYVNTGSNGYVKPKC

SEQ ID NO: 242 - protein, 73 aa, Pseudogymnoascus pannorum
YPINAAGVNCRSGPGTGYAVKKSYAKDHAVTVTCQTAGTSVNGNSIWDKTSDGCYVADYYVNTGSSGYVKPKC

SEQ ID NO: 243 - protein, 74 aa, Pseudogymnoascus pannorum
YPISGTSVNCRSGPATSYKVIKAYKKGQDVKITCQTVGETVSGDNLWDKTTDGCYVADYYVKTGTTGRVVNAEC

SEQ ID NO: 244 - protein, 73 aa, Pseudogymnoascus pannorum; identical to SEQ ID NO: 235

SEQ ID NO: 245 - protein, 73 aa, Pseudogymnoascus pannorum
FPITGDTVNCRSGPGTSYAVKKSYAKGHDVTITCQTGGTSVSGNSIWDKTSDGCYVADYYVKTGSSGYVKPKC

SEQ ID NO: 246 - protein, 64 aa, Pseudogymnoascus pannorum
YPITGKTVNCRAGPGTSYPVKKTYSKGDTVTITCQTSGTKVNGNAIWDLTSDGCYLTDYYVKTG

SEQ ID NO: 247 - protein, 73 aa, Pseudogymnoascus pannorum
FPITGDSVNCRSGPGTSYAVKKSYAKGHDVTITCQTGGTSVSGNSIWDKTSDGCYVADYYVKTGSSGYVKPKC

SEQ ID NO: 248 - protein, 72 aa, Pseudogymnoascus pannorum
FPVTETVNCRSGPGTSYGVKKSYTKGHAVTISCQTGGTSVKGNSIWDKTSDGCYVADYYVKTGSNGYVKPKC

SEQ ID NO: 249 - protein, 72 aa, Pseudogymnoascus pannorum
FPVTATVNCRSGPGTGYAVKKSYTKGNAVTISCQTGGTSVNGNSIWDKTSDGCYVADYYVKTGSSGYVKPKC

SEQ ID NO: 250 - protein, 73 aa, Pseudogymnoascus pannorum
YPINAAGVNCRSGPGTSYAVKKSYAKDHAVTVTCQTAGTSVNGNAIWDKTSDGCYVADYYVNTGSNGYVKPKC

SEQ ID NO: 251 - protein, 73 aa, Pseudogymnoascus pannorum
FPITGATVNCRSGPGTSYAVKKSYAKGHDVTITCQTGGTSVNGNSIWDKTSDGCYVADYYVKTGSSGYVKPKC

SEQ ID NO: 252 - skipped

SEQ ID NO: 253 - protein, 15 aa, synthetic construct
  REGION 1..15: note = Synthetic construct
  REGION 1: note = NON_CONS - The amino acid in position 1 of the conserved motif is leucine or valine.
  REGION 2: note = NON_CONS - The amino acid in position 2 of the conserved motif is asparagine, aspartic acid, serine or threonine.
  REGION 3: note = NON_CONS - The amino acid in position 3 of the conserved motif is any amino acid.
  REGION 5: note = NON_CONS - The amino acid in position 5 of the conserved motif is glutamic acid or glutamine.
  REGION 6: note = NON_CONS - The amino acid in position 6 of the conserved motif is phenylalanine, tyrosine or tryptophan.
  REGION 7: note = NON_CONS - The amino acid in position 7 of the conserved motif is alanine, aspartic acid, asparagine, glycine or serine.
  REGION 10: note = NON_CONS - The amino acid in position 10 of the conserved motif is any amino acid.
  REGION 12: note = NON_CONS - The amino acid in position 12 of the conserved motif is leucine, phenylalanine, tyrosine or tryptophan.
  REGION 13: note = NON_CONS - The amino acid in position 13 of the conserved motif is any amino acid.
  REGION 14: note = NON_CONS - The amino acid in position 14 of the conserved motif is phenylalanine or tyrosine.
XXXNXXXALXSXXXN

SEQ ID NO: 254 - skipped

SEQ ID NO: 255 - DNA, 946 bp, Trichophaea saccata
  sig_peptide 1..51
  mat_peptide 52..943
  source 1..946: mol_type = genomic DNA
  CDS 1..347
  CDS 401..615
  CDS 668..772
  CDS 825..943
atgcacgctctcacccttctcaccgcaaccctcttcggtctcgcagcggcctacccagtgaagaccgaccttcactgccgctcctctcccagcacttccgccagcatcgtccgcacctactccagtggaacggaagtccagatccagtgccagaccacgggcacttcggtccaaggatccaatgtctgggacaagacccagcacggttgctacgtcgcagactactacgtcaagaccgggcattctgggattttcaccaccaagtgcggtagcagctcgggtggaggttcctgcaagcctcccccgatcaatgctgctactgtcgcattgatcaaggagtttgaggggtaagtgacagctctgagtgaggtggtatgaggattaagactgacgaggatagattcgttcctaagcccgccccggatcctattggattgccgaccgtgggatacgggcatctttgcaagactaagggctgcaaagaagtgccttacagcttccctctcacccaggagactgccaccaagttgcttcagagcgatatcaagactttcacctcttgcgttagcaactacgtcaaggactctgttaagctcaacgataaccagtacggaggtgagttccagtgtaacagtgaatttattgatgatattctaagtaattttagctctggcgtcttgggctttcaacgtcggctgcggaaacgtccagacttcttcgctgatcaagagattgaacgctggggagaaccctaacactgtcgctgctcagggtaagatatttatcccggatttgctcttgacacatggctgaaaaagttgcagaactccccaagtggaagtacgctggtggaaaggttatgcctggcttggtccgccgccgcaatgctgaggtcgcgctcttcaagaagcccagcagcgttcaggcccaccctcccaagtgctaa

SEQ ID NO: 256 - protein, 262 aa, Trichophaea saccata
MHALTLLTATLFGLAAAYPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 257 - protein, 245 aa, Trichophaea saccata
YPVKTDLHCRSSPSTSASIVRTYSSGTEVQIQCQTTGTSVQGSNVWDKTQHGCYVADYYVKTGHSGIFTTKCGSSSGGGSCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 258 - DNA, 39 bp, synthetic construct
  misc_feature 1..39: note = Synthetic construct
  source 1..39: mol_type = other DNA
acacaactggggatccaccatgcacgctctcacccttct

SEQ ID NO: 259 - DNA, 38 bp, synthetic construct
  misc_feature 1..38: note = Synthetic construct
  source 1..38: mol_type = other DNA
ctagatctcgagaagcttttagcacttgggagggtggg

SEQ ID NO: 260 - DNA, 20 bp, synthetic construct
  misc_feature 1..20: note = Synthetic construct
  source 1..20: mol_type = other DNA
gcaagggatgccatgcttgg

SEQ ID NO: 261 - DNA, 19 bp, synthetic construct
  misc_feature 1..19: note = Synthetic construct
  source 1..19: mol_type = other DNA
catataaccaattgccctc

SEQ ID NO: 262 - DNA, 923 bp, Chaetomium thermophilum
  sig_peptide 1..54
  mat_peptide 55..920
  source 1..923: mol_type = genomic DNA
  CDS 1..367
  CDS 425..555
  CDS 630..920
atgaagttcgccatcctcgccgccgttgcctccttccttgctcccgccagcgcctatgccattaccggcgacaacgtcaactgtcgcagcggcccaggcactagctacgcggtcaagaaggtctacaagaagggcaccgacgtcaagatcagctgccagactactggcaccaatatcaacggcaacaatctttgggacaagacctctgacggctgctatgtttcggactattacgtcaagactggcagcaacggctatgtgaccagcaagtgcagctctagtggaggcagcacctgcgctgcgccaaagtctaaccaagccactgtcgatctcatcgctgagtttgagggcttccgcgccaatatctgtatgtgatatcaacctcgcattgtacttaagaacaaggctctaactcgtgaacaagacaccgatgcagctggatacgccactgtcggatacggccacaagtgccagaaggctaagtgcgctgaggtcaagtacaagattcccctttccaaggccgatggcaagaagcttctggccgatgatatgagggtatgttcctcccttcataaaaaccataccatctctggaagcctcatcaatttcgactaaccccaaatctacagagcttcgaagtctgcattacgaacatgctcaatagcaaggccaagctgaactacaaccagttcggtgcactggtcagttggtccttcaatgtcggctgcggcgctgccaagtcgtcgactctcatcaagcgcctaaacaacggcgagaatgtcaacaaggttctttccgaggagctgcccaagtggaacaaggctggtggcaaggttcttcaaggccttgtccgccgccgtgctgctgaggtcgctctggctaagaagtctggcagctctcaggctcttcctgttaagtgctga

SEQ ID NO: 263 - protein, 263 aa, Chaetomium thermophilum
MKFAILAAVASFLAPASAYAITGDNVNCRSGPGTSYAVKKVYKKGTDVKISCQTTGTNINGNNLWDKTSDGCYVSDYYVKTGSNGYVTSKCSSSGGSTCAAPKSNQATVDLIAEFEGFRANIYTDAAGYATVGYGHKCQKAKCAEVKYKIPLSKADGKKLLADDMRSFEVCITNMLNSKAKLNYNQFGALVSWSFNVGCGAAKSSTLIKRLNNGENVNKVLSEELPKWNKAGGKVLQGLVRRRAAEVALAKKSGSSQALPVKC

SEQ ID NO: 264 - protein, 249 aa, Chaetomium thermophilum
PASAYAITGDNVNCRSGPGTSYAVKKVYKKGTDVKISCQTTGTNINGNNLWDKTSDGCYVSDYYVKTGSNGYVTSKCSSSGGSTCAAPKSNQATVDLIAEFEGFRANIYTDAAGYATVGYGHKCQKAKCAEVKYKIPLSKADGKKLLADDMRSFEVCITNMLNSKAKLNYNQFGALVSWSFNVGCGAAKSSTLIKRLNNGENVNKVLSEELPKWNKAGGKVLQGLVRRRAAEVALAKKSGSSQALPVKC

SEQ ID NO: 265 - DNA, 801 bp, Trichoderma harzianum
  sig_peptide 1..54
  mat_peptide 55..798
  source 1..801: mol_type = genomic DNA
  CDS 1..798
atgaagactgcctttgctgctctcgttttctccctggcatccatcgtcagcgcctaccccatcactggcgatgttgtcaactgccgcaccggacctggcaccagctacgccatcaagaagtcctacaagaagaaccaagacatctccataagctgtcagacagcgggaaccagcgtcaatggcaacagcatttgggataagaccgccgacggctgctacgtcgccgactactacgtcaagacgggctccagcggctacgtgaccaagaagtgcactgcttcctccggcggcggctcctcgtccagctactgcaagaccatcaacagcgccggcgtcgatctcatcgccaagtgggagggcttcgtcgccagcccaaagcccgaccccattggcctgcccacggttggctacggccatctctgccagcagaagaactgcagagaggtcaagtacaagttccccctgaccaagaccaccgccaaggagctgctgctggacgacctgcccaagtacacaaagtgcctcgccgactacctcaacgacaagcccaagctcaatgccaaccagtgggcggccctgacgtcctgggtcttcaacgtcggctgcggcaacgcaaagacatcgactctcgtgaagcgcctcaacaacggcgaggctgccaacactgttgctgcagaggaactccccaagtggcgcatggcaggaggaaaagtcctgccaggcctcgaggctcgccgaaaggacgaggtcaagctgttcaagacggcgtcatcgaagcaggcctatccaaagtgccagtga

SEQ ID NO: 266 - protein, 266 aa, Trichoderma harzianum
MKTAFAALVFSLASIVSAYPITGDVVNCRTGPGTSYAIKKSYKKNQDISISCQTAGTSVNGNSIWDKTADGCYVADYYVKTGSSGYVTKKCTASSGGGSSSSYCKTINSAGVDLIAKWEGFVASPKPDPIGLPTVGYGHLCQQKNCREVKYKFPLTKTTAKELLLDDLPKYTKCLADYLNDKPKLNANQWAALTSWVFNVGCGNAKTSTLVKRLNNGEAANTVAAEELPKWRMAGGKVLPGLEARRKDEVKLFKTASSKQAYPKCQ

SEQ ID NO: 267 - protein, 248 aa, Trichoderma harzianum
YPITGDVVNCRTGPGTSYAIKKSYKKNQDISISCQTAGTSVNGNSIWDKTADGCYVADYYVKTGSSGYVTKKCTASSGGGSSSSYCKTINSAGVDLIAKWEGFVASPKPDPIGLPTVGYGHLCQQKNCREVKYKFPLTKTTAKELLLDDLPKYTKCLADYLNDKPKLNANQWAALTSWVFNVGCGNAKTSTLVKRLNNGEAANTVAAEELPKWRMAGGKVLPGLEARRKDEVKLFKTASSKQAYPKCQ

SEQ ID NO: 268 - DNA, 585 bp, synthetic construct
  misc_feature 1..585: note = Synthetic construct
  sig_peptide 10..69
  mat_peptide 70..576
  source 1..585: mol_type = other DNA
  CDS 10..576
ggatccaccatggcgaaagtctccactctcaccatcgcattgctcacgatggcgtcgcaggcaagggcacagtgtgtgggctgtaaacctcctccgatcaacgcagccacagtggcgttgatcaaagagttcgaaggcttcgtgcctaaacctgcacccgatccgatcggcctccctacagtcggctacggccatctctgtaaaactaagggatgtaaagaggtcccctactccttccctttgacccaggagactgccacgaaattgctccagtcggatatcaagaccttcacctcctgtgtctccaactacgtcaaagactcggtcaagctcaacgacaaccagtatggagccttggcctcgtgggccttcaacgtgggatgtggcaacgtgcagacatcgtcgctcattaagcgcttgaacgcaggcgaaaaccccaacaccgtcgcagcccaggagttgccgaagtggaagtatgcgggaggcaaggtcatgcctggtctcgtccgtaggaggaacgcagaggtggccctcttcaagaagccttcgtccgtccaggcgcaccctcctaaatgttaaaagctt

SEQ ID NO: 269 - protein, 189 aa, synthetic construct
  REGION 1..189: note = Synthetic Construct
MAKVSTLTIALLTMASQARAQCVGCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 270 - protein, 169 aa, synthetic construct
  REGION 1..169: note = Synthetic construct
QCVGCKPPPINAATVALIKEFEGFVPKPAPDPIGLPTVGYGHLCKTKGCKEVPYSFPLTQETATKLLQSDIKTFTSCVSNYVKDSVKLNDNQYGALASWAFNVGCGNVQTSSLIKRLNAGENPNTVAAQELPKWKYAGGKVMPGLVRRRNAEVALFKKPSSVQAHPPKC

SEQ ID NO: 271 - DNA, 270 bp, synthetic construct
  misc_feature 1..270: note = Synthetic construct
  sig_peptide 1..51
  mat_peptide 52..270
  source 1..270: mol_type = other DNA
  CDS 1..270
atgcacgctctcacccttctcaccgcaaccctcttcggtctcgcagcggcctacccagtgaagaccgaccttcactgccgctcctctcccagcacttccgccagcatcgtccgcacctactccagtggaacggaagtccagatccagtgccagaccacgggcacttcggtccaaggatccaatgtctgggacaagacccagcacggttgctacgtcgcagactactacgtcaagaccgggcattctgggattttcaccaccaagtgcggt

SEQ ID NO: 272 - protein, 90 aa, synthetic construct
  REGION 1..90: note = Synthetic Construct
MHALTLLTATLFGLAAAYPVKTDLHCRSSPSTSASIVRTYSSGTEVQIQCQTTGTSVQGSNVWDKTQHGCYVADYYVKTGHSGIFTTKCG

SEQ ID NO: 273 - protein, 73 aa, Synthetic construct
YPVKTDLHCRSSPSTSASIVRTYSSGTEVQIQCQTTGTSVQGSNVWDKTQHGCYVADYYVKTGHSGIFTTKCG

SEQ ID NO: 274 - DNA, 37 bp, synthetic construct
  misc_feature 1..37: note = Synthetic construct
  source 1..37: mol_type = other DNA
agatctcgagaagcttataccgcacttggtggtgaaa

SEQ ID NO: 275 - DNA, 39 bp, synthetic construct
  misc_feature 1..39: note = Synthetic construct
  source 1..39: mol_type = other DNA
acacaactggggatccaccatgaagactgcctttgctgc

SEQ ID NO: 276 - DNA, 38 bp, synthetic construct
  misc_feature 1..38: note = Synthetic construct
  source 1..38: mol_type = other DNA
agatctcgagaagcttatcactggcactttggataggc

SEQ ID NO: 277 - DNA, 39 bp, synthetic construct
  misc_feature 1..39: note = Synthetic construct
  source 1..39: mol_type = other DNA
acacaactggggatccaccatgaagttcgccatcctcgc

SEQ ID NO: 278 - DNA, 39 bp, synthetic construct
  misc_feature 1..39: note = Synthetic construct
  source 1..39: mol_type = other DNA
agatctcgagaagcttatcagcacttaacaggaagagcc

SEQ ID NO: 279 - protein, 166 aa, Acremonium alcalophilum
QCVGPEVNSASINLIKEFEGWYPDIYVDPAGYPTVGYGHLCSDSSCSDVSYSIPLSEADGENLLRDDITNFQNCITWQTASSVVLNANQYGALVSWAFNVGCGASESSSLIARLNAGEDPNTVAEEELPRWNQGGGQVLPGLVRRRAAEVELHQIPTDVAALPACS

SEQ ID NO: 280 - protein, 157 aa, Acremonium alcalophilum
QCVGPAINSAALNLIKEFEGWRPNIYRDPVGLPTVGYGHLCRDSSCSDVPYPIPLSVANGERLLRSDLATASSVVLNANQYGALVSWAFNVGCGATSTSTLIRRLNAGESPNTVAAQELPRWNKAGGQVLPGLVRRRAAEVELHRTSTSVRALPACS

SEQ ID NO: 281 - skipped

SEQ ID NO: 282 - protein, 8 aa, synthetic construct
  REGION 1..8: note = Synthetic construct
  REGION 2: note = NON_CONS - The amino acid in position 2 of the conserved motif is valine or isoleucine.
  REGION 7: note = NON_CONS - The amino acid in position 7 of the conserved motif is any amino acid.
TXGYGHXC

SEQ ID NO: 283 - protein, 15 aa, synthetic construct
  REGION 1..15: note = Synthetic construct
  REGION 3: note = NON_CONS - The amino acid in position 3 of the conserved motif is any amino acid.
  REGION 5: note = NON_CONS - The amino acid in position 5 of the conserved motif is glutamic acid or glutamine.
  REGION 6: note = NON_CONS - The amino acid in position 6 of the conserved motif is phenylalanine, tryptophan or tyrosine.
  REGION 7: note = NON_CONS - The amino acid in position 7 of the conserved motif is alanine or glycine.
  REGION 10: note = NON_CONS - The amino acid in position 10 of the conserved motif is any amino acid.
  REGION 12: note = NON_CONS - The amino acid in position 12 of the conserved motif is leucine, phenylalanine or tryptophan.
  REGION 13: note = NON_CONS - The amino acid in position 13 of the conserved motif is any amino acid.
  REGION 14: note = NON_CONS - The amino acid in position 14 of the conserved motif is phenylalanine or tyrosine.
LNXNXXXALXSXXXN

SEQ ID NO: 284 - protein, 11 aa, synthetic construct
  REGION 1..11: note = Synthetic construct
  REGION 2: note = NON_CONS - The amino acid in position 2 of the conserved motif is phenylalanine or tyrosine.
  REGION 3: note = NON_CONS - The amino acid in position 3 of the conserved motif is isoleucine or valine.
  REGION 4: note = NON_CONS - The amino acid in position 4 of the conserved motif is alanine, serine or threonine.
  REGION 6: note = NON_CONS - The amino acid in position 6 of the conserved motif is lysine, phenylalanine or tyrosine.
  REGION 7: note = NON_CONS - The amino acid in position 7 of the conserved motif is phenylalanine or tyrosine.
  REGION 8: note = NON_CONS - The amino acid in position 8 of the conserved motif is isoleucine or valine.
  REGION 9: note = NON_CONS - The amino acid in position 9 of the conserved motif is any amino acid.
CXXXDXXXXTG

SEQ ID NO: 285 - protein, 9 aa, synthetic construct
  REGION 1..9: note = Synthetic construct
  REGION 1: note = NON_CONS - The amino acid in position 1 of the conserved motif is glutamic acid, glycine or valine.
  REGION 3: note = NON_CONS - The amino acid in position 3 of the conserved motif is any amino acid.
  REGION 4: note = NON_CONS - The amino acid in position 4 of the conserved motif is any amino acid.
  REGION 7: note = NON_CONS - The amino acid in position 7 of the conserved motif is any amino acid.
  REGION 8: note = NON_CONS - The amino acid in position 8 of the conserved motif is any amino acid.
XLXXRRXXE

SEQ ID NO: 286 - protein, 9 aa, synthetic construct
  REGION 1..9: note = Synthetic construct
  REGION 3: note = NON_CONS - The amino acid in position 3 of the conserved motif is any amino acid.
  REGION 4: note = NON_CONS - The amino acid in position 4 of the conserved motif is any amino acid.
  REGION 7: note = NON_CONS - The amino acid in position 7 of the conserved motif is any amino acid.
  REGION 8: note = NON_CONS - The amino acid in position 8 of the conserved motif is any amino acid.
GLXXRRXXE

SEQ ID NO: 287 - DNA, 961 bp, Trichophaea minuta
  sig_peptide 1..51
  mat_peptide 52..958
  source 1..961: mol_type = genomic DNA
  CDS 1..347
  CDS 410..624
  CDS 683..787
  CDS 840..958
atgcacgccctcgccctcctcgcagcaaccctcttcggtctcgcagcggcctacccagcgaaagttgacctccgctgccgttcctctcccagcacctccgcgagcgtagtccgcacctactccaaaggctccgaaatccagatctcttgccagaccacgggcacttccgtcgaaggctccaatgtctgggacaagacccagcatggctgctacgtcgccgactactacgtcaagaccgggcattctggaatcttcacaaccaagtgcggcagcagctctggtggtggttcttgcaagccgcccccgatcaatgcggctactgtggcgttgattaaggagtttgaggggtaagtggccggcggacttggggagtgagagctatggagctggatgctgacgggactactagatttgttgctaagcccgcaccggatcctattggattgccgactgtgggatatggtcatctttgcaagactaagggctgcaaggaggtgccttacagtttcccgctcacccagactaccgccaccaagttgctgcagagcgatatcaagactttcacctcctgtgttagcaactacgtcaaggactctgttaagcttaacgataaccagttcggaggtaatttccagtattccccgttgatcagtgataatataatgattctgacgaattttagctctatcgtcgtgggctttcaacgttggttgcggaaatatccagacttcttcgctgatcaagagactgaatgctggagagaaccctaacactgttgccgctcagggtaagaccttctcactggagtgaattttggggaggagctgacaaagttgcagaactccctaagtggaaatatgctggtggaaaggttcttcccggcttggtccgccgccggaaggctgaggtcgcgcttttcaagaagcccagcagcgtccaggcccaccctcccaagtgctag

SEQ ID NO: 288 - protein, 262 aa, Trichophaea minuta
MHALALLAATLFGLAAAYPAKVDLRCRSSPSTSASVVRTYSKGSEIQISCQTTGTSVEGSNVWDKTQHGCYVADYYVKTGHSGIFTTKCGSSSGGGSCKPPPINAATVALIKEFEGFVAKPAPDPIGLPTVGYGHLCKTKGCKEVPYSFPLTQTTATKLLQSDIKTFTSCVSNYVKDSVKLNDNQFGALSSWAFNVGCGNIQTSSLIKRLNAGENPNTVAAQELPKWKYAGGKVLPGLVRRRKAEVALFKKPSSVQAHPPKC

SEQ ID NO: 289 - DNA, 789 bp, synthetic construct
  sig_peptide 1..51
  source 1..789: mol_type = other DNA
  mat_peptide 52..789
atgcacgcactcgcgctcttggcagccactctcttcggcctcgcagcagcctatcccgcaaaggtggacctccgatgtcgttcgtccccttccacatccgcctccgtcgtcaggacttattccaagggctccgagatccagatttcgtgtcagaccacgggcacatcggtggaaggctccaacgtgtgggacaagacgcagcacggctgttacgtcgccgattactacgtcaagaccggtcattcgggcatcttcacaaccaagtgtggttcctcgtcgggaggaggctcctgtaaacctcctccgatcaacgcagcaacagtcgccctcatcaaggagttcgaaggcttcgtggcaaaacctgcacccgatcccatcggtttgcccactgtgggatacggccacttgtgtaagacgaaaggctgtaaggaggtgccctactccttcccgttgactcagacaacggcgacgaaattgttgcagtcggacatcaaaactttcacatcctgtgtgtccaactacgtcaaggattcggtcaaactcaacgataaccagttcggagcgttgtcgtcgtgggccttcaacgtcggatgtggcaacatccagacgtcgtcgctcatcaagcgtctcaacgcaggagagaacccgaacactgtcgcagcccaggaactcccgaaatggaagtatgcgggtggcaaggtcctccctggtttggtccgaaggcggaaggcagaagtcgcgttgttcaagaagccgtcgtcggtccaggcacatcctccgaagtgttaa

SEQ ID NO: 290 - protein, 262 aa, synthetic construct; identical to SEQ ID NO: 288
  REGION 1..262: note = Synthetic Construct

SEQ ID NO: 291 - protein, 245 aa, Trichophaea minuta
YPAKVDLRCRSSPSTSASVVRTYSKGSEIQISCQTTGTSVEGSNVWDKTQHGCYVADYYVKTGHSGIFTTKCGSSSGGGSCKPPPINAATVALIKEFEGFVAKPAPDPIGLPTVGYGHLCKTKGCKEVPYSFPLTQTTATKLLQSDIKTFTSCVSNYVKDSVKLNDNQFGALSSWAFNVGCGNIQTSSLIKRLNAGENPNTVAAQELPKWKYAGGKVLPGLVRRRKAEVALFKKPSSVQAHPPKC

SEQ ID NO: 292 - DNA, 935 bp, Chaetomium sp.
  sig_peptide 1..54
  mat_peptide 55..932
  source 1..935: mol_type = genomic DNA
  CDS 1..379
  CDS 448..578
  CDS 642..932
atgaagttcgctatcatcgccgccgtcgttcccttcctggcccccgcctcggcctacaagatctcgggcagctccgtcaactgccgctccgggcccggcaccaactaccccgtcaagaagacctacgccaacggtgacgaggtcaccattagctgccagaccaccggcacgaacgtggagggcaacaacatctgggacaagacccagcacggctgctacgtcgccgacaagtatgtcaagaccggcaaggacggcttcgtcaccaagaagtgcggcagctccggtggtggtggtggtggcaagacctgcaaggcccccaagtccaacgccgctaccgttgatttgattgcttcgttcgagggcttccgggccaatatctgtgagtagacccccgaaagggaaagtggcataagctgtgcagactcctaacgagtgcacccctttcagacacggatgccactggccaccccaccgttggctacggccacatgtgcaccaagtccaggtgcgccgaggtcaagtacaagatcccgctctccaaggccgacggcaagaagctgctcgccgatgacatggcggtgagtcccatccattccctccttcacagactaacttactgacgcgccctctccaatttcaagaaattcgagaagtgcatcaaggagatgctcaactccaaggccaagctcaacctgaaccagtacggcgccctcgtcagctggtccttcaacgtcggctgcggcgccgccaagggctcccagctcgtcagccgcctcaacaagggcgagaaccccaacacggtcctctccaacgagctccccaagtgggtccacggcaacggcaaggtgctgcccggtctcgtccgccgccgcaacgccgagattgccctcgccaagaagagcggcagcggcgccgccctgcccgtcaagtgctag

SEQ ID NO: 293 - protein, 267 aa, Chaetomium sp.
MKFAIIAAVVPFLAPASAYKISGSSVNCRSGPGTNYPVKKTYANGDEVTISCQTTGTNVEGNNIWDKTQHGCYVADKYVKTGKDGFVTKKCGSSGGGGGGKTCKAPKSNAATVDLIASFEGFRANIYTDATGHPTVGYGHMCTKSRCAEVKYKIPLSKADGKKLLADDMAKFEKCIKEMLNSKAKLNLNQYGALVSWSFNVGCGAAKGSQLVSRLNKGENPNTVLSNELPKWVHGNGKVLPGLVRRRNAEIALAKKSGSGAALPVKC

SEQ ID NO: 294 - protein, 249 aa, Chaetomium sp.
YKISGSSVNCRSGPGTNYPVKKTYANGDEVTISCQTTGTNVEGNNIWDKTQHGCYVADKYVKTGKDGFVTKKCGSSGGGGGGKTCKAPKSNAATVDLIASFEGFRANIYTDATGHPTVGYGHMCTKSRCAEVKYKIPLSKADGKKLLADDMAKFEKCIKEMLNSKAKLNLNQYGALVSWSFNVGCGAAKGSQLVSRLNKGENPNTVLSNELPKWVHGNGKVLPGLVRRRNAEIALAKKSGSGAALPVKC

SEQ ID NO: 295 - DNA, 795 bp, Mortierella sp.
  sig_peptide 1..57
  mat_peptide 58..792
  source 1..795: mol_type = genomic DNA
  CDS 1..792
atgaagttcacgctcgcccttgctgccttggctgcctctatccccgccacgctggcctatcccatcacgggtgccgatgctctgcattgccgttccggtcctggtacttcgtaccccatccaaaagacgctgcgtcctcctcaggatatcaagatccaatgccaggagcctggtacggtcgtcaacggcgttagcctgtgggacaagacccagtttggctgctacgtgtccgattattatgtcaagactggtaccggcaactacgtcgcccctcgctgcaacagcggtggcagctctagcgcttgcactggcctgaacgatgctggcatcaacttgatcaaggagtttgaaggcttcgtccctcgccctgcgccggatcccattggcttgcccaccgttggctacggtcatttgtgccagaccaagggttgcggcgaggtcaagtactcattccctcttaccaccgcgaccgccaccgctttgctcaaggacgacctccccaagtacacatcgtgcttggccaaggccttgaatggcaaaccaaaactgaacaagaaccagtgggccgcccttgcctcgtggacgtttaatgtgggctgtggcaacatgaaatcctcgagccttatcacccgcctcaatgcaggtcagaaccccaacactgtcgccaccgaggagctccccaagtggaaactggctggtggcaaggtcctccccggtctggtccgtcgtcgtgctgccgaggtcaagctgttcaagacggccaactcttcccagggttatcctaagtgtgcttaa

SEQ ID NO: 296 - protein, 264 aa, Mortierella sp.
MKFTLALAALAASIPATLAYPITGADALHCRSGPGTSYPIQKTLRPPQDIKIQCQEPGTVVNGVSLWDKTQFGCYVSDYYVKTGTGNYVAPRCNSGGSSSACTGLNDAGINLIKEFEGFVPRPAPDPIGLPTVGYGHLCQTKGCGEVKYSFPLTTATATALLKDDLPKYTSCLAKALNGKPKLNKNQWAALASWTFNVGCGNMKSSSLITRLNAGQNPNTVATEELPKWKLAGGKVLPGLVRRRAAEVKLFKTANSSQGYPKCA

SEQ ID NO: 297 - protein, 245 aa, Mortierella sp.
YPITGADALHCRSGPGTSYPIQKTLRPPQDIKIQCQEPGTVVNGVSLWDKTQFGCYVSDYYVKTGTGNYVAPRCNSGGSSSACTGLNDAGINLIKEFEGFVPRPAPDPIGLPTVGYGHLCQTKGCGEVKYSFPLTTATATALLKDDLPKYTSCLAKALNGKPKLNKNQWAALASWTFNVGCGNMKSSSLITRLNAGQNPNTVATEELPKWKLAGGKVLPGLVRRRAAEVKLFKTANSSQGYPKCA

SEQ ID NO: 298 - DNA, 801 bp, Metarhizium sp.
  sig_peptide 1..57
  mat_peptide 58..798
  source 1..801: mol_type = genomic DNA
  CDS 1..798
atgaagtttctcgctgttgcaaatgtcttggcaatgacatccactgtggtagttgcctacccagtttctgctgatagtctcaactgccgagccgagcccaacaccagctccgccatcaaaacaacctacaagaagggtgaagatgtcaaaatttcctgccagaccgagggtccctccatcaacggcaacaccatctgggacaagacccaagatggttgctacgtcgccgactactacgtcaaaaccggatcctctggctacgtcactggcaaatgcggcggtagcagtcccccaagcggttccggcttttgcaagacggtaaacaaggcgggcctcgatctcatcaccaagtgggaaggctttgtttccagccccaggggcgaccccatcggcctgcctactgttggctatggccatctctgccagaagaagggctgcgccgaagtcaagtacaagttccctttgaccaaggccactgccctgcagctcctcaacgacgatctccccaagtataccggctgcctcggcaagctcctcaacagcaaggtcaagcttaatgacaatcagtgggcggctctgacctcctgggttttcaacgttggatgtggcaacgctcagtcttcctctcttgtcagacgtctcaacaatggcgagaaccccaacacagttgctcccagtgagcttcccaagtggaaaatggccggtggtaaggtgttggagggcttggtcaaacgccgtgctgacgaggtcagactcttcaaggtttcttcttccaagggcgcattccccaaatgccagtag

SEQ ID NO: 299 - protein, 266 aa, Metarhizium sp.
MKFLAVANVLAMTSTVVVAYPVSADSLNCRAEPNTSSAIKTTYKKGEDVKISCQTEGPSINGNTIWDKTQDGCYVADYYVKTGSSGYVTGKCGGSSPPSGSGFCKTVNKAGLDLITKWEGFVSSPRGDPIGLPTVGYGHLCQKKGCAEVKYKFPLTKATALQLLNDDLPKYTGCLGKLLNSKVKLNDNQWAALTSWVFNVGCGNAQSSSLVRRLNNGENPNTVAPSELPKWKMAGGKVLEGLVKRRADEVRLFKVSSSKGAFPKCQ

SEQ ID NO: 300 - protein, 247 aa, Metarhizium sp.
YPVSADSLNCRAEPNTSSAIKTTYKKGEDVKISCQTEGPSINGNTIWDKTQDGCYVADYYVKTGSSGYVTGKCGGSSPPSGSGFCKTVNKAGLDLITKWEGFVSSPRGDPIGLPTVGYGHLCQKKGCAEVKYKFPLTKATALQLLNDDLPKYTGCLGKLLNSKVKLNDNQWAALTSWVFNVGCGNAQSSSLVRRLNNGENPNTVAPSELPKWKMAGGKVLEGLVKRRADEVRLFKVSSSKGAFPKCQ

SEQ ID NO: 301 - DNA, 921 bp, Geomyces auratus
  sig_peptide 1..54
  mat_peptide 55..918
  source 1..921: mol_type = genomic DNA
  CDS 1..382
  CDS 440..570
  CDS 628..918
atgaagctttctcttctcgccgttggcgccgtggcatgctctttcgtgagcgccgctttccccattaccggcagtactgtcaactgccgcactgggccaggaacgagccatggcgtgaagacatcatacaagaagggccacgaagtaacggtatcctgccagaccggaggaaccagtgtgaacggcaattcaatctgggataagacatccgatggctgttatgttgccgattactatgttaagactggttcaagcggctacgtgaagcccaagtgcggctcctcttccggaggtggaggcggaagcagttgtggtgcacctaaatctaacgctgctactgtcaacctgattgccgaatttgagggtttcgttagccacgtttgtaagtgaccctttgtctggatgcgggctgtcggggatactgaccaacttgtgatagataccgacgctactggtcaccctactgtcggctatggccatctctgcagcaactcgaagtgctctggcatcggatactcaattcctatctccaaggccaatgctaagaagcttctcgccaaggacatggctgtaagtagtcaccgatgcttgaagatcccaagtattctaaccattctctatgtatagatcgccgagaagtgcattactgcaatgatcaacaagagccgtacactcaatctgaaccaatacggtgctctggttagctgggctttcaacgaaggatgtggcgcggccaaatcatccaccctcatcaaacgcatcaacaatggcgaaaagcccagcactgtcattcctcaagagcttccaaagtgggtatatggtgggagcagcgtccttcctggtcttgttcgccgtcgtaacgctgagattgcgcttgctaaaaaggctacatcgagcaaggcactgccggctcactgctaa

SEQ ID NO: 302 - protein, 268 aa, Geomyces auratus
MKLSLLAVGAVACSFVSAAFPITGSTVNCRTGPGTSHGVKTSYKKGHEVTVSCQTGGTSVNGNSIWDKTSDGCYVADYYVKTGSSGYVKPKCGSSSGGGGGSSCGAPKSNAATVNLIAEFEGFVSHVYTDATGHPTVGYGHLCSNSKCSGIGYSIPISKANAKKLLAKDMAIAEKCITAMINKSRTLNLNQYGALVSWAFNEGCGAAKSSTLIKRINNGEKPSTVIPQELPKWVYGGSSVLPGLVRRRNAEIALAKKATSSKALPAHC

SEQ ID NO: 303 - protein, 250 aa, Geomyces auratus
AFPITGSTVNCRTGPGTSHGVKTSYKKGHEVTVSCQTGGTSVNGNSIWDKTSDGCYVADYYVKTGSSGYVKPKCGSSSGGGGGSSCGAPKSNAATVNLIAEFEGFVSHVYTDATGHPTVGYGHLCSNSKCSGIGYSIPISKANAKKLLAKDMAIAEKCITAMINKSRTLNLNQYGALVSWAFNEGCGAAKSSTLIKRINNGEKPSTVIPQELPKWVYGGSSVLPGLVRRRNAEIALAKKATSSKALPAHC

SEQ ID NO: 304 - DNA, 902 bp, Ilyonectria rufa
  sig_peptide 1..54
  mat_peptide 55..899
  source 1..902: mol_type = genomic DNA
  CDS 1..352
  CDS 418..548
  CDS 609..899
atgaagttctctctctttgctgtctatgctcttgcagcatccctgactagcgcctataagatcaccggtgacaatgtcaactgccgaagtggtccaggcactagctactccgtaaagaggtccttcaagaagggaactgatgttaccctatcttgtcaaaccaccggagaaaatgttttgggtactagtatctgggacaagacctcatatggctgctatgtctcggactactatgtcaagacaggctccagcggatttgtcgtgaagaagtgcggtacttgcggtgcgccgaagtcgaacgcggctaccgtgaatctcatctccgatttcgagggattcagggcgaatatctgtaagtgttctttgttctttgaagacaataccggaagattaggtatgctaatacgctggcattagacaaggatgctgctggctatcccactgtcggatatgggcacctctgcagcaactcgagatgcaccgacgtcccgtactctatcccactgtctaaggccaatggcaaaaacctcctcgcaactgacatgacagtaagtattctcctctagcattcacttttcacgcactcattgacacctcctcttttacagaaattcgagaagtgcatcacagccatggtcagcagctccgtgactctcaacaagaaccagtatggcgccttggtcagctgggctttcaacatgggttgcggagccactaagacgtctaccctgatcaagcgcctcaaccagggccagaacgtgaacacggtcttgtctactgagctcccgaagtgggtgtacgcaggtggcaagaagctcaatggtcttgttcgccgacgcaacgccgagattgctttggctaagaagaagaccaccgaaaaggctcttccgaacaagtgctag

SEQ ID NO: 305 - protein, 258 aa, Ilyonectria rufa
MKFSLFAVYALAASLTSAYKITGDNVNCRSGPGTSYSVKRSFKKGTDVTLSCQTTGENVLGTSIWDKTSYGCYVSDYYVKTGSSGFVVKKCGTCGAPKSNAATVNLISDFEGFRANIYKDAAGYPTVGYGHLCSNSRCTDVPYSIPLSKANGKNLLATDMTKFEKCITAMVSSSVTLNKNQYGALVSWAFNMGCGATKTSTLIKRLNQGQNVNTVLSTELPKWVYAGGKKLNGLVRRRNAEIALAKKKTTEKALPNKC

SEQ ID NO: 306 - protein, 240 aa, Ilyonectria rufa
YKITGDNVNCRSGPGTSYSVKRSFKKGTDVTLSCQTTGENVLGTSIWDKTSYGCYVSDYYVKTGSSGFVVKKCGTCGAPKSNAATVNLISDFEGFRANIYKDAAGYPTVGYGHLCSNSRCTDVPYSIPLSKANGKNLLATDMTKFEKCITAMVSSSVTLNKNQYGALVSWAFNMGCGATKTSTLIKRLNQGQNVNTVLSTELPKWVYAGGKKLNGLVRRRNAEIALAKKKTTEKALPNKC

(19) United States
(12) Patent Application Publication
(10) Pub. No.: US 2025/0261668 A1
(43) Pub. Date: Aug. 21, 2025

(54) Animal Feed Composition and Uses Thereof

(71) Applicant: Novozymes A/S, Bagsvaerd (DK)

(72) Inventors: Ye LIU, Beijing (CN); Kirk Matthew Schnorr, Holte (DK); Lars Kiemer, Ballerup (DK); Lars Kobberoee Skov, Ballerup (DK); Dorthe Hoej Sandvang, Slangerup (DK); Marianne Thorup Cohn, Copenhagen (DK); Ming Li, Beijing (CN)

(73) Assignee: Novozymes A/S, Bagsvaerd (DK)

(21) Appl. No.: 19/202,706

(22) Filed: May 8, 2025

Related U.S. Application Data
(62) Division of application No. 17/153,096, filed on Jan. 20, 2021, now Pat. No. 12,324,447.
Application No. 17/153,096 is a division of application No. 15/579,769, filed on Dec. 5, 2017, now Pat. No. 10,945,449.

(30) Foreign Application Priority Data
Jul. 2, 2015 (EP) 15175030.4

(51) Int. Cl. A23K 20/189; A23K 20/147; A23K 20/174; A23K 20/28; A23K 50/30; A23K 50/60; A23K 50/75; A23K 50/80; A61K 38/47; C12N 9/36

(52) U.S. Cl. CPC A23K 20/189; A23K 20/147; A23K 20/174; A23K 20/28; A23K 50/30; A23K 50/60; A23K 50/75; A23K 50/80; A61K 38/47; C12N 9/2462; C12Y 302/01017

(57) ABSTRACT

Recombinant host cells comprising a nucleic acid construct or expression vector comprising a polynucleotide encoding a polypeptide having lysozyme activity and at least 80% sequence identity to SEQ ID NO: 264, as well as methods of producing polypeptides having lysozyme activity and at least 80% sequence identity to SEQ ID NO: 264 and compositions comprising polypeptides having lysozyme activity and at least 80% sequence identity to SEQ ID NO: 264.

### CROSS-REFERENCE TO RELATED APPLICATIONS

This application is a continuation of U.S. patent application Ser. No. 17/153,096, filed Jan. 20, 2021, which is a divisional of U.S. patent application Ser. No. 15/579,769, filed Dec. 5, 2017, and issued as U.S. Pat. No. 10,945,449 on Mar. 16, 2021, which is a national stage entry of PCT/CN2016/088362, filed Jul. 4, 2016, which claims priority to European Patent Application No. 15175030.4, filed Jul. 2, 2015. The contents of the aforementioned applications are fully incorporated herein by reference.

### REFERENCE TO A SEQUENCE LISTING

This application contains a Sequence Listing in computer readable form, which is incorporated herein by reference. The name of the file containing the Sequence Listing is “SQ.xml”, which was created on May 7, 2025, and which contains 337,671 bytes.

### BACKGROUND OF THE INVENTION

### Field of the Invention

The present invention relates animal feed or animal feed additives comprising one or more polypeptides having lysozyme activity. The invention also relates to polypeptides having lysozyme activity, polynucleotides encoding the polypeptides nucleic acid constructs, vectors, and host cells comprising the polynucleotides as well as methods of producing and using the polypeptides.

### Description of the Related Art

Lysozyme is an O-glycosyl hydrolase produced as a defensive mechanism against bacteria by many organisms. The enzyme causes the hydrolysis of bacterial cell walls by cleaving the glycosidic bonds of peptidoglycan; an important structural molecule in bacteria. After having their cell walls weakened by lysozyme action, bacterial cells lyse as a result of unbalanced osmotic pressure.

Lysozyme naturally occurs in many organisms such as viruses, plants, insects, birds, reptiles and mammals. In mammals, Lysozyme has been isolated from nasal secretions, saliva, tears, intestinal content, urine and milk. The enzyme cleaves the glycosidic bond between carbon number 1 of N-acetylmuramic acid and carbon number 4 of N-acetyl-D-glucosamine. In vivo, these two carbohydrates are polymerized to form the cell wall polysaccharide of many microorganisms.

Lysozyme has been classified into five different glycoside hydrolase (GH) families (CAZy, cazy.org): hen egg-white lysozyme (GH22), goose egg-white lysozyme (GH23), bacteriophage T4 lysozyme (GH24), Sphingomonas flagellar protein (GH73) and Chalaropsis lysozymes (GH25). Lysozymes from the families GH23 and GH24 are primarily known from bacteriophages and have only recently been identified in fungi. The lysozyme family GH25 has been found to be structurally unrelated to the other lysozyme families.

Lysozyme extracted from hen egg white is the primary product available on the commercial market, but does not cleave N,6-O-diacetylmuramic acid in, e.g., Staphylococcus aureus cell walls and is thus unable to lyse this important human pathogen among others (Masschalck B, Deckers D, Michiels C W (2002), “Lytic and nonlytic mechanism of inactivation of gram-positive bacteria by lysozyme under atmospheric and high hydrostatic pressure”, J Food Prot. 65 (12): 1916-23).

WO 2012/035103 discloses variants of a GH22 lysozyme from the bird Opisthocomus hoazin. WO 2013/076253 and WO 2013/076259 disclose single domain GH24 and GH25 lysozymes for use in animal feed. GB 2002780 discloses the use of a lysozyme in the prophylaxis treatment of chickens. The lysozyme used was the same as isolated by Flemming in 1922 which is a GH22 lysozyme from hen egg white.

CN 101912048 discloses the use of a bacteriophage T4 lysozyme in animal feed. Bacteriophage T4 lysozymes do not comprise the same domain structure as the lysozymes of the present invention. CN 103385366 discloses the use of a lysozyme (unknown type, but based on the activity assay most likely the GH22 lysozyme from hen egg white) for use in animal feed.

Antimicrobial growth promoters (AGP's) have traditionally been used for growth promotion in animals, and probably work by preventing low level infections by pathogens such as Clostridium perfringens. However, AGP's are increasingly being banned worldwide and therefore new solutions to promote animal growth but which are not AGP's are of interest. The object of the present invention is to provide new and effective solutions to this problem.

### SUMMARY OF THE INVENTION

The present invention relates to an animal feed or animal feed additive comprising one or more polypeptides having lysozyme activity, wherein the polypeptide comprises one or more GH24 catalytic domains and one or more lysozyme enhancing domains and wherein:
(a) GH24 catalytic domain gives a domT score of at least 200 when queried using a Profile Hidden Markov Model prepared using SEQ ID NOs: 1 to 122 and hmmbuild software program, and wherein the query is carried out using hmmscan software program with default settings; and
(b) the polypeptide comprises one or more lysozyme enhancing domains, wherein the lysozyme enhancing domain gives a domT score of at least 100 when queried using a Profile Hidden Markov Model prepared using SEQ ID NOs: 123 to 251 and hmmbuild software program, and wherein the query is carried out using the hmmscan software program with default settings.

The invention also relates to a polypeptide having lysozyme activity, selected from the group consisting of:
(a) a polypeptide having at least 80% sequence identity to SEQ ID NO: 257;
(b) a polypeptide having at least 95% sequence identity to SEQ ID NO: 267;
(c) a polypeptide having at least 80% sequence identity to SEQ ID NO: 291;
(d) a polypeptide having at least 80% sequence identity to SEQ ID NO: 294;
(e) a polypeptide having at least 82% sequence identity to SEQ ID NO: 297;
(f) a polypeptide having at least 80% sequence identity to SEQ ID NO: 300;
(g) a polypeptide having at least 80% sequence identity to SEQ ID NO: 303; (g)
(h) a polypeptide having at least 80% sequence identity to SEQ ID NO: 306;
(i) a polypeptide encoded by a polynucleotide that hybridizes under medium-high stringency conditions, high stringency conditions or very-high stringency conditions with:
   (i) the mature polypeptide coding sequence of SEQ ID NO: 255;
   (ii) the mature polypeptide coding sequence of SEQ ID NO: 287;
   (iii) the mature polypeptide coding sequence of SEQ ID NO: 292;
   (iv) the mature polypeptide coding sequence of SEQ ID NO: 295;
   (v) the mature polypeptide coding sequence of SEQ ID NO: 298;
   (vi) the mature polypeptide coding sequence of SEQ ID NO: 301;
   (vii) the mature polypeptide coding sequence of SEQ ID NO: 304;
   (viii) the cDNA sequence thereof; or
   (ix) the full-length complement of (i), (ii), (iii), (iv) (v), (vi), (vii) or (viii);
(j) a polypeptide encoded by a polynucleotide that hybridizes under very-high stringency conditions with:
   (i) the mature polypeptide coding sequence of SEQ ID NO: 265;
   (ii) the full-length complement of (i);
(k) a polypeptide encoded by a polynucleotide having at least 80% sequence identity to the mature polypeptide coding sequence of SEQ ID NO: 255;
(l) a polypeptide encoded by a polynucleotide having at least 95% sequence identity to the mature polypeptide coding sequence of SEQ ID NO: 265;
(m) a polypeptide encoded by a polynucleotide having at least 80% sequence identity to the mature polypeptide coding sequence of SEQ ID NO: 287;
(n) a polypeptide encoded by a polynucleotide having at least 80% sequence identity to the mature polypeptide coding sequence of SEQ ID NO: 292;
(o) a polypeptide encoded by a polynucleotide having at least 80% sequence identity to the mature polypeptide coding sequence of SEQ ID NO: 295;
(p) a polypeptide encoded by a polynucleotide having at least 82% sequence identity to the mature polypeptide coding sequence of SEQ ID NO: 298;
(q) a polypeptide encoded by a polynucleotide having at least 80% sequence identity to the mature polypeptide coding sequence of SEQ ID NO: 301;
(r) a polypeptide encoded by a polynucleotide having at least 80% sequence identity to the mature polypeptide coding sequence of SEQ ID NO: 304;
(s) a variant of SEQ ID NO: 257, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions; and
(t) a variant of SEQ ID NO: 267, wherein the variant has lysozyme activity and comprises one or more substitutions and/or one or more deletions and/or one or more insertions or any combination thereof in 1, 2, 3, 4, 5, 6, 7, 8, 9, 10, 11 or 12 positions; and
(u) a variant of SEQ ID NO: 291, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(v) a variant of SEQ ID NO: 294, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(w) a variant of SEQ ID NO: 297, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(x) a variant of SEQ ID NO: 300, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(y) a variant of SEQ ID NO: 303, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(z) a variant of SEQ ID NO: 306, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions; and
(aa) a fragment of the polypeptide of (a), (b), (c), (d), (e), (f), (g), (h), (i), (j), (k), (l), (m), (n), (o), (p), (q), (r), (s), (t), (u), (v), (w), (x), (y) or (z) that has lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids.

The invention further relates to compositions comprising the lysozyme of the invention; use of the lysozyme of the invention in animal feed, in animal feed additives, in the preparation of a composition for use in animal feed, for improving one or more performance parameters in an animal, for the treatment of Clostridium perfringens infection in an animal; for the treatment of necrotic enteritis, and as a medicament; methods of treatment of a Clostridium perfringens infection and/or necrotic enteritis in an animal; method of improving the performance of an animal; and isolated polynucleotides encoding the polypeptides of the invention.

### Overview of Sequence Listing

SEQ ID NO: 1 is the amino acid sequence of the GH24 catalytic domain of SWISSPROT:A6R5W4 from Ajellomyces Capsulatus.

SEQ ID NO: 2 is the amino acid sequence of the GH24 catalytic domain of SWISSPROT:A8PEU5 from Coprinopsis cinerea.

SEQ ID NO: 3 is the amino acid sequence of the GH24 catalytic domain of SWISSPROT:Q0CMX2 from Aspergillus terreus.

SEQ ID NO: 4 is the amino acid sequence of the GH24 catalytic domain of SWISSPROT:QOD1K1 from Aspergillus terreus.

SEQ ID NO: 5 is the amino acid sequence of the GH24 catalytic domain of SWISSPROT:Q2TXN3 from Aspergillus oryzae.

SEQ ID NO: 6 is the amino acid sequence of the GH24 catalytic domain of SWISSPROT:Q2UFQ2 from Aspergillus oryzae.

SEQ ID NO: 7 is the amino acid sequence of the GH24 catalytic domain of SWISSPROT:Q5B9R 1 from Emericella nidulans.

SEQ ID NO: 8 is the amino acid sequence of the GH24 catalytic domain of SWISSPROT:B0DQF 7 from Laccaria bicolor.

SEQ ID NO: 9 is the amino acid sequence of the GH24 catalytic domain of SWISSPROT:B8NGV0 from Aspergillus flavus.

SEQ ID NO: 10 is the amino acid sequence of the GH24 catalytic domain of SWISSPROT:B8NVG8 from Aspergillus flavus.

SEQ ID NO: 11 is the amino acid sequence of the GH24 catalytic domain of SWISSPROT:C0NHY3 from Ajellomyces capsulatus.

SEQ ID NO: 12 is the amino acid sequence of the GH24 catalytic domain of SWISSPROT:C5FQ03 from Arthroderma otae.

SEQ ID NO: 13 is the amino acid sequence of the GH24 catalytic domain of SWISSPROT:C5FZ72 from Arthroderma otae.

SEQ ID NO: 14 is the amino acid sequence of the GH24 catalytic domain of SWISSPROT:C5GQK2 from Ajellomyces dermatitidis.

SEQ ID NO: 15 is the amino acid sequence of the GH24 catalytic domain of SWISSPROT:C6H5Y9 from Ajellomyces capsulatus.

SEQ ID NO: 16 is the amino acid sequence of the GH24 catalytic domain of SWISSPROT:D4AT26 from Arthroderma benhamiae.

SEQ ID NO: 17 is the amino acid sequence of the GH24 catalytic domain of SWISSPROT:D4CZH0 from Trichophyton verrucosum.

SEQ ID NO: 18 is the amino acid sequence of the GH24 catalytic domain of SWISSPROT:D4DKV0 from Trichophyton verrucosum.

SEQ ID NO: 19 is the amino acid sequence of the GH24 catalytic domain of SWISSPROT:E4UVW7 from Arthroderma gypseum.

SEQ ID NO: 20 is the amino acid sequence of the GH24 catalytic domain of SWISSPROT:E4V546 from Arthroderma gypseum.

SEQ ID NO: 21 is the amino acid sequence of the GH24 catalytic domain of SWISSPROT:G9N4L5 from Hypocrea virens.

SEQ ID NO: 22 is the amino acid sequence of the GH24 catalytic domain of SWISSPROT:E 9F1Z9 from Metarhizium robertsii.

SEQ ID NO: 23 is the amino acid sequence of the GH24 catalytic domain of SWISSPROT:F0UVK8 from Ajellomyces capsulatus.

SEQ ID NO: 24 is the amino acid sequence of the GH24 catalytic domain of SWISSPROT:F2RV71 from Trichophyton tonsurans.

SEQ ID NO: 25 is the amino acid sequence of the GH24 catalytic domain of SWISSPROT:F2PV65 from Trichophyton equinum.

SEQ ID NO: 26 is the amino acid sequence of the GH24 catalytic domain of SWISSPROT:F2SGL6 from Trichophyton rubrum.

SEQ ID NO: 27 is the amino acid sequence of the GH24 catalytic domain of SWISSPROT:F2SJH3 from Trichophyton rubrum.

SEQ ID NO: 28 is the amino acid sequence of the GH24 catalytic domain of SWISSPROT:L7ZJC4 from Serratia marcescens.

SEQ ID NO: 29 is the amino acid sequence of the GH24 catalytic domain of SWISSPROT:J5TH48 from Trichosporon asahii.

SEQ ID NO: 30 is the amino acid sequence of the GH24 catalytic domain of SWISSPROT:J4UH35 from Trichosporon asahii.

SEQ ID NO: 31 is the amino acid sequence of the GH24 catalytic domain of SWISSPROT:K1VMN5 from Trichosporon asahii.

SEQ ID NO: 32 is the amino acid sequence of the GH24 catalytic domain of SWISSPROT:K1WL46 from Trichosporon asahii.

SEQ ID NO: 33 is the amino acid sequence of the GH24 catalytic domain of SWISSPROT:U9T5J9 from Rhizophagus irregularis.

SEQ ID NO: 34 is the amino acid sequence of the GH24 catalytic domain of SWISSPROT:U9TCU3 from Rhizophagus irregularis.

SEQ ID NO: 35 is the amino acid sequence of the GH24 catalytic domain of SWISSPROT:U9UUS9 from Rhizophagus irregularis.

SEQ ID NO: 36 is the amino acid sequence of the GH24 catalytic domain of SWISSPROT:R7Z482 from Coniosporium apollinis.

SEQ ID NO: 37 is the amino acid sequence of the GH24 catalytic domain of SWISSPROT:A0A015K155 from Rhizophagus irregularis.

SEQ ID NO: 38 is the amino acid sequence of the GH24 catalytic domain of SWISSPROT:W6PQ31 from Penicillium roqueforti.

SEQ ID NO: 39 is the amino acid sequence of the GH24 catalytic domain of SWISSPROT:A0A017SCH5 from Aspergillus ruber.

SEQ ID NO: 40 is the amino acid sequence of the GH24 catalytic domain of SWISSPROT:A0A022UIH6 from Trichophyton interdigitale.

SEQ ID NO: 41 is the amino acid sequence of the GH24 catalytic domain of SWISSPROT:A0A015LDX8 from Rhizophagus irregularis.

SEQ ID NO: 42 is the amino acid sequence of the GH24 catalytic domain of SWISSPROT:A0A015L2T0 from Rhizophagus irregularis.

SEQ ID NO: 43 is the amino acid sequence of the GH24 catalytic domain of SWISSPROT:A0A086NFK5 from Metarhizium anisopliae.

SEQ ID NO: 44 is the amino acid sequence of the GH24 catalytic domain of SWISSPROT:A0A0A2V9H7 from Beauveria bassiana.

SEQ ID NO: 45 is the amino acid sequence of the GH24 catalytic domain of SWISSPROT:A0A0A2W020 from Beauveria bassiana.

SEQ ID NO: 46 is the amino acid sequence of the GH24 catalytic domain of SWISSPROT:A0A0A2W5I8 from Beauveria bassiana.

SEQ ID NO: 47 is the amino acid sequence of the GH24 catalytic domain of SWISSPROT:A0A0B4HPE0 from Metarhizium guizhouense.

SEQ ID NO: 48 is the amino acid sequence of the GH24 catalytic domain of SWISSPROT:A0A0B4HHE 7 from Metarhizium majus.

SEQ ID NO: 49 is the amino acid sequence of the GH24 catalytic domain of SWISSPROT:A0A0B4FYT8 from Metarhizium brunneum.

SEQ ID NO: 50 is the amino acid sequence of the GH24 catalytic domain of SWISSPROT:A8PEP0 from Coprinopsis cinerea.

SEQ ID NO: 51 is the amino acid sequence of the GH24 catalytic domain of SWISSPROT:A8PEP 4 from Coprinopsis cinerea.

SEQ ID NO: 52 is the amino acid sequence of the GH24 catalytic domain of SWISSPROT:A8PEQ3 from Coprinopsis cinerea.

SEQ ID NO: 53 is the amino acid sequence of the GH24 catalytic domain of SWISSPROT:B0DQ11 from Laccaria bicolor.

SEQ ID NO: 54 is the amino acid sequence of the GH24 catalytic domain of SWISSPROT:B0DUH4 from Laccaria bicolor.

SEQ ID NO: 55 is the amino acid sequence of the GH24 catalytic domain of SWISSPROT:C7Z8W0 from Nectria haematococca.

SEQ ID NO: 56 is the amino acid sequence of the GH24 catalytic domain of SWISSPROT:C7ZQ20 from Nectria haematococca.

SEQ ID NO: 57 is the amino acid sequence of the GH24 catalytic domain of SWISSPROT:GORP87 from Hypocrea jecorina.

SEQ ID NO: 58 is the amino acid sequence of the GH24 catalytic domain of SWISSPROT:A8PEQ1 from Coprinopsis cinerea.

SEQ ID NO: 59 is the amino acid sequence of the GH24 catalytic domain of SWISSPROT:D6RMD2 from Coprinopsis cinerea.

SEQ ID NO: 60 is the amino acid sequence of the GH24 catalytic domain of SWISSPROT:K91708 from Agaricus bisporus.

SEQ ID NO: 61 is the amino acid sequence of the GH24 catalytic domain of SWISSPROT:E9DUC 1 from Metarhizium acridum.

SEQ ID NO: 62 is the amino acid sequence of the GH24 catalytic domain of SWISSPROT:E9FAC9 from Metarhizium robertsii.

SEQ ID NO: 63 is the amino acid sequence of the GH24 catalytic domain of SWISSPROT:F9GF09 from Fusarium oxysporum.

SEQ ID NO: 64 is the amino acid sequence of the GH24 catalytic domain of SWISSPROT:A0A067NV59 from Pleurotus ostreatus.

SEQ ID NO: 65 is the amino acid sequence of the GH24 catalytic domain of SWISSPROT:A0A067NBT7 from Pleurotus ostreatus.

SEQ ID NO: 66 is the amino acid sequence of the GH24 catalytic domain of SWISSPROT:A0A067NLC3 from Pleurotus ostreatus.

SEQ ID NO: 67 is the amino acid sequence of the GH24 catalytic domain of SWISSPROT:A0A067TC 70 from Galerina marginata.

SEQ ID NO: 68 is the amino acid sequence of the GH24 catalytic domain of SWISSPROT:A0A067T7N7 from Galerina marginata.

SEQ ID NO: 69 is the amino acid sequence of the GH24 catalytic domain of SWISSPROT:K5VNL0 from Agaricus bisporus.

SEQ ID NO: 70 is the amino acid sequence of the GH24 catalytic domain of SWISSPROT:W9Z992 from Fusarium oxysporum.

SEQ ID NO: 71 is the amino acid sequence of the GH24 catalytic domain of SWISSPROT:W 9ZZW9 from Fusarium oxysporum.

SEQ ID NO: 72 is the amino acid sequence of the GH24 catalytic domain of SWISSPROT:X0A5V9 from Fusarium oxysporum.

SEQ ID NO: 73 is the amino acid sequence of the GH24 catalytic domain of SWISSPROT:N4UD22 from Fusarium oxysporum.

SEQ ID NO: 74 is the amino acid sequence of the GH24 catalytic domain of SWISSPROT:N4UKT7 from Fusarium oxysporum.

SEQ ID NO: 75 is the amino acid sequence of the GH24 catalytic domain of SWISSPROT:W9KX02 from Fusarium oxysporum.

SEQ ID NO: 76 is the amino acid sequence of the GH24 catalytic domain of SWISSPROT:X0MM97 from Fusarium oxysporum.

SEQ ID NO: 77 is the amino acid sequence of the GH24 catalytic domain of SWISSPROT:W7N5Q6 from Gibberella moniliformis.

SEQ ID NO: 78 is the amino acid sequence of the GH24 catalytic domain of SWISSPROT:X0BII8 from Fusarium oxysporum.

SEQ ID NO: 79 is the amino acid sequence of the GH24 catalytic domain of SWISSPROT:W9NXR 4 from Fusarium oxysporum.

SEQ ID NO: 80 is the amino acid sequence of the GH24 catalytic domain of SWISSPROT:W9LDD0 from Fusarium oxysporum.

SEQ ID NO: 81 is the amino acid sequence of the GH24 catalytic domain of SWISSPROT:W9HEM8 from Fusarium oxysporum.

SEQ ID NO: 82 is the amino acid sequence of the GH24 catalytic domain of SWISSPROT:S0EP16 from Gibberella fujikuroi.

SEQ ID NO: 83 is the amino acid sequence of the GH24 catalytic domain of SWISSPROT:S3D2D9 from Glarea lozoyensis.

SEQ ID NO: 84 is the amino acid sequence of the GH24 catalytic domain of SWISSPROT:R7Z235 from Coniosporium apollinis.

SEQ ID NO: 85 is the amino acid sequence of the GH24 catalytic domain of SWISSPROT:U4LG 64 from Pyronema omphalodes.

SEQ ID NO: 86 is the amino acid sequence of the GH24 catalytic domain of SWISSPROT:U1HSJ9 from Endocarpon pusillum.

SEQ ID NO: 87 is the amino acid sequence of the GH24 catalytic domain of SWISSPROT:U4LQS4 from Pyronema omphalodes.

SEQ ID NO: 88 is the amino acid sequence of the GH24 catalytic domain of SWISSPROT:X0IVL1 from Fusarium oxysporum.

SEQ ID NO: 89 is the amino acid sequence of the GH24 catalytic domain of SWISSPROT:A0A086T9F0 from Acremonium chrysogenum.

SEQ ID NO: 90 is the amino acid sequence of the GH24 catalytic domain of SWISSPROT:A0A086NUM9 from Metarhizium anisopliae.

SEQ ID NO: 91 is the amino acid sequence of the GH24 catalytic domain of SWISSPROT:A0A0A1TCW0 from Torrubiella hemipterigena.

SEQ ID NO: 92 is the amino acid sequence of the GH24 catalytic domain of SWISSPROT:A0A093ZCQ3 from Pseudogymnoascus pannorum.

SEQ ID NO: 93 is the amino acid sequence of the GH24 catalytic domain of SWISSPROT:A0A0B4HLN8 from Metarhizium majus.

SEQ ID NO: 94 is the amino acid sequence of the GH24 catalytic domain of SWISSPROT:A0A0B4H8G 1 from Metarhizium guizhouense.

SEQ ID NO: 95 is the amino acid sequence of the GH24 catalytic domain of SWISSPROT:A4UCC9 from Magnaporthe oryzae.

SEQ ID NO: 96 is the amino acid sequence of the GH24 catalytic domain of SWISSPROT:Q2GND9 from Chaetomium globosum.

SEQ ID NO: 97 is the amino acid sequence of the GH24 catalytic domain of SWISSPROT:C7Z967 from Nectria haematococca.

SEQ ID NO: 98 is the amino acid sequence of the GH24 catalytic domain of SWISSPROT:C9SY46 from Verticillium alfalfae.

SEQ ID NO: 99 is the amino acid sequence of the GH24 catalytic domain of SWISSPROT:G2RG69 from Thielavia terrestris.

SEQ ID NO: 100 is the amino acid sequence of the GH24 catalytic domain of SWISSPROT:G2QNE9 from Thielavia heterothallica.

SEQ ID NO: 101 is the amino acid sequence of the GH24 catalytic domain of SWISSPROT:J 9NQV9 from Fusarium oxysporum.

SEQ ID NO: 102 is the amino acid sequence of the GH24 catalytic domain of SWISSPROT:F9F2K4 from Fusarium oxysporum.

SEQ ID NO: 103 is the amino acid sequence of the GH24 catalytic domain of SWISSPROT:GORZV2 from Chaetomium thermophilum.

SEQ ID NO: 104 is the amino acid sequence of the GH24 catalytic domain of SWISSPROT:G2XHC2 from Verticillium dahliae.

SEQ ID NO: 105 is the amino acid sequence of the GH24 catalytic domain of SWISSPROT:N1RWA4 from Fusarium oxysporum.

SEQ ID NO: 106 is the amino acid sequence of the GH24 catalytic domain of SWISSPROT:X0B4J3 from Fusarium oxysporum.

SEQ ID NO: 107 is the amino acid sequence of the GH24 catalytic domain of SWISSPROT:A0A084PYR3 from Stachybotrys chartarum.

SEQ ID NO: 108 is the amino acid sequence of the GH24 catalytic domain of SWISSPROT:A0A086TBZ4 from Acremonium chrysogenum.

SEQ ID NO: 109 is the amino acid sequence of the GH24 catalytic domain of SWISSPROT:A0A086SUF8 from Acremonium chrysogenum.

SEQ ID NO: 110 is the amino acid sequence of the GH24 catalytic domain of SWISSPROT:A0A084R 326 from Stachybotrys chlorohalonata.

SEQ ID NO: 111 is the amino acid sequence of the GH24 catalytic domain of SWISSPROT:A0A093Z6Z8 from Pseudogymnoascus pannorum.

SEQ ID NO: 112 is the amino acid sequence of the GH24 catalytic domain of SWISSPROT:A0A094IML3 from Pseudogymnoascus pannorum.

SEQ ID NO: 113 is the amino acid sequence of the GH24 catalytic domain of SWISSPROT:A0A094GY79 from Pseudogymnoascus pannorum.

SEQ ID NO: 114 is the amino acid sequence of the GH24 catalytic domain of SWISSPROT:A0A093XPZ7 from Pseudogymnoascus pannorum.

SEQ ID NO: 115 is the amino acid sequence of the GH24 catalytic domain of SWISSPROT:A0A093XAS9 from Pseudogymnoascus pannorum.

SEQ ID NO: 116 is the amino acid sequence of the GH24 catalytic domain of SWISSPROT:A0A0A1TNV6 from Torrubiella hemipterigena.

SEQ ID NO: 117 is the amino acid sequence of the GH24 catalytic domain of SWISSPROT:A0A094DVF4 from Pseudogymnoascus pannorum.

SEQ ID NO: 118 is the amino acid sequence of the GH24 catalytic domain of SWISSPROT:A0A094IE 25 from Pseudogymnoascus pannorum.

SEQ ID NO: 119 is the amino acid sequence of the GH24 catalytic domain of SWISSPROT:A0A094HNM8 from Pseudogymnoascus pannorum.

SEQ ID NO: 120 is the amino acid sequence of the GH24 catalytic domain of SWISSPROT:A0A094EPJ 7 from Pseudogymnoascus pannorum.

SEQ ID NO: 121 is the amino acid sequence of the GH24 catalytic domain of SWISSPROT:A0A094BWD6 from Pseudogymnoascus pannorum.

SEQ ID NO: 122 is the amino acid sequence of the GH24 catalytic domain of SWISSPROT:A0A093ZTZ8 from Pseudogymnoascus pannorum.

SEQ ID NO: 123 is the amino acid sequence of the lysozyme enhancing domain of SWISSPROT:B2ASY2 from Podospora anserina.

SEQ ID NO: 124 is the amino acid sequence of the lysozyme enhancing domain of SWISSPROT:B6GZX8 from Penicillium chrysogenum.

SEQ ID NO: 125 is the amino acid sequence of the lysozyme enhancing domain of SWISSPROT:C7ZQ22 from Nectria haematococca.

SEQ ID NO: 126 is the amino acid sequence of the lysozyme enhancing domain of SWISSPROT:E9DSA6 from Metarhizium acridum.

SEQ ID NO: 127 is the amino acid sequence of the lysozyme enhancing domain of SWISSPROT:E9F1Z9 from Metarhizium robertsii.

SEQ ID NO: 128 is the amino acid sequence of the lysozyme enhancing domain of SWISSPROT:E9FC42 from Metarhizium robertsii.

SEQ ID NO: 129 is the amino acid sequence of the lysozyme enhancing domain of SWISSPROT:F9F2K5 from Fusarium oxysporum.

SEQ ID NO: 130 is the amino acid sequence of the lysozyme enhancing domain of SWISSPROT:F9GF09 from Fusarium oxysporum.

SEQ ID NO: 131 is the amino acid sequence of the lysozyme enhancing domain of SWISSPROT:G2QV10 from Thielavia terrestris.

SEQ ID NO: 132 is the amino acid sequence of the lysozyme enhancing domain of SWISSPROT:G2QV26 from Thielavia terrestris.

SEQ ID NO: 133 is the amino acid sequence of the lysozyme enhancing domain of SWISSPROT:J 5TH48 from Trichosporon asahii var. Asahii.

SEQ ID NO: 134 is the amino acid sequence of the lysozyme enhancing domain of SWISSPROT:J 4UH35 from Trichosporon asahii var. Asahii.

SEQ ID NO: 135 is the amino acid sequence of the lysozyme enhancing domain of SWISSPROT:J 9NQ28 from Fusarium oxysporum f. sp. Lycopersici.

SEQ ID NO: 136 is the amino acid sequence of the lysozyme enhancing domain of SWISSPROT:J 9NQW0 from Fusarium oxysporum f. sp. Lycopersici.

SEQ ID NO: 137 is the amino acid sequence of the lysozyme enhancing domain of SWISSPROT:K1VMN5 from Trichosporon asahii var. Asahii.

SEQ ID NO: 138 is the amino acid sequence of the lysozyme enhancing domain of SWISSPROT:K1WL46 from Trichosporon asahii var. Asahii.

SEQ ID NO: 139 is the amino acid sequence of the lysozyme enhancing domain of SWISSPROT:W9Z045 from Fusarium oxysporum f. sp. Melonis.

SEQ ID NO: 140 is the amino acid sequence of the lysozyme enhancing domain of SWISSPROT:X0A5V9 from Fusarium oxysporum f. sp. Melonis.

SEQ ID NO: 141 is the amino acid sequence of the lysozyme enhancing domain of SWISSPROT:N1S551 from Fusarium oxysporum f. sp. Cubense.

SEQ ID NO: 142 is the amino acid sequence of the lysozyme enhancing domain of SWISSPROT:N4UD22 from Fusarium oxysporum f. sp. Cubense.

SEQ ID NO: 143 is the amino acid sequence of the lysozyme enhancing domain of SWISSPROT:N4UT47 from Fusarium oxysporum f. sp. Cubense.

SEQ ID NO: 144 is the amino acid sequence of the lysozyme enhancing domain of SWISSPROT:W9KWW4 from Fusarium oxysporum.

SEQ ID NO: 145 is the amino acid sequence of the lysozyme enhancing domain of SWISSPROT:X0MM97 from Fusarium oxysporum f. sp. Vasinfectum.

SEQ ID NO: 146 is the amino acid sequence of the lysozyme enhancing domain of SWISSPROT:W7N5Q6 from Gibberella moniliformis.

SEQ ID NO: 147 is the amino acid sequence of the lysozyme enhancing domain of SWISSPROT:X0BE07 from Fusarium oxysporum f. sp. Raphani.

SEQ ID NO: 148 is the amino acid sequence of the lysozyme enhancing domain of SWISSPROT:X0BII8 from Fusarium oxysporum.

SEQ ID NO: 149 is the amino acid sequence of the lysozyme enhancing domain of SWISSPROT:W9NW59 from Fusarium oxysporum f. sp. Pisi.

SEQ ID NO: 150 is the amino acid sequence of the lysozyme enhancing domain of SWISSPROT:W9NXR4 from Fusarium oxysporum f. sp. Pisi.

SEQ ID NO: 151 is the amino acid sequence of the lysozyme enhancing domain of SWISSPROT:S7ZNE7 from Penicillium oxalicum.

SEQ ID NO: 152 is the amino acid sequence of the lysozyme enhancing domain of SWISSPROT:S7Z5Z6 from Penicillium oxalicum.

SEQ ID NO: 153 is the amino acid sequence of the lysozyme enhancing domain of SWISSPROT:W9LDD0 from Fusarium oxysporum f. sp. Lycopersici.

SEQ ID NO: 154 is the amino acid sequence of the lysozyme enhancing domain of SWISSPROT:W9HEM8 from Fusarium oxysporum.

SEQ ID NO: 155 is the amino acid sequence of the lysozyme enhancing domain of SWISSPROT:W9JDH4 from Fusarium oxysporum.

SEQ ID NO: 156 is the amino acid sequence of the lysozyme enhancing domain of SWISSPROT:S0EP16 from Gibberella fujikuroi.

SEQ ID NO: 157 is the amino acid sequence of the lysozyme enhancing domain of SWISSPROT:W6QNL2 from Penicillium roqueforti.

SEQ ID NO: 158 is the amino acid sequence of the lysozyme enhancing domain of SWISSPROT:U4LJD9 from Pyronema omphalodes.

SEQ ID NO: 159 is the amino acid sequence of the lysozyme enhancing domain of SWISSPROT:U4LG64 from Pyronema omphalodes.

SEQ ID NO: 160 is the amino acid sequence of the lysozyme enhancing domain of SWISSPROT:A0A094AK50 from Pseudogymnoascus pannorum.

SEQ ID NO: 161 is the amino acid sequence of the lysozyme enhancing domain of SWISSPROT:W9JIH2 from Fusarium oxysporum.

SEQ ID NO: 162 is the amino acid sequence of the lysozyme enhancing domain of SWISSPROT:X0FW82 from Fusarium oxysporum f. sp. radicis-lycopersici.

SEQ ID NO: 163 is the amino acid sequence of the lysozyme enhancing domain of SWISSPROT:A0A086TBY7 from Acremonium chrysogenum.

SEQ ID NO: 164 is the amino acid sequence of the lysozyme enhancing domain of SWISSPROT:A0A086T755 from Acremonium chrysogenum.

SEQ ID NO: 165 is the amino acid sequence of the lysozyme enhancing domain of SWISSPROT:A0A086T4C8 from Acremonium chrysogenum.

SEQ ID NO: 166 is the amino acid sequence of the lysozyme enhancing domain of SWISSPROT:A0A086NNR4 from Metarhizium anisopliae.

SEQ ID NO: 167 is the amino acid sequence of the lysozyme enhancing domain of SWISSPROT:A0A086NFK5 from Metarhizium anisopliae.

SEQ ID NO: 168 is the amino acid sequence of the lysozyme enhancing domain of SWISSPROT:A0A094FY19 from Pseudogymnoascus pannorum.

SEQ ID NO: 169 is the amino acid sequence of the lysozyme enhancing domain of SWISSPROT:A0A094G1N0 from Pseudogymnoascus pannorum.

SEQ ID NO: 170 is the amino acid sequence of the lysozyme enhancing domain of SWISSPROT:A0A094GEA0 from Pseudogymnoascus pannorum.

SEQ ID NO: 171 is the amino acid sequence of the lysozyme enhancing domain of SWISSPROT:A0A094GJR5 from Pseudogymnoascus pannorum.

SEQ ID NO: 172 is the amino acid sequence of the lysozyme enhancing domain of SWISSPROT:A0A094G660 from Pseudogymnoascus pannorum.

SEQ ID NO: 173 is the amino acid sequence of the lysozyme enhancing domain of SWISSPROT:A0A093YBN4 from Pseudogymnoascus pannorum.

SEQ ID NO: 174 is the amino acid sequence of the lysozyme enhancing domain of SWISSPROT:A0A094FSZ5 from Pseudogymnoascus pannorum.

SEQ ID NO: 175 is the amino acid sequence of the lysozyme enhancing domain of SWISSPROT:A0A094FBW1 from Pseudogymnoascus pannorum.

SEQ ID NO: 176 is the amino acid sequence of the lysozyme enhancing domain of SWISSPROT:A0A094H0G2 from Pseudogymnoascus pannorum.

SEQ ID NO: 177 is the amino acid sequence of the lysozyme enhancing domain of SWISSPROT:A0A094H7M1 from Pseudogymnoascus pannorum.

SEQ ID NO: 178 is the amino acid sequence of the lysozyme enhancing domain of SWISSPROT:A0A093Y8W3 from Pseudogymnoascus pannorum.

SEQ ID NO: 179 is the amino acid sequence of the lysozyme enhancing domain of SWISSPROT:A0A094GYN9 from Pseudogymnoascus pannorum.

SEQ ID NO: 180 is the amino acid sequence of the lysozyme enhancing domain of SWISSPROT:A0A093XAD4 from Pseudogymnoascus pannorum.

SEQ ID NO: 181 is the amino acid sequence of the lysozyme enhancing domain of SWISSPROT:A0A094H7J6 from Pseudogymnoascus pannorum.

SEQ ID NO: 182 is the amino acid sequence of the lysozyme enhancing domain of SWISSPROT:A0A094E0I1 from Pseudogymnoascus pannorum.

SEQ ID NO: 183 is the amino acid sequence of the lysozyme enhancing domain of SWISSPROT:A0A094CC50 from Pseudogymnoascus pannorum.

SEQ ID NO: 184 is the amino acid sequence of the lysozyme enhancing domain of SWISSPROT:A0A0941195 from Pseudogymnoascus pannorum.

SEQ ID NO: 185 is the amino acid sequence of the lysozyme enhancing domain of SWISSPROT:A0A094IAA0 from Pseudogymnoascus pannorum.

SEQ ID NO: 186 is the amino acid sequence of the lysozyme enhancing domain of SWISSPROT:A0A094IBC0 from Pseudogymnoascus pannorum.

SEQ ID NO: 187 is the amino acid sequence of the lysozyme enhancing domain of SWISSPROT:A0A094E946 from Pseudogymnoascus pannorum.

SEQ ID NO: 188 is the amino acid sequence of the lysozyme enhancing domain of SWISSPROT:A0A094A3A0 from Pseudogymnoascus pannorum.

SEQ ID NO: 189 is the amino acid sequence of the lysozyme enhancing domain of SWISSPROT:A1C4L9 from Aspergillus clavatus.

SEQ ID NO: 190 is the amino acid sequence of the lysozyme enhancing domain of SWISSPROT:A1CBV9 from Aspergillus clavatus.

SEQ ID NO: 191 is the amino acid sequence of the lysozyme enhancing domain of SWISSPROT:A1DA80 from Neosartorya fischeri.

SEQ ID NO: 192 is the amino acid sequence of the lysozyme enhancing domain of SWISSPROT:A1DBW2 from Neosartorya fischeri.

SEQ ID NO: 193 is the amino acid sequence of the lysozyme enhancing domain of SWISSPROT:A1DDF2 from Neosartorya fischeri.

SEQ ID NO: 194 is the amino acid sequence of the lysozyme enhancing domain of SWISSPROT:Q0CED1 from Aspergillus terreus.

SEQ ID NO: 195 is the amino acid sequence of the lysozyme enhancing domain of SWISSPROT:Q0CED2 from Aspergillus terreus.

SEQ ID NO: 196 is the amino acid sequence of the lysozyme enhancing domain of SWISSPROT:Q0CV85 from Aspergillus terreus.

SEQ ID NO: 197 is the amino acid sequence of the lysozyme enhancing domain of SWISSPROT:Q2GND8 from Chaetomium globosum.

SEQ ID NO: 198 is the amino acid sequence of the lysozyme enhancing domain of SWISSPROT:Q2GND9 from Chaetomium globosum.

SEQ ID NO: 199 is the amino acid sequence of the lysozyme enhancing domain of SWISSPROT:Q2H6W7 from Chaetomium globosum.

SEQ ID NO: 200 is the amino acid sequence of the lysozyme enhancing domain of SWISSPROT:Q4WAY2 from Neosartorya fumigata.

SEQ ID NO: 201 is the amino acid sequence of the lysozyme enhancing domain of SWISSPROT:Q4WBR4 from Neosartorya fumigata.

SEQ ID NO: 202 is the amino acid sequence of the lysozyme enhancing domain of SWISSPROT:Q4WVY 3 from Neosartorya fumigata.

SEQ ID NO: 203 is the amino acid sequence of the lysozyme enhancing domain of SWISSPROT:B6H9X5 from Penicillium chrysogenum.
SEQ ID NO: 204 is the amino acid sequence of the lysozyme enhancing domain of SWISSPROT:B6 HR 38 from Penicillium chrysogenum.

SEQ ID NO: 205 is the amino acid sequence of the lysozyme enhancing domain of SWISSPROT:C7Z8W0 from Nectria haematococca.

SEQ ID NO: 206 is the amino acid sequence of the lysozyme enhancing domain of SWISSPROT:C7ZQ20 from Nectria haematococca.

SEQ ID NO: 207 is the amino acid sequence of the lysozyme enhancing domain of SWISSPROT:G0RP87 from Hypocrea jecorina.

SEQ ID NO: 208 is the amino acid sequence of the lysozyme enhancing domain of SWISSPROT:G2RG69 from Thielavia terrestris.

SEQ ID NO: 209 is the amino acid sequence of the lysozyme enhancing domain of SWISSPROT:G2QNE9 from Thielavia heterothallica.

SEQ ID NO: 210 is the amino acid sequence of the lysozyme enhancing domain of SWISSPROT:G0RM22 from Hypocrea jecorina.

SEQ ID NO: 211 is the amino acid sequence of the lysozyme enhancing domain of SWISSPROT:G0SG36 from Chaetomium thermophilum var. Thermophilum.

SEQ ID NO: 212 is the amino acid sequence of the lysozyme enhancing domain of SWISSPROT:G0RZV3 from Chaetomium thermophilum var. Thermophilum.

SEQ ID NO: 213 is the amino acid sequence of the lysozyme enhancing domain of SWISSPROT:J9NQV9 from Fusarium oxysporum f. sp. Lycopersici.

SEQ ID NO: 214 is the amino acid sequence of the lysozyme enhancing domain of SWISSPROT:G2QD02 from Thielavia heterothallica.

SEQ ID NO: 215 is the amino acid sequence of the lysozyme enhancing domain of SWISSPROT:G2QNF0 from Thielavia heterothallica.

SEQ ID NO: 216 is the amino acid sequence of the lysozyme enhancing domain of SWISSPROT:G9MHR1 from Hypocrea virens.

SEQ ID NO: 217 is the amino acid sequence of the lysozyme enhancing domain of SWISSPROT:E9ELX9 from Metarhizium robertsii.

SEQ ID NO: 218 is the amino acid sequence of the lysozyme enhancing domain of SWISSPROT:F9F2K4 from Fusarium oxysporum.

SEQ ID NO: 219 is the amino acid sequence of the lysozyme enhancing domain of SWISSPROT:G0RZV2 from Chaetomium thermophilum var. Thermophilum.

SEQ ID NO: 220 is the amino acid sequence of the lysozyme enhancing domain of SWISSPROT:G2RG70 from Thielavia terrestris.

SEQ ID NO: 221 is the amino acid sequence of the lysozyme enhancing domain of SWISSPROT:W9Z992 from Fusarium oxysporum f. sp. Melonis.

SEQ ID NO: 222 is the amino acid sequence of the lysozyme enhancing domain of SWISSPROT:W9ZZW9 from Fusarium oxysporum f. sp. Melonis.

SEQ ID NO: 223 is the amino acid sequence of the lysozyme enhancing domain of SWISSPROT:W9ZAE8 from Fusarium oxysporum f. sp. Melonis.

SEQ ID NO: 224 is the amino acid sequence of the lysozyme enhancing domain of SWISSPROT:N1RWA4 from Fusarium oxysporum f. sp. Cubense.

SEQ ID NO: 225 is the amino acid sequence of the lysozyme enhancing domain of SWISSPROT:N4UKT7 from Fusarium oxysporum f. sp. Cubense.

SEQ ID NO: 226 is the amino acid sequence of the lysozyme enhancing domain of SWISSPROT:W9KX02 from Fusarium oxysporum.

SEQ ID NO: 227 is the amino acid sequence of the lysozyme enhancing domain of SWISSPROT:X0B4J3 from Fusarium oxysporum f. sp. Raphani.

SEQ ID NO: 228 is the amino acid sequence of the lysozyme enhancing domain of SWISSPROT:W 6QE02 from Penicillium roqueforti.

SEQ ID NO: 229 is the amino acid sequence of the lysozyme enhancing domain of SWISSPROT:W 6R4X8 from Penicillium roqueforti.

SEQ ID NO: 230 is the amino acid sequence of the lysozyme enhancing domain of SWISSPROT:A0A02459B8 from Trichoderma reesei.

SEQ ID NO: 231 is the amino acid sequence of the lysozyme enhancing domain of SWISSPROT:A0A086NN36 from Metarhizium anisopliae.

SEQ ID NO: 232 is the amino acid sequence of the lysozyme enhancing domain of SWISSPROT:A0A094GA03 from Pseudogymnoascus pannorum.

SEQ ID NO: 233 is the amino acid sequence of the lysozyme enhancing domain of SWISSPROT:A0A094C8U1 from Pseudogymnoascus pannorum.

SEQ ID NO: 234 is the amino acid sequence of the lysozyme enhancing domain of SWISSPROT:A0A093Z6Z8 from Pseudogymnoascus pannorum.

SEQ ID NO: 235 is the amino acid sequence of the lysozyme enhancing domain of SWISSPROT:A0A094IML3 from Pseudogymnoascus pannorum.

SEQ ID NO: 236 is the amino acid sequence of the lysozyme enhancing domain of SWISSPROT:A0A094GY79 from Pseudogymnoascus pannorum.

SEQ ID NO: 237 is the amino acid sequence of the lysozyme enhancing domain of SWISSPROT:A0A093XPZ7 from Pseudogymnoascus pannorum.

SEQ ID NO: 238 is the amino acid sequence of the lysozyme enhancing domain of SWISSPROT:A0A093XAS9 from Pseudogymnoascus pannorum.

SEQ ID NO: 239 is the amino acid sequence of the lysozyme enhancing domain of SWISSPROT:A0A09418J6 from Pseudogymnoascus pannorum.

SEQ ID NO: 240 is the amino acid sequence of the lysozyme enhancing domain of SWISSPROT:A0A094FTL0 from Pseudogymnoascus pannorum.

SEQ ID NO: 241 is the amino acid sequence of the lysozyme enhancing domain of SWISSPROT:A0A094AT39 from Pseudogymnoascus pannorum.

SEQ ID NO: 242 is the amino acid sequence of the lysozyme enhancing domain of SWISSPROT:A0A093XSP5 from Pseudogymnoascus pannorum.

SEQ ID NO: 243 is the amino acid sequence of the lysozyme enhancing domain of SWISSPROT:A0A094BAE6 from Pseudogymnoascus pannorum.

SEQ ID NO: 244 is the amino acid sequence of the lysozyme enhancing domain of SWISSPROT:A0A094IE25 from Pseudogymnoascus pannorum.

SEQ ID NO: 245 is the amino acid sequence of the lysozyme enhancing domain of SWISSPROT:A0A094HNM8 from Pseudogymnoascus pannorum.

SEQ ID NO: 246 is the amino acid sequence of the lysozyme enhancing domain of SWISSPROT:A0A094ETJ5 from Pseudogymnoascus pannorum.

SEQ ID NO: 247 is the amino acid sequence of the lysozyme enhancing domain of SWISSPROT:A0A094EPJ7 from Pseudogymnoascus pannorum.

SEQ ID NO: 248 is the amino acid sequence of the lysozyme enhancing domain of SWISSPROT:A0A094E9W0 from Pseudogymnoascus pannorum.

SEQ ID NO: 249 is the amino acid sequence of the lysozyme enhancing domain of SWISSPROT:A0A094BWD6 from Pseudogymnoascus pannorum.

SEQ ID NO: 250 is the amino acid sequence of the lysozyme enhancing domain of SWISSPROT:A0A094BTS1 from Pseudogymnoascus pannorum.

SEQ ID NO: 251 is the amino acid sequence of the lysozyme enhancing domain of SWISSPROT:A0A093ZTZ8 from Pseudogymnoascus pannorum.

SEQ ID NO: 252 is conserved motif I [TAS][VIL][GAC][YFI]GHX[CAYIV].

SEQ ID NO: 253 is conserved motif II [LV][NDST]XN[QE][YFW][GASND]ALXS[WFLY]X[FY]N.

SEQ ID NO: 254 is conserved motif III [CGY][YF][VIL][ASTP][DG]X[YF][VIT]X[TS][GAN].

SEQ ID NO: 255 is the cDNA sequence of the GH24 lysozyme as isolated from Trichophaea saccata.

SEQ ID NO: 256 is the amino acid sequence as deduced from SEQ ID NO: 255.

SEQ ID NO: 257 is the amino acid sequence of the mature GH24 lysozyme from Trichophaea saccata.

SEQ ID NO: 258 is primer F-80470.

SEQ ID NO: 259 is primer R-80470.

SEQ ID NO: 260 is primer 8643.

SEQ ID NO: 261 is primer 8654.

SEQ ID NO: 262 is the cDNA sequence of the GH24 lysozyme as isolated from Chaetomium thermophilum.

SEQ ID NO: 263 is the amino acid sequence as deduced from SEQ ID NO: 262.

SEQ ID NO: 264 is the amino acid sequence of the mature GH24 lysozyme from Chaetomium thermophilum.

SEQ ID NO: 265 is the DNA sequence of the GH24 lysozyme as isolated from Trichoderma harzianum.

SEQ ID NO: 266 is the amino acid sequence as deduced from SEQ ID NO: 265.

SEQ ID NO: 267 is the amino acid sequence of the mature GH24 lysozyme from Trichoderma harzianum.

SEQ ID NO: 268 is the codon optimised DNA sequence of the GH24 catalytic domain from SEQ ID NO: 255 with QCVG-linker and Savinase signal peptide.

SEQ ID NO: 269 is the amino acid sequence as deduced from SEQ ID NO: 268.

SEQ ID NO: 270 is the amino acid sequence of the mature GH24 catalytic domain from Trichophaea saccata with QCVG-linker.

SEQ ID NO: 271 is the DNA sequence of the lysozyme enhancing domain from SEQ ID NO: 255.

SEQ ID NO: 272 is the amino acid sequence as deduced from SEQ ID NO: 271.

SEQ ID NO: 273 is the amino acid sequence of the mature LED from Trichophaea saccata.

SEQ ID NO: 274 is primer P 348F9-R.

SEQ ID NO: 275 is primer A00611-F.

SEQ ID NO: 276 is primer A00611-R.

SEQ ID NO: 277 is primer CBS 14450-F.

SEQ ID NO: 278 is primer CBS 14450-R.

SEQ ID NO: 279 is the amino acid sequence of the mature GH24 lysozyme from Acremonium alcalophilum corresponding to the mature sequence of SEQ ID NO: 4 of WO 2013/076259.

SEQ ID NO: 280 is the amino acid sequence of the mature GH24 lysozyme from Acremonium alcalophilum corresponding to the mature sequence of SEQ ID NO: 6 of WO 2013/076259.

SEQ ID NO: 281 is conserved motif I-B [TAS][VIL][GAC][YFI]GHX[CAYIV].

SEQ ID NO: 282 is conserved motif I-C T[VI]GYGHXC.

SEQ ID NO: 283 is conserved motif II-B LNXN[QE][YFW][GA]ALXS[W FL]X[YF]N.

SEQ ID NO: 284 is conserved motif III-B C[YF][VI][AST]D[YKF][YF][VI]XTG.

SEQ ID NO: 285 is conserved motif IV [GEV]LXXRRXXE.

SEQ ID NO: 286 is conserved motif IV-B GLXXRRXXE.

SEQ ID NO: 287 is the gene sequence of the GH24 lysozyme as isolated from Trichophaea minuta.

SEQ ID NO: 288 is the amino acid sequence as deduced from SEQ ID NO: 287.

SEQ ID NO: 289 is the codon optimised DNA sequence of SEQ ID NO: 287.

SEQ ID NO: 290 is the amino acid sequence as deduced from SEQ ID NO: 287.

SEQ ID NO: 291 is the amino acid sequence of the mature GH24 lysozyme from Trichophaea minuta.

SEQ ID NO: 292 is the gene sequence of the GH24 lysozyme as isolated from Chaetomium sp. ZY 287.

SEQ ID NO: 293 is the amino acid sequence as deduced from SEQ ID NO: 292.

SEQ ID NO: 294 is the amino acid sequence of the mature GH24 lysozyme from Chaetomium sp. ZY 287.

SEQ ID NO: 295 is the gene sequence of the GH24 lysozyme as isolated from Mortierella sp. ZY 002.

SEQ ID NO: 296 is the amino acid sequence as deduced from SEQ ID NO: 295.

SEQ ID NO: 297 is the amino acid sequence of the mature GH24 lysozyme from Mortierella sp. ZY 002.

SEQ ID NO: 298 is the gene sequence of the GH24 lysozyme as isolated from Metarhizium sp. XZ2431.

SEQ ID NO: 299 is the amino acid sequence as deduced from SEQ ID NO: 298.

SEQ ID NO: 300 is the amino acid sequence of the mature GH24 lysozyme from Metarhizium sp. XZ2431.

SEQ ID NO: 301 is the gene sequence of the GH24 lysozyme as isolated from Geomyces auratus.

SEQ ID NO: 302 is the amino acid sequence as deduced from SEQ ID NO: 301.

SEQ ID NO: 303 is the amino acid sequence of the mature GH24 lysozyme from Geomyces auratus.

SEQ ID NO: 304 is the gene sequence of the GH24 lysozyme as isolated from Ilyonectria rufa.

SEQ ID NO: 305 is the amino acid sequence as deduced from SEQ ID NO: 304.

SEQ ID NO: 306 is the amino acid sequence of the mature GH24 lysozyme from Ilyonectria rufa.

### Definitions

50% MHB, pH 6: The term “50% MHB, pH 6” means that the antimicrobial activity of the lysozyme was tested using an RDA plate wherein the media used was ½ Mueller-Hinton broth (MHB) (Sigma/Fluka, 90922) (i.e., adjusted to pH 6 with 4 M HCl and diluted 1:1 with water) with 1.5% agarose.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Animal: The term “animal” refers to any animal except humans. Examples of animals are non-ruminants and ruminants. Ruminant animals include, for example, animals such as sheep, goats, cattle, e.g., beef cattle, cows, and young calves, deer, yank, camel, llama and kangaroo. Non-ruminant animals include monogastric animals, including but not limited to pigs or swine (including, but not limited to, piglets, growing pigs, and sows); poultry such as turkeys, ducks, quail, guinea fowl, geese, pigeons (including squabs) and chicken (including but not limited to broiler chickens (referred to herein as broiles), chicks, layer hens (referred to herein as layers)); horses (including but not limited to hotbloods, coldbloods and warm bloods) crustaceans (including but not limited to shrimps and prawns) and fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish).

Animal feed: The term “animal feed” refers to any compound, preparation, or mixture suitable for, or intended for intake by an animal. Animal feed for a monogastric animal typically comprises concentrates as well as vitamins, minerals, enzymes, direct fed microbial, amino acids and/or other feed ingredients (such as in a premix) whereas animal feed for ruminants generally comprises forage (including roughage and silage) and may further comprise concentrates as well as vitamins, minerals, enzymes direct fed microbial, amino acid and/or other feed ingredients (such as in a premix).

Antimicrobial activity: The term “antimicrobial activity” is defined herein as an activity that kills or inhibits the growth of microorganisms, such as, algae, archea, bacteria, fungi and/or protozoans. The antimicrobial activity can, for example, be bactericidal meaning the killing of bacteria or bacteriostatic meaning the prevention of bacterial growth. The antimicrobial activity can include catalyzing the hydrolysis of 1,4-beta-linkages between N-acetylmuramic acid and N-acetyl-D-glucosamine residues in a peptidoglycan and between N-acetyl-D-glucosamine residues in chitodextrins. Antimicrobial activity can also include the lysozyme binding to the surface of the microorganism and inhibiting its growth. The antimicrobial effect can also include the use of the lysozymes of the present invention for activation of bacterial autolysins, as an immunostimulator, by inhibiting or reducing bacterial toxins and by an opsonin effect.

For the purpose of the present invention, antimicrobial activity is determined according to the antimicrobial assay described in Example 12 (“Determination of antimicrobial activity”). Antimicrobial activity is determined if there is a clearing zone when using 50% Mueller-Hinton broth, pH 6. Preferably the diameter of the clearing zone is 4 mm or more.

Thus, the term “antimicrobial activity of SEQ ID NO: x against Clostridium perfringens using the conditions 50% MHB, pH 6” mean that the polypeptide shows antimicrobial activity (i.e., clearing zone) against Clostridium perfringens DSM756 when the conditions 50% Mueller-Hinton broth, pH 6 are used, when the experiment is performed as described in example 12.

Body Weight Gain: The term “body weight gain” means an increase in live weight of an animal during a given period of time, e.g., the increase in weight from day 1 to day 21.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Concentrates: The term “concentrates” means feed with high protein and energy concentrations, such as fish meal, molasses, oligosaccharides, sorghum, seeds and grains (either whole or prepared by crushing, milling, etc. from, e.g., corn, oats, rye, barley, wheat), oilseed press cake (e.g., from cottonseed, safflower, sunflower, soybean (such as soybean meal), rapeseed/canola, peanut or groundnut), palm kernel cake, yeast derived material and distillers grains (such as wet distillers grains (WDS) and dried distillers grains with solubles (DDGS)).

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

domT score: The term “domT score” or domain score means the score for the best scoring domain when aligning the query sequence to the HMM.

European Production Efficacy Factor (EPEF): The “European Production Efficacy Factor” is a way of comparing the performance of animals. This single-figure facilitates comparison of performance within and among farms and can be used to assess environmental, climatic and managemental variables. The EPEF is calculated as [(liveability (%)×Liveweight (kg))/(Age at depletion (days)×FCR)]×100, wherein livability is the percentage of animals alive at slaughter, Liveweight is the average weight of the animals at slaughter, age of depletion is the age of the animals at slaughter and FCR is the feed conversion ratio at slaughter.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Feed Conversion Ratio (FCR): FCR is a measure of an animal's efficiency in converting feed mass into increases of the desired output. Animals raised for meat—such as swine, poultry and fish—the output is the mass gained by the animal. Specifically FCR is calculated as feed intake divided by weight gain, all over a specified period. Improvement in FCR means reduction of the FCR value. A FCR improvement of 2% means that the FCR was reduced by 2%.

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without said feed additive composition being present.

Forage: The term “forage” as defined herein also includes roughage. Forage is fresh plant material such as hay and silage from forage plants, grass and other forage plants, seaweed, sprouted grains and legumes, or any combination thereof. Examples of forage plants are Alfalfa (lucerne), birdsfoot trefoil, brassica (e.g., kale, rapeseed (canola), rutabaga (swede), turnip), clover (e.g., alsike clover, red clover, subterranean clover, white clover), grass (e.g., Bermuda grass, brome, false oat grass, fescue, heath grass, meadow grasses, orchard grass, ryegrass, Timothy-grass), corn (maize), millet, barley, oats, rye, sorghum, soybeans and wheat and vegetables such as beets. Forage further includes crop residues from grain production (such as corn stover; straw from wheat, barley, oat, rye and other grains); residues from vegetables like beet tops; residues from oilseed production like stems and leaves form soy beans, rapeseed and other legumes; and fractions from the refining of grains for animal or human consumption or from fuel production or other industries.

Fragment: The term “fragment” means a polypeptide having one or more (e.g., several) amino acids absent from the amino and/or carboxyl terminus of a mature polypeptide or domain; wherein the fragment has lysozyme activity. In one aspect, a fragment contains at least 215 amino acid residues (e.g., amino acids 16 to 230 of SEQ ID NO: 257), at least 225 amino acid residues (e.g., amino acids 11 to 235 of SEQ ID NO: 257), or at least 235 amino acid residues (e.g., amino acids 6 to 240 of SEQ ID NO: 257). In another aspect, a fragment contains at least 215 amino acid residues (e.g., amino acids 16 to 230 of SEQ ID NO: 264), at least 225 amino acid residues (e.g., amino acids 11 to 235 of SEQ ID NO: 264), or at least 235 amino acid residues (e.g., amino acids 6 to 240 of SEQ ID NO: 264). In another aspect, a fragment contains at least 218 amino acid residues (e.g., amino acids 16 to 233 of SEQ ID NO: 267), at least 228 amino acid residues (e.g., amino acids 11 to 238 of SEQ ID NO: 267), or at least 238 amino acid residues (e.g., amino acids 6 to 243 of SEQ ID NO: 267).

In one aspect, the fragment comprises at least 90% of the length of the mature polypeptide, such as at least 220 amino acids of SEQ ID NO: 288, at least 220 amino acids of SEQ ID NO: 290, at least 220 amino acids of SEQ ID NO: 291, at least 224 amino acids of SEQ ID NO: 293, at least 224 amino acids of SEQ ID NO: 294, at least 220 amino acids of SEQ ID NO: 296, at least 220 amino acids of SEQ ID NO: 297, at least 222 amino acids of SEQ ID NO: 299, at least 222 amino acids of SEQ ID NO: 300, at least 225 amino acids of SEQ ID NO: 302, at least 225 amino acids of SEQ ID NO: 303, at least 216 amino acids of SEQ ID NO: 305 or at least 216 amino acids of SEQ ID NO: 306.

In another aspect, the fragment comprises at least 92% of the length of the mature polypeptide, such as at least 225 amino acids of SEQ ID NO: 288, at least 225 amino acids of SEQ ID NO: 290, at least 225 amino acids of SEQ ID NO: 291, at least 229 amino acids of SEQ ID NO: 293, at least 229 amino acids of SEQ ID NO: 294, at least 225 amino acids of SEQ ID NO: 296, at least 225 amino acids of SEQ ID NO: 297, at least 227 amino acids of SEQ ID NO: 299, at least 227 amino acids of SEQ ID NO: 300, at least 230 amino acids of SEQ ID NO: 302, at least 230 amino acids of SEQ ID NO: 303, at least 220 amino acids of SEQ ID NO: 305 or at least 220 amino acids of SEQ ID NO: 306.

In another aspect, the fragment comprises at least 94% of the length of the mature polypeptide, such as at least 230 amino acids of SEQ ID NO: 288, at least 230 amino acids of SEQ ID NO: 290, at least 230 amino acids of SEQ ID NO: 291, at least 234 amino acids of SEQ ID NO: 293, at least 234 amino acids of SEQ ID NO: 294, at least 230 amino acids of SEQ ID NO: 296, at least 230 amino acids of SEQ ID NO: 297, at least 232 amino acids of SEQ ID NO: 299, at least 232 amino acids of SEQ ID NO: 300, at least 235 amino acids of SEQ ID NO: 302, at least 235 amino acids of SEQ ID NO: 303, at least 225 amino acids of SEQ ID NO: 305 or at least 225 amino acids of SEQ ID NO: 306.

In another aspect, the fragment comprises at least 96% of the length of the mature polypeptide, such as at least 235 amino acids of SEQ ID NO: 288, at least 235 amino acids of SEQ ID NO: 290, at least 235 amino acids of SEQ ID NO: 291, at least 239 amino acids of SEQ ID NO: 293, at least 239 amino acids of SEQ ID NO: 294, at least 235 amino acids of SEQ ID NO: 296, at least 235 amino acids of SEQ ID NO: 297, at least 237 amino acids of SEQ ID NO: 299, at least 237 amino acids of SEQ ID NO: 300, at least 240 amino acids of SEQ ID NO: 302, at least 240 amino acids of SEQ ID NO: 303, at least 230 amino acids of SEQ ID NO: 305 or at least 230 amino acids of SEQ ID NO: 306.

In another aspect, the fragment comprises at least 98% of the length of the mature polypeptide, such as at least 240 amino acids of SEQ ID NO: 288, at least 240 amino acids of SEQ ID NO: 290, at least 240 amino acids of SEQ ID NO: 291, at least 244 amino acids of SEQ ID NO: 293, at least 244 amino acids of SEQ ID NO: 294, at least 240 amino acids of SEQ ID NO: 296, at least 240 amino acids of SEQ ID NO: 297, at least 242 amino acids of SEQ ID NO: 299, at least 242 amino acids of SEQ ID NO: 300, at least 245 amino acids of SEQ ID NO: 302, at least 245 amino acids of SEQ ID NO: 303, at least 235 amino acids of SEQ ID NO: 305 or at least 235 amino acids of SEQ ID NO: 306.

In another aspect, the fragment comprises at least 99% of the length of the mature polypeptide, such as at least 242 amino acids of SEQ ID NO: 288, at least 242 amino acids of SEQ ID NO: 290, at least 242 amino acids of SEQ ID NO: 291, at least 246 amino acids of SEQ ID NO: 293, at least 246 amino acids of SEQ ID NO: 294, at least 242 amino acids of SEQ ID NO: 296, at least 242 amino acids of SEQ ID NO: 297, at least 244 amino acids of SEQ ID NO: 299, at least 244 amino acids of SEQ ID NO: 300, at least 247 amino acids of SEQ ID NO: 302, at least 247 amino acids of SEQ ID NO: 303, at least 237 amino acids of SEQ ID NO: 305 or at least 237 amino acids of SEQ ID NO: 306.

hmmbuild: The term “hmmbuild” is a program from the package HMMER 3.0 (March 2010) (hmmer.org/) that builds a profile HMM from an input multiple aligment.

hmmscan: The term “hmmscan” is a program from the package HMMER 3.0 (March 2010) (hmmer.org/) that searches a protein sequence against a protein profile HMM database.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Lysozyme activity: The term “lysozyme activity” means the hydrolysis of the 1,4-beta-linkages between N-acetylmuramic acid and N-acetyl-D-glucosamine residues in a peptidoglycan or between N-acetyl-D-glucosamine residues in chitodextrins, resulting in bacteriolysis. Lysozyme belongs to the enzyme class EC 3.2.1.17. Lysozyme activity is typically measured by turbidimetric determination, such as the changes in turbidity of a suspension of Micrococcus luteus ATCC 4698 or Exiguobacterium undea (DSM14481) induced by the lytic action of the lysozyme. In appropriate experimental conditions these changes are proportional to the amount of lysozyme in the medium (c.f. INS 1105 of the Combined Compendium of Food Additive Specifications of the Food and Agriculture Organisation of the UN (fao.org)). For the purpose of the present invention, lysozyme activity is determined according to the turbidity assay described in example 11 (“Determination of Lysozyme Activity”) and the polypeptidehas lysozyme activity if it shows activity against one or more bacteria, such as Micrococcus luteus ATCC 4698 and/or Exiguobacterium undea (DSM14481). In one aspect, the polypeptides of the present invention have at least 20%, e.g., at least 40%, at least 50%, at least 60%, at least 70%, at least 80%, at least 90%, at least 95%, or at least 100% of the lysozyme activity of SEQ ID NO: 257. In another aspect, the polypeptides of the present invention have at least 20%, e.g., at least 40%, at least 50%, at least 60%, at least 70%, at least 80%, at least 90%, at least 95%, or at least 100% of the lysozyme activity of SEQ ID NO: 264. In another aspect, the polypeptides of the present invention have at least 20%, e.g., at least 40%, at least 50%, at least 60%, at least 70%, at least 80%, at least 90%, at least 95%, or at least 100% of the lysozyme activity of SEQ ID NO: 267.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide amino acids 1 to 245 of SEQ ID NO: 257 based on EDMAN N-terminal sequencing data, intact molecular weight analysis and proteomic analysis. In another aspect, the mature polypeptide amino acids 1 to 245 of SEQ ID NO: 264. In another aspect, the mature polypeptide amino acids 1 to 248 of SEQ ID NO: 267.

In one aspect, the mature polypeptide is amino acids 1 to 245 of SEQ ID NO: 288 and amino acids −17 to −1 of SEQ ID NO: 288 are a signal peptide. In another aspect, the mature polypeptide is amino acids 1 to 245 of SEQ ID NO: 290. In an alternative aspect, the mature polypeptide is amino acids 1 to 245 of SEQ ID NO: 291.

In one aspect, the mature polypeptide is amino acids 1 to 249 of SEQ ID NO: 293 and amino acids −18 to −1 of SEQ ID NO: 293 are a signal peptide. In an alternative aspect, the mature polypeptide is amino acids 1 to 249 of SEQ ID NO: 294.

In one aspect, the mature polypeptide is amino acids 1 to 245 of SEQ ID NO: 296 and amino acids −19 to −1 of SEQ ID NO: 296 are a signal peptide. In an alternative aspect, the mature polypeptide is amino acids 1 to 245 of SEQ ID NO: 297.

In one aspect, the mature polypeptide is amino acids 1 to 247 of SEQ ID NO: 299 and amino acids −19 to −1 of SEQ ID NO: 299 are a signal peptide. In an alternative aspect, the mature polypeptide is amino acids 1 to 247 of SEQ ID NO: 300.

In one aspect, the mature polypeptide is amino acids 1 to 250 of SEQ ID NO: 302 and amino acids −18 to −1 of SEQ ID NO: 302 are a signal peptide. In an alternative aspect, the mature polypeptide is amino acids 1 to 250 of SEQ ID NO: 303.

In one aspect, the mature polypeptide is amino acids 1 to 240 of SEQ ID NO: 305 and amino acids −18 to −1 of SEQ ID NO: 305 are a signal peptide. In an alternative aspect, the mature polypeptide is amino acids 1 to 240 of SEQ ID NO: 306.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having lysozyme activity. In one aspect, the mature polypeptide coding sequence is the joined sequence of nucleotides 52 to 347, nucleotides 401 to 615, nucleotides 668 to 772 and nucleotides 825 to 943 of SEQ ID NO: 255 or the cDNA sequence thereof and nucleotides 1 to 51 of SEQ ID NO: 255 are the signal peptide. In another aspect, the mature polypeptide coding sequence is the joined sequence of nucleotides 55 to 367, nucleotides 425 to 555 and nucleotides 630 to 920 of SEQ ID NO: 262 or the cDNA sequence thereof and nucleotides 1 to 54 of SEQ ID NO: 262 are the signal peptide. In another aspect, the mature polypeptide coding sequence is nucleotides 55 to 798 of SEQ ID NO: 265 and nucleotides 1 to 54 of SEQ ID NO: 265 are the signal peptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Obtained or obtainable from: The term “obtained or obtainable from” means that the polypeptide may be found in an organism from a specific taxonomic rank. In one embodiment, the polypeptide is obtained or obtainable from the kingdom Fungi, wherein the term kingdom is the taxonomic rank. In a preferred embodiment, the polypeptide is obtained or obtainable from the phylum Ascomycota, wherein the term phylum is the taxonomic rank. In another preferred embodiment, the polypeptide is obtained or obtainable from the subphylum Pezizomycotina, wherein the term subphylum is the taxonomic rank.

If the taxonomic rank of a polypeptide is not known, it can easily be determined by a person skilled in the art by performing a BLASTP search of the polypeptide (using, e.g., the National Center for Biotechnology Information (NCIB) website: ncbi.nlm.nih.gov/) and comparing it to the closest homologues. An unknown polypeptide which is a fragment of a known polypeptide is considered to be of the same taxonomic species. An unknown natural polypeptide or artificial variant which comprises a substitution, deletion and/or insertion in up to 10 positions is considered to be from the same taxonomic species as the known polypeptide.

Operably linked: The term “operably linked” means a configuration in which a control sequence is placed at an appropriate position relative to the coding sequence of a polynucleotide such that the control sequence directs expression of the coding sequence.

Profile Hidden Markov Model: “Profile Hidden Markov Model” are statistical models of multiple sequence alignments. They capture position-specific information about how conserved each column of the alignment is, and which residues are likely (see ftp://ftp.hgc.jp/pub/mirror/wustl/hmmer3/3.1b1/Userguide.pdf).

Roughage: The term “roughage” means dry plant material with high levels of fiber, such as fiber, bran, husks from seeds and grains and crop residues (such as stover, copra, straw, chaff, sugar beet waste).

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix. The output of Needle labelled “longest identity” (obtained using the-nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labelled “longest identity” (obtained using the-nobrief option) is used as the percent identity and is calculated as follows:

(Identical Deoxyribonucleotides×100)/(Length of Alignment−Total Number of Gaps in Alignment)

Stringency conditions: The different stringency conditions are defined as follows.

The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.8×SSC, 0.2% SDS at 55° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.8×SSC, 0.2% SDS at 60° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.8×SSC, 0.2% SDS at 65° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.8×SSC, 0.2% SDS at 70° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.4×SSC, 0.2% SDS at 70° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.4×SSC, 0.2% SDS at 75° C.

Subsequence: The term “subsequence” means a polynucleotide having one or more (e.g., several) nucleotides absent from the 5′ and/or 3′ end of a mature polypeptide coding sequence; wherein the subsequence encodes a fragment having lysozyme activity. In one aspect, a subsequence contains at least 645 nucleotides (e.g., the joined sequence of nucleotides 97 to 347, nucleotides 401 to 615, nucleotides 668 to 772 and nucleotides 825 to 898 of SEQ ID NO: 255 or the cDNA sequence thereof), at least 675 nucleotides (e.g., the joined sequence of nucleotides 82 to 347, nucleotides 401 to 615, nucleotides 668 to 772 and nucleotides 825 to 913 of SEQ ID NO: 255 or the cDNA sequence thereof), or at least 705 nucleotides (e.g., the joined sequence of nucleotides 67 to 347, nucleotides 401 to 615, nucleotides 668 to 772 and nucleotides 825 to 928 of SEQ ID NO: 255 or the cDNA sequence thereof).

Substantially pure polypeptide: The term “substantially pure polypeptide” means a preparation that contains at most 10%, at most 8%, at most 6%, at most 5%, at most 4%, at most 3%, at most 2%, at most 1%, and at most 0.5% by weight of other polypeptide material with which it is natively or recombinantly associated. Preferably, the polypeptide is at least 92% pure, e.g., at least 94% pure, at least 95% pure, at least 96% pure, at least 97% pure, at least 98% pure, at least 99%, at least 99.5% pure, and 100% pure by weight of the total polypeptide material present in the preparation. The polypeptides of the present invention are preferably in a substantially pure form. This can be accomplished, for example, by preparing the polypeptide by well known recombinant methods or by classical purification methods.

Variant: The term “variant” means a polypeptide having lysozyme activity comprising an alteration, i.e., a substitution, insertion, and/or deletion, of one or more (several) amino acid residues at one or more (e.g., several) positions. A substitution means replacement of the amino acid occupying a position with a different amino acid; a deletion means removal of the amino acid occupying a position; and an insertion means adding 1, 2, or 3 amino acids adjacent to and immediately following the amino acid occupying the position.

In one aspect, a lysozyme variant according to the invention may comprise from 1 to 5; from 1 to 10; from 1 to 15; from 1 to 20; from 1 to 25; from 1 to 30; from 1 to 35; from 1 to 40; from 1 to 45; or from 1-50, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 alterations and have at least 20%, e.g., at least 40%, at least 50%, at least 60%, at least 70%, at least 80%, at least 90%, at least 95%, or at least 100% of the lysozyme activity of the parent lysozyme, such as SEQ ID NO: 257, SEQ ID NO: 264 or SEQ ID NO: 267.

### Nomenclature

For purposes of the present invention, the nomenclature [E/Q] means that the amino acid at this position may be a glutamic acid (Glu, E) or a glutamine (Gln, Q). Likewise the nomenclature [V/G/A/I] means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

### DETAILED DESCRIPTION OF THE INVENTION

### Animal Feed and Animal Feed Additives Comprising Polypeptides Having Lysozyme Activity

The inventors have discovered that some polypeptides that comprise one or more Glycosyl Hydrolase family 24 (GH24) catalytic domains also comprise sections of polypeptide which until now has not been known to have any function and have therefore not been annotated. The inventors have herein annotated these sections of polypeptide, which is referred to herein as a lysozyme enhancing domain (LED), and have surprisingly discovered that this lysozyme enhancing domain significantly enhances antimicrobial activity against Clostridium perfringens.

For example, the polypeptide of SEQ ID NO: 257 which comprises a GH24 domain and a lysozyme enhancing domain has significant activity against Clostridium perfringens (see table 3 of example 12). However, when the polypeptide is expressed without the lysozyme enhancing domain being present (SEQ ID NO: 270), there is no activity against Clostridium perfringens using the conditions 50% MHB, PH 6.

Two other polypeptides comprising a GH24 domain and a lysozyme enhancing domain (SEQ ID NO: 264 and 267) also have significant activity against Clostridium perfringens (see table 3) using the conditions 50% MHB, pH 6. However, two known GH24 lysozymes (SEQ ID NO: 279 and SEQ ID NO: 280) which do not comprise a lysozyme enhancing domain do not have any activity against Clostridium perfringens.

The inventors have also discovered that the LED protein (SEQ ID NO: 273) binds to M. lysodiektikus cells under the buffer conditions tested while crystalline cellulose did not bind the LED protein. This demonstrates that the LED protein is an important feature of the lysozymes of the invention and possibly explains why said lysozymes are more active against Clostridium perfringens under the tested conditions compared to GH24 lysozymes lacking the LED.

Thus, in one aspect, the invention relates to an animal feed or animal feed additive comprising one or more polypeptides having lysozyme activity, wherein the polypeptide comprises one or more GH24 catalytic domains and one or more lysozyme enhancing domains and wherein:
(a) GH24 catalytic domain gives a domT score of at least 200 when queried using a Profile Hidden Markov Model prepared using SEQ ID NOs: 1 to 122 and hmmbuild software program, and wherein the query is carried out using hmmscan software program with default settings; and
(b) the polypeptide comprises one or more lysozyme enhancing domains, wherein the lysozyme enhancing domain gives a domT score of at least 100 when queried using a Profile Hidden Markov Model prepared using SEQ ID NOs: 123 to 251 and hmmbuild software program, and wherein the query is carried out using the hmmscan software program with default settings.

In another aspect, the present invention relates to an animal feed or animal feed additive comprising one or more polypeptides having lysozyme activity, wherein:
(a) the polypeptide comprises one or more GH24 catalytic domains, wherein the GH24 catalytic domain gives a domT score of 200 or more when queried using a Profile Hidden Markov Model prepared using SEQ ID NOS: 1 to 122 inclusive using the software program hmmbuild, the query being carried out using the hmmscan software program with default settings; and
(b) the polypeptide comprises one or more lysozyme enhancing domains, wherein the lysozyme enhancing domain gives a domT score of 100 or more when queried using a Profile Hidden Markov Model prepared using SEQ ID NOs: 123 to 251 inclusive using the software program hmmbuild, the query being carried out using the hmmscan software program with default settings.

In another aspect, the present invention relates to an animal feed or animal feed additive comprising one or more vitamins and one or more polypeptides having lysozyme activity, wherein the polypeptide comprises one or more GH24 catalytic domains and one or more lysozyme enhancing domains and wherein:
(a) GH24 catalytic domain gives a domT score of at least 200 when queried using a Profile Hidden Markov Model prepared using SEQ ID NOs: 1 to 122 and hmmbuild software program, and wherein the query is carried out using hmmscan software program with default settings; and
(b) the polypeptide comprises one or more lysozyme enhancing domains, wherein the lysozyme enhancing domain gives a domT score of at least 100 when queried using a Profile Hidden Markov Model prepared using SEQ ID NOs: 123 to 251 and hmmbuild software program, and wherein the query is carried out using the hmmscan software program with default settings.

In another aspect, the present invention relates to an animal feed or animal feed additive comprising one or more minerals and one or more polypeptides having lysozyme activity, wherein the polypeptide comprises one or more GH24 catalytic domains and one or more lysozyme enhancing domains and wherein:
(a) GH24 catalytic domain gives a domT score of at least 200 when queried using a Profile Hidden Markov Model prepared using SEQ ID NOS: 1 to 122 and hmmbuild software program, and wherein the query is carried out using hmmscan software program with default settings; and
(b) the polypeptide comprises one or more lysozyme enhancing domains, wherein the lysozyme enhancing domain gives a domT score of at least 100 when queried using a Profile Hidden Markov Model prepared using SEQ ID NOs: 123 to 251 and hmmbuild software program, and wherein the query is carried out using the hmmscan software program with default settings.

In another aspect, the present invention relates to an animal feed or animal feed additive comprising one or more amino acids and one or more polypeptides having lysozyme activity, wherein the polypeptide comprises one or more GH24 catalytic domains and one or more lysozyme enhancing domains and wherein:
(a) GH24 catalytic domain gives a domT score of at least 200 when queried using a Profile Hidden Markov Model prepared using SEQ ID NOs: 1 to 122 and hmmbuild software program, and wherein the query is carried out using hmmscan software program with default settings; and
(b) the polypeptide comprises one or more lysozyme enhancing domains, wherein the lysozyme enhancing domain gives a domT score of at least 100 when queried using a Profile Hidden Markov Model prepared using SEQ ID NOs: 123 to 251 and hmmbuild software program, and wherein the query is carried out using the hmmscan software program with default settings.

In another aspect, the present invention relates to an animal feed or animal feed additive comprising one or more polypeptides having lysozyme activity, wherein the polypeptide comprises one or more GH24 catalytic domains and one or more lysozyme enhancing domains and wherein:
(a) GH24 catalytic domain gives a domT score of at least 200 when queried using a Profile Hidden Markov Model prepared using SEQ ID NOs: 1 to 122 and hmmbuild software program, and wherein the query is carried out using hmmscan software program with default settings; and
(b) the polypeptide comprises one or more lysozyme enhancing domains, wherein the lysozyme enhancing domain gives a domT score of at least 100 when queried using a Profile Hidden Markov Model prepared using SEQ ID NOs: 123 to 251 and hmmbuild software program, and wherein the query is carried out using the hmmscan software program with default settings; and
one or more more components selected from the list consisting of:
   one or more additional enzymes;
   one or more microbes;
   one or more vitamins;
   one or more minerals;
   one or more amino acids; and
   one or more other feed ingredients.

In another aspect, the present invention relates to a pelleted animal feed comprising plant based material and one or more polypeptides having lysozyme activity, wherein the polypeptide comprises one or more GH24 catalytic domains and one or more lysozyme enhancing domains and wherein:
(a) GH24 catalytic domain gives a domT score of at least 200 when queried using a Profile Hidden Markov Model prepared using SEQ ID NOs: 1 to 122 and hmmbuild software program, and wherein the query is carried out using hmmscan software program with default settings; and
(b) the polypeptide comprises one or more lysozyme enhancing domains, wherein the lysozyme enhancing domain gives a domT score of at least 100 when queried using a Profile Hidden Markov Model prepared using SEQ ID NOs: 123 to 251 and hmmbuild software program, and wherein the query is carried out using the hmmscan software program with default settings.

The theory behind Profile HMMs as described in Durbin et al. (Biological sequence analysis: probabilistic models of proteins and nucleic acids, Cambridge University Press, 1998) and Krogh et al. (J. Mol. Biol. 235:1501-1531 (1994)), both incorporated herein by reference, is characterization of a set of proteins based on the probability of each amino acid occurring at each position in the alignment of the proteins of the set.

Specifically, profile HMMs are statistical models of multiple sequence alignments, or even of single sequences. They capture position-specific information about how conserved each column of the alignment is, and which residues are likely. All profile methods are more or less statistical descriptions of the consensus of a multiple sequence alignment. They use position-specific scores for amino acids or nucleotides (residues) and position specific penalties for opening and extending an insertion or deletion. Traditional pairwise alignment (for example, BLAST, FASTA or the Smith/Waterman algorithm) uses position-independent scoring parameters. This property of profiles captures important information about the degree of conservation at various positions in the multiple alignment, and the varying degree to which gaps and insertions are permitted.

The advantage of using HMMs is that HMMs have a formal probabilistic basis. Probability theory is used to guide how all the scoring parameters should be set. One of the most important aspect is that HMMs have a consistent theory for setting position-specific gap and insertion scores. The methods are consistent and therefore highly automatable, allowing hundreds of profile HMMs to be applied to, e.g., whole genome analysis. An example of a protein domain model database is Pfam (Sonnhammer et al., 1997, ‘A comprehensive database of protein families based on seed alignments’, Proteins 28:405-420; Finn et al., 2010, ‘The Pfam protein families database’, Nucl. Acids Res. 38: D211-D222), which is a significant part of the Interpro protein domain annotation system. The construction and use of Pfam is tightly tied to the HMMER software package (see en.wikipedia.org/wiki/HMMER).

The GH24 domain is defined in the following manner. SEQ ID NOs: 1 to 122, which are partial sequences of the UniProt entries as explained in the ‘overview of sequence listing’ section herein, are aligned using the software program MUSCLE v3.8.31 with the default settings. Using this alignment, a hidden Markov model (HMM) is built for the GH24 domain. The HMM is constructed using the software program ‘hmmbuild’ from the package HMMER 3.0 (March 2010) (hmmer.org/) and the software is invoked using the default settings.

A GH24 domain is defined to match the above mentioned HMM using the software program ‘hmmscan’ from the package HMMER 3.0 (March 2010) (hmmer.org/) using the default settings if the domT score is at least 200. In a preferred embodiment, the domT score is at least 220, preferably at least 230, more preferably at least 240, even more preferably at least 250, even more preferably at least 255, or most preferably at least 260.

The HMM profile of the GH24 domain as generated using SEQ ID NOs: 1 to 122 according to the procedure above is given in example 15. The HMM profile can be copied into a text file which is subsequently loaded into the software program ‘hmmscan’ so that other polypeptides can be tested to see whether said polypeptide comprises one or more GH24 catalytic domains.

The Lysozyme Enhancing Domain (LED) is defined in the following manner. SEQ ID NOs: 123 to 251, which are partial sequences of the Uniprot entries as explained in the ‘overview of sequence listing’ section herein, are aligned using the software program MUSCLE v3.8.31 with the default settings. Using this alignment, a hidden Markov model (HMM) is built for the LED. The HMM is constructed using the software program ‘hmmbuild’ from the package HMMER 3.0 (March 2010) (hmmer.org/) and the software is invoked using the default settings.

A LED is defined to match the above mentioned HMM using the software program ‘hmmscan’ from the package HMMER 3.0 (March 2010) (hmmer.org/) using the default settings if the domT score is at least 100. In a preferred embodiment, the domT score is at least 103, preferably at least 106, more preferably at least 109, more preferably at least 112, more preferably at least 115, more preferably at least 118, even more preferably at least 121, or most preferably at least 124.

The HMM profile of the LED as generated using SEQ ID NOs: 123 to 251 according to the procedure above is given in example 16. The HMM profile can be copied into a text file which is subsequently loaded into the software program ‘hmmscan’ so that other polypeptides can be tested to see whether said polypeptide comprises one or more LED.

In an embodiment, the GH24 catalytic domain gives a domT score of at least 220 and the lysozyme enhancing domain gives a domT score of at least 100. In an embodiment, the GH24 catalytic domain gives a domT score of at least 230 and the lysozyme enhancing domain gives a domT score of at least 100. In an embodiment, the GH24 catalytic domain gives a domT score of at least 240 and the lysozyme enhancing domain gives a domT score of at least 100. In an embodiment, the GH24 catalytic domain gives a domT score of at least 250 and the lysozyme enhancing domain gives a domT score of at least 100.

In an embodiment, the GH24 catalytic domain gives a domT score of at least 220 and the lysozyme enhancing domain gives a domT score of at least 103. In an embodiment, the GH24 catalytic domain gives a domT score of at least 230 and the lysozyme enhancing domain gives a domT score of at least 103. In an embodiment, the GH24 catalytic domain gives a domT score of at least 240 and the lysozyme enhancing domain gives a domT score of at least 103. In an embodiment, the GH24 catalytic domain gives a domT score of at least 250 and the lysozyme enhancing domain gives a domT score of at least 103.

In an embodiment, the GH24 catalytic domain gives a domT score of at least 220 and the lysozyme enhancing domain gives a domT score of at least 106. In an embodiment, the GH24 catalytic domain gives a domT score of at least 230 and the lysozyme enhancing domain gives a domT score of at least 106. In an embodiment, the GH24 catalytic domain gives a domT score of at least 240 and the lysozyme enhancing domain gives a domT score of at least 106. In an embodiment, the GH24 catalytic domain gives a domT score of at least 250 and the lysozyme enhancing domain gives a domT score of at least 106.

In an embodiment, the GH24 catalytic domain gives a domT score of at least 220 and the lysozyme enhancing domain gives a domT score of at least 109. In an embodiment, the GH24 catalytic domain gives a domT score of at least 230 and the lysozyme enhancing domain gives a domT score of at least 109. In an embodiment, the GH24 catalytic domain gives a domT score of at least 240 and the lysozyme enhancing domain gives a domT score of at least 109. In an embodiment, the GH24 catalytic domain gives a domT score of at least 250 and the lysozyme enhancing domain gives a domT score of at least 109.

In an embodiment, the GH24 catalytic domain gives a domT score of at least 220 and the lysozyme enhancing domain gives a domT score of at least 112. In an embodiment, the GH24 catalytic domain gives a domT score of at least 230 and the lysozyme enhancing domain gives a domT score of at least 112. In an embodiment, the GH24 catalytic domain gives a domT score of at least 240 and the lysozyme enhancing domain gives a domT score of at least 112. In an embodiment, the GH24 catalytic domain gives a domT score of at least 250 and the lysozyme enhancing domain gives a domT score of at least 112.

In an embodiment, the GH24 catalytic domain gives a domT score of at least 220 and the lysozyme enhancing domain gives a domT score of at least 115. In an embodiment, the GH24 catalytic domain gives a domT score of at least 230 and the lysozyme enhancing domain gives a domT score of at least 115. In an embodiment, the GH24 catalytic domain gives a domT score of at least 240 and the lysozyme enhancing domain gives a domT score of at least 115. In an embodiment, the GH24 catalytic domain gives a domT score of at least 250 and the lysozyme enhancing domain gives a domT score of at least 115.

In an embodiment, the GH24 catalytic domain gives a domT score of at least 220 and the lysozyme enhancing domain gives a domT score of at least 118. In an embodiment, the GH24 catalytic domain gives a domT score of at least 230 and the lysozyme enhancing domain gives a domT score of at least 118. In an embodiment, the GH24 catalytic domain gives a domT score of at least 240 and the lysozyme enhancing domain gives a domT score of at least 118. In an embodiment, the GH24 catalytic domain gives a domT score of at least 250 and the lysozyme enhancing domain gives a domT score of at least 118.

In an embodiment, the polypeptide having lysozyme activity is obtained or obtainable from the kingdom Fungi, preferably from the phylum Ascomycota.

In an embodiment, the polypeptide having lysozyme activity has at least 30% of the antimicrobial activity of SEQ ID NO: 257 against Clostridium perfringens using the conditions 50% MHB, pH 6. In an embodiment, the polypeptide having lysozyme activity has at least 50% of the antimicrobial activity of SEQ ID NO: 257 against Clostridium perfringens using the conditions 50% MHB, pH 6. In an embodiment, the polypeptide having lysozyme activity has at least 60% of the antimicrobial activity of SEQ ID NO: 257 against Clostridium perfringens using the conditions 50% MHB, pH 6.

In an embodiment, the polypeptide having lysozyme activity has at least 70% of the antimicrobial activity of SEQ ID NO: 257 against Clostridium perfringens using the conditions 50% MHB, pH 6. In an embodiment, the polypeptide having lysozyme activity has at least 75% of the antimicrobial activity of SEQ ID NO: 257 against Clostridium perfringens using the conditions 50% MHB, pH 6. In an embodiment, the polypeptide having lysozyme activity has at least 80% of the antimicrobial activity of SEQ ID NO: 257 against Clostridium perfringens using the conditions 50% MHB, pH 6.

In an embodiment, the polypeptide having lysozyme activity has at least 85% of the antimicrobial activity of SEQ ID NO: 257 against Clostridium perfringens using the conditions 50% MHB, pH 6. In an embodiment, the polypeptide having lysozyme activity has at least 90% of the antimicrobial activity of SEQ ID NO: 257 against Clostridium perfringens using the conditions 50% MHB, pH 6. In an embodiment, the polypeptide having lysozyme activity has at least 95% of the antimicrobial activity of SEQ ID NO: 257 against Clostridium perfringens using the conditions 50% MHB, pH 6. In an embodiment, the polypeptide having lysozyme activity has at least 100% of the antimicrobial activity of SEQ ID NO: 257 against Clostridium perfringens using the conditions 50% MHB, PH 6.

In one aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the GH24 catalytic domain comprises one or more motif I: [TAS][VIL][GAC][YFI]GHX[CAYIV] (SEQ ID NO: 252) and/or one or more motif II [LV][NDST]XN[QE][YFW][GASND]ALXS[FLY]X[FY]N (SEQ ID NO: 253); and
(b) the lysozyme enhancing domain comprises one or more motif III:

(SEQ ID NO: 254)
[CGY][YF][VIL][ASTP][DG]X[YF][VIT]X[TS][GAN].

In one aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the catalytic GH24 domain comprises one or more motif I: [TAS][VIL][GAC][YFI]GHX[CAYIV] (SEQ ID NO: 281) and/or one or more motif II LNXN[QE][YFW][GA]ALXS[WFL]X[YF]N (SEQ ID NO: 283); and
(b) the lysozyme enhancing domain comprises one or more motif III:

(SEQ ID NO: 284)
C[YF][VI][AST]D[YKF][YF][VI]XTG.

In one aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the GH24 catalytic domain comprises one or more motif I: T[VI]GYGHXC (SEQ ID NO: 282) and/or one or more motif II LNXN[QE][YFW][GA]ALXS[WFL]X[YF]N (SEQ ID NO: 283); and
(b) the lysozyme enhancing domain comprises one or more motif III:

(SEQ ID NO: 284)
C[YF][VI][AST]D[YKF][YF][VI]XTG.

In one aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the GH24 catalytic domain comprises one or more motif I: [TAS][VIL][GAC][YFI]GHX[CAYIV] (SEQ ID NO: 252), one or more motif I [LV][NDST]XN[QE][YFW][GASND]ALXS[WFLY]X[FY]N (SEQ ID NO: 253) and/or one or more motif IV [GEV]LXXRRXXE (SEQ ID NO: 285); and
(b) the lysozyme enhancing domain comprises one or more motif III:

(SEQ ID NO: 254)
[CGY][YF][VIL][ASTP][DG]X[YF][VIT]X[TS][GAN].

In one aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the GH24 catalytic domain comprises one or more motif I: [TAS][VIL][GAC][YFI]GHX[CAYIV] (SEQ ID NO: 281), one or more motif II LNXN[QE][YFW][GA]ALXS[WFL]X[YF]N (SEQ ID NO: 283) and/or one or more motif IV GLXXRRXXE (SEQ ID NO: 286); and
(b) the lysozyme enhancing domain comprises one or more motif III:

(SEQ ID NO: 284)
C[YF][VI][AST]D[YKF][YF][VI]XTG.

In one aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the GH24 catalytic domain comprises one or more motif 1: [TAS][VIL][GAC][YFI]GHX[CAYIV] (SEQ ID NO: 252) and one or more motif II [LV][NDST]XN[QE][YFW][GASND]ALXS[WFLY]X[FY]N (SEQ ID NO: 253); and
(b) the lysozyme enhancing domain comprises one or more motif III:

(SEQ ID NO: 254)
[CGY][YF][VIL][ASTP][DG]X[YF][VIT]X[TS][GAN].

In one aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the GH24 catalytic domain comprises one or more motif I [TAS][VIL][GAC][YFI]GHX[CAYIV] (SEQ ID NO: 281) and one or more motif II LNXN[QE][YFW][GA]ALXS[WFL]X[YF]N (SEQ ID NO: 283); and
(b) the lysozyme enhancing domain comprises one or more motif III:

(SEQ ID NO: 284)
C[YF][VI][AST]D[YKF][YF][VI]XTG.

In one aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the GH24 catalytic domain comprises one or more motif I: T [VI]GYGHXC (SEQ ID NO: 282) and one or more motif II LNXN[QE][YFW][GA]ALXS[WFL]X[YF]N (SEQ ID NO: 283); and
(b) the lysozyme enhancing domain comprises one or more motif III:

(SEQ ID NO: 284)
C[YF][VI][AST]D[YKF][YF][VI]XTG.

In one aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the GH24 catalytic (a) domain comprises one or more motif I: [TAS][VIL][GAC][YFI]GHX[CAYIV] (SEQ ID NO: 252), more motif II [LV][NDST]XN[QE][YFW][GASND]ALXS[WFLY]X[FY]N (SEQ ID NO: 253) and one or more motif IV [GEV]LXXRRXXE (SEQ ID NO: 285); and
(b) the lysozyme enhancing domain comprises one or more motif III:

(SEQ ID NO: 254)
[CGY][Y F][VIL][ASTP][DG]X[YF][VIT]X[TS][GAN].

In one aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the GH24 catalytic domain comprises one or more motif I: [TAS][VIL][GAC][YFI]GHX[CAYIV] (SEQ ID NO: 281), one or more motif II LNXN[QE][YFW][GA]ALXS[WFL]X[YF]N (SEQ ID NO: 283) and one or more motif IV GLXXRRXXE (SEQ ID NO: 286); and
(b) the lysozyme enhancing domain comprises one or more motif III:

(SEQ ID NO: 284)
C[YF][VI][AST]D[YKF][YF][VI]XTG.

In an embodiment, the polypeptide having lysozyme activity is obtained or obtainable from the kingdom Fungi, preferably from the phylum Ascomycota.

In an embodiment, the polypeptide having lysozyme activity has at least 30% of the antimicrobial activity of SEQ ID NO: 257 against Clostridium perfringens using the conditions 50% MHB, pH 6. In an embodiment, the polypeptide having lysozyme activity has at least 50% of the antimicrobial activity of SEQ ID NO: 257 against Clostridium perfringens using the conditions 50% MHB, pH 6. In an embodiment, the polypeptide having lysozyme activity has at least 60% of the antimicrobial activity of SEQ ID NO: 257 against Clostridium perfringens using the conditions 50% MHB, PH 6.

In an embodiment, the polypeptide having lysozyme activity has at least 70% of the antimicrobial activity of SEQ ID NO: 257 against Clostridium perfringens using the conditions 50% MHB, pH 6. In an embodiment, the polypeptide having lysozyme activity has at least 75% of the antimicrobial activity of SEQ ID NO: 257 against Clostridium perfringens using the conditions 50% MHB, pH 6. In an embodiment, the polypeptide having lysozyme activity has at least 80% of the antimicrobial activity of SEQ ID NO: 257 against Clostridium perfringens using the conditions 50% MHB, pH 6.

In an embodiment, the polypeptide having lysozyme activity has at least 85% of the antimicrobial activity of SEQ ID NO: 257 against Clostridium perfringens using the conditions 50% MHB, pH 6. In an embodiment, the polypeptide having lysozyme activity has at least 90% of the antimicrobial activity of SEQ ID NO: 257 against Clostridium perfringens using the conditions 50% MHB, pH 6. In an embodiment, the polypeptide having lysozyme activity has at least 95% of the antimicrobial activity of SEQ ID NO: 257 against Clostridium perfringens using the conditions 50% MHB, pH 6. In an embodiment, the polypeptide having lysozyme activity has at least 100% of the antimicrobial activity of SEQ ID NO: 257 against Clostridium perfringens using the conditions 50% MHB, pH 6.

In another aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(a) the GH24 catalytic domain comprises one or more motif I: [TAS][VIL][GAC][YFI]GHX[CAYIV] (SEQ ID NO: 252), one or more motif II [LV][NDST]XN[QE][YFW][GASND]ALXS[WFLY]X[FY]N (SEQ ID NO: 253) and has at least 50%, e.g., at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 14; and
(b) the GH24 lysozyme enhancing domain comprises one or more motif I: [TAS][VIL][GAC][YFI]GHX[CAYIV] (SEQ ID NO: 252), one or more motif II [LV][NDST]XN[QE][YFW][GASND]ALXS[WFLY]X[FY]N (SEQ ID NO: 253) and has at least 45%, e.g., at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 180.

In an embodiment, the polypeptide having lysozyme activity is obtained or obtainable from the kingdom Fungi, preferably from the phylum Ascomycota.

In an embodiment, the polypeptide having lysozyme activity has at least 30% of the antimicrobial activity of SEQ ID NO: 257 against Clostridium perfringens using the conditions 50% MHB, pH 6. In an embodiment, the polypeptide having lysozyme activity has at least 50% of the antimicrobial activity of SEQ ID NO: 257 against Clostridium perfringens using the conditions 50% MHB, pH 6. In an embodiment, the polypeptide having lysozyme activity has at least 60% of the antimicrobial activity of SEQ ID NO: 257 against Clostridium perfringens using the conditions 50% MHB, pH 6.

In an embodiment, the polypeptide having lysozyme activity has at least 70% of the antimicrobial activity of SEQ ID NO: 257 against Clostridium perfringens using the conditions 50% MHB, pH 6. In an embodiment, the polypeptide having lysozyme activity has at least 75% of the antimicrobial activity of SEQ ID NO: 257 against Clostridium perfringens using the conditions 50% MHB, pH 6. In an embodiment, the polypeptide having lysozyme activity has at least 80% of the antimicrobial activity of SEQ ID NO: 257 against Clostridium perfringens using the conditions 50% MHB, pH 6.

In an embodiment, the polypeptide having lysozyme activity has at least 85% of the antimicrobial activity of SEQ ID NO: 257 against Clostridium perfringens using the conditions 50% MHB, pH 6. In an embodiment, the polypeptide having lysozyme activity has at least 90% of the antimicrobial activity of SEQ ID NO: 257 against Clostridium perfringens using the conditions 50% MHB, pH 6. In an embodiment, the polypeptide having lysozyme activity has at least 95% of the antimicrobial activity of SEQ ID NO: 257 against Clostridium perfringens using the conditions 50% MHB, pH 6. In an embodiment, the polypeptide having lysozyme activity has at least 100% of the antimicrobial activity of SEQ ID NO: 257 against Clostridium perfringens using the conditions 50% MHB, pH 6.

In another aspect, the invention relates to an animal feed or animal feed additive comprising one or more polypeptides having lysozyme activity, wherein the polypeptide having lysozyme activity comprises one or more GH24 catalytic domains and one or more lysozyme enhancing domains, wherein:
(b) the GH24 catalytic domain comprises one or more motif I: [TAS][VIL][GAC][YFI]GHX[CAYIV] (SEQ ID NO: 252), one or more motif II [LV][NDST]XN[QE][YFW][GASND]ALXS[WFLY]X[FY]N (SEQ ID NO: 253) and has at least 65%, e.g., at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to one or more of SEQ ID NO: 1 to 122; and
(b) the lysozyme enhancing domain comprises one or more motif I: [TAS][VIL][GAC][YFI]GHX[CAYIV] (SEQ ID NO: 252), one or more motif II [LV][NDST]XN[QE][YFW][GASND]ALXS[WFLY]X[FY]N (SEQ ID NO: 253) and has at least 65%, e.g., at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to one or more of SEQ ID NO: 123 to 251.

In an embodiment, the polypeptide having lysozyme activity is obtained or obtainable from the kingdom Fungi, preferably from the phylum Ascomycota.

In an embodiment, the polypeptide having lysozyme activity has at least 30% of the antimicrobial activity of SEQ ID NO: 257 against Clostridium perfringens using the conditions 50% MHB, pH 6. In an embodiment, the polypeptide having lysozyme activity has at least 50% of the antimicrobial activity of SEQ ID NO: 257 against Clostridium perfringens using the conditions 50% MHB, pH 6. In an embodiment, the polypeptide having lysozyme activity has at least 60% of the antimicrobial activity of SEQ ID NO: 257 against Clostridium perfringens using the conditions 50% MHB, pH 6.

In an embodiment, the polypeptide having lysozyme activity has at least 70% of the antimicrobial activity of SEQ ID NO: 257 against Clostridium perfringens using the conditions 50% MHB, pH 6. In an embodiment, the polypeptide having lysozyme activity has at least 75% of the antimicrobial activity of SEQ ID NO: 257 against Clostridium perfringens using the conditions 50% MHB, pH 6. In an embodiment, the polypeptide having lysozyme activity has at least 80% of the antimicrobial activity of SEQ ID NO: 257 against Clostridium perfringens using the conditions 50% MHB, pH 6.

In an embodiment, the polypeptide having lysozyme activity has at least 85% of the antimicrobial activity of SEQ ID NO: 257 against Clostridium perfringens using the conditions 50% MHB, pH 6. In an embodiment, the polypeptide having lysozyme activity has at least 90% of the antimicrobial activity of SEQ ID NO: 257 against Clostridium perfringens using the conditions 50% MHB, pH 6. In an embodiment, the polypeptide having lysozyme activity has at least 95% of the antimicrobial activity of SEQ ID NO: 257 against Clostridium perfringens using the conditions 50% MHB, pH 6. In an embodiment, the polypeptide having lysozyme activity has at least 100% of the antimicrobial activity of SEQ ID NO: 257 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 257.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 257 and at least 70% of the antimicrobial activity of SEQ ID NO: 257 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 257 and at least 80% of the antimicrobial activity of SEQ ID NO: 257 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 257 and at least 90% of the antimicrobial activity of SEQ ID NO: 257 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 257 and at least 95% of the antimicrobial activity of SEQ ID NO: 257 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 257 and at least 100% of the antimicrobial activity of SEQ ID NO: 257 against Clostridium perfringens using the conditions 50% MHB, pH 6.

In a further embodiment, the animal feed or animal feed additive comprises one or more polypeptides having lysozyme activity, wherein the polypeptide having lysozyme activity differs by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 257. In an embodiment, the polypeptide has at least 70%, e.g., at least 80%, at least 90%, at least 95% or at least 100% of the antimicrobial activity of SEQ ID NO: 257 against Clostridium perfringens using the conditions 50% MHB, pH 6.

In a preferred embodiment, the animal feed or animal feed additive comprises one or more polypeptides having lysozyme activity, wherein the polypeptide having lysozyme activity comprising or consists of the amino acid sequence of SEQ ID NO: 257 or an allelic variant thereof having lysozyme activity; comprises or consists of the amino acid sequence of SEQ ID NO: 257 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In a preferred embodiment, the animal feed or animal feed additive comprises one or more polypeptides having lysozyme activity, wherein the polypeptide having lysozyme activity comprising or consists of amino acids 1 to 245 of SEQ ID NO: 257 and has lysozyme activity.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 264.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 264 and at least 70% of the antimicrobial activity of SEQ ID NO: 264 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 264 and at least 80% of the antimicrobial activity of SEQ ID NO: 264 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 264 and at least 90% of the antimicrobial activity of SEQ ID NO: 264 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 264 and at least 95% of the antimicrobial activity of SEQ ID NO: 264 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 264 and at least 100% of the antimicrobial activity of SEQ ID NO: 264 against Clostridium perfringens using the conditions 50% MHB, pH 6.

In a further embodiment, the animal feed or animal feed additive comprises one or more polypeptides having lysozyme activity, wherein the polypeptide having lysozyme activity differs by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 264. In an embodiment, the polypeptide has at least 70%, e.g., at least 80%, at least 90%, at least 95% or at least 100% of the antimicrobial activity of SEQ ID NO: 264 against Clostridium perfringens using the conditions 50% MHB, pH 6.

In a preferred embodiment, the animal feed or animal feed additive comprises one or more polypeptides having lysozyme activity, wherein the polypeptide having lysozyme activity comprising or consists of the amino acid sequence of SEQ ID NO: 264 or an allelic variant thereof having lysozyme activity; comprises or consists of the amino acid sequence of SEQ ID NO: 264 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In a preferred embodiment, the animal feed or animal feed additive comprises one or more polypeptides having lysozyme activity, wherein the polypeptide having lysozyme activity comprising or consists of amino acids 1 to 245 of SEQ ID NO: 264 and has lysozyme activity.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 267.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 267 and at least 70% of the antimicrobial activity of SEQ ID NO: 267 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 267 and at least 80% of the antimicrobial activity of SEQ ID NO: 267 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 267 and at least 90% of the antimicrobial activity of SEQ ID NO: 267 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 267 and at least 95% of the antimicrobial activity of SEQ ID NO: 267 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 267 and at least 100% of the antimicrobial activity of SEQ ID NO: 267 against Clostridium perfringens using the conditions 50% MHB, pH 6.

In a further embodiment, the animal feed or animal feed additive comprises one or more polypeptides having lysozyme activity, wherein the polypeptide having lysozyme activity differs by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 267. In an embodiment, the polypeptide has at least 70%, e.g., at least 80%, at least 90%, at least 95% or at least 100% of the antimicrobial activity of SEQ ID NO: 267 against Clostridium perfringens using the conditions 50% MHB, pH 6.

In a preferred embodiment, the animal feed or animal feed additive comprises one or more polypeptides having lysozyme activity, wherein the polypeptide having lysozyme activity comprising or consists of the amino acid sequence of SEQ ID NO: 267 or an allelic variant thereof having lysozyme activity; comprises or consists of the amino acid sequence of SEQ ID NO: 267 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In a preferred embodiment, the animal feed or animal feed additive comprises one or more polypeptides having lysozyme activity, wherein the polypeptide having lysozyme activity comprising or consists of amino acids 1 to 248 of SEQ ID NO: 267 and has lysozyme activity.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 291.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 291 and at least 70% of the antimicrobial activity of SEQ ID NO: 291 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 291 and at least 80% of the antimicrobial activity of SEQ ID NO: 291 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 291 and at least 90% of the antimicrobial activity of SEQ ID NO: 291 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 291 and at least 95% of the antimicrobial activity of SEQ ID NO: 291 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 291 and at least 100% of the antimicrobial activity of SEQ ID NO: 291 against Clostridium perfringens using the conditions 50% MHB, pH 6.

In a further embodiment, the animal feed or animal feed additive comprises one or more polypeptides having lysozyme activity, wherein the polypeptide having lysozyme activity differs by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 291. In an embodiment, the polypeptide has at least 70%, e.g., at least 80%, at least 90%, at least 95% or at least 100% of the antimicrobial activity of SEQ ID NO: 291 against Clostridium perfringens using the conditions 50% MHB, pH 6.

In a preferred embodiment, the animal feed or animal feed additive comprises one or more polypeptides having lysozyme activity, wherein the polypeptide having lysozyme activity comprising or consists of the amino acid sequence of SEQ ID NO: 291 or an allelic variant thereof having lysozyme activity; comprises or consists of the amino acid sequence of SEQ ID NO: 291 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In a preferred embodiment, the animal feed or animal feed additive comprises one or more polypeptides having lysozyme activity, wherein the polypeptide having lysozyme activity comprising or consists of amino acids 1 to 245 of SEQ ID NO: 291 and has lysozyme activity.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 294.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 294 and at least 70% of the antimicrobial activity of SEQ ID NO: 294 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 294 and at least 80% of the antimicrobial activity of SEQ ID NO: 294 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 294 and at least 90% of the antimicrobial activity of SEQ ID NO: 294 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 294 and at least 95% of the antimicrobial activity of SEQ ID NO: 294 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 294 and at least 100% of the antimicrobial activity of SEQ ID NO: 294 against Clostridium perfringens using the conditions 50% MHB, pH 6.

In a further embodiment, the animal feed or animal feed additive comprises one or more polypeptides having lysozyme activity, wherein the polypeptide having lysozyme activity differs by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 294. In an embodiment, the polypeptide has at least 70%, e.g., at least 80%, at least 90%, at least 95% or at least 100% of the antimicrobial activity of SEQ ID NO: 294 against Clostridium perfringens using the conditions 50% MHB, pH 6.

In a preferred embodiment, the animal feed or animal feed additive comprises one or more polypeptides having lysozyme activity, wherein the polypeptide having lysozyme activity comprising or consists of the amino acid sequence of SEQ ID NO: 294 or an allelic variant thereof having lysozyme activity; comprises or consists of the amino acid sequence of SEQ ID NO: 294 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In a preferred embodiment, the animal feed or animal feed additive comprises one or more polypeptides having lysozyme activity, wherein the polypeptide having lysozyme activity comprising or consists of amino acids 1 to 245 of SEQ ID NO: 294 and has lysozyme activity.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 297.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 297 and at least 70% of the antimicrobial activity of SEQ ID NO: 297 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 297 and at least 80% of the antimicrobial activity of SEQ ID NO: 297 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 297 and at least 90% of the antimicrobial activity of SEQ ID NO: 297 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 297 and at least 95% of the antimicrobial activity of SEQ ID NO: 297 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 297 and at least 100% of the antimicrobial activity of SEQ ID NO: 297 against Clostridium perfringens using the conditions 50% MHB, pH 6.

In a further embodiment, the animal feed or animal feed additive comprises one or more polypeptides having lysozyme activity, wherein the polypeptide having lysozyme activity differs by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 297. In an embodiment, the polypeptide has at least 70%, e.g., at least 80%, at least 90%, at least 95% or at least 100% of the antimicrobial activity of SEQ ID NO: 297 against Clostridium perfringens using the conditions 50% MHB, PH 6.

In a preferred embodiment, the animal feed or animal feed additive comprises one or more polypeptides having lysozyme activity, wherein the polypeptide having lysozyme activity comprising or consists of the amino acid sequence of SEQ ID NO: 297 or an allelic variant thereof having lysozyme activity; comprises or consists of the amino acid sequence of SEQ ID NO: 297 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In a preferred embodiment, the animal feed or animal feed additive comprises one or more polypeptides having lysozyme activity, wherein the polypeptide having lysozyme activity comprising or consists of amino acids 1 to 249 of SEQ ID NO: 297 and has lysozyme activity.

In a further embodiment, the invention relates to an animal feed or animal feed additive comprising one or more polypeptides having lysozyme activity, wherein the polypeptide having lysozyme activity has at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 300.

In a further embodiment, the invention relates to an animal feed or animal feed additive comprising one or more polypeptides having lysozyme activity, wherein the polypeptide having lysozyme activity has at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 300 and at least 70% of the antimicrobial activity of SEQ ID NO: 300 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 300 and at least 80% of the antimicrobial activity of SEQ ID NO: 300 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 300 and at least 90% of the antimicrobial activity of SEQ ID NO: 300 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 300 and at least 95% of the antimicrobial activity of SEQ ID NO: 300 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 300 and at least 100% of the antimicrobial activity of SEQ ID NO: 300 against Clostridium perfringens using the conditions 50% MHB, pH 6.

In a further embodiment, the animal feed or animal feed additive comprises one or more polypeptides having lysozyme activity, wherein the polypeptide having lysozyme activity differs by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 300. In an embodiment, the polypeptide has at least 70%, e.g., at least 80%, at least 90%, at least 95% or at least 100% of the antimicrobial activity of SEQ ID NO: 300 against Clostridium perfringens using the conditions 50% MHB, PH 6.

In a preferred embodiment, the animal feed or animal feed additive comprises one or more polypeptides having lysozyme activity, wherein the polypeptide having lysozyme activity comprising or consists of the amino acid sequence of SEQ ID NO: 300 or an allelic variant thereof having lysozyme activity; comprises or consists of the amino acid sequence of SEQ ID NO: 300 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In a preferred embodiment, the animal feed or animal feed additive comprises one or more polypeptides having lysozyme activity, wherein the polypeptide having lysozyme activity comprising or consists of amino acids 1 to 247 of SEQ ID NO: 300 and has lysozyme activity.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 303.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 303 and at least 70% of the antimicrobial activity of SEQ ID NO: 303 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 303 and at least 80% of the antimicrobial activity of SEQ ID NO: 303 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 303 and at least 90% of the antimicrobial activity of SEQ ID NO: 303 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 303 and at least 95% of the antimicrobial activity of SEQ ID NO: 303 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 303 and at least 100% of the antimicrobial activity of SEQ ID NO: 303 against Clostridium perfringens using the conditions 50% MHB, pH 6.

In a further embodiment, the animal feed or animal feed additive comprises one or more polypeptides having lysozyme activity, wherein the polypeptide having lysozyme activity differs by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 303. In an embodiment, the polypeptide has at least 70%, e.g., at least 80%, at least 90%, at least 95% or at least 100% of the antimicrobial activity of SEQ ID NO: 303 against Clostridium perfringens using the conditions 50% MHB, PH 6.

In a preferred embodiment, the animal feed or animal feed additive comprises one or more polypeptides having lysozyme activity, wherein the polypeptide having lysozyme activity comprising or consists of the amino acid sequence of SEQ ID NO: 303 or an allelic variant thereof having lysozyme activity; comprises or consists of the amino acid sequence of SEQ ID NO: 303 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In a preferred embodiment, the animal feed or animal feed additive comprises one or more polypeptides having lysozyme activity, wherein the polypeptide having lysozyme activity comprising or consists of amino acids 1 to 250 of SEQ ID NO: 303 and has lysozyme activity.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 306.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 306 and at least 70% of the antimicrobial activity of SEQ ID NO: 306 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 306 and at least 80% of the antimicrobial activity of SEQ ID NO: 306 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 306 and at least 90% of the antimicrobial activity of SEQ ID NO: 306 against Clostridium perfringens using the conditions 50% MHB, pH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 306 and at least 95% of the antimicrobial activity of SEQ ID NO: 306 against Clostridium perfringens using the conditions 50% MHB, PH 6.

In a further embodiment, the invention relates to an animal feed or animal feed additive comprising one or more polypeptides having lysozyme activity, wherein the polypeptide having lysozyme activity has at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 306 and at least 100% of the antimicrobial activity of SEQ ID NO: 306 against Clostridium perfringens using the conditions 50% MHB, pH 6.

In a further embodiment, the animal feed or animal feed additive comprises one or more polypeptides having lysozyme activity, wherein the polypeptide having lysozyme activity differs by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 306. In an embodiment, the polypeptide has at least 70%, e.g., at least 80%, at least 90%, at least 95% or at least 100% of the antimicrobial activity of SEQ ID NO: 306 against Clostridium perfringens using the conditions 50% MHB, PH 6.

In a preferred embodiment, the animal feed or animal feed additive comprises one or more polypeptides having lysozyme activity, wherein the polypeptide having lysozyme activity comprising or consists of the amino acid sequence of SEQ ID NO: 306 or an allelic variant thereof having lysozyme activity; comprises or consists of the amino acid sequence of SEQ ID NO: 306 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In a preferred embodiment, the animal feed or animal feed additive comprises one or more polypeptides having lysozyme activity, wherein the polypeptide having lysozyme activity comprising or consists of amino acids 1 to 240 of SEQ ID NO: 306 and has lysozyme activity.

### Novel Lysozymes of the Invention

The invention further relates to polypeptides having a sequence identity to the mature polypeptide of SEQ ID NO: 256 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have lysozyme activity. In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from the mature polypeptide of SEQ ID NO: 256. In an embodiment, the polypeptide has been isolated. In an embodiment, the polypeptide has at least at least 70%, e.g., at least 80%, at least 90%, at least 95% or at least 100% of the lysozyme activity of the mature polypeptide of SEQ ID NO: 256.

A polypeptide of the present invention preferably comprises or consists of the amino acid sequence of SEQ ID NO: 256 or an allelic variant thereof; comprises or consists of the amino acid sequence of SEQ ID NO: 256 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of the mature polypeptide of SEQ ID NO: 256. In another aspect, the polypeptide comprises or consists of amino acids 1 to 245 of SEQ ID NO: 256.

The present invention further relates to polypeptides having a sequence identity to SEQ ID NO: 257 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 70% of the lysozyme activity of SEQ ID NO: 257.

In a particular embodiment, the invention relates to polypeptides having a sequence identity to SEQ ID NO: 257 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 80% of the lysozyme activity of SEQ ID NO: 257.

In a particular embodiment, the invention relates to polypeptides having a sequence identity to SEQ ID NO: 257 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 90% of the lysozyme activity of SEQ ID NO: 257.

In a particular embodiment, the invention relates to polypeptides having a sequence identity to SEQ ID NO: 257 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 95% of the lysozyme activity of SEQ ID NO: 257.

In a particular embodiment, the invention relates to polypeptides having a sequence identity to SEQ ID NO: 257 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 100% of the lysozyme activity of SEQ ID NO: 257.

In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 257. In an embodiment, the polypeptide has at least at least 70%, e.g., at least 80%, at least 90%, at least 95% or at least 100% of the lysozyme activity of SEQ ID NO: 257.

A polypeptide of the present invention preferably comprises or consists of the amino acid sequence of SEQ ID NO: 257 or an allelic variant thereof having lysozyme activity; comprises or consists of the amino acid sequence of SEQ ID NO: 257 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of amino acids 1 to 245 of SEQ ID NO: 257 and has lysozyme activity.

In another aspect, the present invention relates to a polypeptide having lysozyme activity encoded by a polynucleotide that hybridizes under medium-high stringency conditions, high stringency conditions, or very high stringency conditions with (i) the mature polypeptide coding sequence of SEQ ID NO: 255, (ii) the cDNA sequence thereof; or (iii) the full-length complement of (i) or (ii) (Sambrook et al., 1989, Molecular Cloning, A Laboratory Manual, 2d edition, Cold Spring Harbor, New York). In an embodiment, the polypeptide has been isolated.

In another embodiment, the present invention relates to a polypeptide having lysozyme activity encoded by a polynucleotide having a sequence identity to the mature polypeptide coding sequence of SEQ ID NO: 255 or the cDNA sequence thereof of at least 80%, e.g., at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 257 comprising a substitution, and/or deletion, and/or insertion at one or more (e.g., several) positions. In an embodiment, the number of positions comprising one or more amino acid substitutions, and/or one or more amino acid deletions, and/or one or more amino acid insertions or any combination thereof in SEQ ID NO: 257 is not more than 49, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49. In another embodiment, the number of positions comprising one or more amino acid substitutions, and/or one or more amino acid deletions, and/or one or more amino acid insertions or any combination thereof in SEQ ID NO: 257 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 257 is not more than 10, e.g., 1, 2, 3, 4, 5, 6, 7, 8, 9 or 10. In another embodiment, the number of substitutions and/or deletions and/or insertions in SEQ ID NO: 257 is not more than 10, e.g., 1, 2, 3, 4, 5, 6, 7, 8, 9 or 10. In a further embodiment, the number of substitutions in SEQ ID NO: 257 is not more than 10, e.g., 1, 2, 3, 4, 5, 6, 7, 8, 9 or 10. In a further embodiment, the number of conservative substitutions in SEQ ID NO: 257 is not more than 10, e.g., 1, 2, 3, 4, 5, 6, 7, 8, 9 or 10. In an embodiment, the variant has at least at least 70%, e.g., at least 80%, at least 90%, at least 95% or at least 100% of the lysozyme activity of SEQ ID NO: 257.

The invention further relates to polypeptides having a sequence identity to the mature polypeptide of SEQ ID NO: 266 of at least 95%, e.g., at least 96%, at least 97%, at least 98%, at least 99%, or 100%, which have lysozyme activity. In one aspect, the polypeptides differ by up to 12 amino acids, e.g., between 1 and 12 amino acids, such as 1-10 or 1-5 amino acids, or 1, 2, 3, 4, 5, 6, 7, 8, 9, 10, 11 or 12 amino acids from the mature polypeptide of SEQ ID NO: 266. In an embodiment, the polypeptide has been isolated. In an embodiment, the polypeptide has at least at least 70%, e.g., at least 80%, at least 90%, at least 95% or at least 100% of the lysozyme activity of the mature polypeptide of SEQ ID NO: 266.

A polypeptide of the present invention preferably comprises or consists of the amino acid sequence of SEQ ID NO: 266 or an allelic variant thereof; comprises or consists of the amino acid sequence of SEQ ID NO: 266 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids, at least 240 amino acids or at least 245 amino acids. In another aspect, the polypeptide comprises or consists of the mature polypeptide of SEQ ID NO: 266. In another aspect, the polypeptide comprises or consists of amino acids 1 to 248 of SEQ ID NO: 266.

The present invention further relates to polypeptides having a sequence identity to SEQ ID NO: 267 of at least 95%, e.g., at least 96%, at least 97%, at least 98%, at least 99%, or 100%, and wherein the polypeptide has at least at least 70% of the lysozyme activity of SEQ ID NO: 267.

The present invention further relates to polypeptides having a sequence identity to SEQ ID NO: 267 of at least 95%, e.g., at least 96%, at least 97%, at least 98%, at least 99%, or 100%, and wherein the polypeptide has at least at least 80% of the lysozyme activity of SEQ ID NO: 267.

The present invention further relates to polypeptides having a sequence identity to SEQ ID NO: 267 of at least 95%, e.g., at least 96%, at least 97%, at least 98%, at least 99%, or 100%, and wherein the polypeptide has at least at least 90% of the lysozyme activity of SEQ ID NO: 267.

The present invention further relates to polypeptides having a sequence identity to SEQ ID NO: 267 of at least 95%, e.g., at least 96%, at least 97%, at least 98%, at least 99%, or 100%, and wherein the polypeptide has at least at least 95% of the lysozyme activity of SEQ ID NO: 267.

The present invention further relates to polypeptides having a sequence identity to SEQ ID NO: 267 of at least 95%, e.g., at least 96%, at least 97%, at least 98%, at least 99%, or 100%, and wherein the polypeptide has at least at least 100% of the lysozyme activity of SEQ ID NO: 267.

In one aspect, the polypeptides differ by up to 49 amino acids, e.g., between 1 and 12 amino acids, such as 1-10 or 1-5 amino acids, or 1, 2, 3, 4, 5, 6, 7, 8, 9, 10, 11 or 12 amino acids from SEQ ID NO: 267. In an embodiment, the polypeptide has at least at least 70%, e.g., at least 80%, at least 90%, at least 95% or at least 100% of the lysozyme activity of SEQ ID NO: 267.

A polypeptide of the present invention preferably comprises or consists of the amino acid sequence of SEQ ID NO: 267 or an allelic variant thereof having lysozyme activity; comprises or consists of the amino acid sequence of SEQ ID NO: 267 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids, at least 240 amino acids or at least 245 amino acids. In another aspect, the polypeptide comprises or consists of amino acids 1 to 248 of SEQ ID NO: 267 and has lysozyme activity.

In another aspect, the present invention relates to a polypeptide having lysozyme activity encoded by a polynucleotide that hybridizes under very high stringency conditions with (i) the mature polypeptide coding sequence of SEQ ID NO: 265, (ii) the cDNA sequence thereof; or (iii) the full-length complement of (i) or (ii) (Sambrook et al., 1989, Molecular Cloning, A Laboratory Manual, 2d edition, Cold Spring Harbor, New York). In an embodiment, the polypeptide has been isolated.

In another embodiment, the present invention relates to a polypeptide having lysozyme activity encoded by a polynucleotide having a sequence identity to the mature polypeptide coding sequence of SEQ ID NO: 265 or the cDNA sequence thereof of at least 95%, e.g., at least 96%, at least 97%, at least 98%, at least 99%, or 100%. In a further embodiment, the polypeptide has been isolated.

In another embodiment, the present invention relates to variants of the mature polypeptide of SEQ ID NO: 267 comprising a substitution, and/or deletion, and/or insertion at one or more (e.g., several) positions. In an embodiment, the number of positions comprising one or more amino acid substitutions, and/or one or more amino acid deletions, and/or one or more amino acid insertions or any combination thereof in SEQ ID NO: 267 is not more than 12, e.g., 1, 2, 3, 4, 5, 6, 7, 8, 9, 10, 11 or 12. In another embodiment, the number of positions comprising one or more amino acid substitutions, and/or one or more amino acid deletions, and/or one or more amino acid insertions or any combination thereof in SEQ ID NO: 267 is between 1 and 12, such as 1-10 or 1-5 positions. In an embodiment, the number of positions comprising one or more amino acid substitutions, and/or one or more amino acid deletions, and/or one or more amino acid insertions or any combination thereof in SEQ ID NO: 267 is not more than 10, e.g., 1, 2, 3, 4, 5, 6, 7, 8, 9 or 10. In another embodiment, the number of substitutions and/or deletions and/or insertions in SEQ ID NO: 267 is not more than 10, e.g., 1, 2, 3, 4, 5, 6, 7, 8, 9 or 10. In a further embodiment, the number of substitutions in SEQ ID NO: 267 is not more than 10, e.g., 1, 2, 3, 4, 5, 6, 7, 8, 9 or 10. In a further embodiment, the number of conservative substitutions in SEQ ID NO: 267 is not more than 10, e.g., 1, 2, 3, 4, 5, 6, 7, 8, 9 or 10. In an embodiment, the variant has at least at least 70%, e.g., at least 80%, at least 90%, at least 95% or at least 100% of the lysozyme activity of SEQ ID NO: 267.

The amino acid changes in the mature polypeptide of SEQ ID NO: 256, SEQ ID NO: 257, the mature polypeptide of SEQ ID NO: 266 or the mature polypeptide of SEQ ID NO: 267 described above may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant mutant molecules are tested for lysozyme activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labelling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g., Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; S vetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

The invention further relates to polypeptides having a sequence identity to the mature polypeptide of SEQ ID NO: 288 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have lysozyme activity. In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from the mature polypeptide of SEQ ID NO: 288. In an embodiment, the polypeptide has been isolated. In an embodiment, the polypeptide has at least at least 70%, e.g., at least 80%, at least 90%, at least 95% or at least 100% of the lysozyme activity of the mature polypeptide of SEQ ID NO: 288.

A polypeptide of the present invention preferably comprises or consists of the amino acid sequence of SEQ ID NO: 288 or an allelic variant thereof; comprises or consists of the amino acid sequence of SEQ ID NO: 288 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of the mature polypeptide of SEQ ID NO: 288. In another aspect, the polypeptide comprises or consists of amino acids 1 to 245 of SEQ ID NO: 288.

The present invention further relates to polypeptides having a sequence identity to SEQ ID NO: 291 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 70% of the lysozyme activity of SEQ ID NO: 291.

In a particular embodiment, the invention relates to polypeptides having a sequence identity to SEQ ID NO: 291 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 80% of the lysozyme activity of SEQ ID NO: 291.

In a particular embodiment, the invention relates to polypeptides having a sequence identity to SEQ ID NO: 291 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 90% of the lysozyme activity of SEQ ID NO: 291.

In a particular embodiment, the invention relates to polypeptides having a sequence identity to SEQ ID NO: 291 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 95% of the lysozyme activity of SEQ ID NO: 291.

In a particular embodiment, the invention relates to polypeptides having a sequence identity to SEQ ID NO: 291 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 100% of the lysozyme activity of SEQ ID NO: 291.

In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 291. In an embodiment, the polypeptide has at least at least 70%, e.g., at least 80%, at least 90%, at least 95% or at least 100% of the lysozyme activity of SEQ ID NO: 291.

A polypeptide of the present invention preferably comprises or consists of the amino acid sequence of SEQ ID NO: 291 or an allelic variant thereof having lysozyme activity; comprises or consists of the amino acid sequence of SEQ ID NO: 291 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of amino acids 1 to 245 of SEQ ID NO: 291 and has lysozyme activity.

In another aspect, the present invention relates to a polypeptide having lysozyme activity encoded by a polynucleotide that hybridizes under medium-high stringency conditions, high stringency conditions, or very high stringency conditions with (i) the mature polypeptide coding sequence of SEQ ID NO: 287, (ii) the cDNA sequence thereof; or (iii) the full-length complement of (i) or (ii) (Sambrook et al., 1989, Molecular Cloning, A Laboratory Manual, 2d edition, Cold Spring Harbor, New York). In an embodiment, the polypeptide has been isolated.

In another embodiment, the present invention relates to a polypeptide having lysozyme activity encoded by a polynucleotide having a sequence identity to the mature polypeptide coding sequence of SEQ ID NO: 287 or the cDNA sequence thereof of at least 80%, e.g., at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 291 comprising a substitution, and/or deletion, and/or insertion at one or more (e.g., several) positions. In an embodiment, the number of positions comprising one or more amino acid substitutions, and/or one or more amino acid deletions, and/or one or more amino acid insertions or any combination thereof in SEQ ID NO: 291 is not more than 49, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49. In another embodiment, the number of positions comprising one or more amino acid substitutions, and/or one or more amino acid deletions, and/or one or more amino acid insertions or any combination thereof in SEQ ID NO: 291 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 291 is not more than 10, e.g., 1, 2, 3, 4, 5, 6, 7, 8, 9 or 10. In another embodiment, the number of substitutions and/or deletions and/or insertions in SEQ ID NO: 291 is not more than 10, e.g., 1, 2, 3, 4, 5, 6, 7, 8, 9 or 10. In a further embodiment, the number of substitutions in SEQ ID NO: 291 is not more than 10, e.g., 1, 2, 3, 4, 5, 6, 7, 8, 9 or 10. In a further embodiment, the number of conservative substitutions in SEQ ID NO: 291 is not more than 10, e.g., 1, 2, 3, 4, 5, 6, 7, 8, 9 or 10. In an embodiment, the variant has at least at least 70%, e.g., at least 80%, at least 90%, at least 95% or at least 100% of the lysozyme activity of SEQ ID NO: 291.

The invention further relates to polypeptides having a sequence identity to the mature polypeptide of SEQ ID NO: 293 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have lysozyme activity. In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from the mature polypeptide of SEQ ID NO: 293. In an embodiment, the polypeptide has been isolated. In an embodiment, the polypeptide has at least at least 70%, e.g., at least 80%, at least 90%, at least 95% or at least 100% of the lysozyme activity of the mature polypeptide of SEQ ID NO: 293.

A polypeptide of the present invention preferably comprises or consists of the amino acid sequence of SEQ ID NO: 293 or an allelic variant thereof; comprises or consists of the amino acid sequence of SEQ ID NO: 293 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of the mature polypeptide of SEQ ID NO: 293. In another aspect, the polypeptide comprises or consists of amino acids 1 to 249 of SEQ ID NO: 293.

The present invention further relates to polypeptides having a sequence identity to SEQ ID NO: 294 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 70% of the lysozyme activity of SEQ ID NO: 294.

In a particular embodiment, the invention relates to polypeptides having a sequence identity to SEQ ID NO: 294 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 80% of the lysozyme activity of SEQ ID NO: 294.

In a particular embodiment, the invention relates to polypeptides having a sequence identity to SEQ ID NO: 294 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 90% of the lysozyme activity of SEQ ID NO: 294.

In a particular embodiment, the invention relates to polypeptides having a sequence identity to SEQ ID NO: 294 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 95% of the lysozyme activity of SEQ ID NO: 294.

In a particular embodiment, the invention relates to polypeptides having a sequence identity to SEQ ID NO: 294 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 100% of the lysozyme activity of SEQ ID NO: 294.

In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 294. In an embodiment, the polypeptide has at least at least 70%, e.g., at least 80%, at least 90%, at least 95% or at least 100% of the lysozyme activity of SEQ ID NO: 294.

A polypeptide of the present invention preferably comprises or consists of the amino acid sequence of SEQ ID NO: 294 or an allelic variant thereof having lysozyme activity; comprises or consists of the amino acid sequence of SEQ ID NO: 294 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of amino acids 1 to 249 of SEQ ID NO: 294 and has lysozyme activity.

In another aspect, the present invention relates to a polypeptide having lysozyme activity encoded by a polynucleotide that hybridizes under medium-high stringency conditions, high stringency conditions, or very high stringency conditions with (i) the mature polypeptide coding sequence of SEQ ID NO: 292, (ii) the cDNA sequence thereof; or (iii) the full-length complement of (i) or (ii) (Sambrook et al., 1989, Molecular Cloning, A Laboratory Manual, 2d edition, Cold Spring Harbor, New York). In an embodiment, the polypeptide has been isolated.

In another embodiment, the present invention relates to a polypeptide having lysozyme activity encoded by a polynucleotide having a sequence identity to the mature polypeptide coding sequence of SEQ ID NO: 292 or the cDNA sequence thereof of at least 80%, e.g., at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 294 comprising a substitution, and/or deletion, and/or insertion at one or more (e.g., several) positions. In an embodiment, the number of positions comprising one or more amino acid substitutions, and/or one or more amino acid deletions, and/or one or more amino acid insertions or any combination thereof in SEQ ID NO: 294 is not more than 49, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49. In another embodiment, the number of positions comprising one or more amino acid substitutions, and/or one or more amino acid deletions, and/or one or more amino acid insertions or any combination thereof in SEQ ID NO: 294 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 294 is not more than 10, e.g., 1, 2, 3, 4, 5, 6, 7, 8, 9 or 10. In another embodiment, the number of substitutions and/or deletions and/or insertions in SEQ ID NO: 294 is not more than 10, e.g., 1, 2, 3, 4, 5, 6, 7, 8, 9 or 10. In a further embodiment, the number of substitutions in SEQ ID NO: 294 is not more than 10, e.g., 1, 2, 3, 4, 5, 6, 7, 8, 9 or 10. In a further embodiment, the number of conservative substitutions in SEQ ID NO: 294 is not more than 10, e.g., 1, 2, 3, 4, 5, 6, 7, 8, 9 or 10. In an embodiment, the variant has at least at least 70%, e.g., at least 80%, at least 90%, at least 95% or at least 100% of the lysozyme activity of SEQ ID NO: 294.

The invention further relates to polypeptides having a sequence identity to the mature polypeptide of SEQ ID NO: 296 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have lysozyme activity. In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from the mature polypeptide of SEQ ID NO: 296. In an embodiment, the polypeptide has been isolated. In an embodiment, the polypeptide has at least at least 70%, e.g., at least 80%, at least 90%, at least 95% or at least 100% of the lysozyme activity of the mature polypeptide of SEQ ID NO: 296.

A polypeptide of the present invention preferably comprises or consists of the amino acid sequence of SEQ ID NO: 296 or an allelic variant thereof; comprises or consists of the amino acid sequence of SEQ ID NO: 296 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of the mature polypeptide of SEQ ID NO: 296. In another aspect, the polypeptide comprises or consists of amino acids 1 to 245 of SEQ ID NO: 296.

The present invention further relates to polypeptides having a sequence identity to SEQ ID NO: 297 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 70% of the lysozyme activity of SEQ ID NO: 297.

In a particular embodiment, the invention relates to polypeptides having a sequence identity to SEQ ID NO: 297 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 80% of the lysozyme activity of SEQ ID NO: 297.

In a particular embodiment, the invention relates to polypeptides having a sequence identity to SEQ ID NO: 297 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 90% of the lysozyme activity of SEQ ID NO: 297.

In a particular embodiment, the invention relates to polypeptides having a sequence identity to SEQ ID NO: 297 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 95% of the lysozyme activity of SEQ ID NO: 297.

In a particular embodiment, the invention relates to polypeptides having a sequence identity to SEQ ID NO: 297 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 100% of the lysozyme activity of SEQ ID NO: 297.

In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 297. In an embodiment, the polypeptide has at least at least 70%, e.g., at least 80%, at least 90%, at least 95% or at least 100% of the lysozyme activity of SEQ ID NO: 297.

A polypeptide of the present invention preferably comprises or consists of the amino acid sequence of SEQ ID NO: 297 or an allelic variant thereof having lysozyme activity; comprises or consists of the amino acid sequence of SEQ ID NO: 297 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of amino acids 1 to 245 of SEQ ID NO: 297 and has lysozyme activity.

In another aspect, the present invention relates to a polypeptide having lysozyme activity encoded by a polynucleotide that hybridizes under medium-high stringency conditions, high stringency conditions, or very high stringency conditions with (i) the mature polypeptide coding sequence of SEQ ID NO: 295, (ii) the cDNA sequence thereof; or (iii) the full-length complement of (i) or (ii) (Sambrook et al., 1989, Molecular Cloning, A Laboratory Manual, 2d edition, Cold Spring Harbor, New York). In an embodiment, the polypeptide has been isolated.

In another embodiment, the present invention relates to a polypeptide having lysozyme activity encoded by a polynucleotide having a sequence identity to the mature polypeptide coding sequence of SEQ ID NO: 295 or the cDNA sequence thereof of at least 80%, e.g., at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 297 comprising a substitution, and/or deletion, and/or insertion at one or more (e.g., several) positions. In an embodiment, the number of positions comprising one or more amino acid substitutions, and/or one or more amino acid deletions, and/or one or more amino acid insertions or any combination thereof in SEQ ID NO: 297 is not more than 49, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49. In another embodiment, the number of positions comprising one or more amino acid substitutions, and/or one or more amino acid deletions, and/or one or more amino acid insertions or any combination thereof in SEQ ID NO: 297 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 297 is not more than 10, e.g., 1, 2, 3, 4, 5, 6, 7, 8, 9 or 10. In another embodiment, the number of substitutions and/or deletions and/or insertions in SEQ ID NO: 297 is not more than 10, e.g., 1, 2, 3, 4, 5, 6, 7, 8, 9 or 10. In a further embodiment, the number of substitutions in SEQ ID NO: 297 is not more than 10, e.g., 1, 2, 3, 4, 5, 6, 7, 8, 9 or 10. In a further embodiment, the number of conservative substitutions in SEQ ID NO: 297 is not more than 10, e.g., 1, 2, 3, 4, 5, 6, 7, 8, 9 or 10. In an embodiment, the variant has at least at least 70%, e.g., at least 80%, at least 90%, at least 95% or at least 100% of the lysozyme activity of SEQ ID NO: 297.

The invention further relates to polypeptides having a sequence identity to the mature polypeptide of SEQ ID NO: 299 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have lysozyme activity. In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from the mature polypeptide of SEQ ID NO: 299. In an embodiment, the polypeptide has been isolated. In an embodiment, the polypeptide has at least at least 70%, e.g., at least 80%, at least 90%, at least 95% or at least 100% of the lysozyme activity of the mature polypeptide of SEQ ID NO: 299.

A polypeptide of the present invention preferably comprises or consists of the amino acid sequence of SEQ ID NO: 299 or an allelic variant thereof; comprises or consists of the amino acid sequence of SEQ ID NO: 299 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of the mature polypeptide of SEQ ID NO: 299. In another aspect, the polypeptide comprises or consists of amino acids 1 to 247 of SEQ ID NO: 299.

The present invention further relates to polypeptides having a sequence identity to SEQ ID NO: 300 of at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 70% of the lysozyme activity of SEQ ID NO: 300.

In a particular embodiment, the invention relates to polypeptides having a sequence identity to SEQ ID NO: 300 of at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 80% of the lysozyme activity of SEQ ID NO: 300.

In a particular embodiment, the invention relates to polypeptides having a sequence identity to SEQ ID NO: 300 of at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 90% of the lysozyme activity of SEQ ID NO: 300.

In a particular embodiment, the invention relates to polypeptides having a sequence identity to SEQ ID NO: 300 of at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 95% of the lysozyme activity of SEQ ID NO: 300.

In a particular embodiment, the invention relates to polypeptides having a sequence identity to SEQ ID NO: 300 of at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 100% of the lysozyme activity of SEQ ID NO: 300.

In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 300. In an embodiment, the polypeptide has at least at least 70%, e.g., at least 80%, at least 90%, at least 95% or at least 100% of the lysozyme activity of SEQ ID NO: 300.

A polypeptide of the present invention preferably comprises or consists of the amino acid sequence of SEQ ID NO: 300 or an allelic variant thereof having lysozyme activity; comprises or consists of the amino acid sequence of SEQ ID NO: 300 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of amino acids 1 to 247 of SEQ ID NO: 300 and has lysozyme activity.

In another aspect, the present invention relates to a polypeptide having lysozyme activity encoded by a polynucleotide that hybridizes under medium-high stringency conditions, high stringency conditions, or very high stringency conditions with (i) the mature polypeptide coding sequence of SEQ ID NO: 298, (ii) the cDNA sequence thereof; or (iii) the full-length complement of (i) or (ii) (Sambrook et al., 1989, Molecular Cloning, A Laboratory Manual, 2d edition, Cold Spring Harbor, New York). In an embodiment, the polypeptide has been isolated.

In another embodiment, the present invention relates to a polypeptide having lysozyme activity encoded by a polynucleotide having a sequence identity to the mature polypeptide coding sequence of SEQ ID NO: 298 or the cDNA sequence thereof of at least 80%, e.g., at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 300 comprising a substitution, and/or deletion, and/or insertion at one or more (e.g., several) positions. In an embodiment, the number of positions comprising one or more amino acid substitutions, and/or one or more amino acid deletions, and/or one or more amino acid insertions or any combination thereof in SEQ ID NO: 300 is not more than 49, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49. In another embodiment, the number of positions comprising one or more amino acid substitutions, and/or one or more amino acid deletions, and/or one or more amino acid insertions or any combination thereof in SEQ ID NO: 300 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 300 is not more than 10, e.g., 1, 2, 3, 4, 5, 6, 7, 8, 9 or 10. In another embodiment, the number of substitutions and/or deletions and/or insertions in SEQ ID NO: 300 is not more than 10, e.g., 1, 2, 3, 4, 5, 6, 7, 8, 9 or 10. In a further embodiment, the number of substitutions in SEQ ID NO: 300 is not more than 10, e.g., 1, 2, 3, 4, 5, 6, 7, 8, 9 or 10. In a further embodiment, the number of conservative substitutions in SEQ ID NO: 300 is not more than 10, e.g., 1, 2, 3, 4, 5, 6, 7, 8, 9 or 10. In an embodiment, the variant has at least at least 70%, e.g., at least 80%, at least 90%, at least 95% or at least 100% of the lysozyme activity of SEQ ID NO: 300.

The invention further relates to polypeptides having a sequence identity to the mature polypeptide of SEQ ID NO: 302 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have lysozyme activity. In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from the mature polypeptide of SEQ ID NO: 302. In an embodiment, the polypeptide has been isolated. In an embodiment, the polypeptide has at least at least 70%, e.g., at least 80%, at least 90%, at least 95% or at least 100% of the lysozyme activity of the mature polypeptide of SEQ ID NO: 302.

A polypeptide of the present invention preferably comprises or consists of the amino acid sequence of SEQ ID NO: 302 or an allelic variant thereof; comprises or consists of the amino acid sequence of SEQ ID NO: 302 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of the mature polypeptide of SEQ ID NO: 302. In another aspect, the polypeptide comprises or consists of amino acids 1 to 250 of SEQ ID NO: 302.

The present invention further relates to polypeptides having a sequence identity to SEQ ID NO: 303 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 70% of the lysozyme activity of SEQ ID NO: 303.

In a particular embodiment, the invention relates to polypeptides having a sequence identity to SEQ ID NO: 303 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 80% of the lysozyme activity of SEQ ID NO: 303.

In a particular embodiment, the invention relates to polypeptides having a sequence identity to SEQ ID NO: 303 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 90% of the lysozyme activity of SEQ ID NO: 303.

In a particular embodiment, the invention relates to polypeptides having a sequence identity to SEQ ID NO: 303 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 95% of the lysozyme activity of SEQ ID NO: 303.

In a particular embodiment, the invention relates to polypeptides having a sequence identity to SEQ ID NO: 303 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 100% of the lysozyme activity of SEQ ID NO: 303.

In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 303. In an embodiment, the polypeptide has at least at least 70%, e.g., at least 80%, at least 90%, at least 95% or at least 100% of the lysozyme activity of SEQ ID NO: 303.

A polypeptide of the present invention preferably comprises or consists of the amino acid sequence of SEQ ID NO: 303 or an allelic variant thereof having lysozyme activity; comprises or consists of the amino acid sequence of SEQ ID NO: 303 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of amino acids 1 to 250 of SEQ ID NO: 303 and has lysozyme activity.

In another aspect, the present invention relates to a polypeptide having lysozyme activity encoded by a polynucleotide that hybridizes under medium-high stringency conditions, high stringency conditions, or very high stringency conditions with (i) the mature polypeptide coding sequence of SEQ ID NO: 301, (ii) the cDNA sequence thereof; or (iii) the full-length complement of (i) or (ii) (Sambrook et al., 1989, Molecular Cloning, A Laboratory Manual, 2d edition, Cold Spring Harbor, New York). In an embodiment, the polypeptide has been isolated.

In another embodiment, the present invention relates to a polypeptide having lysozyme activity encoded by a polynucleotide having a sequence identity to the mature polypeptide coding sequence of SEQ ID NO: 301 or the cDNA sequence thereof of at least 80%, e.g., at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 303 comprising a substitution, and/or deletion, and/or insertion at one or more (e.g., several) positions. In an embodiment, the number of positions comprising one or more amino acid substitutions, and/or one or more amino acid deletions, and/or one or more amino acid insertions or any combination thereof in SEQ ID NO: 303 is not more than 49, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49. In another embodiment, the number of positions comprising one or more amino acid substitutions, and/or one or more amino acid deletions, and/or one or more amino acid insertions or any combination thereof in SEQ ID NO: 303 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 303 is not more than 10, e.g., 1, 2, 3, 4, 5, 6, 7, 8, 9 or 10. In another embodiment, the number of substitutions and/or deletions and/or insertions in SEQ ID NO: 303 is not more than 10, e.g., 1, 2, 3, 4, 5, 6, 7, 8, 9 or 10. In a further embodiment, the number of substitutions in SEQ ID NO: 303 is not more than 10, e.g., 1, 2, 3, 4, 5, 6, 7, 8, 9 or 10. In a further embodiment, the number of conservative substitutions in SEQ ID NO: 303 is not more than 10, e.g., 1, 2, 3, 4, 5, 6, 7, 8, 9 or 10. In an embodiment, the variant has at least at least 70%, e.g., at least 80%, at least 90%, at least 95% or at least 100% of the lysozyme activity of SEQ ID NO: 303.

The invention further relates to polypeptides having a sequence identity to the mature polypeptide of SEQ ID NO: 305 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, which have lysozyme activity. In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from the mature polypeptide of SEQ ID NO: 305. In an embodiment, the polypeptide has been isolated. In an embodiment, the polypeptide has at least at least 70%, e.g., at least 80%, at least 90%, at least 95% or at least 100% of the lysozyme activity of the mature polypeptide of SEQ ID NO: 305.

A polypeptide of the present invention preferably comprises or consists of the amino acid sequence of SEQ ID NO: 305 or an allelic variant thereof; comprises or consists of the amino acid sequence of SEQ ID NO: 305 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of the mature polypeptide of SEQ ID NO: 305. In another aspect, the polypeptide comprises or consists of amino acids 1 to 240 of SEQ ID NO: 305.

The present invention further relates to polypeptides having a sequence identity to SEQ ID NO: 306 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 70% of the lysozyme activity of SEQ ID NO: 306.

In a particular embodiment, the invention relates to polypeptides having a sequence identity to SEQ ID NO: 306 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 80% of the lysozyme activity of SEQ ID NO: 306.

In a particular embodiment, the invention relates to polypeptides having a sequence identity to SEQ ID NO: 306 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 90% of the lysozyme activity of SEQ ID NO: 306.

In a particular embodiment, the invention relates to polypeptides having a sequence identity to SEQ ID NO: 306 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 95% of the lysozyme activity of SEQ ID NO: 306.

In a particular embodiment, the invention relates to polypeptides having a sequence identity to SEQ ID NO: 306 of at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, and wherein the polypeptide has at least at least 100% of the lysozyme activity of SEQ ID NO: 306.

In one aspect, the polypeptides differ by up to 49 amino acids, e.g., between 1 and 49 amino acids, such as 1-45, 1-40, 1-35, 1-30, 1-25, 1-20, 1-15, 1-10 or 1-5 amino acids, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 amino acids from SEQ ID NO: 306. In an embodiment, the polypeptide has at least at least 70%, e.g., at least 80%, at least 90%, at least 95% or at least 100% of the lysozyme activity of SEQ ID NO: 306.

A polypeptide of the present invention preferably comprises or consists of the amino acid sequence of SEQ ID NO: 306 or an allelic variant thereof having lysozyme activity; comprises or consists of the amino acid sequence of SEQ ID NO: 306 and a N-terminal and/or C-terminal His-tag and/or HQ-tag; or is a fragment thereof having lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids. In another aspect, the polypeptide comprises or consists of amino acids 1 to 240 of SEQ ID NO: 306 and has lysozyme activity.

In another aspect, the present invention relates to a polypeptide having lysozyme activity encoded by a polynucleotide that hybridizes under medium-high stringency conditions, high stringency conditions, or very high stringency conditions with (i) the mature polypeptide coding sequence of SEQ ID NO: 304, (ii) the cDNA sequence thereof; or (iii) the full-length complement of (i) or (ii) (Sambrook et al., 1989, Molecular Cloning, A Laboratory Manual, 2d edition, Cold Spring Harbor, New York). In an embodiment, the polypeptide has been isolated.

In another embodiment, the present invention relates to a polypeptide having lysozyme activity encoded by a polynucleotide having a sequence identity to the mature polypeptide coding sequence of SEQ ID NO: 304 or the cDNA sequence thereof of at least 80%, e.g., at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 306 comprising a substitution, and/or deletion, and/or insertion at one or more (e.g., several) positions. In an embodiment, the number of positions comprising one or more amino acid substitutions, and/or one or more amino acid deletions, and/or one or more amino acid insertions or any combination thereof in SEQ ID NO: 306 is not more than 49, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49. In another embodiment, the number of positions comprising one or more amino acid substitutions, and/or one or more amino acid deletions, and/or one or more amino acid insertions or any combination thereof in SEQ ID NO: 306 is between 1 and 45, such as 1-40, 1-35, 1-30, 1-25, 1-20, 1-15, 1-10 or 1-5 positions. In an embodiment, the number of positions comprising one or more amino acid substitutions, and/or one or more amino acid deletions, and/or one or more amino acid insertions or any combination thereof in SEQ ID NO: 306 is not more than 10, e.g., 1, 2, 3, 4, 5, 6, 7, 8, 9 or 10. In another embodiment, the number of substitutions and/or deletions and/or insertions in SEQ ID NO: 306 is not more than 10, e.g., 1, 2, 3, 4, 5, 6, 7, 8, 9 or 10. In a further embodiment, the number of substitutions in SEQ ID NO: 306 is not more than 10, e.g., 1, 2, 3, 4, 5, 6, 7, 8, 9 or 10. In a further embodiment, the number of conservative substitutions in SEQ ID NO: 306 is not more than 10, e.g., 1, 2, 3, 4, 5, 6, 7, 8, 9 or 10. In an embodiment, the variant has at least at least 70%, e.g., at least 80%, at least 90%, at least 95% or at least 100% of the lysozyme activity of SEQ ID NO: 306.

### Sources of Polypeptides Having Lysozyme Activity

A polypeptide having lysozyme activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a fungal polypeptide. In one aspect, the polypeptide is a polypeptide having lysozyme activity from a fungus of the class Pezizomycetes, such as from the order Pezizales, or from the family Pyronemataceae, or from the genus Trichophaea or from the species Trichophaea saccata or Trichophaea minuta. In another aspect, the polypeptide is a polypeptide having lysozyme activity from a fungus of the class Sordariomycetes, such as from the order Hypocreales, or from the family Hypocreaceae, or from the genus Trichoderma or from the species Trichoderma harzianum.

In another aspect, the polypeptide is a polypeptide having lysozyme activity from a fungus of the class Sordariomycetes, such as from the order Sordariales, or from the family Chaetomiaceae, or from the genus Chaetomium or from the species Chaetomium sp. ZY 287.

In another aspect, the polypeptide is a polypeptide having lysozyme activity from a fungus of the order Mortierellales, or from the family Mortierellaceae, or from the genus Mortierella or from the species Mortierella sp. ZY 002.

In another aspect, the polypeptide is a polypeptide having lysozyme activity from a fungus of the class Sordariomycetes, such as from the order Hypocreales, or from the family Clavicipitaceae, or from the genus Metarhizium or from the species Metarhizium sp. XZ2431.

In another aspect, the polypeptide is a polypeptide having lysozyme activity from a fungus of the class Leotiomycetes, such as from the family Pseudeurotiaceae, or from the genus Geomyces or from the species Geomyces auratus.

In another aspect, the polypeptide is a polypeptide having lysozyme activity from a fungus of the class Sordariomycetes, such as from the order Hypocreales, or from the family Nectriaceae, or from the genus Ilyonectria or from the species Ilyonectria rufa.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to polynucleotides encoding a polypeptide of the present invention, as described herein. In an embodiment, the polynucleotide encoding the polypeptide of the present invention has been isolated.

The techniques used to isolate or clone a polynucleotide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Trichophaea or a strain of Trichoderma, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 255, SEQ ID NO: 265, SEQ ID NO: 287, SEQ ID NO: 292, SEQ ID NO: 295, SEQ ID NO: 298, SEQ ID NO: 301, SEQ ID NO: 304, or the cDNA sequence thereof, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2:95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (E gon et al., 1988, Gene 69:301-315), Streptomyces coelicolor agarase gene (dagA), and prokaryotic beta-lactamase gene (Villa-Kamaroff et al., 1978, Proc. Natl. Acad. Sci. USA 75:3727-3731), as well as the tac promoter (DeBoer et al., 1983, Proc. Natl. Acad. Sci. USA 80:21-25). Further promoters are described in “Useful proteins from recombinant bacteria” in Gilbert et al., 1980, Scientific American 242:74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP 1), and Saccharomyces cerevisiae 3-phosphoglycerate kinase. Other useful promoters for yeast host cells are described by Romanos et al., 1992, Yeast 8:423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rmnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP 82 gene (Hue et al., 1995, Journal of Bacteriology 177:3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15:5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, subtilisin, Bacillus licheniformis Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57:109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra. The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR 322, pUC 19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98:61-67; Cullen et al., 1987, Nucleic Acids Res. 15:9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168:111-115), competent cell transformation (see, e.g., Young and Spizizen, 1961, J. Bacteriol. 81:823-829, or Dubnau and Davidoff-Abelson, 1971, J. Mol. Biol. 56:209-221), electroporation (see, e.g., Shigekawa and Dower, 1988, Biotechniques 6:742-751), or conjugation (see, e.g., Koehler and Thorne, 1987, J. Bacteriol. 169:5271-5278). The introduction of DNA into an E. coli cell may be effected by protoplast transformation (see, e.g., Hanahan, 1983, J. Mol. Biol. 166:557-580) or electroporation (see, e.g., Dower et al., 1988, Nucleic Acids Res. 16:6127-6145). The introduction of DNA into a Streptomyces cell may be effected by protoplast transformation, electroporation (see, e.g., Gong et al., 2004, Folia Microbiol. (Praha) 49:399-405), conjugation (see, e.g., Mazodier et al., 1989, J. Bacteriol. 171:3583-3585), or transduction (see, e.g., Burke et al., 2001, Proc. Natl. Acad. Sci. USA 98:6289-6294). The introduction of DNA into a Pseudomonas cell may be effected by electroporation (see, e.g., Choi et al., 2006, J. Microbiol. Methods 64:391-397) or conjugation (see, e.g., Pinedo and Smets, 2005, Appl. Environ. Microbiol. 71:51-57). The introduction of DNA into a Streptococcus cell may be effected by natural competence (see, e.g., Perry and Kuramitsu, 1981, Infect. Immun. 32:1295-1297), protoplast transformation (see, e.g., Catt and Jollick, 1991, Microbios 68:189-207), electroporation (see, e.g., Buckley et al., 1999, Appl. Environ. Microbiol. 65:3800-3804), or conjugation (see, e.g., Clewell, 1981, Microbiol. Rev. 45:409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81:1470-1474, and Christensen et al., 1988, Bio/Technology 6:1419-1422. Suitable methods for transforming Fusarium species are described by Malardier et al., 1989, Gene 78:147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153:163; and Hinnen et al., 1978, Proc. Natl. Acad. Sci. USA 75:1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Trichophaea cell. In another aspect, the cell is a Trichophaea saccata cell. In another aspect, the cell is a Trichoderma cell. In another aspect, the cell is a Trichoderma harzianum cell. In another aspect, the cell is a Trichoderma minuta cell. In another aspect, the cell is a Chaetomium cell. In another aspect, the cell is a Chaetomium sp. ZY287 cell. In another aspect, the cell is a Mortierellaceae cell. In another aspect, the cell is a Mortierella sp. ZY 002 cell. In another aspect, the cell is a Metarhizium cell. In another aspect, the cell is a Metarhizium sp. XZ2431 cell. In another aspect, the cell is a Geomyces cell. In another aspect, the cell is a Geomyces auratus cell. In another aspect, the cell is a Ilyonectria cell. In another aspect, the cell is a Ilyonectria rufa cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermenters in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Production in Plants

The present invention also relates to isolated plants, e.g., a transgenic plant, plant part, or plant cell, comprising a polynucleotide of the present invention so as to express and produce a polypeptide or domain in recoverable quantities. The polypeptide or domain may be recovered from the plant or plant part. Alternatively, the plant or plant part containing the polypeptide or domain may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems.

Plant cells and specific plant cell compartments, such as chloroplasts, apoplasts, mitochondria, vacuoles, peroxisomes and cytoplasm are also considered to be a plant part. Also included within the scope of the present invention are the progeny of such plants, plant parts, and plant cells.

The transgenic plant or plant cell expressing the polypeptide or domain may be constructed in accordance with methods known in the art.

The present invention also relates to methods of producing a polypeptide or domain of the present invention comprising (a) cultivating a transgenic plant or a plant cell comprising a polynucleotide encoding the polypeptide or domain under conditions conducive for production of the polypeptide or domain; and (b) recovering the polypeptide or domain.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising a lysozyme of the present invention. Preferably, the compositions are enriched in the lysozyme of the invention. The term “enriched” indicates that the lysozyme activity of the composition has been increased, e.g., with an enrichment factor of at least 1.1, such as at least 1.2, at least 1.3, at least 1.4, at least 1.5, at least 2.0, at least 3.0, at least 4.0, at least 5.0, at least 10.

The compositions may comprise a polypeptide of the present invention as the major enzymatic component, e.g., a mono-component composition. Such a composition may further comprise a formulating agent, as described below. Alternatively, the compositions may comprise multiple enzymatic activities.

In an embodiment, the composition comprises the polypeptide of the invention and one or more formulating agents as described below.

### Formulating Agent

The enzyme of the invention may be formulated as a liquid or a solid. For a liquid formulation, the formulating agent may comprise a polyol (such as, e.g., glycerol, ethylene glycol or propylene glycol), a salt (such as, e.g., sodium chloride, sodium benzoate, potassium sorbate) or a sugar or sugar derivative (such as, e.g., dextrin, glucose, sucrose, and sorbitol). Thus in one embodiment, the composition is a liquid composition comprising the polypeptide of the invention and one or more formulating agents selected from the list consisting of glycerol, ethylene glycol, 1,2-propylene glycol, 1,3-propylene glycol, sodium chloride, sodium benzoate, potassium sorbate, dextrin, glucose, sucrose, and sorbitol. The liquid formulation may be sprayed onto the feed after it has been pelleted or may be added to drinking water given to the animals.

For a solid formulation, the formulation may be for example as a granule, spray dried powder or agglomerate. The formulating agent may comprise a salt (organic or inorganic zinc, sodium, potassium or calcium salts such as, e.g., calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as, e.g., sucrose, dextrin, glucose, lactose, sorbitol).

In an embodiment, the solid composition is in granulated form. The granule may have a matrix structure where the components are mixed homogeneously. However, the granule typically comprises a core particle and one or more coatings, which typically are salt and/or wax coatings. Examples of waxes are polyethylene glycols; polypropylenes; Carnauba wax; Candelilla wax; bees wax; hydrogenated plant oil or animal tallow such as hydrogenated ox tallow, hydrogenated palm oil, hydrogenated cotton seeds and/or hydrogenated soy bean oil; fatty acid alcohols; mono-glycerides and/or di-glycerides, such as glyceryl stearate, wherein stearate is a mixture of stearic and palmitic acid; micro-crystalline wax; paraffin's; and fatty acids, such as hydrogenated linear long chained fatty acids and derivatives thereof. A preferred wax is palm oil or hydrogenated palm oil. The core particle can either be a homogeneous blend of lysozyme of the invention optionally combined with one or more additional enzymes and optionally together with one or more salts or an inert particle with the lysozyme of the invention optionally combined with one or more additional enzymes applied onto it.

In an embodiment, the material of the core particles are selected from the group consisting of inorganic salts (such as calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate, zinc sulfate), starch or a sugar or sugar derivative (such as, e.g., sucrose, dextrin, glucose, lactose, sorbitol), sugar or sugar derivative (such as, e.g., sucrose, dextrin, glucose, lactose, sorbitol), small organic molecules, starch, flour, cellulose and minerals and clay minerals (also known as hydrous aluminium phyllosilicates). In a preferred embodiment, the core comprises a clay mineral such as kaolinite or kaolin.

The salt coating is typically at least 1 μm thick and can either be one particular salt or a mixture of salts, such as Na₂SO₄, K₂SO₄, MgSO₄ and/or sodium citrate. Other examples are those described in, e.g., WO 2008/017659, WO 2006/034710, WO 97/05245, WO 98/54980, WO 98/55599, WO 00/70034 or polymer coating such as described in WO 01/00042.

In another embodiment, the composition is a solid composition comprising the lysozyme of the invention and one or more formulating agents selected from the list consisting of sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch and cellulose. In a preferred embodiment, the formulating agent is selected from one or more of the following compounds: sodium sulfate, dextrin, cellulose, sodium thiosulfate and calcium carbonate. In a preferred embodiment, the solid composition is in granulated form. In an embodiment, the solid composition is in granulated form and comprises a core particle, an enzyme layer comprising the lysozyme of the invention and a salt coating.

In a further embodiment, the formulating agent is selected from one or more of the following compounds: glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, kaolin and cellulose. In a preferred embodiment, the formulating agent is selected from one or more of the following compounds: 1,2-propylene glycol, 1,3-propylene glycol, sodium sulfate, dextrin, cellulose, sodium thiosulfate, kaolin and calcium carbonate.

### Animal Feed and Animal Feed Additives

The present invention also relates to animal feed compositions and animal feed additives. Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore, such fish diets usually have a crude fat content of 200-310 g/kg.

An animal feed composition according to the invention has a crude protein content of 50-800 g/kg, and furthermore comprises at least one lysozyme as described herein or more than one lysozyme as described herein.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e., Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington DC).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen bv, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains at least one vegetable protein as defined above.

The animal feed composition of the invention may also contain animal protein, such as Meat and Bone Meal, Feather meal, and/or Fish Meal, typically in an amount of 0-25%. The animal feed composition of the invention may also comprise Dried Distillers Grains with Solubles (DDGS), typically in amounts of 0-30%.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-25% fish meal; and/or 0-25% meat and bone meal; and/or 0-20% whey.

The animal feed may comprise vegetable proteins. In particular embodiments, the protein content of the vegetable proteins is at least 10, 20, 30, 40, 50, 60, 70, 80, or 90% (w/w). Vegetable proteins may be derived from vegetable protein sources, such as legumes and cereals, for example, materials from plants of the families Fabaceae (Leguminosae), Cruciferaceae, Chenopodiaceae, and Poaceae, such as soy bean meal, lupin meal, rapeseed meal, and combinations thereof.

In a particular embodiment, the vegetable protein source is material from one or more plants of the family Fabaceae, e.g., soybean, lupine, pea, or bean. In another particular embodiment, the vegetable protein source is material from one or more plants of the family Chenopodiaceae, e.g., beet, sugar beet, spinach or quinoa. Other examples of vegetable protein sources are rapeseed, and cabbage. In another particular embodiment, soybean is a preferred vegetable protein source. Other examples of vegetable protein sources are cereals such as barley, wheat, rye, oat, maize (corn), rice, and sorghum.

Animal diets can, e.g., be manufactured as mash feed (non-pelleted) or pelleted feed. Typically, the milled feed-stuffs are mixed and sufficient amounts of essential vitamins and minerals are added according to the specifications for the species in question. Enzymes can be added as solid or liquid enzyme formulations. For example, for mash feed a solid or liquid enzyme formulation may be added before or during the ingredient mixing step. For pelleted feed the (liquid or solid) lysozyme/enzyme preparation may also be added before or during the feed ingredient step. Typically, a liquid lysozyme/enzyme preparation comprises the lysozyme of the invention optionally with a polyol, such as glycerol, ethylene glycol or propylene glycol, and is added after the pelleting step, such as by spraying the liquid formulation onto the pellets. The enzyme may also be incorporated in a feed additive or premix.

Alternatively, the lysozyme can be prepared by freezing a mixture of liquid enzyme solution with a bulking agent such as ground soybean meal, and then lyophilizing the mixture.

In an embodiment, the composition comprises one or more additional enzymes. In an embodiment, the composition comprises one or more microbes. In an embodiment, the composition comprises one or more vitamins. In an embodiment, the composition comprises one or more minerals. In an embodiment, the composition comprises one or more amino acids. In an embodiment, the composition comprises one or more other feed ingredients.

In another embodiment, the composition comprises one or more of the polypeptides of the invention, one or more formulating agents and one or more additional enzymes. In an embodiment, the composition comprises one or more of the polypeptides of the invention, one or more formulating agents and one or more microbes. In an embodiment, the composition comprises one or more of the polypeptides of the invention, one or more formulating agents and one or more vitamins. In an embodiment, the composition comprises one or more of the polypeptides of the invention and one or more minerals. In an embodiment, the composition comprises the polypeptide of the invention, one or more formulating agents and one or more amino acids. In an embodiment, the composition comprises one or more of the polypeptides of the invention, one or more formulating agents and one or more other feed ingredients.

In a further embodiment, the composition comprises one or more of the polypeptides of the invention, one or more formulating agents and one or more components selected from the list consisting of: one or more additional enzymes; one or more microbes; one or more vitamins; one or more minerals; one or more amino acids; and one or more other feed ingredients.

The final lysozyme concentration in the diet is within the range of 0.01-200 ppm enzyme protein per kg animal feed, such as 0.1 to 150 ppm, 0.5 to 100 ppm, 1 to 75 ppm, 2 to 50 ppm, 3 to 25 ppm, 2 to 80 ppm, 5 to 60 ppm, 8 to 40 ppm or 10 to 30 ppm enzyme protein per kg animal feed, or any combination of these intervals.

It is at present contemplated that the lysozyme is administered in one or more of the following amounts (dosage ranges): 0.01-200; 0.01-100; 0.5-100; 1-50; 5-100; 5-50; 10-100; 0.05-50; 5-25; or 0.10-10-all these ranges being in mg lysozyme per kg feed (ppm).

For determining mg lysozyme protein per kg feed, the lysozyme is purified from the feed composition, and the specific activity of the purified lysozyme is determined using a relevant assay (see under lysozyme activity). The lysozyme activity of the feed composition as such is also determined using the same assay, and on the basis of these two determinations, the dosage in mg lysozyme protein per kg feed is calculated.

In a particular embodiment, the animal feed additive of the invention is intended for being included (or prescribed as having to be included) in animal diets or feed at levels of 0.01 to 10.0%; more particularly 0.05 to 5.0%; or 0.2 to 1.0% (% meaning g additive per 100 g feed). This is so in particular for premixes.

The same principles apply for determining mg lysozyme protein in feed additives. Of course, if a sample is available of the lysozyme used for preparing the feed additive or the feed, the specific activity is determined from this sample (no need to purify the lysozyme from the feed composition or the additive).

### Additional Enzymes

In another embodiment, the compositions described herein optionally include one or more enzymes. Enzymes can be classified on the basis of the handbook Enzyme Nomenclature from NC-IUBMB, 1992), see also the ENZYME site at the internet: expasy.ch/enzyme/. ENZYME is a repository of information relative to the nomenclature of enzymes. It is primarily based on the recommendations of the Nomenclature Committee of the International Union of Biochemistry and Molecular Biology (IUB-MB), Academic Press, Inc., 1992, and it describes each type of characterized enzyme for which an EC (Enzyme Commission) number has been provided (Bairoch, 2000, “The ENZYME Database”, Nucleic Acids Res. 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, xylanase, galactanase, mannanase, dextranase, lysozyme and galactosidase is described in Henrissat et al, “The carbohydrate-active enzymes database (CAZy) in 2013”, Nucl. Acids Res. (1 Jan. 2014) 42 (D1): D490-D495; see also cazy.org.

Thus the composition of the invention may also comprise at least one other enzyme selected from the group comprising of phytase (EC 3.1.3.8 or 3.1.3.26); xylanase (EC 3.2.1.8); galactanase (EC 3.2.1.89); alpha-galactosidase (EC 3.2.1.22); protease (EC 3.4); phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); arabinofuranosidase (EC 3.2.1.55); beta-xylosidase (EC 3.2.1.37); acetyl xylan esterase (EC 3.1.1.72); feruloyl esterase (EC 3.1.1.73); cellulase (EC 3.2.1.4); cellobiohydrolases (EC 3.2.1.91); beta-glucosidase (EC 3.2.1.21); pullulanase (EC 3.2.1.41), alpha-mannosidase (EC 3.2.1.24), mannanase (EC 3.2.1.25) and beta-glucanase (EC 3.2.1.4 or EC 3.2.1.6), or any mixture thereof.

In a particular embodiment, the composition of the invention comprises a phytase (EC 3.1.3.8 or 3.1.3.26). Examples of commercially available phytases include Bio-Feed™ Phytase (Novozymes), Ronozyme® P, Ronozyme® NP and Ronozyme® HiPhos (DSM Nutritional Products), Natuphos™ (BASF), Finase® and Quantum® Blue (AB Enzymes), OptiPhos® (Huvepharma) Phyzyme® XP (Verenium/DuPont) and Axtra® PHY (DuPont). Other preferred phytases include those described in, e.g., WO 98/28408, WO 00/43503, and WO 03/066847.

In a particular embodiment, the composition of the invention comprises a xylanase (EC 3.2.1.8). Examples of commercially available xylanases include Ronozyme® WX and Ronozyme® G2 (DSM Nutritional Products), Econase® XT and Barley (AB Vista), Xylathin® (Verenium), Hostazym® X (Huvepharma) and Axtra® XB (Xylanase/beta-glucanase, DuPont).

In a particular embodiment, the composition of the invention comprises a protease (EC 3.4). Examples of commercially available proteases include Ronozyme® ProAct (DSM Nutritional Products).

### Microbes

In an embodiment, the animal feed composition further comprises one or more additional microbes. In a particular embodiment, the animal feed composition further comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

In a preferred embodiment, animal feed composition further comprises a bacterium from one or more of the following strains: Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Enterococcus faecium, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Propionibacterium thoenii, Lactobacillus farciminus, Lactobacillus rhamnosus, Clostridium butyricum, Bifidobacterium animalis ssp. animalis, Lactobacillus reuteri, Lactobacillus salivarius ssp. salivarius, Megasphaera elsdenii, Propionibacteria sp.

In a more preferred embodiment, composition, animal feed additive or animal feed further comprises a bacterium from one or more of the following strains of Bacillus subtilis: 3A-P4 (PTA-6506), 15A-P4 (PTA-6507), 22C-P1 (PTA-6508), 2084 (NRRL B-500130), LSSA01 (NRRL-B-50104), BS27 (NRRL B-501 05), BS 18 (NRRL B-50633), BS 278 (NRRL B-50634), DSM 29870, DSM 29871, NRRL B-50136, NRRL B-50605, NRRL B-50606, NRRL B-50622 and PTA-7547.

In a more preferred embodiment, composition, animal feed additive or animal feed further comprises a bacterium from one or more of the following strains of Bacillus pumilus: NRRL B-50016, ATCC 700385, NRRL B-50885 or NRRL B-50886.

In a more preferred embodiment, composition, animal feed additive or animal feed further comprises a bacterium from one or more of the following strains of Bacillus lichenformis: NRRL B 50015, NRRL B-50621 or NRRL B-50623.

In a more preferred embodiment, composition, animal feed additive or animal feed further comprises a bacterium from one or more of the following strains of Bacillus amyloliquefaciens: DSM 29869, DSM 29869, NRRL B 50607, PTA-7543, PTA-7549, NRRL B-50349, NRRL B-50606, NRRL B-50013, NRRL B-50151, NRRL B-50141, NRRL B-50147 or NRRL B-50888.

The bacterial count of each of the bacterial strains in the animal feed composition is between 1×10⁴ and 1×10¹⁴ CFU/kg of dry matter, preferably between 1×10⁶ and 1×10¹² CFU/kg of dry matter, and more preferably between 1×10⁷ and 1×10¹¹ CFU/kg of dry matter. In a more preferred embodiment the bacterial count of each of the bacterial strains in the animal feed composition is between 1×10⁸ and 1×10¹⁰ CFU/kg of dry matter.

The bacterial count of each of the bacterial strains in the animal feed composition is between 1×10⁵ and 1×10¹⁵ CFU/animal/day, preferably between 1×10⁷ and 1×10¹³ CFU/animal/day, and more preferably between 1×10⁸ and 1×10¹² CFU/animal/day. In a more preferred embodiment the bacterial count of each of the bacterial strains in the animal feed composition is between 1×10⁹ and 1×10¹¹ CFU/animal/day.

In another embodiment, the one or more bacterial strains are present in the form of a stable spore.

### Premix

In an embodiment, the animal feed may include a premix, comprising, e.g., vitamins, minerals, enzymes, amino acids, preservatives, antibiotics, other feed ingredients or any combination thereof which are mixed into the animal feed.

### Amino Acids

The composition of the invention may further comprise one or more amino acids. Examples of amino acids which are used in animal feed are lysine, alanine, beta-alanine, threonine, methionine and tryptophan.

### Vitamins and Minerals

In another embodiment, the animal feed may include one or more vitamins, such as one or more fat-soluble vitamins and/or one or more water-soluble vitamins. In another embodiment, the animal feed may optionally include one or more minerals, such as one or more trace minerals and/or one or more macro minerals.

Usually fat- and water-soluble vitamins, as well as trace minerals form part of a so-called premix intended for addition to the feed, whereas macro minerals are usually separately added to the feed.

Non-limiting examples of fat-soluble vitamins include vitamin A, vitamin D3, vitamin E, and vitamin K, e.g., vitamin K3.

Non-limiting examples of water-soluble vitamins include vitamin B12, biotin and choline, vitamin B1, vitamin B2, vitamin B6, niacin, folic acid and panthothenate, e.g., Ca-D-panthothenate.

Non-limiting examples of trace minerals include boron, cobalt, chloride, chromium, copper, fluoride, iodine, iron, manganese, molybdenum, selenium and zinc.

Non-limiting examples of macro minerals include calcium, magnesium, potassium and sodium.

The nutritional requirements of these components (exemplified with poultry and piglets/pigs) are listed in Table A of WO 01/58275. Nutritional requirement means that these components should be provided in the diet in the concentrations indicated.

In the alternative, the animal feed additive of the invention comprises at least one of the individual components specified in Table A of WO 01/58275. At least one means either of, one or more of, one, or two, or three, or four and so forth up to all thirteen, or up to all fifteen individual components. More specifically, this at least one individual component is included in the additive of the invention in such an amount as to provide an in-feed-concentration within the range indicated in column four, or column five, or column six of Table A.

In a still further embodiment, the animal feed additive of the invention comprises at least one of the below vitamins, preferably to provide an in-feed-concentration within the ranges specified in the below Table 1 (for piglet diets, and broiler diets, respectively).

TABLE 1
Typical vitamin recommendations

| Vitamin | Piglet diet | | Broiler diet | |
|---|---|---|---|---|
| Vitamin A | 10,000-15,000 | IU/kg feed | 8-12,500 | IU/kg feed |
| Vitamin D3 | 1800-2000 | IU/kg feed | 3000-5000 | IU/kg feed |
| Vitamin E | 60-100 | mg/kg feed | 150-240 | mg/kg feed |
| Vitamin K3 | 2-4 | mg/kg feed | 2-4 | mg/kg feed |
| Vitamin B1 | 2-4 | mg/kg feed | 2-3 | mg/kg feed |
| Vitamin B2 | 6-10 | mg/kg feed | 7-9 | mg/kg feed |
| Vitamin B6 | 4-8 | mg/kg feed | 3-6 | mg/kg feed |
| Vitamin B12 | 0.03-0.05 | mg/kg feed | 0.015-0.04 | mg/kg feed |
| Niacin | 30-50 | mg/kg feed | 50-80 | mg/kg feed |
| (Vitamin B3) | | | | |
| Pantothenic | 20-40 | mg/kg feed | 10-18 | mg/kg feed |
| acid | | | | |
| Folic acid | 1-2 | mg/kg feed | 1-2 | mg/kg feed |
| Biotin | 0.15-0.4 | mg/kg feed | 0.15-0.3 | mg/kg feed |
| Choline | 200-400 | mg/kg feed | 300-600 | mg/kg feed |
| chloride | | | | |

### Other Feed Ingredients

The composition of the invention may further comprise colouring agents, stabilisers, growth improving additives and aroma compounds/flavourings, polyunsaturated fatty acids (PUFAs); reactive oxygen generating species, anti-microbial peptides and anti-fungal polypeptides.

Examples of colouring agents are carotenoids such as beta-carotene, astaxanthin, and lutein.

Examples of aroma compounds/flavourings are creosol, anethol, deca-, undeca- and/or dodeca-lactones, ionones, irone, gingerol, piperidine, propylidene phatalide, butylidene phatalide, capsaicin and tannin.

Examples of stabilizing agents (e.g., acidifiers) are organic acids. Examples of these are benzoic acid (VevoVitall®, DSM Nutritional Products), formic acid, butyric acid, fumaric acid and propionic acid.

Examples of antimicrobial peptides (AMP's) are CAP18, Leucocin A, Tritrpticin, Protegrin-1, Thanatin, Defensin, Lactoferrin, Lactoferricin, and Ovispirin such as Novispirin (Robert Lehrer, 2000), Plectasins, and Statins, including the compounds and polypeptides disclosed in WO 03/044049 and WO 03/048148, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Examples of polyunsaturated fatty acids are C18, C20 and C22 polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

The composition of the invention may further comprise at least one amino acid. Examples of amino acids which are used in animal feed are lysine, alanine, beta-alanine, threonine, methionine and tryptophan.

### Uses

### Use in Animal Feed

A lysozyme of the invention may also be used in animal feed, wherein the term “animal” refers to all animals except humans. Examples of animals are non-ruminants, and ruminants. Ruminant animals include, for example, animals such as sheep, goats, cattle, e.g., beef cattle, cows, and young calves, deer, yank, camel, llama and kangaroo. Non-ruminant animals include mono-gastric animals, e.g., pigs or swine (including, but not limited to, piglets, growing pigs, and sows); poultry such as turkeys, ducks and chicken (including but not limited to broiler chicks, layers); horses (including but not limited to hotbloods, coldbloods and warm bloods), young calves; fish (including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish); and crustaceans (including but not limited to shrimps and prawns).

In the use according to the invention the lysozymes can be fed to the animal before, after, or simultaneously with the diet. The latter is preferred.

In a particular embodiment, the lysozyme, in the form in which it is added to the feed, or when being included in a feed additive, is well-defined. Well-defined means that the lysozyme preparation is at least 50% pure as determined by Size-exclusion chromatography (see Example 12 of WO 01/58275). In other particular embodiments the lysozyme preparation is at least 60, 70, 80, 85, 88, 90, 92, 94, or at least 95% pure as determined by this method.

A well-defined lysozyme preparation is advantageous. For instance, it is much easier to dose correctly to the feed a lysozyme that is essentially free from interfering or contaminating other lysozymes. The term dose correctly refers in particular to the objective of obtaining consistent and constant results, and the capability of optimizing dosage based upon the desired effect.

For the use in animal feed, however, the lysozyme need not be pure; it may, e.g., include other enzymes, in which case it could be termed a lysozyme preparation.

The lysozyme preparation can be (a) added directly to the feed, or (b) it can be used in the production of one or more intermediate compositions such as feed additives or premixes that is subsequently added to the feed (or used in a treatment process). The degree of purity described above refers to the purity of the original lysozyme preparation, whether used according to (a) or (b) above.

### Methods of Improving Animal Performance

In an embodiment, the present invention also relates to a method of improving the performance of an animal comprising administering to the animal the animal feed or an animal feed additive of the invention.

In a preferred embodiment, the method of improving the performance of an animal comprises administering to the animal an animal feed or an animal feed additive comprising the lysozyme of SEQ ID NO: 257, SEQ ID NO: 264, SEQ ID NO: 267, SEQ ID NO: 291, SEQ ID NO: 294, SEQ ID NO: 297, SEQ ID NO: 300, SEQ ID NO: 303 and/or SEQ ID NO: 306.

In an embodiment, the present invention also relates to the use of the animal feed or an animal feed additive of the invention for improving the performance of an animal. In another embodiment, the invention relates to the use of one or more lysozymes of the invention for improving the performance of an animal.

In one embodiment, ‘improving the performance of an animal’ means that there is an increase in body weight gain. In another embodiment, ‘improving the performance of an animal’ means that there is an improved feed conversion ratio. In a further embodiment, ‘improving the performance of an animal’ means that there is an increased feed efficiency. In a further embodiment, ‘improving the performance of an animal’ means that there is an increase in body weight gain and/or an improved feed conversion ratio and/or an increased feed efficiency.

### Methods of Preparing an Animal Feed

In an embodiment, the present invention provides a method for preparing an animal feed comprising adding one or more lysozymes of the present invention to one or more animal feed ingredients. Animal feed ingredients include, but are not limited to, concentrates (as defined herein), forage (as defined herein), enzymes, microbe, vitamins, minerals and amino acids.

In a preferred embodiment, the method of preparing an animal feed comprises mixing the lysozyme of SEQ ID NO: 257, SEQ ID NO: 264, SEQ ID NO: 267, SEQ ID NO: 291, SEQ ID NO: 294, SEQ ID NO: 297, SEQ ID NO: 300, SEQ ID NO: 303 and/or SEQ ID NO: 306 with concentrate and/or forage.

Methods of Treatment of Clostridium perfringens and/or Necrotic enteritis

In another aspect, the present invention provides a method of treatment of necrotic enteritis comprising the steps of administering the animal feed or animal feed additive to one or more animals with necrotic enteritis. In an embodiment, the present invention provides a method of treatment of a Clostridium perfringens infection comprising the steps of administering the animal feed or animal feed additive to one or more animals with a Clostridium perfringens infection.

In another aspect, the present invention provides a method of treatment of necrotic enteritis comprising the steps of administering one or more lysozymes of the invention to one or more animals with necrotic enteritis. In an embodiment, the present invention provides a method of treatment of a Clostridium perfringens infection comprising the steps of administering one or more lysozymes of the invention to one or more animals with a Clostridium perfringens infection.

In a further aspect, the invention relates to the animal feed or animal feed additive of the invention for the treatment of necrotic enteritis in an animal. In an embodiment, the invention relates to composition comprising the lysozyme of SEQ ID NO: 257, SEQ ID NO: 264, SEQ ID NO: 267, SEQ ID NO: 291, SEQ ID NO: 294, SEQ ID NO: 297, SEQ ID NO: 300, SEQ ID NO: 303 and/or SEQ ID NO: 306 for the treatment of Necrotic enteritis in an animal.

In a further aspect, the invention relates to the animal feed or animal feed additive of the invention for the treatment of a Clostridium perfringens infection in an animal. In an embodiment, the invention relates to composition comprising the lysozyme of SEQ ID NO: 257, SEQ ID NO: 264, SEQ ID NO: 267, SEQ ID NO: 291, SEQ ID NO: 294, SEQ ID NO: 297, SEQ ID NO: 300, SEQ ID NO: 303 and/or SEQ ID NO: 306 for the treatment of a Clostridium perfringens infection in an animal.

Antimicrobial activity towards Clostridium perfringens can be determined according to the antimicrobial assay described in Example 12.

### PREFERRED EMBODIMENTS

Herein follows a list if preferred embodiments of the invention.

1. An animal feed or animal feed additive comprising one or more polypeptides having lysozyme activity, wherein the polypeptide comprises one or more GH24 catalytic domains and one or more lysozyme enhancing domains and wherein:
(a) GH24 catalytic domain gives a domT score of at least 200 when queried using a Profile Hidden Markov Model prepared using SEQ ID NOs: 1 to 122 and hmmbuild software program, and wherein the query is carried out using hmmscan software program with default settings; and
(b) the polypeptide comprises one or more lysozyme enhancing domains, wherein the lysozyme enhancing domain gives a domT score of at least 100 when queried using a Profile Hidden Markov Model prepared using SEQ ID NOs: 123 to 251 and hmmbuild software program, and wherein the query is carried out using the hmmscan software program with default settings.
   2. The animal feed or animal feed additive of item 1, wherein the GH24 catalytic domain gives a domT score of 220 or more and the lysozyme enhancing domain gives a domT score of 100 or more.
   3. The animal feed or animal feed additive of item 1, wherein the GH24 catalytic domain gives a domT score of 230 or more and the lysozyme enhancing domain gives a domT score of 100 or more.
   4. The animal feed or animal feed additive of item 1, wherein the GH24 catalytic domain gives a domT score of 240 or more and the lysozyme enhancing domain gives a domT score of 100 or more.
   5. The animal feed or animal feed additive of item 1, wherein the GH24 catalytic domain gives a domT score of 250 or more and the lysozyme enhancing domain gives a domT score of 103 or more.
   6. An animal feed or animal feed additive comprising one or more polypeptides having lysozyme activity, wherein:
(a) the polypeptide comprises one or more GH24 catalytic domain comprising one or more motif I: [TAS][VIL][GAC][YFI]GHX[CAYIV] (SEQ ID NO: 252) and/or one or more motif II [LV][NDST]XN[QE][YFW][GASND]ALXS[WFLY]X[FY]N (SEQ ID NO: 253); and
(b) the polypeptide comprises one or more lysozyme enhancing domain comprising one or more motif III: [CGY][YF][VIL][ASTP][DG]X[YF][VIT]X[TS][GAN] (SEQ ID NO: 254).
   7. An animal feed or animal feed additive comprising one or more polypeptides having lysozyme activity, wherein:
(a) the polypeptide comprises one or more GH24 catalytic domain comprising one or more motif I: [TAS][VIL][GAC][YFI]GHX[CAYIV] (SEQ ID NO: 252) and one or more motif II [LV][NDST]XN[QE][YFW][GASND]ALXS[WFLY]X[FY]N (SEQ ID NO: 253); and
(b) the polypeptide comprises one or more lysozyme enhancing domain comprising one or more motif III: [CGY][YF][VIL][ASTP][DG]X[YF][VIT]X[TS][GAN] (SEQ ID NO: 254).
   8. The animal feed or animal feed additive of item 6 or 7, wherein the one or more motif I is [TAS][VIL][GAC][YFI]GHX[CAYIV] (SEQ ID NO: 281).
   9. The animal feed or animal feed additive item 6 or 7, wherein the one or more motif I is T[VI]GYGHXC (SEQ ID NO: 282).
   10. The animal feed or animal feed additive of any of items 6 to 9, wherein the one or more motif II LNXN[QE][YFW][GA]ALXS[WFL]X[YF]N (SEQ ID NO: 283).
   11. The animal feed or animal feed additive of any of items 6 to 10, wherein the one or more motif III C [YF][VI][AST]D[YKF][YF][VI]XTG (SEQ ID NO: 284).
   12. The animal feed or animal feed additive of any of items 6 to 11, wherein the GH24 catalytic domain further comprises one or more motif IV: [GEV]LXXRRXXE (SEQ ID NO: 285).
   13. The animal feed or animal feed additive of any of items 6 to 12, wherein the GH24 catalytic domain further comprises one or more motif IV: GLXXRRXXE (SEQ ID NO: 286).
   14. The animal feed or animal feed additive of any of items 1 to 13, wherein the lysozyme enhancing domain has at least 45% sequence identity to SEQ ID NO: 180.
   15. The animal feed or animal feed additive of any of items 1 to 14, wherein the lysozyme enhancing domain has at least 65% sequence identity to one or more of SEQ ID NO: 123 to 251.
   16. The animal feed or animal feed additive of any of items 1 to 15, wherein the GH24 catalytic domain has at least 55% sequence identity to SEQ ID NO: 14.
   17. The animal feed or animal feed additive of any of items 1 to 16, wherein the GH24 catalytic domain has at least 65% sequence identity to one or more of SEQ ID NO: 1 to 122.
   18. The animal feed or animal feed additive of any of items 1 to 17, wherein the polypeptide is obtained or obtainable from the kingdom Fungi.
   19. The animal feed or animal feed additive of any of items 1 to 18, wherein the polypeptide is obtained or obtainable from the phylum Ascomycota.
   20. The animal feed or animal feed additive of any of items 1 to 19, wherein the polypeptide is selected from the group consisting of:
(a) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 257;
(b) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 264;
(c) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 267;
(d) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 291;
(e) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 294;
(f) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 297;
(g) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 300;
(h) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 303;
(i) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 306;
(j) a variant of SEQ ID NO: 257, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(k) a variant of SEQ ID NO: 264, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
l) a variant of SEQ ID NO: 267, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(m) a variant of SEQ ID NO: 291, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(n) a variant of SEQ ID NO: 294, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(o) a variant of SEQ ID NO: 297, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(p) a variant of SEQ ID NO: 300, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(q) a variant of SEQ ID NO: 303, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(r) a variant of SEQ ID NO: 306, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(s) a fragment of the polypeptide of (a), (b), (c), (d), (e), (f), (g), (h), (i), (j), (k), (1), (m), (n), (o), (p), (q) or (r) that has lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids.
   21. The animal feed or animal feed additive of any of items 1 to 20, wherein the polypeptide having lysozyme activity has at least 30% of the antimicrobial activity of SEQ ID NO: 257 against Clostridium perfringens using the conditions 50% MHB, pH 6.
   22. The animal feed or animal feed additive of any of items 1 to 20, wherein the polypeptide having lysozyme activity has at least 30%, e.g., at least 50%, at least 60%, at least 70%, at least 80%, at least 90%, at least 95%, or at least 100% of the antimicrobial activity of SEQ ID NO: 257 against Clostridium perfringens using the conditions 50% MHB, pH 6.
   23 The animal feed or animal feed additive of any of items 1 to 20, wherein the polypeptide having lysozyme activity has at least 30%, e.g., at least 50%, at least 60%, at least 70%, at least 80%, at least 90%, at least 95%, or at least 100% of the antimicrobial activity of SEQ ID NO: 264 against Clostridium perfringens using the conditions 50% MHB, PH 6.
   24. The animal feed or animal feed additive of any of items 1 to 20, wherein the polypeptide having lysozyme activity has at least 30%, e.g., at least 50%, at least 60%, at least 70%, at least 80%, at least 90%, at least 95%, or at least 100% of the antimicrobial activity of SEQ ID NO: 267 against Clostridium perfringens using the conditions 50% MHB, pH 6.
   25. The animal feed or animal feed additive of any of items 1 to 24 further comprising one or more components selected from the list consisting of:
one or more additional enzymes;
one or more microbes;
one or more vitamins;
one or more minerals;
one or more amino acids; and
one or more other feed ingredients.
   26. The animal feed or animal feed additive of item 25, wherein the one or more additional enzymes is selected from the group consisting of phytase, xylanase, galactanase, alpha-galactosidase, protease, phospholipase A1, phospholipase A2, lysophospholipase, phospholipase C, phospholipase D, amylase, lysozyme, arabinofuranosidase, beta-xylosidase, acetyl xylan esterase, feruloyl esterase, cellulase, cellobiohydrolases, beta-glucosidase, pullulanase, and beta-glucanase or any combination thereof.
   27. The animal feed or animal feed additive of item 25, wherein the one or more microbes is selected from the group consisting of Bacillus subtilis, Bacillus licheniformis, Bacillus amyloliquefaciens, Bacillus cereus, Bacillus pumilus, Bacillus polymyxa, Bacillus megaterium, Bacillus coagulans, Bacillus circulans, Bifidobacterium bifidum, Bifidobacterium animalis, Bifidobacterium sp., Carnobacterium sp., Clostridium butyricum, Clostridium sp., Enterococcus faecium, Enterococcus sp., Lactobacillus sp., Lactobacillus acidophilus, Lactobacillus farciminus, Lactobacillus rhamnosus, Lactobacillus reuteri, Lactobacillus salivarius, Lactococcus lactis, Lactococcus sp., Leuconostoc sp., Megasphaera elsdenii, Megasphaera sp., Pediococsus acidilactici, Pediococcus sp., Propionibacterium thoenii, Propionibacterium sp. and Streptococcus sp. or any combination thereof.
   28. A pelleted animal feed comprising plant based material and the animal feed or animal feed additive of any of items 1 to 27.
   29. The pelleted animal feed of item 28, wherein the plant based material comprises oats, rye, barley, wheat, maize, corn, sorghum, switchgrass, millet, pearl millet, foxtail millet, soybean, wild soybean, beans, lupin, tepary bean, scarlet runner bean, slimjim bean, lima bean, French bean, Broad bean (fava bean), chickpea, lentil, peanut, Spanish peanut, canola, rapeseed (oilseed rape) or pea, in a processed form thereof or any combination thereof.
   30. A polypeptide having lysozyme activity, selected from the group consisting of:
(a) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 257;
(b) a polypeptide having at least 95%, e.g., at least 96%, at least 97%, at least 98%, at least 99%, or 100% sequence identity to SEQ ID NO: 267;
(c) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 291;
(d) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 294;
(e) a polypeptide having at least 82%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 297;
(f) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 300;
(g) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 303;
(h) a polypeptide having at least 80%, e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 306;
(i) a polypeptide encoded by a polynucleotide that hybridizes under medium-high stringency conditions, high stringency conditions or very-high stringency conditions with:
   (i) the mature polypeptide coding sequence of SEQ ID NO: 255;
   (ii) the mature polypeptide coding sequence of SEQ ID NO: 287;
   (iii) the mature polypeptide coding sequence of SEQ ID NO: 292;
   (iv) the mature polypeptide coding sequence of SEQ ID NO: 295;
   (v) the mature polypeptide coding sequence of SEQ ID NO: 298;
   (vi) the mature polypeptide coding sequence of SEQ ID NO: 301;
   (vii) the mature polypeptide coding sequence of SEQ ID NO: 304;
   (viii) the cDNA sequence thereof; or
   (ix) the full-length complement of (i), (ii), (iii), (iv) (v), (vi), (vii) or (viii);
(j) a polypeptide encoded by a polynucleotide that hybridizes under very-high stringency conditions with:
   (i) the mature polypeptide coding sequence of SEQ ID NO: 265;
   (ii) the full-length complement of (i);
(k) a polypeptide encoded by a polynucleotide having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 255;
(l) a polypeptide encoded by a polynucleotide having at least 95%, e.g., at least 96%, at least 97%, at least 98%, at least 99%, or 100% sequence identity to the mature polypeptide coding sequence of SEQ ID NO: 265;
(m) a polypeptide encoded by a polynucleotide having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 287;
(n) a polypeptide encoded by a polynucleotide having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 292;
(o) a polypeptide encoded by a polynucleotide having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 295;
(p) a polypeptide encoded by a polynucleotide having at least 82%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 298;
(q) a polypeptide encoded by a polynucleotide having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 301;
(r) a polypeptide encoded by a polynucleotide having at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of SEQ ID NO: 304;
(s) a variant of SEQ ID NO: 257, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions; and
(t) a variant of SEQ ID NO: 267, wherein the variant has lysozyme activity and comprises one or more substitutions and/or one or more deletions and/or one or more insertions or any combination thereof in 1, 2, 3, 4, 5, 6, 7, 8, 9, 10, 11 or 12 positions; and
(u) a variant of SEQ ID NO: 291, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(v) a variant of SEQ ID NO: 294, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
w) a variant of SEQ ID NO: 297, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(x) a variant of SEQ ID NO: 300, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(y) a variant of SEQ ID NO: 303, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions;
(z) a variant of SEQ ID NO: 306, wherein the variant has lysozyme activity and comprises one or more substitutions and/or one or more deletions and/or one or more insertions or any combination thereof in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 positions; and
(aa) a fragment of the polypeptide of (a), (b), (c), (d), (e), (f), (g), (h), (i), (j), (k), (l), (m), (n), (o), (p), (q), (r), (s), (t), (u), (v), (w), (x), (y) or (z) that has lysozyme activity wherein the fragment comprises at least 200 amino acids, such as at least 210 amino acids, at least 215 amino acids, at least 220 amino acids, at least 225 amino acids, at least 230 amino acids, at least 235 amino acids or at least 240 amino acids.
   31. The polypeptide according to item 30, wherein the polypeptide comprises or consists of amino acids 1 to 245 of SEQ ID NO: 257, amino acids 1 to 248 of SEQ ID NO: 267, amino acids 1 to 245 of SEQ ID NO: 291, amino acids 1 to 249 of SEQ ID NO: 294, amino acids 1 to 245 of SEQ ID NO: 297, amino acids 1 to 247 of SEQ ID NO: 300, amino acids 1 to 250 of SEQ ID NO: 303 or amino acids 1 to 240 of SEQ ID NO: 306.
   32. A composition comprising one or more polypeptides of any of items 30 to 31 and one or more formulating agents.
   33. The composition of item 32 wherein the formulating agent comprises one or more of the following compounds: glycerol, ethylene glycol, 1,2-propylene glycol or 1, 3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, glucose, sucrose, sorbitol, lactose, wheat flour, wheat bran, corn gluten meal, starch, kaolin and cellulose or any combination thereof.
   34. A granule comprising one or more polypeptides of item 30 or 31 or the composition of item 32 or 33.
   35 The granule of item 34 wherein the granule is coated.
   36. The granule of item 35 wherein the coating comprises a salt and/or wax and/or a flour.
   37. Use of the polypeptide of item 30 or 31, the animal feed or animal feed additive of any of items 1 to 27, the pelleted animal feed of item 28 or 29, the composition of item 32 or 33 or the granule of any of items 34 to 36:
in animal feed;
in animal feed additives;
in the preparation of a composition for use in animal feed;
for improving one or more performance parameters in an animal;
for the treatment of Clostridium perfringens infection in an animal; and/or for the treatment of necrotic enteritis in an animal.
   38. A method of treatment of a Clostridium perfringens infection and/or necrotic enteritis in an animal comprising the steps of administering the polypeptide of item 30 or 31, the animal feed or animal feed additive of any of items 1 to 27, the pelleted animal feed of item 28 or 29, the composition of item 32 or 33 or the granule of any of items 34 to 36 to one or more animals.
   39. The polypeptide of item 30 or 31, the animal feed or animal feed additive of any of items 1 to 27, the pelleted animal feed of item 28 or 29, the composition of item 32 or 33 or the granule of any of items 34 to 36 for use as a medicament.
   40. The polypeptide of item 30 or 31, the animal feed or animal feed additive of any of items 1 to 27, the pelleted animal feed of item 28 or 29, the composition of item 32 or 33 or the granule of any of items 34 to 36 for use in treatment of a Clostridium perfringens infection and/or necrotic enteritis in an animal.
   41. A method of improving the performance of an animal comprising administering to the animal the polypeptide of item 30 or 31, the animal feed or animal feed additive of any of items 1 to 27, the pelleted animal feed of item 28 or 29, the composition of item 32 or 33 or the granule of any of items 34 to 36.
   42. An isolated polynucleotide encoding the polypeptide of item 30 or 31.
   43. A nucleic acid construct or expression vector comprising the polynucleotide of item 42 operably linked to one or more control sequences that direct the production of the polypeptide in an expression host cell.
   44. A recombinant expression host cell comprising the polynucleotide of item 42 operably linked to one or more control sequences that direct the production of the polypeptide.
   45. A method of producing the polypeptide of item 30 or 31, comprising:
(a) cultivating a cell, which in its wild-type form produces the polypeptide, under conditions conductive for production of the polypeptide; and
(b) recovering the polypeptide.
   46. A method of producing the polypeptide of item 30 or 31, comprising:
(a) cultivating a host cell of item 44 under conditions conducive for production of the polypeptide; and
(b) recovering the polypeptide.
   47. A transgenic plant, plant part or plant cell transformed with a polynucleotide encoding the polypeptide of item 30 or 31.
   48. A whole broth formulation or cell culture composition comprising a polypeptide of item 30 or 31.

The present invention is further described by the following examples that should not be construed as limiting the scope of the invention.

### EXAMPLES

### Strains

Trichophaea saccata CBS804.70 was purchased from the Centraalbureau voor Schimmelcultures (Utrecht, the Netherlands). According to CBS, the strain was obtained from coal spoil tip soil, with high surface temperatures from Staffordshire, England in May 1968.

Trichoderma harzianum, originally named A00611 was deposited at the Centraalbureau voor Schimmelcultures, Ultrecht, Netherlands under the code: CBS223.93.

Chaetomium thermophilum var. thermophilum was purchased from Centraalbureau voor Schimmelcultures, Ultrecht, Netherlands under the code CBS 144.50. According to CBS, the strain was obtained from decaying wheat straw from Leeds, England on or before 1950.

Chaetomium sp. ZY 287 was obtained from a soil sample from Beijing, China in August 2013. Mortierella sp. ZY 002 were obtained from a soil sample from Shanxi province, China in December 2012. Metarhizium sp. XZ2431 and Geomyces auratus were obtained from soil samples from Guizhou province, China in July 2014. Ilyonectria rufa was obtained from Tibet in August 2011.

### Media and Solutions

YP+2% glucose medium was composed of 1% yeast extract, 2% peptone and 2% glucose.

YP+2% maltodextrin medium was composed of 1% yeast extract, 2% peptone and 2% maltodextrin.

PDA agar plates were composed of potato infusion (potato infusion was made by boiling 300 g of sliced (washed but unpeeled) potatoes in water for 30 minutes and then decanting or straining the broth through cheesecloth. Distilled water was then added until the total volume of the suspension was one liter, followed by 20 g of dextrose and 20 g of agar powder. The medium was sterilized by autoclaving at 15 psi for 15 minutes (Bacteriological Analytical Manual, 8th Edition, Revision A, 1998).

LB plates were composed of 10 g of Bacto-Tryptone, 5 g of yeast extract, 10 g of sodium chloride, 15 g of Bacto-agar, and deionized water to 1 liter.

LB medium was composed of 10 g of Bacto-Tryptone, 5 g of yeast extract, 10 g of sodium chloride, and deionized water to 1 liter.

COVE sucrose plates were composed of 342 g of sucrose, 20 g of agar powder, 20 ml of COVE salts solution, and deionized water to 1 liter. The medium was sterilized by autoclaving at 15 psi for 15 minutes (Bacteriological Analytical Manual, 8th Edition, Revision A, 1998). The medium was cooled to 60° C. and 10 mM acetamide, 15 mM CsCl, TRITON® X-100 (50 μl/500 ml) were added.

COVE salts solution was composed of 26 g of MgSO4·7H2O, 26 g of KCL, 26 g of KH2PO4, 50 ml of COVE trace metals solution, and deionized water to 1 liter. COVE trace metals solution was composed of 0.04 g of Na₂B₄O₇·10H₂O, 0.4 g of CuSO₄·5H₂O, 1.2 g of FeSO₄·7H₂O, 0.7 g of MnSO₄·H₂O, 0.8 g of Na₂MoO₄·2H₂O, 10 g of ZnSO₄·7H₂O, and deionized water to 1 liter.

Dap-4C medium was composed of 20 g Dextrose, 10 g Maltose, 11 g MgSO₄·7H₂O, 1 g KH₂PO₄, 2 g Citric Acid, 5.2 g K₃PO₄·H₂O, 0.5 g Yeast Extract (Difco), 1 ml Dowfax 63N10 (Dow Chemical Company), 0.5 ml KU6 trace metals solution, 2.5 g CaCO₃, and deionized water to 1 liter. The medium was sterilized by autoclaving at 15 psi for 15 minutes (Bacteriological Analytical Manual, 8th Edition, Revision A, 1998). Before use, Dap-4C medium was added 3.5 ml sterile 50% (NH₄)₂HPO₄ and 5 ml sterile 20% Lactic Acid per 150 ml medium.

Dap-2C medium was composed of 20 g maltodextrin, 11 g MgSO₄·7H₂O, 1 g KH₂PO₄, 2 g Citric Acid, 5.2 g K₃PO₄·H₂O, 0.5 g Yeast Extract (Difco), 1 ml Dowfax 63N10 (Dow Chemical Company), 0.5 ml KU6 trace metals solution, 2.5 g CaCO₃, and deionized water to 1 liter. The medium was sterilized by autoclaving at 15 psi for 15 minutes (Bacteriological Analytical Manual, 8th Edition, Revision A, 1998). Before use, Dap-4C medium was added 3.5 ml sterile 50% (NH₄)₂HPO₄ and 5 ml sterile 20% Lactic Acid per 150 ml medium.

KU6 trace metals solution was composed of 0.13 g NiCl₂, 2.5 g CuSO₄·5H₂O, 13.9 g FeSO₄·7H₂O, 8.45 g MnSO₄·H₂O, 6.8 g ZnCl₂, 3 g Citric Acid, and deionized water to 1 liter.

PDA medium was composed of 39 g of potato dextrose agar and deionized water to 1 liter.

Horikoshi medium contained 10 g of glucose, 5 g of peptone, 5 g of yeast extract, 1 g of K₂HPO₄, 0.2 g of MgSO₄ 7H₂O, 15 g of agar in 900 ml of distilled water. Autoclave at 121° C. for 15 minutes. After autoclaving, aseptically add 100.0 ml of sterile 10% Na₂CO₃ to the medium. Adjust for final pH of 10.0.

YPM medium contained 1% of Yeast extract, 2% of Peptone and 2% of Maltose.

Selective medium was composed of 342 g of sucrose, 20 ml of salt solution, 20 g of agar.

Salt solution was composed of 26 g of KCl, 26 g of MgSO₄ 7H₂O, 76 g of KH₂PO₄, 50 ml of trace element solution, in water with final volume of 1 L.

Trace element solution was composed of 400 mg of Na₂B₄O₇ 10H₂O, 400 mg of CuSO₄ 5H₂O, 800 mg of FeSO₄ 7H₂O, 800 mg of MnSO₄ 2H₂O, 800 mg of Na₂MoO₄ 2H₂O, 8 g of ZnSO₄ 7H₂O in water with final volume of 1 L.

Slant medium was composed of 30 g of sucrose, 20 ml of salt solution, 20 g of agar.

### Example 1: Expression of the GH24 Lysozyme from Trichophaea saccata

The fungal strain was cultivated in 100 ml of YP+2% glucose medium in 1000 ml Erlenmeyer shake flasks for 5 days at 20° C. Mycelia were harvested from the flasks by filtration of the medium through a Buchner vacuum funnel lined with MIRACLOTH® (EMD Millipore, Billerica, MA, USA). Mycelia were frozen in liquid nitrogen and stored at −80° C. until further use. Genomic DNA was isolated using a DNEASY® Plant Maxi Kit (QIAGEN GMBH, Hilden Germany) according to the manufacturer's instructions.

Genomic sequence information was generated by Illumina MySeq (Illumina Inc., San Diego, CA). The polypeptide coding sequence for the entire coding region was cloned from Trichophaea saccata CBS804.70 genomic DNA by PCR using the primers F-80470 and R-80470 (SEQ ID NO: 258 and SEQ ID NO: 259 respectively) as described below.

(SEQ ID NO: 258)
5′-ACACAACTGGGGATCCACCATGCACGCTCTCACCCTTCT-3′
(SEQ ID NO: 259)
5′-CTAGATCTCGAGAAGCTTTTAGCACTTGGGAGGGTGGG-3′

Bold letters represent Trichophaea saccata enzyme coding sequence. Restriction sites are underlined. The sequence to the left of the restriction sites is homologous to the insertion sites of pDau109 (WO 2005/042735).

Extensor HIFI PCR mix, 2× concentration (Thermo Scientific cat no AB-0795) was used for experiment.

The amplification reaction (25 μl) was performed according to the manufacturer's instructions (Thermo Scientific cat no AB-0795) with the following final concentrations:

### PCR Mix:

0.5 μM Primer F-80470
0.5 μM Primer R-80470
12.5 μl Extensor HIFI PCR mix, 2×conc.
11.0 μl H₂O
10 ng of Trichophaea saccata CBS 804.70 genomic DNA.

The PCR reaction was incubated in a DYAD® Dual-Block Thermal Cycler (BioRad, USA) programmed for 1 cycle at 94° C. for 30 seconds; 30 cycles each at 94° C. for 30 seconds, 52° C. for 30 seconds and 68° C. for 60 seconds followed by 1 cycle at 68° C. for 6 minutes. Samples were cooled to 10° C. before removal and further processing.

Three μl of the PCR reaction were analyzed by 1% agarose gel electrophoresis using 40 mM Tris base, 20 mM sodium acetate, 1 mM disodium EDTA (TAE) buffer. A major band of about 946 bp was observed. The remaining PCR reaction was purified directly with an ILLUSTRA™ GFX™ PCR DNA and Gel Band Purification Kit (GE Healthcare, Piscataway, NJ, USA) according to the manufacturer's instructions.

Two μg of plasmid pDau109 was digested with Bam HI and Hind III and the digested plasmid was run on a 1% agarose gel using 50 mM Tris base-50 mM boric acid-1 mM disodium EDTA (TBE) buffer in order to remove the stuffer fragment from the restricted plasmid. The bands were visualized by the addition of SYBR® Safe DNA gel stain (Life Technologies Corporation, Grand Island, NY, USA) and use of a 470 nm wavelength transilluminator. The band corresponding to the restricted plasmid was excised and purified using an ILLUSTRA™ GFX™ PCR DNA and Gel Band Purification Kit. The plasmid was eluted into 10 mM Tris pH 8.0 and its concentration adjusted to 20 ng per μl. An IN-FUSION® PCR Cloning Kit (Clontech Laboratories, Inc., Mountain View, CA, USA) was used to clone the 983 bp PCR fragment into pDau109 digested with Bam HI and Hind III (20 ng). The IN-FUSION® total reaction volume was 10 μl. The IN-FUSION® total reaction volume was 10 μl. The IN-FUSION® reaction was transformed into FUSION-BLUE™ E. coli cells (Clontech Laboratories, Inc., Mountain View, CA, USA) according to the manufacturer's protocol and plated onto LB agar plates supplemented with 50 μg of ampicillin per ml. After incubation overnight at 37° C., transformant colonies were observed growing under selection on the LB plates supplemented with 50 μg of ampicillin per ml.

Several colonies were selected for analysis by colony PCR using the pDau10⁹ vector primers described below. Four colonies were transferred from the LB plates supplemented with 50 μg of ampicillin per ml with a yellow inoculation pin (Nunc A/S, Denmark) to new LB plates supplemented with 50 μg of ampicilin per ml and incubated overnight at 37° C.

Primer 8653:
(SEQ ID NO: 260)
5′-GCAAGGGATGCCATGCTTGG-3′
Primer 8654:
(SEQ ID NO: 261)
5′-CATATAACCAATTGCCCTC-3′

Each of the three colonies were transferred directly into 200 μl PCR tubes composed of 5 μl of 2× Extensor HIFI PCR mix, (Thermo Fisher Scientific, Rockford, IL, USA), 0.5 μl of primer 8653 (10 pm/μl), 0.5 μl of primer 8654 (10 pm/μl), and 4 μl of deionized water. Each colony PCR was incubated in a DYAD® Dual-Block Thermal Cycler programmed for 1 cycle at 94° C. for 60 seconds; 30 cycles each at 95° C. for 30 seconds, 60° C. for 45 seconds, 72° C. for 60 seconds, 68° C. for 10 minutes, and 10° C. for 10 minutes.

Three μl of each completed PCR reaction were submitted to 1% agarose gel electrophoresis using TAE buffer. All four E. coli transformants showed a PCR band of about 980 bp. Plasmid DNA was isolated from each of the four colonies using a QIAprep Spin Miniprep Kit (QIAGEN GMBH, Hilden Germany). The resulting plasmid DNA was sequenced with primers 8653 and 8654 (SEQ ID NO: 260 and 261) using an Applied Biosystems Model 3730 Automated DNA Sequencer using version 3.1 BIG-DYE™ terminator chemistry (Applied Biosystems, Inc., Foster City, CA, USA). One plasmid, designated pKKSC0312-2, was chosen for transforming Aspergillus oryzae MT3568. A. oryzae MT3568 is an amdS (acetamidase) disrupted gene derivative of Aspergillus oryzae JaL355 (WO 2002/40694) in which pyrG auxotrophy was restored by inactivating the A. oryzae amdS gene. Protoplasts of A. oryzae MT3568 were prepared according to the method described in EP 0238023, pages 14-15.

E. coli 3701 containing pKKSC0312-2 was grown overnight according to the manufacturer's instructions (Genomed) and plasmid DNA of pKKSC0312-2 was isolated using a Plasmid Midi Kit (Genomed J ETquick kit, cat.nr. 400250, GENOMED GmbH, Germany) according to the manufacturer's instructions. The purified plasmid DNA was transformed into Aspergillus oryzae MT3568. A. oryzae MT3568 protoplasts were prepared according to the method of Christensen et al., 1988, Bio/Technology 6:1419-1422. The selection plates consisted of COVE sucrose with+10 mM acetamide+15 mM CsCl+TRITON® X-100 (50 μl/500 ml). The plates were incubated at 37° C. Briefly, 8 μl of plasmid DNA representing 3 ugs of DNA was added to 100 μl MT3568 protoplasts. 250 μl of 60% PEG solution was added and the tubes were gently mixed and incubate at 37° for 30 minutes. The mix was added to 10 ml of pre melted Cove top agarose (The top agarose melted and then the temperature equilibrated to 40 C in a warm water bath before being added to the protoplast mixture). The combined mixture was then plated on two Cove-sucrose selection petri plates with 10 mM Acetamide. The plates were incubated at 37° C. for 4 days. Single Aspergillus transformed colonies were identified by growth on plates using the selection Acetimide as a carbon source. Each of the four A. oryzae transformants were inoculated into 750 μl of YP medium supplemented with 2% glucose and also 750 μl of 2% maltodextrin and also DAP4C in 96 well deep plates and incubated at 37° C. stationary for 4 days. At the same time the four transformants were restreaked on COVE-2 sucrose agar medium.

Culture broth from the Aspergillus oryzae transformants were then analyzed for production of the GH24 polypeptide by SDS-PAGE using NUPAGE® 10% Bis-Tris SDS gels (Invitrogen, Carlsbad, CA, USA) according to the manufacturer's recommendations. A protein band at approximately 27 kDa was observed for each of the Aspergillus oryzae transformants. One A. oryzae transformant was cultivated in 1000 ml Erlenmeyer shake flasks containing 100 ml of DAP4C medium at 26° C. for 4 days with agitation at 85 rpm and purified as described in example 2.

### Example 2: Purification of the GH24 Lysozyme from Trichophaea saccata

The fermentation supernatant with the GH24 lysozyme was filtered through a Fast PES Bottle top filter with a 0.22 μm cut-off. The resulting solution was diafiltrated with 5 mM Na-acetate, pH 4.5 and concentrated (volume reduced by a factor of 10) on an Ultra Filtration Unit (Sartorius) with a 10 kDa cut-off membrane.

After pretreatment about 275 ml of the lysozyme containing solution was purified by chromatography on SP Sepharose (approximately 60 mL) in a XK26 column eluting the bound lysozyme with 0 to 100% gradient of buffer A (50 mM Na-acetate pH 4.5) and buffer B (50 mM Na-acetate+1 M NaCl pH 4.5) over 10 column volumes. The fractions from the column were pooled based on the chromatogram (absorption at 280 and 254 nm) and SDS-PAGE analysis.

The molecular weight, as estimated from SDS-PAGE, was approximately 27 kDa and the purity was >90%.

Characteristics for the GH24 Lysozyme from Trichophaea saccata

Determination of the N-terminal sequence was: YPVKTDL.

The calculated molecular weight from this mature sequence is 26205.5 Da (M+H)⁺.

The molecular weight determined by intact molecular weight analysis was 26205.3 Da. (M+H)⁺.

The mature sequence (from EDMAN N-terminal sequencing data, intact molecular weight analysis and proteomic analysis with 92% amino acid coverage):

(SEQ ID NO: 257)
YPVKTDLHCRSSPSTSASIVRTYSSGTEVQIQCQTTGTSVQGSNV
WDKTQHGCYVADYYVKTGHSGIFTTKCGSSSGGGSCKPPPINAAT
VALIKEFEGFVPKPAPDPIGLPTVGYGHLCKTKGCKEVPYSFPLT
QETATKLLQSDIKTFTSCVSNYVKDSVKLNDNQYGALASWAFNVG
CGNVQTSSLIKRLNAGENPNTVAAQELPKWKYAGGKVMPGLVRRR
NAEVALFKKPSSVQAHPPKC.

### Example 3: Expression of the LED Alone from Trichophaea saccata

In order to establish the properties of the newly defined lysozyme enhancing domain, an expression construct in which the N-terminal portion of the GH24 polypeptide coding sequence from example 1 was expressed.

The amino acid sequence shown below represents the signal peptide and the NZ4 domain. The signal peptide is underlined.

(SEQ ID NO: 272)
MHALTLLTATLFGLAAAYPVKTDLHCRSSPSTSASIVRTYSSGTE
VQIQCQTTGTSVQGSNVWDKTQHGCYVADYYVKTGHSGIFTTKCG

The DNA representing SEQ ID NO: 272 is shown below as SEQ ID NO: 271 wherein the signal peptide is underlined.

(SEQ ID NO: 271)
ATGCACGCTCTCACCCTTCTCACCGCAACCCTCTTCGGTCTCGCA
GCGGCCTACCCAGTGAAGACCGACCTTCACTGCCGCTCCTCTCCC
AGCACTTCCGCCAGCATCGTCCGCACCTACTCCAGTGGAACGGAA
GTCCAGATCCAGTGCCAGACCACGGGCACTTCGGTCCAAGGATCC
AATGTCTGGGACAAGACCCAGCACGGTTGCTACGTCGCAGACTAC
TACGTCAAGACCGGGCATTCTGGGATTTTCACCACCAAGTGCGGT

PCR primers were designed in order to amplify the DNA fragment SEQ ID NO: 271 from pKKSC0312-2 of example 1. The forward primer from example 1 (SEQ ID NO: 258) could be reused. For the reverse primer, a TAA stop codon was added to the DNA sequence of the above and then the Infusion cloning site for the HindIII site was added to create the primer below:

(SEQ ID NO: 274)
AGATCTCGAGAAGCTTATACCGCACTTGGTGGTGAAA.

The amplification reaction (25 μl) was performed according example 1. The 270 base pair PCR product was cloned into vector pDau222 also as described in example 1.

Culture broth from the Aspergillus oryzae transformants were then analyzed for production of the LED polypeptide by SDS-PAGE using NUPAGE® 8% Bis-Tris SDS gels (Invitrogen, Carlsbad, CA, USA) according to the manufacturer. A band at approximately 14 kDa was observed for each of the Aspergillus oryzae transformants. The actual size appears larger than the predicted size of 8.4 kDa possibly because of post translational modification. One A. oryzae transformant producing the LED polypeptide was cultivated in 1000 ml Erlenmeyer shake flasks containing 100 ml of DAP4C medium at 30° C. for 4 days with agitation at 150 rpm and purified as described in example 4.

### Example 4: Expression of the LED Alone from Trichophaea saccata

The fermentation supernatant with the LED alone from Trichophaea saccata was filtered through a Fast PES Bottle top filter with a 0.22 μm cut-off. The resulting solution was desalted on a G25 Sephadex column (approximately 500 mL) into 50 mM Na-acetate, pH 4.5. Following this the protein containing solution (approximately 125 ml) was purified by chromatography on SP Sepharose (approximately 50 mL) in a XK26 column eluting the bound lysozyme with 0 to 100% gradient of buffer A (50 mM Na-acetate pH 4.5) and buffer B (50 mM Na-acetate+1 M NaCl pH 4.5) over 10 column volumes. The fractions from the column were pooled based on the chromatogram (absorption at 280 and 254 nm) and SDS-PAGE analysis.

The molecular weight, as estimated from SDS-PAGE, was 6-10 kDa and the purity was >95%. The calculated molecular weight from this mature sequence is 7906.64 Da.

The mature sequence (from proteomic analysis with 97% amino acid coverage) was determined to be:

(SEQ ID NO: 273)
YPVKTDLHCRSSPSTSASIVRTYSSGTEVQIQCQTTGTSVQGSNV
WDKTQHGCYVADYYVKTGHSGIFTTKCG.

### Example 5: Expression of the GH24 Domain of Trichophaea saccata CBS804.70

In order to determine if the lysozyme enhancing domain has functional or enhancing properties for the associated GH24 lysozyme, a construct was cloned and protein expressed in which the LED had been removed from the GH24 lysozyme. The construct was made as follows:

A synthetic gene was created as described below.

Based on multiple sequence alignments, QCVG or similar appears to be fairly well conserved in the GH24 fungal lysozymes without the LED domain. Therefor the signal and the sequence downstream QCVG sequence from SEQ ID NO: 279 (GH24 from Acremonium acalophilum) was used in part of the construct. This peptide sequence was then added directly to the mature peptide fragment containing only the GH24 lysozyme region of SEQ ID NO: 257 resulting in the amino acid sequence below:

(SEQ ID NO: 269)
MAKVSTLTIALLTMASQARAQCVGCKPPPINAATVALIKEFEGFV
PKPAP DPIGLPTVGYGHLCKTKGCKEVPYSFPLTQETATKLLQS
DIKTFTSCVSNYVKDSVKLNDNQYGALASWAFNVGCGNVQTSSLI
KRLNAGENPNTVAAQELPKWKYAGGKVMPGLVRRRNAEVALFKKP
SSVQAHPPKC.

A synthetic DNA (SEQ ID NO: 268) that had been codon optimised for expression in Aspergillus oryzae was ordered from GeneArt® Gene Synthesis, Life Technologies. The synthetic DNA conveniently had BamHI and HindIII restriction sites added to the ends so as to make a BamHI-HindIII restricted fragment compatible with the cloning vector pDau109 used in example 1.

The fragment was received from GeneArt in kanamycin resistant vector pMK and the plasmid was dissolved DNA in 50 μls in 10 mM Tris, pH7.5. 20 μls of the mixture with BamHI-HindIII according to the protocol below:

| | | |
|---|---|---|
| Plasmid | 20 | μl |
| Cut smart buffer: 10x #B7204S | 4.0 | μl |
| Milli-Q H₂O: | 14.0 | μl |
| Hind III HF: NEB #R3104S | 1 | μl |
| BamH HF: NEB #R3136S | 1 | μl |
| Total volume | 40 | μl |

The reaction was incubated for 3 hours at 37° C. The restriction digest was purified with the GFX PCR DNA and Gel Band purification Kit (Cat. no 28-9034-71) from GE Healthcare and eluted in 40 μls 10 mM Tris, pH 7.5. The purified restriction digest was then used in a simple ligation with BamHI-HindIII restricted pDau109 (example 1) according to the T4 DNA ligase manufacturer's instructions (New England Biolabs., neb.com).

2.5 μl of the 10 μl ligation was used to transform 25 μl Stellar™ (Life Technologies) Competent cells according to the manufacturer's instructions and the treated cells plated on LB agar plates with 50 mg/ml ampicillin. Colony PCR of select transformants was performed according to example 1 and the PCR fragments sequenced. A single plasmid, selected as being PCR error free, was transformed into Aspergillus oryzae MT3568 according to example 1. Among the several transformants that produced the secreted product of about 20 kDa, one was chosen and cultivated in 1000 ml Erlenmeyer shake flasks containing 100 ml of DAP4C medium at 30° C. for 4 days with agitation at 150 rpm and purified as described in example 6.

### Example 6: Purification of the GH24 Domain of Trichophaea saccata CBS804.70

The fermentation supernatant with the GH24 domain was filtered through a Fast PES Bottle top filter with a 0.22 μm cut-off. The resulting solution was diafiltrated with 50 mM Na-acetate, pH 4.5 on an Ultra Filtration Unit (Sartorius) with a 10 kDa cut-off membrane.

After pretreatment about 500 ml of the lysozyme containing solution was purified by chromatography on SP Sepharose (approximately 50 mL) in a XK26 column eluting the bound protein with 0 to 100% gradient of buffer A (50 mM Na-acetate pH 4.5) and buffer B (50 mM Na-acetate+1 M NaCl pH 4.5) over 10 column volumes. The fractions from the column were pooled based on the chromatogram (absorption at 280 and 254 nm) and SDS-PAGE analysis.

The molecular weight, as estimated from SDS-PAGE, was approximately 20 kDa and the purity was >90%.

The calculated molecular weight from this mature sequence is 18182.86 Da.

The mature sequence (from proteomic analysis with 72% amino acid coverage) was determined to be:

(SEQ ID NO: 270)
QCVGCKPPPINAATVALIKEFEGFVPKPAPDPIGLPTVGYGHLCK
TKGCKEVPYSFPLTQETATKLLQSDIKTFTSCVSNYVKDSVKLND
NQYGALASWAFNVGCGNVQTSSLIKRLNAGENPNTVAAQELPKWK
YAGGKVMPGLVRRRNAEVALFKKPSSVQAHPPKC.

### Example 7: Cloning and Expression of GH24 Lysozyme from Trichoderma harzianum A00611

Trichoderma harzianum, originally named A00611 was deposited at the Centraalbureau voor Schimmelcultures, Ultrecht, Netherlands under the code: CBS223.93. Trichoderma harzianum A00611 was grown on Potato Dextrose Agar at 26° C. for several days. Mycelia was harvested directly from the inoculated PDA agar plate and the DNA prepared as in example 1.

The polypeptide coding sequence for the entire coding region was cloned from Trichoderma harzianum A00611 genomic DNA by PCR using primers SEQ ID NO: 275 and SEQ ID NO: 276 as given below.

F
SEQ ID NO: 275
5′-ACACAACTGGGGATCCACCATGAAGACTGCCTTTGCTGC-3′
R
SEQ ID NO : 276
5′-AGATCTCGAGAAGCTTATCACTGGCACTTTGGATAGGC-3′

Bold letters represent Trichoderma harzianum enzyme coding sequence. Restriction sites are underlined. The sequence to the left of the restriction sites is homologous to the insertion sites of pDau109 (WO 2005/042735).

Cloning of the 800 bp PCR fragment, transformation into Aspergillus oryzae and production of culture fluid for purification of the GH24 lysozyme was performed according to example 1.

A band at approximately 27 kDa was observed for each of the Aspergillus oryzae transformants. One A. oryzae transformant producing the polypeptide was chosen and cultivated in 1000 ml Erlenmeyer shake flasks containing 100 ml of DAP4C medium at 30° C. for 4 days with agitation at 150 rpm and purified as described in example 8.

### Example 8: Purification of the GH24 Lysozyme from Trichoderma harzianum A00611

The fermentation supernatant with the GH24 lysozyme from Trichoderma harzianum A00611 was filtered through a Fast PES Bottle top filter with a 0.22 μm cut-off. About one liter was obtained and the pH was adjusted to 4.5 with diluted acetic acid. The sample was diluted to two liters with Milli-Q water.

After pretreatment about 1000 ml of the lysozyme containing solution was purified by chromatography on SP Sepharose (approximately 30 mL) in a XK26 column eluting the bound lysozyme with 0 to 100% gradient of buffer A (50 mM Na-acetate pH 4.5) and buffer B (50 mM Na-acetate+1 M NaCl pH 4.5) over 10 column volumes. The fractions from the column were pooled based on the chromatogram (absorption at 280 and 254 nm) and SDS-PAGE analysis.

The molecular weight, as estimated from SDS-PAGE, was approximately 27 kDa and the purity was >90%.

### Example 9: Cloning and Expression of the GH24 Lysozyme from Chaetomium thermophilum var. thermophilum

Chaetomium thermophilum var. thermophilum was purchased from Centraalbureau voor Schimmelcultures, Ultrecht, Netherlands under the code CBS 144.50. The strain was grown on Potato Dextrose Agar at 37° C. for several days. Mycelia was harvested directly from the inoculated PDA agar plate and the DNA prepared as in example 1.

The polypeptide coding sequence for the entire coding region was cloned from Chaetomium thermophilum CBS 144.50 genomic DNA by PCR using primers SEQ ID NO: 277 and SEQ ID NO: 278 as given below.

F
SEQ ID NO: 277
5′-ACACAACTGGGGATCCACCATGAAGTTCGCCATCCTCGC-3′
R
SEQ ID NO: 278
5′-AGATCTCGAGAAGCTTATCAGCACTTAACAGGAAGAGCC-3′

Bold letters represent GH24 enzyme coding sequence. Restriction sites are underlined. The sequence to the left of the restriction sites is homologous to the insertion sites of pDau109 (WO 2005/042735).

Cloning of the 960 bp PCR fragment, transformation into Aspergillus oryzae and production of culture fluid for purification of the GH24 lysozyme was performed according to example 1. A band at approximately 26 kDa was observed for each of the Aspergillus oryzae transformants. One A. oryzae transformant producing the polypeptide was chosen and cultivated in 1000 ml Erlenmeyer shake flasks containing 100 ml of DAP4C medium at 30° C. for 4 days with agitation at 150 rpm and purified as described in example 10.

### Example 10: Purification of the GH24 Lysozyme from Chaetomium thermophilum Var. thermophilum

The fermentation supernatant with the GH24 lysozyme from Chaetomium thermophilum var. thermophilum was filtered through a Fast PES Bottle top filter with a 0.22 μm cut-off. The resulting solution was diafiltrated with 50 mM Na-acetate, pH 4.5 and concentrated (volume reduced by a factor of 10) on an Ultra Filtration Unit (Sartorius) with a 10 kDa cut-off membrane.

After pretreatment about 300 mL of the lysozyme containing solution was purified by chromatography on SP Sepharose (approximately 60 mL) in a XK26 column eluting the bound lysozyme with 0 to 100% gradient of buffer A (50 mM Na-acetate pH 4.5) and buffer B (50 mM Na-acetate+1 M NaCl pH 4.5) over 10 column volumes. The fractions from the column were pooled based on the chromatogram (absorption at 280 and 254 nm) and SDS-PAGE analysis.

The molecular weight, as estimated from SDS-PAGE, was approximately 27 kDa and the purity was >90%.

### Example 11: Determination of Lysozyme Activity

Lysozyme activity was determined by measuring the decrease (drop) in absorbance/optical density of a solution of resuspended Micrococcus lysodeikticus ATTC No. 4698 (Sigma-Aldrich M3770) or Exiguobacterium undea (DSM14481) measured in a spectrophotometer at 540 nm.

Preparation of Micrococcus lysodeikticus Substrate

Before use, the cells were resuspended in citric acid-phosphate buffer pH 6.5 to a concentration of 0.5 mg cells/mL and the optical density (OD) at 540 nm was measured. The cell suspension was then adjusted so that the cell concentration equalled an OD540=1.0. The adjusted cell suspension was then stored cold before use. Resuspended cells were used within 4 hours.

Preparation of Dried Cells of Exiquobacterium undae Substrate

A culture of E. undae (DSM14481) was grown in 100 mL LB medium (Fluka 51208, 25 g/L) in a 500 mL shake-flask at 30° C., 250 rpm overnight. The overnight culture was then centrifuged at 20° C. and 5000 g for 10 minutes, and the pellet was then washed twice with sterile milliQ water, and resuspended in Milli-Q water. The washed cells were centrifuged for 1 minute at 13000 rpm and as much as possible of the supernatant was decanted. The washed cells were dried in a vacuum centrifuge for 1 hour. The cell pellet was resuspended in citric acid-phosphate buffer pH 4, 5 or 6 so that the optical density (OD) at 540 nm=1.

### Measurement of Lysozyme Antimicrobial Activity in the Turbidity Assay

The lysozyme sample to be measured was diluted to a concentration of 100-200 mg enzyme protein/L in citric acid-phosphate buffer pH 4, 5 or 6, and kept on ice until use. In a 96 well microtiterplate (Nunc) 200 μL of the substrate was added to each well, and the plate was incubated at 37° C. for 5 minutes in a VERSAmax microplate reader (Molecular Devices). Following incubation, the absorbance of each well was measured at 540 nm (start value). To start the activity measurement, 20 μL of the diluted lysozyme sample was added to each substrate (200 μL) and kinetic measurement of absorbance at 540 nm was initiated for minimum 30 minutes up to 24 hours at 37° C. The measured absorbance at 540 nm was monitored for each well and over time a drop in absorbance is seen if the lysozyme has lysozyme activity. The results are presented in table 2 below.

TABLE 2
Lysozyme Activity against Micrococcus lysodeikticus and
Exiguobacterium undea as measured by Optical Density Drop

| GH24 | Micrococcus | | Exiguobacterium | |
|---|---|---|---|---|
| Lysozyme | lysodeikticus¹ | | undae¹ | |
| GH24 Lysozyme from | ++ | (pH 6) | ++ | (pH 6) |
| Trichophaea saccata | | | | |
| (SEQ ID NO: 257) | | | | |
| GH24 lysozyme from | ++++ | (pH 5) | ++++ | (pH 6) |
| Chaetomium thermophilum | | | | |
| (SEQ ID NO: 264) | | | | |
| GH24 lysozyme from | − | (pH 6) | Not tested | |
| Trichophaea saccata | | | | |
| lacking LED | | | | |
| (SEQ ID NO: 270) | | | | |
| GH24 lysozyme from | + | (pH 4.5) | − | (pH 3-7) |
| Acremonium alcalophilum | | | | |
| (SEQ ID NO: 280) | | | | |

¹Means no significant effect; + means small effect; ++ means medium effect; +++ means large effect; ++++ means very large effect.
The pH value in the brackets lists the assay pH based on lysozyme-substrate combination.

The data shows that the two GH24 lysozymes that naturally comprise the lysozyme enhancing domain (LED) (SEQ ID NO: 257 and 264) showed good activity against both substrates. In comparison, SEQ ID NO: 270 (which is SEQ ID NO: 257 but lacking the LED) demonstrated no lysozyme activity against Micrococcus lysodeikticus and the GH24 lysozyme which does not natively comprise a LED (SEQ ID NO: 280) showed a small amount of activity.

### Example 12: Determination of Antimicrobial Activity

The antimicrobial activity of 3 GH24 lysozymes also comprising the lysozyme enhancing domain (LED) (SEQ ID NO: 257, 264 and 267), two GH24 lysozymes which natively do not comprise the LED (SEQ ID NO: 279 and 280) and the GH24 lysozyme of SEQ ID NO. 257 for which the LED was removed (resulting in SEQ ID NO: 269) against Clostridium perfringens DSM756 was tested using an RDA as described previously by Lehrer et al. (“Ultrasensitive assays for endogenous antimicrobial polypeptides”, J. Immunol. Methods 137:167-73 (1991)), but with several modifications.

Briefly, RDA bacteria were prepared by streaking C. perfringens DSM756 from freeze stocks on Luria-Bertani agar plates (Sigma L3027) and the plates were incubated overnight at 37° C. under anaerobic conditions (Anaerogen, Oxoid) in a jar. The following day colonies were suspended in 0.9% NaCl and the suspensions were adjusted to McFarland std. 1. 87% sterile glycerol was added to give a final glycerol concentration of 20% and the cells were frozen at −80° C. until use. For estimation of colony forming units (CFU) per milliliter of the RDA bacteria 10-fold dilution series were prepared of the freeze stock in 0.9% NaCl and 100 μl of the dilutions were plated on Luria-Bertani agar plates (Sigma L3027) and incubated overnight at 37° C. under anaerobic conditions (Anaerogen, Oxoid) in a jar.

When preparing the RDA plates broth media with agar was melted and cooled to 42° C. Two media were tested in the experiment:
a) ½ Mueller-Hinton broth (MHB) (Sigma/Fluka, 90922) (i.e., adjusted to pH 6 with 4 M HCl and diluted 1:1 with water) with 1.5% agarose, and
b) 1/10 Mueller-Hinton broth (MHB) (Sigma/Fluka, 90922) (i.e., diluted 1:9 with water) with 1% agarose.

For each assay plate 30 ml of melted media was added to achieve around 5.0×10⁵ cfu/mL C. perfringens DSM756 and this was poured into a single-well omnitray (Nunc) plate. The omnitray plate was overlaid with a TSP plate (Nunc) and left to solidify (at room temperature or below). Afterwards, the TSP plate was removed; leaving 96 wells, in which 10 μL of the compound of interest could be tested.

10 μl of the test solutions were spotted pr. well and the plates were incubated over night at 37° C. in a jar under anaerobic condition (Anaerogen, Oxoid). The following day a clearing zone indicated inhibition of growth of test bacteria and thereby antimicrobial activity. For the RDA plates with ½ MHB, the clearing zones were visualized by coloring with MTT (3-(4,5-dimethylthiazol-2-yl)-2,5-diphenyltetrazolium bromide, a yellow tertrazole), that is reduced to purple formazan in living cells (Mosmann, 1983, “Rapid colorimetric assay for cellular growth and survival: application to proliferation and cytotoxicity assays”, Journal of Immunological Methods 65 (1-2): 55-63). This coloring provides for a dark coloring of living cells and no coloring of the clearing zones without living cells.

Bacitracin zinc salt (Sigma B-8800) (50 μg/ml) was included as a positive control and lysozymes were tested using a solution of 100 μg/ml. The results are presented in table 3 below.

TABLE 3
Antimicrobial Activity against Clostridium perfringens
as measured by RDA

| | Diameter of clearing zone (mm) | | |
|---|---|---|---|
| Experiment | 1 | 2 | 1 |
| Lysozyme | 1/10 MHB | 1/10 MHB | 1/2 MHB |
| | pH 7 | pH 7 | pH 6 |
| GH24 Lysozyme from | 9 | 8 | 11 |
| Trichophaea saccata | | | |
| (SEQ ID NO: 257) | | | |
| GH24 lysozyme from | 9 | 7 | 10 |
| Chaetomium thermophilum | | | |
| (SEQ ID NO: 264) | | | |
| GH24 lysozyme from | 10 | 8 | 12 |
| Trichoderma harzianum | | | |
| (SEQ ID NO: 267) | | | |
| GH24 lysozyme from | (8)* | (5)* | 0 |
| Trichophaea saccata | | | |
| lacking LED | | | |
| (SEQ ID NO: 270) | | | |
| GH24 lysozyme from | 0 | 0 | 0 |
| Acremonium alcalophilum | | | |
| (SEQ ID NO: 279) | | | |
| GH24 lysozyme from | 0 | 0 | 0 |
| Acremonium alcalophilum | | | |
| (SEQ ID NO: 280) | | | |
| Bacitracin zinc salt | 22 | 18 | 11 |

*Incomplete inhibition of growth visible after MTT colouring

The 3 GH24 lysozymes also comprising the lysozyme enhancing domain (LED) (SEQ ID NO: 257, 264 and 267) all showed antimicrobial activity against viable cells of C. perfringens DSM756 under both tested conditions. In comparison, the two GH24 lysozymes which natively do not comprise the LED (SEQ ID NO: 279 and 280) did not inhibit growth under the test conditions. Furthermore, the GH24 lysozyme for which the LED was removed (SEQ ID NO: 269) also showed no inhibition of growth in ½ MHB, pH 6, although incomplete inhibition of growth was observed when using 1/10 MBH, PH 7.

Thus, the results show that GH24 lysozymes tested which comprise a LED show significant activity Clostridium perfringens using the conditions 50% MHB, pH 6 whereas the GH24 lysozymes without the LED (either naturally or when the LED has been removed) do not show activity Clostridium perfringens using the conditions 50% MHB, PH 6.

### Example 13: Temperature Stability

Temperature stability was determined for the GH24 lysozyme from Trichophaea saccata (SEQ ID NO: 257) by incubating the lysozyme at different temperatures (60-99° C.) for a short period of time (30, 60 and 90 seconds) after which the residual activity was measured for 1 h at 37° C. using M. lysodeikticus substrate prepared as described in Example 11.

### Heat Treatment

The samples to be evaluated were diluted to a lysozyme concentration of 0.05 mg enzyme protein/mL in milliQ water. Initially 40 μL of each lysozyme sample was pipetted into PCR tubes (3 replicates) and the closed tubes were then kept on ice. The PCR tubes were placed in a preheated PCR machine for 30, 60 or 90 seconds at 60, 65, 70, 75, 80, 85, 90, 95 or 99° C. and then put back on ice for instant cooling. Non heated control samples corresponding to the heated samples were prepared and kept on ice.

### Activity Measurement

The OD drop assay using M. lysodeikticus as substrate was used to determine lysozyme activity in heated and non-heated samples. Initially 20 μL of each sample (control and heat treated) was transferred to a 96-well microtiter plate, then 180 μL assay buffer (pH 6) and 20 μL M. lysodeikticus substrate was added to each well. The plate was immediately placed in an absorbance reader preheated to 37° C. and OD540 recorded for 1 hour. The difference between the absorbance at T=0 and the time points was calculated as the AOD. The percentage residual activity was calculated by comparing the AOD of the heat treated sample with the corresponding AOD of the sample which was not heat treated (Table 5).

TABLE 5
Residual activity (%) of the GH24 lysozyme from
Trichophaea saccata (SEQ ID NO: 257) measured by
OD drop after heat treatment at different temperatures

| | Temperature (° C.) | | | | | | | | |
|---|---|---|---|---|---|---|---|---|---|
| Time (Sec) | 60 | 65 | 70 | 75 | 80 | 85 | 90 | 95 | 99 |
| 30 | 93 | 99 | 94 | 101 | 94 | 98 | 94 | 98 | 93 |
| 60 | 99 | 97 | 93 | 96 | 98 | 94 | 99 | 94 | 97 |
| 90 | 96 | 99 | 98 | 98 | 97 | 100 | 97 | 102 | 89 |

The results show that the GH24 lysozyme from Trichophaea saccata (SEQ ID NO: 257) is stable for at least 90 seconds at temperatures as high as 99° C.

### Example 13: Thermostability Determined Using DSC

The thermostability of GH24 lysozyme from Trichophaea saccata (SEQ ID NO: 257) at different pH values was determined by Differential Scanning calorimetry (DSC) using a VP-capillary DSC instrument (MicroCal Inc., Piscataway, NJ, USA) equipped with an auto sampler. Aliquots of the GH24 lysozyme, purified as described in Example 2, were buffer-changed (see buffer in table 6 below) using prepacked NAP-5 columns. The samples were diluted with the corresponding buffer to approximately 0.5 mg/ml and the buffer was used as reference solution. Sample and reference solutions (approx. 0.5 ml) were thermally pre-equilibrated for 10 minutes at 20° C. and the DSC scan was performed from 20 to 100° C. at a scan rate of 200 K/hour. Data-handling was performed using the MicroCal Origin software (version 7.0383). The thermal denaturation temperature, Td (° C.), was taken as the top of denaturation peak (major endothermic peak) in thermograms (Cp vs. T) obtained after heating the lysozyme solution in the buffer at a constant programmed heating rate. Denaturation temperatures were determined at an accuracy of approximately +/−0.5° C. and are given in table 6 below.

TABLE 6
Denaturation Temperature of the GH24 lysozyme from
Trichophaea saccata (SEQ ID NO: 257) at various pH's

| Buffer | Td (° C.) |
|---|---|
| 50 mM Na-acetate, pH 4.5 | 70.0 |
| 50 mM Na-acetate, pH 5.5 | 70.8 |
| 50 mM Na-acetate, pH 6.5 | 69.7 |

### Example 14: Lysozyme Enhancing Domain Binds to Bacterial Cells

250 mg Micrococcus lysodeiktikus ATCC No. 4698 (Sigma M3770) cells were resuspended in 2.5 ml distilled H₂O with 0.1% Tween 80. Cells were soaked at 4° C. overnight.

Avicel® PH-101 is a microcrystalline cellulose powder trademarked by FMC Corporation (Philadelphia, Pennsylvania) and sold by Sigma Aldrich (cat no. 11365). 250 mg Avicel was suspended in H₂O with 0.1% Tween 80. This was also left hydrating overnight.

After overnight hydrations, 50 μl of each suspension was taken out and this was washed once in 50 μl H₂O with 0.1% Tween 80. The purified LED (SEQ ID NO: 273) had a concentration of 0.23 mg/ml in a buffer consisting of 50 mM Na-Acetate, pH 4.5+50 mM NaCl. For the experiment, 50 μl of Avicel suspension or 50 μl of M. lysodeiktikus suspension were aliqouted into 1.5 ml Eppendorf tubes. 50 μl (11.5 mgs) of purified LED protein were then added to each tube, mixed by vortexing and then incubated at room temperature for 30 minutes. The samples were then centrifuged, and the liquid decanted to a 1.5 ml Eppendorf tube.

For each sample, 8 μl 4×E-PAGE Loading Buffer (EPBUF-01, Life Technologies), 1 μl (10×) NuPAGE® Sample Reducing Agent (Life Technologies), were added to 24 μl supernatant. The two samples were then vortex mixed and heated in a heating block at 70° C. for 10 minutes. 20 μl of each prepared sample were then loaded on to a Criterion XT 8-16% gradient BIS-Tris SDS gel and run in Criterion XT MOPS buffer according to the manufacturer's instructions (BioRad Laboratories). A Rainbow recombinant molecular weight marker was also run in the gel (RPN800, GE Healthcare). The SDS gel was stained with Simply Blue Coomassie stain (Life Technologies) and the results visualized as shown in table 7.

TABLE 7
Binding of LED to Avicel or M. lysodeiktikus suspension

| | Lysozyme | | |
|---|---|---|---|
| Sample | enhancing domain | Suspension | Result |
| 1 | SEQ ID NO: 273 | Avicel | +++ |
| 2 | SEQ ID NO: 273 | M. lysodeiktikus | + |
| 3 | SEQ ID NO: 273 | None | +++ |

+ represents band intensity on an SDS gel.

The results show that the LED protein migrates on the SDS gel at about 8 kDa as expected. The LED protein SDS band intensity is approximately equal in the Avicel and the untreated samples (samples 1 and 3) while the SDS band intensity was substantially reduced in the M. lysodiektikus cells treated sample (sample 2). This means that the M. lysodiektikus cells were able to bind the LED protein under the buffer conditions tested (25 mM Na-Acetate, pH 4.5, 25 mM NaCl) while crystalline cellulose could not bind LED protein. It should be noted that it is unknown which component of the Micrococcus cells that LED binds to.

### Example 15: The Lysozyme Enhancing Domain HMM

SEQ ID NOs: 123 to 251 were aligned using the software program MUSCLE v3.8.31 with the default settings. Using this alignment, the HMM was constructed using the software program ‘hmmbuild’ from the package HMMER 3.0 (March 2010) (hmmer.org/) and the software was invoked using the default settings. The lysozyme enhancing domain HMM profile thereby generated for subsequent loading into the software program ‘hmmscan’ is given below.

HMMER3/b [3.0 | March 2010]
NAME lysozyme_enhancing_domain
LENG 73
ALPH amino
RF no
CS no
MAP yes
DATE Tue Feb 3 15:29:15 2015
NSEQ 129
EFFN 1.263702
CKSUM 3302514446
STATS LOCAL MSV −9.1036 0.71868
STATS LOCAL VITERBI −9.7357 0.71868
STATS LOCAL FORWARD −3.7686 0.71868
HMM A C D E F G H I K L M N

P Q R S T V W Y

m−>m m−>i m−>d i−>m i−>i d−>m d−>d

COMPO 2.64236 3.16005 2.87141 2.79417 3.60706 2.63596 3.86157 2.94229

2.65279 2.95816 3.97690 3.11757 3.46392 3.12498 3.11011 2.56828 2.58627 2.58086
4.17029 3.04296

2.68618 4.42225 2.77519 2.73123 3.46354 2.40513 3.72494 3.29354 2.67741

2.69355 4.24690 2.90347 2.73739 3.18146 2.89801 2.37887 2.77519 2.98518 4.58477
3.61503

| 0.17958 4.32413 1.88959 0.61958 0.77255 0.00000 * | |
|---|---|
| 1 | 3.80107 5.04040 4.67499 4.39045 1.81828 4.48873 3.56285 3.50991 |

4.23379 2.82560 4.11380 4.16131 4.81340 4.22617 4.28030 3.87997 4.03091
3.43577 3.70270 0.73371 1 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02327 4.16783 4.89017 0.61958 0.77255 0.67437 0.71228 |
|---|---|
| 2 | 2.33952 4.44593 3.23890 3.02979 4.35083 3.16321 4.20776 3.67603 |

3.02584 3.40031 4.31198 3.32021 1.10553 3.46227 3.39562 2.64660 2.86963
3.24503 5.70138 4.44714 2 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02327 4.16783 4.89017 0.61958 0.77255 0.58149 0.81886 |
|---|---|
| 3 | 2.99224 4.47028 5.00531 4.50059 3.72470 4.59877 5.18674 1.10701 |

4.40105 2.19903 3.51509 4.69439 4.89181 4.65354 4.58483 3.98375 3.44715
1.21838 5.67709 4.47722 3 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02218 4.21548 4.93783 0.61958 0.77255 0.62351 0.76800 |
|---|---|
| 4 | 2.67119 4.76820 3.04982 2.54195 4.12069 3.43519 3.75916 3.51149 |

2.19874 3.13703 3.97428 2.96845 3.89558 2.93360 2.89915 2.57877 1.61288
3.10281 5.38435 4.08520 4 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02491 4.21548 4.62159 0.61958 0.77255 0.52151 0.90048 |
|---|---|
| 5 | 2.37234 5.08464 2.49824 2.21575 4.41120 1.99198 3.58111 3.86677 |

2.52882 3.41126 4.20120 2.81325 3.86140 2.84200 3.02691 2.42476 2.83819
3.48001 5.60144 4.21122 5 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02114 4.26301 4.98536 0.61958 0.77255 0.56183 0.84436 |
|---|---|
| 6 | 2.63301 5.17310 2.09015 2.17272 4.49463 3.27178 3.65545 3.97097 |

2.38584 3.47241 4.23433 2.52660 3.34330 2.76874 2.94084 2.41690 2.44683
3.55393 5.62559 4.21427 6 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.07099 4.26555 2.90975 0.61958 0.77255 0.56422 0.84119 |
|---|---|
| 7 | 2.61111 4.85146 2.70126 2.41033 4.02944 2.66646 3.65077 3.52514 |

2.43178 3.11934 3.92649 2.77281 3.83428 2.77300 2.91547 2.42718 2.41739 2.79685
5.35415 3.75427 7 - -

2.68619 4.42226 2.77521 2.73124 3.46355 2.40511 3.72496 3.29355

2.67742 2.69356 4.24691 2.90348 2.73741 3.18147 2.89802 2.37888 2.77504 2.98519
4.58478 3.61504

| | 0.09494 2.48394 4.93906 0.38374 1.14353 0.52245 0.89910 |
|---|---|
| 8 | 3.16563 4.52406 5.00410 4.47860 3.59477 4.61318 5.11093 1.75130 |

4.36399 1.66390 3.39589 4.68330 4.88103 4.58179 4.52924 3.98371 3.48258 0.98654
5.56065 4.39116 9 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02108 4.26555 4.98790 0.61958 0.77255 0.56422 0.84119 |
|---|---|
| 9 | 2.74568 5.18103 2.62071 2.37499 4.50785 3.44148 3.43386 3.97009 |

2.14487 3.46738 4.24084 1.77122 3.86903 2.77625 2.44752 2.65464 2.97757 3.56615
5.60976 4.22640 10 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02108 4.26555 4.98790 0.61958 0.77255 0.43965 1.03356 |
|---|---|
| 10 | 3.21633 0.35479 4.79708 4.65548 4.57914 3.72194 5.20787 3.82938 |

4.49870 3.70850 4.85167 4.53077 4.45916 4.82584 4.53365 3.48556 3.72159 3.53505
5.86530 4.85631 11 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 11 | 3.30119 5.31389 3.73668 3.09114 4.51121 3.86969 3.13217 4.16018 |

2.10415 3.58751 4.49408 3.45992 4.26052 2.99924 0.83233 3.31264 3.47550 3.85705
5.50391 4.26079 12 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 12 | 2.24421 4.51545 3.30473 2.81428 4.39051 3.23670 4.14928 3.77491 |

3.04692 3.44962 4.27960 3.30747 3.89875 3.36729 3.42822 1.05895 2.51437 3.31859
5.70279 4.43964 13 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 13 | 2.69468 4.75034 2.89429 2.56341 4.66767 0.94912 4.20148 4.12822 |

3.15097 3.75743 4.60332 3.20872 3.96757 3.42394 3.55601 2.47312 3.12631 3.62410
5.94365 4.63448 14 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 14 | 2.67424 4.69741 3.65691 3.53445 4.63811 3.40395 4.65858 4.09486 |

3.63150 3.77181 4.76683 3.75211 0.59051 3.99224 3.88152 2.99980 3.31792 3.64480
5.92609 4.77080 15 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 15 | 2.26323 4.51933 3.21961 2.94036 4.47411 1.38422 4.14822 3.91245 |

3.06085 3.54037 4.34588 2.97288 3.87499 3.35219 3.46854 1.96711 2.52323 3.40350
5.76384 4.49002 16 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 16 | 2.20668 4.36145 3.82003 3.56662 4.41076 3.20360 4.55272 3.51597 |

3.53768 3.41304 4.35967 3.64187 3.96341 3.86648 3.78744 2.68079 0.81042 3.10831
5.83931 4.64399 17 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 17 | 2.61570 5.10849 2.62653 2.38205 4.43252 2.78078 3.60811 3.89018 |

2.47426 3.42628 4.20945 2.71231 3.87390 2.61734 2.99949 1.64993 2.94705 3.49936
5.60799 4.21799 18 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 18 | 2.58292 4.58689 3.02231 2.68583 3.00245 3.32285 2.97170 3.14564 |

2.67302 2.81683 3.67939 2.99852 3.79428 3.00246 3.09283 2.65865 2.82740 2.88473
5.11794 2.25478 19 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 19 | 2.02971 5.05961 2.68453 2.36019 4.37471 3.26819 3.65420 3.83090 |

2.10672 3.36092 4.13132 2.92980 3.45060 2.77060 2.82369 2.16905 2.88648 3.43886
5.53518 4.15201 20 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 20 | 3.16684 4.47509 4.98151 4.48359 3.78153 4.52597 5.13073 1.23357 |

4.37208 2.38350 3.60515 4.64947 4.86068 4.63385 4.54770 3.62858 3.43521 1.03896
5.65865 4.43929 21 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 21 | 2.67985 4.50015 3.40866 2.83947 3.66095 3.60615 3.61051 2.50038 |

2.12953 2.71143 3.59755 3.27678 3.98763 2.81347 3.06211 2.85030 2.83675 1.92294
5.06195 3.81377 22 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 22 | 2.57540 5.16214 3.25351 2.63376 4.54584 3.62294 3.69980 3.93939 |

1.30635 3.43432 4.24813 3.13299 4.00544 2.78831 2.11440 2.89381 2.74985 3.58326
5.55169 4.27942 23 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 23 | 2.54455 4.73655 3.02733 2.59505 4.05985 3.44683 3.70095 3.46466 |

2.60056 3.09418 3.92399 3.08417 3.89235 2.67009 3.04116 2.11836 1.78482 2.95974
5.35882 4.04770 24 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 24 | 3.40982 4.75103 4.67192 4.22624 1.97308 4.35966 3.81417 3.04888 |

4.07862 2.38151 3.72579 4.16721 4.67838 4.14717 4.17038 3.68839 3.63576 2.47365
4.02768 0.99393 25 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 25 | 2.33607 5.05721 2.94517 2.24042 4.36369 3.44673 3.55543 3.81754 |

1.82729 3.31948 4.11325 2.80831 3.17764 2.75227 2.78214 2.58400 2.65726 3.32087
5.51479 4.13659 26 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 26 | 2.36555 5.04925 3.04993 2.35049 4.35720 3.51989 3.68369 3.71551 |

1.45376 3.06828 4.12214 3.00561 3.91721 2.74243 2.72409 2.73518 2.95391 3.42066
5.49677 4.16815 27 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 27 | 2.61202 4.80098 2.75023 2.62858 4.42906 1.32629 3.93435 3.86483 |

2.78346 3.46292 4.27988 3.09911 3.91159 3.10786 2.85440 2.43819 2.91018 3.44468
5.68001 4.35178 28 - -

2.68618 4.42225 2.77519 2.73123 3.46354 2.40513 3.72494 3.29354

2.67741 2.69355 4.24690 2.90347 2.73739 3.18146 2.89801 2.37887 2.77519
2.98518 4.58477 3.61503

| | 0.01987 4.32413 5.04648 0.61958 0.77255 0.48576 0.95510 |
|---|---|
| 28 | 2.39518 5.06692 2.42469 2.35324 4.15968 3.22653 3.04826 3.83188 |

2.39351 3.35617 4.12096 2.82671 3.83411 2.52507 2.87979 2.43622 2.35726 3.41027
5.52582 4.13893 29 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 29 | 2.56098 5.10975 1.84700 2.22610 4.41970 3.43432 3.64564 3.82090 |

2.31577 3.39892 4.16246 2.88269 3.84241 2.68525 2.69153 2.62766 2.81515 3.48193
5.56120 3.47482 30 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 30 | 2.71289 4.20867 4.05384 3.08944 3.32534 3.70711 4.11635 2.10460 |

3.37170 2.31376 3.31881 3.74286 4.18005 3.62760 3.59433 3.09939 2.94743 1.40821
4.85949 3.10435 31 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 31 | 2.56066 3.98356 2.83917 2.26935 4.26245 3.45019 3.65582 3.70207 |

1.96206 3.26063 4.04582 2.95679 3.84786 2.75603 2.89654 2.30158 2.29348 3.33728
5.46026 4.09624 32 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 32 | 2.98261 4.34033 4.74407 4.16222 2.74015 4.19374 4.48870 1.33722 |

4.00888 1.70186 3.16247 4.27271 4.50036 4.14592 4.09048 3.51345 3.21313 1.97755
4.93581 3.15765 33 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 33 | 2.50714 4.52249 3.32761 2.73441 3.99413 2.87824 3.93263 3.37611 |

2.83014 3.05077 3.89907 3.20556 3.90546 3.15647 3.23473 2.09891 1.66153 2.58887
5.34950 4.08041 34 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 34 | 2.73372 0.86170 4.21080 3.82223 3.72250 3.51687 4.43487 3.14732 |

3.50989 2.93675 3.97687 3.88251 4.17078 3.93344 3.44432 2.99564 3.14856 2.89652
5.25169 3.70577 35 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 35 | 2.78221 4.93521 3.15849 2.72873 3.66991 3.61431 3.78500 3.57206 |

2.57323 3.13265 4.05241 3.20038 4.05162 1.36867 2.92156 2.93601 3.12692 3.29834
5.09768 2.57435 36 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 36 | 2.39232 4.68655 3.16574 2.46000 3.89951 3.51768 3.74742 2.86223 |

2.34831 2.93724 3.78511 3.11031 3.91433 2.90991 3.00803 2.69966 1.85059 2.95170
5.23226 3.93826 37 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 37 | 2.38712 4.95526 2.95139 2.14292 4.22581 3.30071 3.48757 3.66208 |

2.41922 3.22834 4.01725 2.96015 3.42523 2.64575 2.85382 2.40915 2.33462 3.08668
5.43599 3.59495 38 - -

2.68618 4.42225 2.77519 2.73123 3.46354 2.40513 3.72494 3.29354

2.67741 2.69355 4.24690 2.90347 2.73739 3.18146 2.89801 2.37887 2.77519
2.98518 4.58477 3.61503

| | 0.01987 4.32413 5.04648 0.61958 0.77255 0.48576 0.95510 |
|---|---|
| 38 | 3.32493 5.03100 4.08842 4.05521 5.14287 0.28143 5.12256 4.87721 |

4.29611 4.46333 5.45951 4.25516 4.43330 4.58529 4.45932 3.51325 3.84001 4.31766
6.09414 5.26815 39 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 39 | 2.77845 5.16916 2.50798 1.90461 4.47239 3.42576 3.73520 3.92893 |

2.55535 3.46812 4.26082 2.91770 3.47218 2.74953 3.05304 2.74879 1.68968 3.54385
5.65411 4.25701 40 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 40 | 2.55637 3.36322 2.86419 2.49420 4.07854 3.43147 3.53651 3.49427 |

2.36324 3.10007 3.91410 2.51497 3.86778 2.83786 2.95074 2.24490 2.43049 3.00556
5.34651 3.93774 41 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 41 | 3.32262 4.56891 5.24679 4.77118 3.84175 4.81012 5.48421 1.14831 |

4.68143 2.30177 3.60891 4.94823 5.08094 4.93596 4.85577 4.22682 3.58792 0.99460
5.88795 4.67682 42 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 42 | 2.65825 5.10541 2.72992 2.15627 3.98363 3.31023 3.53625 3.89170 |

2.18852 3.23731 4.15424 2.23857 3.82936 2.65748 2.86654 2.25743 2.79796 3.48165
5.55380 4.15800 43 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 43 | 2.61182 4.52680 3.60621 3.51889 4.78008 0.61583 4.68006 4.23876 |

3.72062 3.95294 4.83622 3.66170 4.02536 4.00392 3.97409 2.79132 2.85714 3.64017
6.05621 4.90618 44 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 44 | 2.79049 5.22667 2.25221 2.33027 4.53369 3.27947 3.53614 4.00930 |

2.56097 3.52971 4.31450 1.59788 3.89352 2.86397 3.06987 2.39242 3.03945 3.49407
5.69864 4.13140 45 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 45 | 2.49378 3.36932 2.74714 2.42229 4.10830 3.46961 3.68039 3.52770 |

2.44255 3.12654 3.93602 2.51463 3.86384 2.82770 2.94369 2.19290 2.51568 3.13211
5.36588 3.76024 46 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 46 | 2.69732 4.16858 4.15640 3.57668 3.28068 3.80998 4.13366 1.64485 |

3.45403 1.84904 3.26899 3.79253 3.96991 3.68559 3.64246 2.81777 2.62992 2.15853
4.82146 3.52063 47 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 47 | 4.19233 5.38393 4.84605 4.67977 3.27817 4.29458 4.57157 4.26819 |

4.41284 3.57367 4.87507 4.74845 4.85287 4.77414 4.45256 4.39169 4.52083 4.16862
0.32020 3.26075 48 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 48 | 3.20149 5.67973 0.78816 2.34486 4.76934 3.43076 3.17601 4.55895 |

3.07436 4.04716 4.94146 2.92732 4.07402 3.21129 3.65781 3.08955 3.50632 4.14006
6.03658 4.51417 49 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 49 | 2.76346 4.98544 3.15831 2.56957 4.01057 3.55403 3.68400 3.67589 |

1.47331 2.89158 4.05158 3.07047 3.93382 2.56322 2.45400 2.78078 2.98070 3.34485
5.41794 3.49426 50 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 50 | 2.64257 4.43619 3.56736 3.08202 3.77537 3.52079 4.05811 2.42620 |

3.00927 2.79176 3.72810 2.99222 4.03030 3.35753 3.34645 2.85418 1.35329 2.62966
5.23131 3.98292 51 - -

2.68619 4.42226 2.77521 2.73124 3.46340 2.40514 3.72496 3.29355

2.67742 2.69356 4.24691 2.90348 2.73741 3.18148 2.89802 2.37888 2.77521 2.98520
4.58478 3.61472

| | 0.08832 2.54971 5.04648 0.37639 1.15942 0.48576 0.95510 |
|---|---|
| 51 | 2.52068 4.47414 3.35565 2.74391 3.33968 3.48613 3.81115 3.00228 |

2.76056 2.69538 3.20591 3.24034 3.68619 2.82417 3.12828 2.08694 2.32290 2.59916
4.31681 3.78415 53 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 52 | 2.77860 4.87789 1.42239 2.56155 3.13731 3.54366 3.74371 3.49507 |

2.67769 3.09585 3.97167 3.06454 3.97169 2.99708 3.12924 2.82286 3.01884 3.20614
4.04928 3.59798 54 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 53 | 2.94931 5.28328 2.08563 2.39481 4.84632 1.16425 4.00151 4.36230 |

3.01231 3.90219 4.74326 2.45548 3.99074 3.18038 3.56729 2.91382 3.29175 3.89959
6.06470 4.62009 55 - -

2.68621 4.42228 2.77522 2.73126 3.46357 2.40509 3.72497 3.29357

2.67727 2.69358 4.24693 2.90346 2.73742 3.18149 2.89804 2.37878 2.77522 2.98521
4.58480 3.61506

| | 0.08509 2.58845 5.04648 0.73284 0.65497 0.48576 0.95510 |
|---|---|
| 54 | 3.21633 0.35479 4.79708 4.65548 4.57914 3.72194 5.20787 3.82938 |

4.49870 3.70850 4.85167 4.53077 4.45916 4.82584 4.53365 3.48556 3.72159 3.53505
5.86530 4.85631 59 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 55 | 4.07603 5.22780 4.94333 4.71701 1.81696 4.70604 3.64405 3.73694 |

4.54415 3.00155 4.31962 4.34626 5.01267 4.44587 4.52415 4.10863 4.29875 3.67538
3.75731 0.60600 60 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 56 | 3.29932 4.57114 5.13914 4.65306 3.79025 4.73170 5.36042 1.62171 |

4.54191 2.21256 3.57888 4.84627 5.01963 4.80796 4.72647 4.13701 3.56571 0.76183
5.80720 4.58938 61 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 57 | 1.40302 4.31490 3.77731 3.44350 4.52474 3.10962 4.46137 3.90675 |

3.45708 3.61908 4.43729 3.53676 3.86941 3.73557 3.76041 1.18441 2.55005 3.34107
5.86901 4.67381 62 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 58 | 3.16638 5.52729 0.80512 2.42994 5.07620 2.30005 4.12607 4.65042 |

3.22647 4.16769 5.05164 2.99972 4.08759 3.32219 3.82773 3.09290 3.51563 4.17606
6.23444 4.80691 63 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 59 | 3.11990 4.36544 4.32127 3.81191 2.17745 4.09367 3.78203 3.04592 |

3.23806 2.64903 3.67717 3.91110 4.44159 3.85035 3.84946 3.39383 3.34718 2.77912
3.74216 1.06936 64 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01987 4.32413 5.04648 0.61958 0.77255 0.48576 0.95510 |
|---|---|
| 60 | 3.51146 4.91649 4.45325 4.08004 2.02567 4.31163 3.69403 3.46524 |

3.96389 2.92976 4.07889 4.05659 4.68281 4.07537 4.10672 2.99801 3.75887 3.31559
3.91189 0.78971 65 - -

2.68618 4.42225 2.77519 2.73123 3.46354 2.40513 3.72494 3.29354 2.67741

2.69355 4.24690 2.90347 2.73739 3.18146 2.89801 2.37887 2.77519 2.98518 4.58477
3.61503

| | 0.01987 4.32413 5.04648 0.61958 0.77255 0.48576 0.95510 |
|---|---|
| 61 | 2.72553 4.31253 4.16671 3.66674 3.67680 3.72642 4.41126 2.29394 |

3.56150 2.58708 3.60951 3.86666 4.24179 3.84789 3.80334 2.76599 2.38224 1.10833
5.25833 4.04936 66 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02985 4.32413 4.12501 0.61958 0.77255 0.48576 0.95510 |
|---|---|
| 62 | 2.51216 5.16569 2.33791 2.35923 4.49437 3.19374 3.66109 3.96480 |

1.64210 3.46558 4.22781 2.83561 3.85394 2.71011 2.84192 2.54559 2.88860 3.55105
5.61534 4.21583 67 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02007 4.31435 5.03670 0.61958 0.77255 0.49963 0.93333 |
|---|---|
| 63 | 2.49188 4.38955 3.59551 3.16295 4.09719 3.28177 4.17679 3.38911 |

3.04040 3.15453 4.02841 3.43067 3.92288 3.44894 3.44644 2.11805 1.20176 2.68117
5.48692 4.25411 68 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02007 4.31435 5.03670 0.61958 0.77255 0.49963 0.93333 |
|---|---|
| 64 | 1.89508 4.31720 3.70048 3.48388 4.63634 0.95179 4.55159 4.00807 |

3.58485 3.73760 4.55934 3.55250 3.87506 3.84242 3.86415 2.56592 2.50453 3.40204
5.97883 4.79808 69 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.03953 4.31435 3.67355 0.61958 0.77255 0.49963 0.93333 |
|---|---|
| 65 | 2.63597 3.39258 3.14566 2.45530 3.91792 3.49097 3.54341 3.31288 |

2.40023 2.95698 3.79792 3.08785 3.89316 2.92081 3.00148 1.97899 2.29007 2.96915
5.24220 3.94276 70 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02046 4.29528 5.01763 0.61958 0.77255 0.52575 0.89432 |
|---|---|
| 66 | 2.59137 4.97540 2.68695 2.29850 4.05453 3.34590 3.64946 3.59380 |

2.42308 3.26160 4.04572 2.57932 3.83844 2.77492 2.90216 1.98570 2.44689 3.22530
5.46068 4.09334 71 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02046 4.29528 5.01763 0.61958 0.77255 0.52575 0.89432 |
|---|---|
| 67 | 2.36239 3.82492 3.07537 2.52645 4.05085 1.94430 3.77137 3.45853 |

2.53920 3.08721 3.91418 2.86394 3.87403 2.94691 3.05153 2.46988 2.65768 3.12923
5.35184 4.04208 72 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02046 4.29528 5.01763 0.61958 0.77255 0.52575 0.89432 |
|---|---|
| 68 | 2.51473 4.23017 3.73065 3.15738 2.96324 3.66009 3.93184 2.62617 |

3.08387 2.43082 2.74387 3.49391 3.33993 3.35931 3.14679 2.64784 2.86016 2.49302
4.81896 2.22311 73 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02046 4.29528 5.01763 0.61958 0.77255 0.52575 0.89432 |
|---|---|
| 69 | 2.33893 4.26290 4.21737 3.66773 3.27773 3.87203 4.32579 2.12233 |

3.56960 2.41061 3.43058 3.89614 4.28394 3.82133 3.79298 2.87144 3.03565 1.21453
5.06701 3.86005 74 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02293 4.29528 4.70670 0.61958 0.77255 0.52575 0.89432 |
|---|---|
| 70 | 2.49593 4.60476 3.23381 2.52485 3.79749 3.53767 3.76569 3.14297 |

2.26081 2.40764 3.70182 3.15412 3.92810 2.98301 3.03430 2.76728 2.09050 2.55846
5.16115 3.88294 75 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02051 4.29287 5.01521 0.61958 0.77255 0.52898 0.88967 |
|---|---|
| 71 | 2.60687 5.12624 2.52016 2.23382 4.44833 2.55208 3.64005 3.91884 |

2.12896 3.37075 4.18386 2.86694 3.03933 2.74997 2.89501 2.33562 2.83629 3.50672
5.58015 4.17955 76 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02051 4.29287 5.01521 0.61958 0.77255 0.52898 0.88967 |
|---|---|
| 72 | 2.57816 5.12677 2.87095 2.24649 4.45454 3.41210 3.40077 3.91086 |

1.59193 3.41242 4.17909 2.90547 3.85681 2.60853 2.68264 2.65653 2.67555 3.50960
5.55782 4.18309 77 - -

2.68618 4.42225 2.77520 2.73117 3.46354 2.40513 3.72495 3.29354

2.67741 2.69355 4.24690 2.90347 2.73740 3.18147 2.89801 2.37887 2.77520 2.98519
4.58477 3.61503

| | 0.09497 3.41028 2.85473 0.52137 0.90068 0.52898 0.88967 |
|---|---|
| 73 | 3.08258 0.42515 4.66004 4.49479 4.42412 3.61311 5.06351 3.64868 |

4.33208 3.53771 4.67608 4.38809 4.34603 4.66309 4.38494 3.35266 3.58099 3.36315
5.74041 4.70439 79 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

0.01461 4.23357 * 0.61958 0.77255 0.00000 *

//

### Example 16: The GH24 Catalytic Domain HMM

SEQ ID NOS: 1 to 122 were aligned using the software program MUSCLE v3.8.31 with the default settings. Using this alignment, the HMM was constructed using the software program ‘hmmbuild’ from the package HMMER 3.0 (March 2010) (hmmer.org/) and the software was invoked using the default settings. The GH24 catalytic domain HMM profile thereby generated for subsequent loading into the software program ‘hmmscan’ is given below.

HMMER3/b [3.0 | March 2010]
NAME GH24_catalytic_domain
LENG 161
ALPH amino
RF no
CS no
MAP yes
DATE Tue Apr 14 10:23:27 2015
NSEQ 122
EFFN 1.567444
CKSUM 3253961472
STATS LOCAL MSV −10.1203 0.70833
STATS LOCAL VITERBI −10.9320 0.70833
STATS LOCAL FORWARD −4.4787 0.70833
HMM A C D E F G H I K L M N

P Q R S T V W Y

m->m m->i m->d i->m i->i d->m d->d

COMPO 2.44048 3.93467 3.01702 2.62301 3.43295 2.77445 3.69252 3.04117

2.65511 2.46463 3.77377 2.92042 3.46380 3.02561 2.94926 2.63867 2.77262 2.69908
4.58415 3.55522

2.68613 4.42197 2.77528 2.73121 3.46362 2.40507 3.72503 3.29362

2.67741 2.69347 4.24698 2.90355 2.73748 3.18155 2.89786 2.37893 2.77517 2.98499
4.58485 3.61511

| | 0.42529 2.02566 1.53938 0.94684 0.49097 0.00000 * |
|---|---|
| 1 | 2.72350 4.81830 3.16936 2.62649 4.11581 3.51252 3.71942 3.50389 |

2.24998 2.85999 3.95362 3.10351 1.86600 2.88649 2.42995 2.74652 2.82871 3.19574
5.34619 4.06251 6 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02197 4.22496 4.94731 0.61958 0.77255 0.75070 0.63873 |
|---|---|
| 2 | 2.54350 5.09272 2.65636 2.29098 4.41602 2.99758 3.47346 3.88723 |

2.05017 3.38851 4.14035 2.65877 3.36878 2.57545 2.80210 2.49808 2.60450 3.36099
5.53830 4.14000 7 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02165 4.23928 4.96162 0.61958 0.77255 0.55400 0.85483 |
|---|---|
| 3 | 2.29319 4.21840 3.95914 3.39016 3.37768 3.73291 4.08404 2.12760 |

3.30131 2.30451 3.36148 3.66722 3.96097 3.55864 3.55098 2.44506 2.67959 1.73447
4.88856 3.68459 8 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01947 4.34420 5.06654 0.61958 0.77255 0.61386 0.77927 |
|---|---|
| 4 | 2.74772 4.85695 2.87194 2.73668 4.63295 3.30941 4.14345 4.20984 |

3.05642 3.80763 4.64806 1.00956 3.98834 3.35260 3.47426 2.10780 3.15897 3.70197
5.91048 4.53618 9 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01917 4.35956 5.08190 0.61958 0.77255 0.63061 0.75986 |
|---|---|
| 5 | 2.12850 5.12616 2.51993 2.17045 4.44916 3.31679 3.63665 3.80796 |

2.17180 3.42224 4.17456 2.91475 3.69539 2.47321 2.87406 2.49141 2.67432 3.46760
5.57266 4.17399 10 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01917 4.35956 5.08190 0.61958 0.77255 0.63061 0.75986 |
|---|---|
| 6 | 1.46486 5.07291 2.62203 2.24830 4.37269 3.31665 3.74063 3.81552 |

2.50756 3.37686 4.17647 2.86466 3.60033 2.87945 2.92672 2.74276 2.99812 3.44901
5.58151 4.20704 11 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01917 4.35956 5.08190 0.61958 0.77255 0.59794 0.79839 |
|---|---|
| 7 | 2.17549 4.36144 3.73727 3.42594 4.53253 1.83798 4.46965 3.92664 |

3.46881 3.62766 4.44736 3.54831 3.90736 3.74497 3.77756 2.51903 1.12529 3.37293
5.87792 4.67921 12 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01891 4.37321 5.09556 0.61958 0.77255 0.61277 0.78055 |
|---|---|
| 8 | 3.21972 4.50196 5.08099 4.54710 3.67073 4.61619 5.10917 1.48214 |

4.43636 1.72834 3.48636 4.70971 4.89148 4.64075 4.57635 3.97953 3.10048 1.11742
5.56354 4.38821 13 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01891 4.37321 5.09556 0.61958 0.77255 0.61277 0.78055 |
|---|---|
| 9 | 2.35795 5.16270 2.30078 2.19496 4.49371 3.33251 3.64118 3.79980 |

2.21875 3.46186 4.20895 2.47286 3.84197 2.71978 2.70252 2.33251 2.91273 3.54614
5.60290 4.19720 14 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01891 4.37321 5.09556 0.61958 0.77255 0.47193 0.97763 |
|---|---|
| 10 | 3.10702 4.52363 4.59128 3.76793 2.82202 4.24456 4.31533 2.53549 |

3.86137 0.84608 2.77098 4.22984 4.53874 4.04360 4.00636 3.55360 3.33165 2.56243
4.98174 3.78454 15 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01780 4.43314 5.15549 0.61958 0.77255 0.52727 0.89212 |
|---|---|
| 11 | 3.43901 4.67793 5.35556 4.83217 3.64816 4.91195 5.41930 0.86797 |

4.72910 1.79405 3.18234 5.01743 5.11165 4.88087 4.84705 4.30046 3.68194 1.65293
5.72096 4.59690 16 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01780 4.43314 5.15549 0.61958 0.77255 0.52727 0.89212 |
|---|---|
| 12 | 2.25468 5.11708 2.95136 2.31419 4.43742 2.93886 3.67050 3.89862 |

1.79270 3.41419 4.17655 2.95628 3.57244 2.54460 2.89046 2.49672 2.70342 3.49694
5.57506 4.18857 17 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01780 4.43314 5.15549 0.61958 0.77255 0.52727 0.89212 |
|---|---|
| 13 | 2.67371 5.19058 2.65415 1.77278 4.52357 2.97916 3.37968 4.00320 |

2.29480 3.45227 4.23628 2.89789 3.86090 2.69304 2.74143 2.20291 2.82697 3.57508
5.62881 4.22137 18 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01780 4.43314 5.15549 0.61958 0.77255 0.49458 0.94117 |
|---|---|
| 14 | 2.81579 4.22364 4.47018 3.89188 1.48290 3.93522 4.09383 2.62540 |

3.73020 1.92295 3.15573 3.97226 4.29103 3.88937 3.82500 2.90865 3.04647 2.32749
3.69273 3.08017 19 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01758 4.44584 5.16819 0.61958 0.77255 0.50746 0.92136 |
|---|---|
| 15 | 3.18472 5.59003 2.51116 0.73356 4.97137 3.49635 4.05433 4.46680 |

2.91869 4.01079 4.89285 3.02135 4.12423 3.23170 3.44861 2.94448 3.49165 4.05785
6.14660 4.69845 20 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01758 4.44584 5.16819 0.61958 0.77255 0.50746 0.92136 |
|---|---|
| 16 | 2.63285 4.65582 3.05809 2.95774 4.52790 0.99566 4.22427 3.96361 |

3.15170 3.35882 4.43654 3.32975 3.98358 3.44306 3.48471 2.26849 2.95946 3.48867
5.83543 4.54966 21 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01758 4.44584 5.16819 0.61958 0.77255 0.50746 0.92136 |
|---|---|
| 17 | 2.85632 3.37564 4.60322 4.02460 1.39485 3.99023 4.14489 2.59011 |

3.84632 2.15449 3.18679 4.06005 4.34070 3.98159 3.90687 3.30078 3.08762 2.25178
3.05451 3.20082 22 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01758 4.44584 5.16819 0.61958 0.77255 0.50746 0.92136 |
|---|---|
| 18 | 2.66729 4.65691 3.26612 2.63248 3.59907 3.57768 3.79390 3.22463 |

2.58508 2.88889 3.74552 3.18306 3.96288 2.76398 2.21227 2.49586 2.92028 2.02229
5.19804 3.45436 23 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01758 4.44584 5.16819 0.61958 0.77255 0.50746 0.92136 |
|---|---|
| 19 | 2.15329 4.93377 2.72392 2.45038 4.18602 3.46029 3.70858 3.61065 |

2.39726 2.85937 4.00284 3.02169 2.29781 2.84985 2.92083 2.61534 2.92011 3.27400
5.42832 3.51133 24 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01758 4.44584 5.16819 0.61958 0.77255 0.50746 0.92136 |
|---|---|
| 20 | 2.60039 5.14177 2.40424 2.39661 4.46473 3.46452 3.22687 3.82146 |

2.34703 3.43742 4.18905 2.53509 3.85931 2.76069 2.48112 2.15943 2.56736 3.52065
5.58781 3.98848 25 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01758 4.44584 5.16819 0.61958 0.77255 0.50746 0.92136 |
|---|---|
| 21 | 2.67842 4.25530 3.93960 3.30296 2.65434 3.76986 4.06743 2.34859 |

3.16231 2.41816 3.35928 3.66250 1.99709 3.53911 3.53118 3.04669 2.94335 1.99782
4.87521 3.66773 26 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01758 4.44584 5.16819 0.61958 0.77255 0.50746 0.92136 |
|---|---|
| 22 | 2.35693 4.72401 3.20434 2.57805 3.89878 3.59542 3.83415 3.30513 |

2.45060 2.96490 3.82939 3.20409 4.00721 3.02410 3.01463 2.73830 2.99846 3.03825
5.25060 1.65974 27 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.04783 4.44584 3.35315 0.61958 0.77255 0.50746 0.92136 |
|---|---|
| 23 | 2.67429 5.03904 2.71552 2.42815 4.32871 3.47139 3.66503 3.56736 |

2.12738 3.11366 4.09499 2.80474 2.49274 2.58565 2.81632 2.65515 2.70589 3.31018
5.50599 3.32956 28 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01811 4.41613 5.13847 0.61958 0.77255 0.46783 0.98447 |
|---|---|
| 24 | 2.80258 3.80314 1.07544 2.73853 4.22664 3.51752 4.00892 3.12191 |

2.89547 3.24952 4.16749 3.22350 4.04459 3.20256 3.34582 2.88238 3.10901 3.14052
5.59573 4.24511 29 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02905 4.45025 4.07730 0.61958 0.77255 0.50044 0.93208 |
|---|---|
| 25 | 2.25512 4.65074 3.26511 2.72356 3.93527 3.14982 3.83757 3.32426 |

2.65175 2.98376 3.43732 3.00233 1.89086 3.04409 3.13107 2.60570 2.91688 2.73040
5.28256 3.99674 30 - -

2.68618 4.42225 2.77520 2.73123 3.46354 2.40510 3.72495 3.29354

2.67741 2.69355 4.24690 2.90347 2.73740 3.18146 2.89801 2.37887 2.77520 2.98518
4.58477 3.61503

| | 0.09269 3.75476 2.73149 0.54795 0.86306 0.48896 0.95001 |
|---|---|
| 26 | 2.20760 4.20273 3.79874 3.31128 3.16594 3.51238 4.00110 2.04422 |

3.04865 2.40223 3.06460 3.46501 4.08831 3.47948 3.47692 2.91568 2.45698 2.12142
4.80701 3.37658 32 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01860 4.38973 5.11208 0.61958 0.77255 0.44606 1.02206 |
|---|---|
| 27 | 2.89429 4.80561 3.58134 3.54304 4.97183 0.46686 4.80272 4.62849 |

3.86637 4.26878 5.14633 3.40192 4.22316 4.13479 4.14184 3.05434 3.41253 3.99006
6.18155 5.02899 33 - -

2.68620 4.42227 2.77521 2.73125 3.46355 2.40514 3.72472 3.29356

2.67742 2.69356 4.24691 2.90348 2.73732 3.18134 2.89802 2.37888 2.77521 2.98520
4.58478 3.61505

| | 0.04293 3.31454 5.17259 0.77558 0.61699 0.50044 0.93208 |
|---|---|
| 28 | 2.68018 4.37382 3.60152 3.03271 3.37749 3.67013 2.87447 2.64539 |

2.70815 1.83610 3.47768 2.80710 4.04839 3.27708 3.31669 2.91878 2.91175 2.35117
4.95200 3.30164 37 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01750 4.45025 5.17259 0.61958 0.77255 0.50044 0.93208 |
|---|---|
| 29 | 2.59450 4.66219 3.27040 2.59506 3.84655 3.58175 3.79708 3.14848 |

2.36788 2.60330 3.75125 3.18753 2.01907 2.93847 3.06156 2.80580 2.85294 2.96388
3.37709 3.92228 38 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01750 4.45025 5.17259 0.61958 0.77255 0.47669 0.96978 |
|---|---|
| 30 | 2.38332 4.46240 3.96391 3.74958 4.56460 3.29443 4.72276 3.67520 |

3.71927 3.57098 4.53007 3.78193 4.07155 4.04991 3.94677 2.78605 0.65157 3.25220
5.99515 4.80793 39 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 31 | 2.99096 4.42944 4.70942 4.15218 3.57105 4.31482 4.71076 1.65507 |

4.01113 2.14494 3.46003 4.34534 4.64055 3.56162 4.17763 3.64279 3.30622 1.03756
5.29753 4.10999 40 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 32 | 2.68716 3.00522 4.44310 4.24828 4.84005 0.50716 5.03457 4.26978 |

4.19629 4.02594 4.89944 4.06659 4.16496 4.45125 4.33862 2.90519 3.23060 3.67814
6.12241 5.07039 41 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 33 | 3.88807 5.12914 4.96349 4.62971 2.07646 4.68858 3.80768 2.95192 |

4.45489 2.84188 4.11936 4.39032 4.99014 4.43288 4.48511 4.04965 4.11066 3.40338
3.94943 0.62936 42 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 34 | 3.55261 5.22999 4.34030 4.32810 5.38495 0.21258 5.36943 5.16848 |

4.58491 4.72792 5.73557 4.50771 4.63301 4.86357 4.71836 3.74664 4.07606 4.58314
6.28543 5.51964 43 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 35 | 3.87327 5.50886 3.92890 3.78212 3.89799 4.10572 0.35257 4.67985 |

3.62123 4.07577 5.19561 4.15242 4.70480 4.16744 3.83107 3.95140 4.21270 4.40275
5.33871 3.84015 44 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 36 | 2.55850 4.67610 2.85436 2.69895 3.86374 3.57559 3.81417 3.24443 |

2.55253 1.61527 3.72651 3.02186 3.97376 2.93710 3.10915 2.81523 2.56702 2.97733
5.22462 3.94128 45 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 37 | 2.36682 1.08021 4.17330 3.72135 3.82769 2.87004 4.41678 2.93056 |

3.59066 2.91000 3.83458 3.78442 4.07632 3.86713 3.80034 2.83912 2.97220 2.78923
5.31748 3.88653 46 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 38 | 2.44867 5.13209 2.74120 2.37534 4.45129 3.13883 3.37571 3.81639 |

2.09231 3.25691 4.17977 2.88681 3.86250 2.39570 2.69695 2.32020 2.78438 3.04671
5.58043 4.18603 47 - -

2.68605 4.42230 2.77519 2.73128 3.46359 2.40494 3.72500 3.29359

2.67735 2.69360 4.24695 2.90338 2.73745 3.18151 2.89806 2.37892 2.77525 2.98523
4.58482 3.61508

| | 0.20624 2.21113 2.56670 0.73716 0.65099 0.48576 0.95510 |
|---|---|
| 39 | 2.38824 5.13667 2.46225 2.33995 4.46125 3.44638 3.64321 3.93304 |

1.96184 3.43315 4.18470 2.59657 3.84499 2.58355 2.85687 2.53095 2.55813 3.25538
5.58195 4.18304 52 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01868 4.38546 5.10781 0.61958 0.77255 0.59629 0.80040 |
|---|---|
| 40 | 2.52969 5.03587 2.97654 2.42115 4.32785 3.46441 3.53193 3.77619 |

2.02445 3.31736 4.09206 2.74547 2.84178 2.54513 2.88744 2.19787 2.76245 3.39795
4.74099 3.94803 53 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01868 4.38546 5.10781 0.61958 0.77255 0.52092 0.90134 |
|---|---|
| 41 | 2.60675 5.16003 2.52146 2.30324 4.48997 2.73454 3.64845 3.96623 |

2.17370 3.45896 4.20601 2.47959 3.85066 2.56867 2.84590 2.41657 2.61213 3.27322
5.60178 4.19843 54 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01810 4.41655 5.13890 0.61958 0.77255 0.55221 0.85726 |
|---|---|
| 42 | 2.65287 1.19275 3.40429 3.38996 3.71368 3.53484 4.23292 2.66477 |

3.32728 2.80149 3.73937 3.41343 4.09824 3.63311 3.60222 2.91375 2.99822 2.67046
5.20557 3.95872 55 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01810 4.41655 5.13890 0.61958 0.77255 0.55221 0.85726 |
|---|---|
| 43 | 2.11988 4.99095 2.69229 2.46032 4.29867 3.20836 3.70173 3.73910 |

2.41197 3.30074 4.08762 2.99012 3.49720 2.83041 2.93503 1.83660 2.75199 3.37089
5.50311 3.91158 56 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01810 4.41655 5.13890 0.61958 0.77255 0.44635 1.02155 |
|---|---|
| 44 | 2.97807 5.54822 1.79883 1.40119 4.86180 2.66411 3.85669 4.37053 |

2.55019 3.84442 4.63047 2.91417 3.99223 2.98875 3.29382 2.51970 3.23747 3.92819
5.98719 4.51765 57 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.03941 4.45929 3.60927 0.61958 0.77255 0.48576 0.95510 |
|---|---|
| 45 | 2.61240 4.24725 4.16179 3.58472 3.39505 3.87860 4.19991 1.92349 |

3.14424 2.34125 3.37288 3.82971 4.24684 3.71715 3.49381 3.16925 2.66754 1.39907
4.93254 3.73395 58 - -

2.68614 4.42226 2.77520 2.73124 3.46355 2.40514 3.72495 3.29355

2.67742 2.69356 4.24691 2.90347 2.73740 3.18139 2.89802 2.37888 2.77515 2.98519
4.58478 3.61504

| | 0.25671 3.65255 1.60702 0.72234 0.66479 0.46471 0.98972 |
|---|---|
| 46 | 2.58330 4.92802 2.97235 2.46773 4.31534 2.58226 3.69110 3.75176 |

1.87863 3.30755 4.09886 2.98123 2.63799 2.82333 2.81087 2.53970 2.76486 3.37093
5.49792 4.14980 62 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02147 4.24787 4.97022 0.61958 0.77255 0.55284 0.85640 |
|---|---|
| 47 | 2.77665 4.24510 4.17142 3.60737 2.13407 3.57711 3.73827 2.35069 |

3.48227 2.34958 3.36160 3.78414 4.21156 3.69055 3.54299 3.02842 3.00774 2.51180
4.48419 1.75453 63 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.03412 4.34809 3.88173 0.61958 0.77255 0.48868 0.95045 |
|---|---|
| 48 | 2.64793 5.13223 2.68703 2.40086 4.44996 3.45949 3.57898 3.91234 |

1.96012 3.42866 4.19439 2.88878 2.24549 2.73197 2.91333 2.37138 2.77466 3.51110
5.59115 4.20105 64 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.04971 4.40314 3.31713 0.61958 0.77255 0.57163 0.83150 |
|---|---|
| 49 | 2.37672 4.18835 3.56611 3.33942 2.17161 3.71892 4.00701 2.08833 |

3.02370 2.38402 3.30016 3.62106 4.09164 3.49901 3.49065 2.99304 2.64183 2.38390
4.79262 3.11868 65 - -

2.68620 4.42227 2.77521 2.73125 3.46356 2.40514 3.72496 3.29356

2.67728 2.69357 4.24691 2.90348 2.73728 3.18148 2.89802 2.37888 2.77521 2.98520
4.58479 3.61505

| | 0.10921 2.32965 5.09470 0.32898 1.27175 0.41324 1.08324 |
|---|---|
| 50 | 2.36036 4.48294 3.68434 3.32162 3.85852 3.33505 4.35926 3.56334 |

3.31819 3.31552 4.22296 3.55561 0.98977 3.63840 3.64294 2.75832 2.58704 3.18303
5.67820 4.43472 67 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 51 | 3.33618 4.66932 5.03266 4.45397 3.21894 4.56404 4.81904 1.85917 |

4.29340 0.83078 3.04538 4.61993 4.79371 4.38871 4.36938 3.89649 3.55725 2.44200
5.15478 3.19773 68 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 52 | 2.63866 4.70838 2.85148 2.85343 4.48197 3.34287 4.11038 3.90212 |

3.00292 3.53870 4.35671 3.26563 3.96589 3.30138 3.43429 1.35458 1.61544 3.45306
5.76856 4.46367 69 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 53 | 2.60942 5.07136 2.96029 1.86628 4.36808 3.48244 3.67378 3.41756 |

2.01165 2.96551 4.12545 2.81630 3.87448 2.70253 2.69783 2.64352 2.83702 3.14160
5.53327 3.97650 70 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 54 | 1.73678 5.05920 2.76019 2.19606 4.34746 3.40699 3.38009 3.79477 |

2.40682 3.34131 4.11991 2.97912 3.88458 2.75025 2.94159 2.52476 2.67210 3.17492
5.53093 4.15658 71 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 55 | 2.74043 5.24631 2.40465 1.98907 4.58049 3.38842 3.68829 4.06538 |

2.30593 3.54649 4.29373 2.38561 3.88263 2.43061 2.89904 2.64727 2.26054 3.63184
5.68162 4.26599 72 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 56 | 1.08787 3.11680 4.26466 3.92702 4.56744 1.40507 4.74478 3.93820 |

3.86529 3.68181 4.51094 3.79343 3.96845 4.09724 4.08049 2.63816 2.94685 3.37322
5.95490 4.80790 73 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 57 | 2.41449 5.12942 2.78064 2.08572 4.44698 3.23509 3.61872 3.91373 |

2.08441 3.01887 4.17771 2.90972 3.86400 2.67091 2.76247 2.56463 2.37385 3.34773
5.57871 4.18533 74 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 58 | 2.35618 5.15466 2.90683 2.02302 4.48230 3.47099 3.65791 3.95381 |

1.95514 3.38590 3.74147 2.87799 3.86418 2.42507 2.64313 2.58222 2.72135 3.53606
5.59647 4.19971 75 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 59 | 2.82048 4.31441 3.59911 3.58912 3.26417 3.91044 4.18415 2.46874 |

3.46903 1.09245 3.07229 3.84330 4.26181 3.60727 3.67712 3.19562 3.05032 2.49869
4.07413 3.66480 76 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 60 | 3.81742 5.05913 5.53372 4.96642 2.73032 5.13111 5.29889 2.41979 |

4.82530 0.53749 2.78402 5.19788 5.17474 4.75171 4.83127 4.50985 4.01876 2.71504
5.35596 4.27056 77 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 61 | 2.18353 5.08325 2.76302 2.42575 4.38435 3.47806 3.51762 3.56262 |

2.08507 3.12651 3.85720 2.82327 3.87031 2.36589 2.66671 2.51935 2.91708 3.18798
5.54292 4.16091 78 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 62 | 2.69830 5.20991 2.17874 2.32825 4.54673 3.35646 3.67029 4.02682 |

1.91955 3.50995 4.25567 2.93721 3.87314 2.41655 2.73200 2.29314 2.77110 3.59671
5.64478 4.23801 79 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 63 | 3.48091 5.78050 0.48398 2.64029 5.32183 3.22187 4.38302 4.97741 |

3.56135 4.48508 5.42901 3.21673 4.30207 3.60605 4.17838 3.38965 3.84278 4.50650
6.42296 5.06153 80 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 64 | 2.89612 4.42362 4.83116 4.24658 3.32561 4.29064 4.63708 2.02632 |

4.09588 1.20988 2.20498 4.37598 4.59093 4.23666 4.18320 3.48664 3.29831 1.90263
5.10096 3.96543 81 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 65 | 2.20468 5.00834 3.02460 2.46700 4.28538 3.22809 3.68956 3.72332 |

2.07471 3.22745 4.06994 2.99846 3.13955 2.71422 2.40530 2.55367 2.81763 2.91995
5.48516 3.93946 82 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02786 4.45929 4.14114 0.61958 0.77255 0.48576 0.95510 |
|---|---|
| 66 | 2.57606 4.97328 3.01298 2.37658 4.23859 2.99122 3.69147 3.38775 |

2.24029 3.15189 3.89305 2.89169 3.40567 2.76365 2.64345 2.34850 2.54841 2.73885
5.45700 4.10045 83 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44895 5.17130 0.61958 0.77255 0.50251 0.92889 |
|---|---|
| 67 | 2.13166 4.25308 3.90762 3.34403 2.05039 3.72688 4.02347 2.73125 |

3.25999 2.46542 3.38226 3.63057 3.61807 3.51702 3.51516 2.92597 2.67380 2.52551
4.81288 2.35249 84 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44895 5.17130 0.61958 0.77255 0.50251 0.92889 |
|---|---|
| 68 | 2.51295 5.09054 2.98184 1.72812 4.39612 3.48435 3.67785 3.73881 |

2.41954 3.37739 4.14803 2.97394 3.88052 2.28202 2.67212 2.68772 2.46700 3.25830
5.55010 4.17304 85 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44895 5.17130 0.61958 0.77255 0.47543 0.97185 |
|---|---|
| 69 | 2.66132 5.17285 2.66986 2.39540 4.50725 3.46839 3.65551 3.76967 |

1.96543 3.47208 4.21733 2.37317 3.86241 2.33907 2.51152 2.47816 2.84664 3.35597
5.61061 4.20948 86 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 70 | 2.40476 1.44095 3.96481 3.44698 3.67421 2.80450 4.21141 3.02108 |

3.36628 2.76284 3.66000 3.64693 4.04722 3.63180 3.63000 2.52077 2.78604 2.59026
5.14211 3.50924 87 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 71 | 3.14464 4.45336 5.03021 4.46417 3.35819 4.45790 4.86159 1.42980 |

4.32230 1.69292 2.86587 4.38080 4.74701 4.47359 4.40409 3.79830 3.38213 1.35681
5.29927 4.14289 88 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 72 | 2.30011 3.73407 3.03761 2.41231 4.07806 3.34217 3.55224 3.48929 |

2.54009 3.10364 3.92326 2.86753 3.54726 2.88785 2.87570 2.44080 2.13433 3.07048
5.35921 3.53834 89 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 73 | 2.14048 5.14694 2.67449 2.30248 4.47134 3.40113 3.65777 3.94253 |

2.17125 2.97837 4.13264 2.74458 3.76417 2.57571 2.52099 2.46181 2.82539 3.52649
5.59184 4.19432 90 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 74 | 2.33987 3.97918 2.71800 2.68393 3.66274 3.56899 3.79046 3.23575 |

2.52458 2.58207 2.74593 2.97884 3.95502 2.69024 3.09550 2.49450 2.91310 2.83865
5.20705 3.47237 91 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 75 | 2.72508 4.24130 3.63475 3.45984 3.36239 3.81026 4.11532 2.14147 |

3.36542 1.74492 3.34959 3.73067 3.64897 3.61353 3.59970 3.09084 2.24848 1.97147
4.88118 3.68107 92 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.11347 4.45929 2.34657 0.61958 0.77255 0.48576 0.95510 |
|---|---|
| 76 | 2.44605 5.08758 2.70732 2.38750 4.40669 3.21874 3.64649 3.86979 |

2.21274 3.38705 4.14749 2.21702 3.28711 2.75638 2.85771 2.36598 2.63424 3.26702
5.55021 4.16021 93 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.09636 4.36489 2.53639 0.61958 0.77255 0.62371 0.76777 |
|---|---|
| 77 | 2.52140 5.19229 1.93533 2.18979 4.51146 3.30655 3.67579 3.98696 |

2.38932 3.49150 4.25802 2.75360 3.76248 2.79221 2.91679 2.09509 2.83845 3.57261
5.64795 4.23568 94 - -

2.68619 4.42226 2.77512 2.73124 3.46355 2.40511 3.72495 3.29355

2.67742 2.69356 4.24691 2.90347 2.73740 3.18147 2.89802 2.37886 2.77520 2.98519
4.58478 3.61504

| | 0.13745 2.78775 2.70509 0.42399 1.06256 0.37095 1.17143 |
|---|---|
| 78 | 2.36341 5.11547 2.88923 2.38557 4.43588 3.12293 3.65240 3.90110 |

1.86565 3.41125 4.16867 2.81154 3.56873 2.61972 2.78323 2.22914 2.70573 3.49407
5.56793 4.17668 96 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01833 4.40410 5.12644 0.61958 0.77255 0.56252 0.84344 |
|---|---|
| 79 | 2.28762 4.29763 3.88020 3.31128 3.45344 3.77121 4.08077 2.37703 |

2.80959 2.48145 3.28647 3.63489 3.92261 3.50644 3.49947 3.04725 2.88061 1.42058
4.95908 3.74730 97 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01827 4.40717 5.12952 0.61958 0.77255 0.49049 0.94759 |
|---|---|
| 80 | 2.49336 4.91866 3.06468 2.50610 4.16905 3.50622 3.47926 3.59090 |

2.04052 3.18152 3.98708 2.87458 3.40359 2.84700 2.63414 2.70907 2.30092 2.61912
5.41218 3.71728 98 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01772 4.43760 5.15995 0.61958 0.77255 0.46471 0.98972 |
|---|---|
| 81 | 3.45413 4.80651 5.06351 4.52310 3.14668 4.66315 5.03497 2.37248 |

4.37178 0.65498 3.13154 4.73925 4.90613 4.49133 4.48329 4.02717 3.33601 2.26220
5.35929 4.25227 99 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 82 | 2.73939 4.83330 3.00723 2.83344 4.71960 3.34320 4.20357 4.19656 |

3.09807 3.81073 4.63710 0.93081 4.01975 3.40262 3.51075 2.36222 2.79342 3.68571
5.97881 4.64405 100 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 83 | 2.04424 5.17228 2.45633 2.07839 4.50311 3.46548 3.65902 3.97943 |

2.19774 3.32071 4.21748 2.93804 3.60950 2.58020 2.72229 2.50774 2.92631 3.55540
5.61278 4.10566 101 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 84 | 2.94884 5.24796 2.66241 2.49038 4.31917 3.52589 3.86118 3.97970 |

2.71091 3.53375 4.37214 1.14648 4.03010 2.78537 3.17219 2.92497 3.20366 3.62731
5.61132 3.34532 102 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 85 | 3.04248 5.24204 2.96062 2.60820 4.65653 3.59476 3.94929 4.07361 |

2.56705 3.61817 4.49759 3.18876 4.11995 0.96272 2.89157 3.04418 2.94698 3.72218
5.79098 4.47565 103 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 86 | 3.15528 4.57797 4.44504 3.93193 1.85137 4.15122 3.86565 3.06977 |

3.40619 2.70190 3.70726 4.00202 4.49914 3.94620 3.42969 3.27245 3.38207 2.89093
2.96247 1.23585 104 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 87 | 1.91388 4.74994 2.64175 2.83276 4.65908 1.23026 4.17545 4.11077 |

3.11125 3.71800 4.52370 3.25060 3.97313 3.36530 3.56454 2.36795 3.06739 3.59931
5.92594 4.60838 105 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 88 | 0.42105 4.60486 4.18172 4.04773 4.75980 3.40644 4.98013 3.98668 |

4.08316 3.83537 4.82707 4.00997 4.20267 4.37016 4.26066 2.96393 3.28352 3.52613
6.11697 5.00536 106 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 89 | 2.94731 4.79718 4.85811 4.40161 3.41462 4.43006 5.01217 2.43392 |

4.20104 0.64102 3.23863 4.59387 4.80786 4.42809 4.33978 3.84144 3.64619 2.39900
5.44657 4.32673 107 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 90 | 2.69295 3.98202 4.44440 3.88309 3.49385 3.95542 4.41654 1.88586 |

3.76101 2.45692 3.45424 4.04259 4.36196 3.98345 3.92955 2.88509 2.53539 1.21450
5.07315 3.87854 108 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 91 | 2.23020 4.52728 3.33461 3.29998 4.71681 3.23321 4.50469 4.14576 |

3.50163 3.83741 4.66077 3.52242 3.99178 3.76238 3.84265 0.70612 3.04592 3.56218
6.04302 4.79944 109 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 92 | 4.05064 5.18728 5.18918 4.93015 1.30934 4.79876 3.74116 3.69305 |

4.72772 2.64779 4.25890 4.46765 5.07773 4.56292 4.64571 4.16664 4.25926 3.61933
1.17959 1.95218 110 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 93 | 1.28536 3.49665 3.98287 3.48773 3.94001 3.37174 4.32499 3.24593 |

3.42157 3.01829 3.90186 3.64596 4.01208 3.68988 3.70737 2.34460 1.97075 2.43625
5.38426 4.18438 111 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 94 | 4.32746 5.37697 5.24755 5.08968 0.67847 4.92310 3.72282 3.90894 |

4.89259 3.12203 4.46331 4.52796 5.19934 4.66186 4.77457 4.32624 4.53605 3.86568
3.81270 1.39600 112 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 95 | 2.96563 5.03414 2.96147 2.89269 4.69467 3.45494 4.31635 4.30858 |

3.22879 3.95664 4.87223 0.68215 4.15542 3.55820 3.60524 3.03479 3.16772 3.84396
5.99932 4.61384 113 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 96 | 2.69438 4.19269 4.25987 3.56878 3.31680 3.86774 4.19014 2.37254 |

3.54681 2.01345 2.82578 3.75808 4.23400 3.76804 3.71685 2.96995 2.77903 1.36393
4.85178 3.66032 114 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 97 | 2.84079 3.93256 4.40616 4.26397 4.98754 0.39355 5.11403 4.49447 |

4.25980 4.21665 5.11067 4.15445 4.26046 4.53706 4.40205 3.05226 3.38675 3.87725
6.18922 5.18548 115 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 98 | 2.56290 1.46178 3.90788 3.37708 3.63161 3.17695 4.15838 2.97148 |

3.29073 2.63206 3.61432 3.36809 3.48256 3.56931 3.49641 2.86742 2.68116 2.40505
5.09846 3.89390 116 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 99 | 2.54148 5.04654 2.70539 2.37565 4.49775 1.34409 3.89783 3.94239 |

2.76174 3.51789 4.32880 2.93324 3.97003 3.05438 3.18788 2.57266 2.88542 3.54943
5.73092 4.36307 117 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 100 | 1.70557 4.79988 3.14657 2.65009 4.21466 2.82353 3.84500 3.42042 |

2.59544 3.24327 4.06100 1.96080 3.93084 3.00954 3.10889 2.73130 2.86940 3.18113
5.48983 4.17058 118 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 101 | 2.00483 4.12604 4.25448 3.66225 2.77296 3.75391 4.11010 2.48130 |

3.52384 2.03969 2.75516 3.82391 4.16685 3.59884 3.66689 2.88608 2.76467 1.94131
4.74141 3.24653 119 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.03303 4.45929 3.86691 0.61958 0.77255 0.48576 0.95510 |
|---|---|
| 102 | 2.39869 4.21435 2.90223 2.24202 4.43514 2.98780 3.65947 3.89880 |

2.09993 3.22037 4.16856 2.93058 3.86386 2.09592 2.58555 2.64666 2.91791 3.49428
5.56867 4.17940 120 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.03161 4.44387 3.94438 0.61958 0.77255 0.51058 0.91665 |
|---|---|
| 103 | 2.65435 5.11517 2.88355 2.35449 4.19901 3.13016 3.36182 3.89436 |

2.23601 3.40681 4.16467 2.87691 3.85914 2.63441 2.47196 1.93702 2.57645 3.48979
5.56559 4.17529 121 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01786 4.43012 5.15246 0.61958 0.77255 0.48058 0.96345 |
|---|---|
| 104 | 2.41340 4.51586 3.64272 3.20902 3.96433 3.40133 4.18341 3.53981 |

3.13494 3.21340 4.09895 3.49607 4.02251 3.49050 3.46406 0.91626 3.00878 3.18189
4.01968 4.06003 122 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01750 4.45022 5.17257 0.61958 0.77255 0.47667 0.96981 |
|---|---|
| 105 | 2.54090 4.85452 2.71842 2.54794 4.08758 3.52137 3.73124 3.35427 |

2.53920 3.11249 3.93155 3.06305 3.91306 2.75440 2.85634 2.18869 1.95817 3.18450
3.86953 4.03850 123 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 106 | 2.89313 4.34581 4.36083 3.78021 3.00250 4.01068 3.73906 2.43845 |

3.64357 1.03448 3.21890 3.99265 4.35163 3.85444 3.81380 3.30755 2.87425 2.34644
4.89798 3.69665 124 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 107 | 2.38886 4.16785 4.23962 3.65212 3.27942 3.49631 4.14953 1.93787 |

3.52117 1.96618 3.08994 3.83908 4.20151 3.73674 3.19737 3.12692 2.88453 1.75092
4.80522 3.61535 125 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 108 | 2.18860 5.09773 3.01081 2.41645 4.40890 3.02601 3.68034 3.70590 |

1.98541 3.38487 4.15581 2.98738 3.88981 2.71530 2.19915 2.40384 2.85715 3.47177
5.55249 4.18095 126 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 109 | 2.88848 5.01007 3.36359 2.70440 4.28892 3.67769 3.76628 3.67046 |

2.13438 2.83550 3.27792 3.21417 4.04788 2.78880 1.37591 2.92736 2.91052 3.36660
5.44614 4.18169 127 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 110 | 3.01550 4.50647 4.29320 3.35689 3.38082 4.10807 4.41212 2.46129 |

3.56171 0.92330 3.04014 4.02324 4.44436 3.85149 3.77877 3.41276 2.95071 2.45376
5.11441 3.95652 128 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 111 | 2.93246 5.26208 2.83845 2.51972 4.59855 3.53909 3.83421 4.05395 |

2.41041 3.41162 4.39541 1.22702 4.02054 2.58739 2.94782 2.68984 3.17979 3.67765
5.73531 4.37438 129 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01734 4.45929 5.18164 0.61958 0.77255 0.48576 0.95510 |
|---|---|
| 112 | 1.88527 5.12970 3.02339 2.46506 4.44856 3.51148 3.58652 3.89850 |

1.94584 3.41349 3.98691 2.52080 3.90280 2.52008 2.59054 2.68296 2.96264 3.50769
5.57002 4.20272 130 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02789 4.45929 4.13926 0.61958 0.77255 0.48576 0.95510 |
|---|---|
| 113 | 2.74124 4.71833 3.14766 2.56164 4.15454 1.48155 3.79728 3.56948 |

2.55233 3.04403 4.01264 2.75713 3.96322 2.96545 2.76272 2.79611 2.99003 3.24999
5.18738 4.10961 131 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44892 5.17127 0.61958 0.77255 0.50256 0.92882 |
|---|---|
| 114 | 2.78829 5.27067 2.39544 1.43884 3.52342 3.39995 3.72836 4.06474 |

2.49027 3.56292 4.32709 2.73652 3.90878 2.43899 3.02346 2.74367 3.02799 3.64666
5.71376 4.29711 132 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44892 5.17127 0.61958 0.77255 0.50256 0.92882 |
|---|---|
| 115 | 2.58656 5.08616 1.97465 2.39675 4.38521 3.47529 3.67325 3.84095 |

2.19218 3.37163 4.14005 2.49894 3.87169 2.78755 2.91440 2.60276 2.70141 3.45224
4.35740 3.44083 133 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44892 5.17127 0.61958 0.77255 0.50256 0.92882 |
|---|---|
| 116 | 2.36408 4.93399 3.05515 2.41820 4.19006 3.28886 3.70715 3.53115 |

2.35589 3.20126 4.00416 3.02568 2.28335 2.65312 2.79254 2.53358 2.91948 2.72092
5.42832 4.08366 134 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.07281 4.44892 2.83821 0.61958 0.77255 0.50256 0.92882 |
|---|---|
| 117 | 2.47921 5.15398 2.61824 2.37031 4.47665 3.08217 3.69410 3.94314 |

2.47045 3.45936 4.22669 1.75974 3.87627 2.54232 2.92200 2.53173 2.86250 3.45021
5.62343 4.22533 135 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.16248 4.39462 1.98324 0.61958 0.77255 0.58364 0.81613 |
|---|---|
| 118 | 2.47352 4.43682 3.41083 2.85816 3.65814 3.53539 3.85691 2.85096 |

2.62235 2.70516 3.52326 3.27951 3.95603 3.10614 3.16295 2.71586 1.69391 2.42145
5.07488 3.82615 136 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02134 4.25348 4.97583 0.61958 0.77255 0.75230 0.63730 |
|---|---|
| 119 | 3.12393 4.58092 4.64216 4.33325 3.86300 4.10675 5.08066 2.11413 |

4.20840 2.49287 3.78444 4.45840 4.65799 4.54804 4.38259 3.64748 3.48352 0.64916
5.70632 4.45559 137 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02134 4.25348 4.97583 0.61958 0.77255 0.36777 1.17856 |
|---|---|
| 120 | 1.52970 4.31242 4.27636 3.70359 3.15057 3.97419 4.29716 1.75553 |

3.59501 2.15161 3.34066 3.94036 4.33244 3.82618 3.79879 3.27010 3.08534 2.21733
4.97587 3.57672 138 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44892 5.17127 0.61958 0.77255 0.50256 0.92882 |
|---|---|
| 121 | 2.00921 3.05304 3.07103 2.43399 4.07708 3.27326 3.62893 3.48743 |

2.55372 3.10420 3.92537 3.02189 3.60524 2.90180 2.72153 2.18932 2.85663 3.17238
5.36146 4.03760 139 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44892 5.17127 0.61958 0.77255 0.50256 0.92882 |
|---|---|
| 122 | 2.38800 5.20369 2.45901 1.93151 4.53281 3.41096 3.67622 4.01159 |

2.41230 3.32407 4.25203 2.80676 3.87283 2.05963 2.92660 2.50851 2.91530 3.52839
5.64462 4.23622 140 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44892 5.17127 0.61958 0.77255 0.50256 0.92882 |
|---|---|
| 123 | 2.80442 5.57008 2.32768 1.01424 4.88079 2.93753 3.89581 4.38320 |

2.81015 3.87205 4.67758 2.92022 4.01617 2.76513 3.36230 2.93852 3.29333 3.95302
6.02758 4.55781 141 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44892 5.17127 0.61958 0.77255 0.50256 0.92882 |
|---|---|
| 124 | 3.65291 4.92987 5.28841 4.75290 2.09973 4.85851 4.79616 2.49842 |

4.59706 0.68663 3.08443 4.86508 5.00895 4.57855 4.61939 4.21526 3.86249 2.75251
4.95107 3.43578 142 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44892 5.17127 0.61958 0.77255 0.50256 0.92882 |
|---|---|
| 125 | 2.68163 4.78103 3.25910 2.74134 4.08738 3.29345 3.84411 3.27298 |

2.47216 3.00438 3.96065 3.20761 1.46271 3.03322 2.77484 2.83542 3.00539 3.16979
5.37921 4.09799 143 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01752 4.44892 5.17127 0.61958 0.77255 0.50256 0.92882 |
|---|---|
| 126 | 2.97837 5.31590 3.32617 2.68999 4.72425 3.69304 3.27951 4.12761 |

1.41978 3.15207 4.36950 2.95852 4.05870 2.29333 1.88668 2.95013 3.17244 3.74769
5.64414 4.36966 144 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02051 4.44892 4.75533 0.61958 0.77255 0.50256 0.92882 |
|---|---|
| 127 | 3.24053 4.68626 4.42354 3.93390 2.40064 4.19011 3.87268 3.13732 |

3.77092 2.69167 3.77020 4.01866 4.54932 3.96154 3.93737 3.50591 2.78389 2.98596
1.09473 2.38377 145 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01757 4.44599 5.16833 0.61958 0.77255 0.50723 0.92171 |
|---|---|
| 128 | 2.69906 4.81576 3.08565 2.57557 4.04005 3.53963 3.74869 3.22744 |

2.40032 3.06681 3.89789 1.97993 3.93159 2.91813 2.73517 2.75677 2.74275 2.22386
5.33397 4.02105 146 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02207 4.44599 4.59455 0.61958 0.77255 0.50723 0.92171 |
|---|---|
| 129 | 2.67905 4.57309 3.32994 2.76622 3.73883 3.59300 3.19938 3.01028 |

2.15094 2.56209 3.22488 3.09606 3.97668 3.06418 2.85128 2.81906 2.63816 2.82377
4.30953 2.58633 147 - -

2.68618 4.42225 2.77515 2.73123 3.46354 2.40513 3.72495 3.29354

2.67741 2.69355 4.24690 2.90347 2.73740 3.18146 2.89801 2.37887 2.77520 2.98518
4.58477 3.61503

| | 0.02949 3.75743 5.16391 0.54795 0.86306 0.51420 0.91124 |
|---|---|
| 130 | 1.37236 4.40095 3.68215 3.15295 3.75592 2.52271 4.08407 2.92930 |

3.10149 2.47905 3.70955 3.48452 4.03143 3.40599 3.44185 2.76304 2.93611 2.46504
5.20046 3.97096 149 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01765 4.44156 5.16391 0.61958 0.77255 0.51420 0.91124 |
|---|---|
| 131 | 2.92571 5.41476 2.40274 2.25750 4.73426 1.59137 3.83505 4.22032 |

2.46966 3.72284 4.51462 2.12442 3.97518 2.96959 3.22426 2.64902 3.18355 3.80226
5.88395 4.44532 150 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01765 4.44156 5.16391 0.61958 0.77255 0.51420 0.91124 |
|---|---|
| 132 | 2.91871 5.13022 2.71064 2.58530 4.61395 1.08952 3.96281 4.05904 |

2.60237 3.62616 4.46295 2.92155 4.04062 3.13339 2.89835 2.92929 3.22159 3.66933
5.81419 4.46718 151 - -

2.68618 4.42225 2.77520 2.73124 3.46354 2.40513 3.72495 3.29354

2.67741 2.69355 4.24690 2.90347 2.73732 3.18147 2.89801 2.37887 2.77520 2.98519
4.58477 3.61503

| | 0.08528 3.78188 2.83085 0.63047 0.76001 0.51420 0.91124 |
|---|---|
| 133 | 2.55415 5.12827 2.99700 2.37401 4.45513 3.42989 3.22630 3.91005 |

1.66425 3.41408 4.09517 2.82365 3.87578 2.41406 2.56728 2.66166 2.93780 3.15862
5.56110 4.18910 154 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01865 4.38685 5.10920 0.61958 0.77255 0.59439 0.80274 |
|---|---|
| 134 | 2.68757 4.69832 3.24582 2.59317 3.89741 3.57072 3.77065 3.17298 |

2.28857 2.93278 3.78681 3.16345 3.22761 2.80999 2.72847 2.80064 2.93185 1.75884
5.22736 3.94700 155 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01865 4.38685 5.10920 0.61958 0.77255 0.59439 0.80274 |
|---|---|
| 135 | 2.66483 4.14616 4.10629 3.52046 3.11519 3.76324 4.05895 2.53934 |

3.39922 1.42345 3.09375 3.35986 4.13201 3.56974 3.58549 2.72565 2.89692 2.25748
3.72072 3.54295 156 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01865 4.38685 5.10920 0.61958 0.77255 0.59439 0.80274 |
|---|---|
| 136 | 2.54947 5.15494 2.81817 2.27071 4.47196 3.45438 3.67320 3.93783 |

2.18117 3.44723 4.21076 2.61053 2.15770 2.48927 2.90923 2.63558 2.81969 3.53198
5.60426 4.20964 157 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01865 4.38685 5.10920 0.61958 0.77255 0.46370 0.99143 |
|---|---|
| 137 | 2.48424 4.64364 3.23983 2.74683 4.45646 0.95630 4.24608 3.78460 |

3.16682 3.50936 4.37248 3.36772 4.01139 3.48004 3.54078 2.79397 3.07496 2.94872
5.79263 4.52277 158 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01765 4.44156 5.16391 0.61958 0.77255 0.51420 0.91124 |
|---|---|
| 138 | 3.30920 4.75420 4.61173 4.11127 3.12041 4.38915 4.69609 2.54917 |

3.90653 0.68230 3.16405 4.37109 4.69955 3.81654 4.07332 3.74605 3.29083 2.64075
5.18905 3.99579 159 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01765 4.44156 5.16391 0.61958 0.77255 0.51420 0.91124 |
|---|---|
| 139 | 2.64297 4.51540 3.13915 2.58066 3.67426 3.43890 3.84982 2.79669 |

2.71873 2.72061 3.40686 3.27646 3.99414 3.11806 3.19182 2.71823 2.35387 1.80992
5.08364 3.82661 160 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02149 4.44156 4.65848 0.61958 0.77255 0.51420 0.91124 |
|---|---|
| 140 | 2.64854 5.18440 3.10682 2.54595 4.52943 3.57236 3.59991 3.96071 |

2.19829 3.42661 4.24551 2.43253 3.96199 2.65206 1.54852 2.67222 2.94271 3.57966
5.59352 4.26198 161 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01772 4.43780 5.16014 0.61958 0.77255 0.52008 0.90256 |
|---|---|
| 141 | 3.44190 5.32458 4.00047 3.46399 4.96977 3.41557 4.22078 4.46813 |

2.52378 3.92894 4.85429 3.79058 4.42887 3.40774 0.52734 3.51113 3.71233 4.11175
5.83836 4.76025 162 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01772 4.43780 5.16014 0.61958 0.77255 0.52008 0.90256 |
|---|---|
| 142 | 3.44190 5.32458 4.00047 3.46399 4.96977 3.41557 4.22078 4.46813 |

2.52378 3.92894 4.85429 3.79058 4.42887 3.40774 0.52734 3.51113 3.71233 4.11175
5.83836 4.76025 163 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02519 4.43780 4.33842 0.61958 0.77255 0.52008 0.90256 |
|---|---|
| 143 | 1.90653 5.22931 2.62762 2.14830 4.55055 3.46127 3.70649 4.02504 |

2.07812 3.52458 4.28677 2.20792 3.89414 2.79487 2.97073 2.72114 2.99769 3.55680
5.67510 4.26740 164 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01785 4.43045 5.15280 0.61958 0.77255 0.53138 0.88623 |
|---|---|
| 144 | 1.66693 4.90314 2.79419 2.43720 4.14970 3.50399 3.70716 3.38051 |

2.26779 3.16514 3.57851 2.96848 3.89556 2.85254 2.83650 2.70887 2.91240 3.07112
5.40141 4.06201 165 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02074 4.43045 4.75450 0.61958 0.77255 0.53138 0.88623 |
|---|---|
| 145 | 3.57909 5.69383 2.89268 0.43731 5.13396 3.74413 4.42879 4.71520 |

3.39551 4.25239 5.24212 3.42136 4.38610 3.65781 3.83675 3.52400 3.90016 4.35367
6.18972 4.96810 166 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02361 4.42762 4.47474 0.61958 0.77255 0.53569 0.88012 |
|---|---|
| 146 | 2.53815 4.23541 3.89256 3.31415 3.35365 3.43314 4.01926 2.27305 |

3.13182 2.36020 3.34809 3.61467 4.10930 3.48366 2.78779 3.00792 2.91165 1.57725
4.84488 3.36475 167 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01800 4.42201 5.14436 0.61958 0.77255 0.54412 0.86835 |
|---|---|
| 147 | 1.80943 5.17035 2.62157 2.07449 4.49100 3.42292 3.67419 3.96118 |

2.17043 3.46432 4.22212 2.93630 3.87161 2.56527 2.84559 2.64670 2.88453 3.50978
5.61623 4.21716 168 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01800 4.42201 5.14436 0.61958 0.77255 0.54412 0.86835 |
|---|---|
| 148 | 3.71568 4.98323 5.39542 4.83916 2.79356 5.00269 5.20401 2.41032 |

4.69620 0.57295 2.93926 5.06411 5.09914 4.67653 4.73417 4.37793 3.92780 2.46918
5.32073 4.20270 169 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.01800 4.42201 5.14436 0.61958 0.77255 0.54412 0.86835 |
|---|---|
| 149 | 1.99327 2.70853 4.08451 3.52864 1.85792 3.42232 3.91516 2.71735 |

3.41997 2.46283 3.37905 3.72050 4.10390 3.65301 3.62210 2.80539 2.90591 2.50317
4.85301 3.64388 170 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.03954 4.42201 3.62092 0.61958 0.77255 0.54412 0.86835 |
|---|---|
| 150 | 2.52034 5.07675 2.86686 2.34451 4.38139 3.46473 3.58228 3.83719 |

1.82596 2.91213 3.78433 2.90755 3.85707 2.33901 2.80894 2.51619 2.74142 3.44564
5.53279 4.15171 171 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.05269 4.40086 3.24268 0.61958 0.77255 0.57487 0.82732 |
|---|---|
| 151 | 2.56841 4.80930 3.10084 2.47522 4.03593 3.39038 3.70967 3.30280 |

2.05479 3.06154 3.82459 3.05201 3.89470 2.71798 2.95896 2.58812 1.96719 3.08959
4.39727 4.00056 172 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.03303 4.36720 3.92179 0.61958 0.77255 0.62070 0.77126 |
|---|---|
| 152 | 2.00712 4.80326 3.04608 2.51290 4.16635 3.12701 3.47637 3.58373 |

2.58766 3.19402 4.01252 3.03841 2.06845 2.94568 3.05479 2.54694 2.74358 3.23947
5.43985 4.11489 173 - -

2.68621 4.42228 2.77522 2.73117 3.46357 2.40515 3.72476 3.29338

2.67743 2.69358 4.24692 2.90349 2.73734 3.18149 2.89803 2.37884 2.77522 2.98521
4.58480 3.61506

| | 0.11549 2.35531 4.25417 0.54335 0.86941 0.63840 0.75107 |
|---|---|
| 153 | 2.16030 4.62913 3.21138 2.59079 4.25901 2.76297 3.94367 3.67658 |

2.80049 3.30378 4.12023 3.18454 3.88643 3.12635 3.23166 1.76272 1.88963 3.27351
5.54965 4.25166 176 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.03127 4.34547 4.02732 0.61958 0.77255 0.64844 0.73995 |
|---|---|
| 154 | 2.66792 5.11177 2.56371 2.33718 4.43052 3.12411 3.63758 3.76704 |

2.24610 3.40665 4.16392 2.51360 3.83618 2.72460 2.75418 2.00063 2.60055 3.48912
5.56277 4.16722 177 - -

2.68618 4.42225 2.77520 2.73123 3.46354 2.40510 3.72495 3.29354

2.67741 2.69355 4.24690 2.90347 2.73740 3.18146 2.89801 2.37887 2.77520 2.98518
4.58477 3.61503

| | 0.20910 3.75741 1.79972 0.56129 0.84507 0.66269 0.72456 |
|---|---|
| 155 | 2.55143 4.81591 2.81468 2.31661 3.92133 3.34474 3.63569 3.47575 |

2.37389 3.07571 3.88637 2.95949 3.82254 2.66149 2.66956 2.47706 2.52334 2.53039
5.31632 3.83266 179 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.03176 4.16074 4.15646 0.61958 0.77255 0.76695 0.62442 |
|---|---|
| 156 | 2.55523 4.93315 2.94860 2.24014 4.20806 3.11633 3.48685 3.19520 |

2.09238 3.20688 3.99407 2.92490 3.24954 2.58362 2.75119 2.50501 2.80774 3.04779
5.40950 4.04862 180 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02276 4.19001 4.91235 0.61958 0.77255 0.81337 0.58584 |
|---|---|
| 157 | 1.20660 4.40759 3.35604 2.93272 3.95893 3.20449 3.52467 3.34303 |

2.90686 3.04070 3.90878 3.27859 3.58791 3.25408 3.26247 2.65317 2.85764 2.78550
5.33854 4.07865 181 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02276 4.19001 4.91235 0.61958 0.77255 0.68268 0.70372 |
|---|---|
| 158 | 2.67140 4.29203 3.70746 3.13942 2.86196 3.68163 2.96310 2.76890 |

3.05716 1.69286 3.39392 3.48105 3.86490 2.82577 3.35631 2.94264 2.90223 2.56219
3.97407 3.25518 182 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.02128 4.25624 4.97858 0.61958 0.77255 0.74947 0.63982 |
|---|---|
| 159 | 2.63798 4.48996 3.51982 3.04228 3.81419 3.47372 4.04467 3.03664 |

2.92137 2.64518 3.23372 3.40240 1.41997 3.31178 3.25344 2.82205 2.74199 2.80698
5.29179 4.03651 183 - -

2.68618 4.42225 2.77519 2.73123 3.46354 2.40513 3.72494 3.29354

2.67741 2.69355 4.24690 2.90347 2.73739 3.18146 2.89801 2.37887 2.77519 2.98518
4.58477 3.61503

| | 0.14151 4.25624 2.13899 0.61958 0.77255 0.74947 0.63982 |
|---|---|
| 160 | 2.46470 3.83575 3.23523 2.67393 3.66999 3.49642 3.73458 3.04690 |

2.43613 2.64675 3.59308 3.01880 3.25014 2.84868 2.98138 2.59083 2.70116 2.25801
5.05478 3.78583 184 - -

2.68617 4.42231 2.77511 2.73129 3.46360 2.40503 3.72497 3.29360

2.67725 2.69361 4.24696 2.90353 2.73733 3.18150 2.89795 2.37891 2.77523 2.98525
4.58483 3.61509

| | 0.49613 0.96883 4.45773 0.15588 1.93562 0.80876 0.58953 |
|---|---|
| 161 | 2.60601 0.74299 4.28430 4.02298 4.16889 3.28957 4.69398 3.40236 |

3.85046 3.26155 4.28085 3.92541 3.45640 4.17512 3.98128 2.85611 3.09926 3.06230
5.57309 4.44623 186 - -

2.68617 4.42226 2.77521 2.73125 3.46355 2.40514 3.72496 3.29355

2.67738 2.69356 4.24691 2.90348 2.73741 3.18131 2.89802 2.37887 2.77517 2.98520
4.58478 3.61504

0.09408 2.41030 * 0.42020 1.06978 0.00000 *

//

### Example 17: In Vivo Broiler Trial

### Materials and Methods

The trial was performed at the Research Center for Animal Nutrition (DSM Nutritional Products France, F-68305 Village-Neuf) according to the official French guidelines for experiments with live animals. Day-old male broiler chickens (“ROSS PM3”), were supplied by a commercial hatchery (Joseph Grelier S. A., Elevage avicole de la Bohadière, F-49290 Saint-Laurent de la Plaine, France).

### Animals and Housing

On the day of arrival (day 1), the chickens were divided by weight into groups of 20 birds. Each group was placed in one floor-pen littered with wood shavings and allocated to one of the different treatments.

Each treatment was replicated with 8 groups. The chickens were housed in an environmentally controlled room. The room temperature was adapted to the age of the birds. In the first few days an additional infra-red electric heating lamp was placed in each pen. Moreover, in the first week feed was offered to the birds as crumbled pellets, afterwards as pelleted feed. The birds had free access to feed and water.

### Feeding and Treatments

The experimental diets (Starter, Grower and Finisher) were based on soybean meal, wheat and rye (12%) as main ingredients (Table 8). The diets were formulated to contain 225 g crude protein and 12.5 MJ/kg MEN for the starter period, 215 g crude protein and 12.8 MJ/kg MEN for the grower period and 205 g crude protein and 13.0 MJ/kg MEN for the finisher period.

TABLE 8
Composition and nutrient contents of the basal experimental diets

| | Starter | Grower | Finisher |
|---|---|---|---|
| | (d 1-22) | (d 22-36) | (d 36-42) |
| Ingredients (%) | | | |
| Soybean meal | 38.00 | 35.40 | 33.00 |
| Corn | 22.55 | 20.20 | 21.40 |
| Wheat | 20.00 | 24.50 | 25.00 |
| Rye | 12.00 | 12.00 | 12.00 |
| Soya oil | 3.80 | 4.30 | 4.85 |
| DL-Methionine | 0.20 | 0.15 | 0.15 |
| NaCl | 0.15 | 0.15 | 0.15 |
| DCP | 1.70 | 1.85 | 1.95 |
| CaCO3 | 0.54 | 0.39 | 0.34 |
| Premix¹ | 1.00 | 1.00 | 1.00 |
| Coccidiostat | 0.06 | — | — |
| Calculated content | | | |
| Crude protein (%) | 22.5 | 21.5 | 20.5 |
| Metabolizable energy | 12.6 | 12.8 | 13.0 |
| (MJ/kg)² | | | |
| Analyzed content | | | |
| Crude protein (%) | 21.9 | 21.5 | 20.8 |
| Metabolizable energy | 12.5 | 12.6 | 12.9 |
| (MJ/kg)³ | | | |

¹Vitamin-mineral premix provided per kilogram of diet: Vitamin A: 10'000 I.U.; vitamin E: 40 I.U.; vitamin K3: 3.0 mg; vitamin C: 100 mg; vitamin B1: 2.50 mg; vitamin B2: 8.00 mg; vitamin B6: 5.00 mg; vitamin B12: 0.03 mg; niacin: 50.0 mg; pantothenate calcium: 12.0 mg; folic acid: 1.50 mg; biotin 0.15 mg; cholin: 450 mg; ethoxyquine: 54 mg; Na: 1.17 g; Mg: 0.8 g; Mn: 80 mg; Fe: 60 mg; Cu: 30 mg; Zn: 54 mg; I: 1.24 mg; Co: 0.6 mg; Se: 0.3 mg.
¹Without coccidiostat;
²Calculated with EC-equation;
³Calculated with EC-equation based on analysed crude nutrients.

The diets were fed either unsupplemented (negative control, C), supplemented with the GH24 lysozyme (SEQ ID NO: 257) at 25 mg per kg feed or with Avilamycin at an inclusion level of 10 mg/kg. No additional enzymes (e.g., phytase) were added to the feed.

Appropriate amounts of the solid product (Avilamycin) was mixed with a small quantity of the basal feed as a premix which was then added to the feed to get the final concentration, according to the treatment. After mixing the feed was pelleted (3×25 mm) at about 70° C.

Appropriate amount of the liquid preparations of Lysozyme was diluted in water and sprayed onto the respective pelleted feed to get the final concentrations in the feed corresponding to the different treatments. For procedural balance of all treatments the same volume of water were also sprayed onto the pellets of the control diets.

### Experimental Parameters and Analyses

For the two experiments, the birds were weighed (as replicate group) on days 1, 22 and 36. The feed consumption for the intermediate periods was determined. Body weight gain and feed conversion ratio (feed/gain) were calculated.

The analyses of the nutrient content in the feed samples were performed according to standard methods (VDLUFA 1976). Nitrogen analysis was carried out with a Leco N analyzer (CP=N*6.25).

### Statistical Analysis

For the statistical evaluation of performance data, a one-factorial analysis of variance (factor: treatment) was carried out. The software ‘Stat Box Pro Agri’, version 7.1.9 (Grimmer soft, 1985-2011) was used. Where significant treatment effects (p<0.05) were indicated, the differences among treatment means were subsequently determined with the Newman-Keuls test.

### Results and Discussion

Based on the analyzed chemical compositions of the diets, the content of crude protein was close to the calculated content but the metabolizable energy was higher than expected in all the three diets (starter-grower and finisher) (Table 8).

The results of the growth performance are summarized in table 9 for the two periods (starter period, day 1-22; grower period, day 22-36, finisher period, day 36-42) and for the whole experimental period from day 1 to day 42 (table 10).

TABLE 9
Growth performance data of male broiler chickens fed graded inclusion levels of microbial lysozyme

| | Starter (d 1-22) | | | Grower (d 22-36) | | | Finisher (d 36-42) | | |
|---|---|---|---|---|---|---|---|---|---|
| | Weight | Feed | | Weight | Feed | | Weight | Feed | |
| | gain | intake | | gain | intake | | gain | intake | |
| Treatment/Product | (g/b) | (g/b) | FCR | (g/b) | (g/b) | FCR | (g/b) | (g/b) | FCR |
| Control (C) | 1096 | 1578 | 1.440 | 1328 | 2285 | 1.723 | 761 | 1487 | 1.960 |
| Avilamycin | 1102 | 1593 | 1.452 | 1334 | 2242 | 1.683 | 738 | 1460 | 1.982 |
| (10 mg/kg) | | | | | | | | | |
| Relative to C (%) | 100.6 | 101.0 | 100.8 | 100.4 | 98.1 | 97.7 | 97.0 | 98.2 | 101.1 |
| SEQ ID NO: 257 | 1133 | 1603 | 1.414 | 1364 | 2336 | 1.714 | 800 | 1524 | 1.919 |
| (25 mg/kg) | | | | | | | | | |
| Relative to C (%) | 103.4 | 101.6 | 98.1 | 102.7 | 102.2 | 99.5 | 105.1 | 102.5 | 97.9 |

TABLE 10
Growth performance summary of male broiler chickens
fed graded inclusion levels of microbial lysozyme

| | Whole period | | | | |
|---|---|---|---|---|---|
| | Weight | Feed | | | |
| Treatment/ | gain | intake | | | |
| Product | (g/b) | (g/b) | FCR | Motality | EPEF |
| Control (C) | 3186 | 5351 | 1.680 | 7.5 | 418 |
| Avilamycin | 3184 | 5312 | 1.669 | 12.5 | 397 |
| (10 mg/kg) | | | | | |
| Relative to C (%) | 99.9 | 99.3 | 99.3 | | 95.0 |
| SEQ ID NO: 257 | 3290 | 5450 | 1.657 | 10.0 | 425 |
| (25 mg/kg) | | | | | |
| Relative to C (%) | 103.3 | 101.9 | 98.6 | | 101.7 |

Over the whole period, the GH24 supplemented at 25 mg/kg resulted in a FCR improvement of 1.4% compared to NC diet. Furthermore, the GH24 lysozyme supplemented at 25 mg/kg resulted in an EPEF improvements of 1.7% compared to NC diet. Whilst the positive control Avilamycin showed a slight FCR improvement of 0.7%, the EPEF was worse by 5.0%.

### Conclusion

The results obtained in the study showed that the inclusion of a microbial lysozyme at 25 mg/kg was effective in improving the FCR and the EPEF of broilers fed diets formulated with coccidiostat in the starter period and based on soybean meal, corn, wheat and rye. In addition, the microbial lysozyme at 25 mg/kg was markedly better in improving the EPEF over the positive control (Avilamycin).

### Example 18: In Vivo Piglet Trial

### Materials and Methods

The trial was performed from Oct. 23 to Dec. 4, 2014 at the Research Center for Animal Nutrition (DSM Nutritional Products France, F-68305 Village-Neuf) according to the official French guidelines for experiments with live animals.

### Animals and Housing

One hundred and four castrated male crossbred (Large-White (female)×Redon (male)) weaned piglets having 28 days of age supplied by the commercial farm “Elevage de la Plaine du Rhin” located in Balgau (France), were used in a 42-day experiment. The initial bodyweight of the piglets was 7.88±0.675 kg. They were sorted by body weight into 32 groups of 3 or 4 piglets and randomly allotted to each dietary treatment. Each group of animals was placed in one flat-deck cages and allocated to one of the different treatments.

Each treatment was replicated with 8 cages using a total of 26 animals (6 cages of 3 animals/treatment and 2 cages of 4 animals/treatment) per treatment. Each cage had a plastic-coated welded wire floor and was equipped with two water nipples and two stainless-steel individualised feeders. Animals were housed in an environmentally controlled room. Room temperature was initially 27° C. and was lowered weekly by about 2° C. until 21-22° C. and relative humidity percentage was 50%.

### Feeding and Treatments

The experimental diets (Pre-Starter and Starter) were fed ad libitum in two feeding phases from day 0 to 14 (phase 1, Pre-starter) and day 14 to 42 (phase 2, Starter). The ingredient composition and the calculated nutrient levels of the experimental diets for phases 1 and 2 are presented in table 11. The analysed content is presented in table 12. Both diets were formulated to meet the animals' requirements according NRC (2012) and were fed in pelleted form. Pelleting conditions were at 70° C. for 30 seconds.

TABLE 11
Composition and nutrient contents of the basal experimental diets

| | Pre-starter (%) | Starter (%) |
|---|---|---|
| Ingredients | | |
| Barley | 38.00 | 38.00 |
| Wheat | 22.10 | 17.50 |
| Soybean meal 48% | 24.00 | 22.00 |
| Maize | 6.00 | 16.20 |
| Soybean oil | 1.00 | 2.00 |
| Dried whey | 5.00 | — |
| Vermiculite | — | 1.00 |
| Calcium carbonate | 0.30 | 0.30 |
| L-Lysine HCl | 0.10 | — |
| Vitamin-mineral Premix 3136¹ | 3.50 | 3.00 |
| Estimated nutrient content | | |
| Crude protein (%) | 19.47 | 17.95 |
| Lysine (%) | 1.24 | 1.03 |
| Threonine (%) | 0.67 | 0.61 |
| Methionine + cysteine (%) | 0.70 | 0.66 |
| Total P (%) | 0.71 | 0.64 |
| Total Ca (%) | 0.84 | 0.71 |
| Estimated digestible energy (MJ/kg) | 13.62 | 13.87 |

¹Vitamin-mineral premix 3136 provided per kilogram of diet: Vitamin A: 20'000 I.U.; Vitamin E: 100 mg.; Vitamin K: 4.0 mg; Vitamin C: 200 mg; Vitamin B1: 5.00 mg; Vitamin B2: 10.00 mg; Vitamin B6: 8.00 mg; Vitamin B12: 0.07 mg; Niacin: 60.0 mg; Pantothenic acid: 40.0 mg; Folic acid: 3.00 mg; Biotin 0.4 mg; Choline: 800 mg; Mn: 60.5 mg; Fe: 162 mg; Cu: 9.5 mg; Zn: 100 mg; I: 0.9 mg; Se: 0.3 mg

TABLE 12
Analysed nutrient contents of the basal diets

| Analyzed nutrient content | Pre-starter | Starter |
|---|---|---|
| Dry matter (%) | 87.89 | 87.42 |
| Crude protein (% DM) | 21.69 | 19.63 |
| Crude Ash (% DM) | 6.41 | 6.37 |
| Fat (% DM) | 4.15 | 5.42 |
| Starch (% DM) | 44.63 | 49.54 |
| Total P (mg/g DM) | 8.24 | 7.50 |
| Total Ca (mg/g DM) | 9.01 | 7.69 |
| Total Zn (mg/g DM) | 0.28 | 0.25 |
| Gross energy (MJ/kg DM) | 18.42 | 18.67 |

The diets were fed either unsupplemented (negative control) or supplemented with the GH24 lysozyme (SEQ ID NO: 257) at 50 mg per kg feed or VevoVital at 5000 mg per kg feed. No additional enzymes (e.g., phytase) were added to the feed.

| Treatment | Product | Inclusion level (mg/kg) |
|---|---|---|
| A | Negative control | — |
| B | VevoVital | 5000 |
| C | SEQ ID NO: 257 | 50 |

VevoVital was mixed to the premixed mash diet before pelleting the diet.

Appropriate amount of the liquid preparations of lysozyme was diluted in water and sprayed onto the respective pelleted feed to get the final concentrations in the feed corresponding to the different treatments. For procedural balance of all treatments the same volume of water were also sprayed onto the pellets of the control diets.

### Experimental Parameters and Analyses

The health status of the animals was controlled daily.

Body weight of the individual animals and feed consumption per pen were recorded on days 14 and 42 of the study. Performance, average daily weight gain (ADWG), average daily feed intake (ADFI) and feed conversion ratio (FCR) was calculated for phases 1 and 2, and the whole experimental period.

### Statistical Analysis

The experimental unit was the piglet, except for ADFI and FCR which were measured by cage, and in both cases, treatment was used as class variable.

Statistical analyses were performed using the StatGraphics Centurion XVI statistical software package (Manugistics, Rockville, MD).

One-factorial ANOVA and Student-Newman-Keuls test was used to assess differences among means in treatment groups.

Variability in the data was expressed as the pooled standard error. In all instances, differences were reported as significant at P<0.05.

### Results and Discussion

Based on the analyzed chemical compositions of the diets, the content of crude protein (19.07% and 17.16% as is, in pre-starter and starter periods, respectively) was close to the calculated content (19.47% and 17.95% as is, in pre-starter and starter periods, respectively) (Table 12).

All piglets remained healthy throughout the study. During the enzyme supplementation, none of the animals showed any symptoms of illness or toxicosis due to the test compounds. Mortality rate was equal to zero.

Results of the growth performance are summarized for the two periods (pre-starter period, days 0-14, and starter period, days 14-42) and for the whole experimental periods from day 0 to day 42 (Table 12).

In general, excellent animal growth performance was obtained for all treatments.

No significant difference among the supplemented treatments was recorded in terms of body weight. However, the supplementation the GH24 lysozyme (SEQ ID NO: 257) at 50 mg/kg resulted in numerical improvement of the ADWG by 11.8% during the starter period, and 7.8% during the whole period, compared to the negative control diet.

Over the pre-starter, starter and whole periods the supplementation of VevoVital resulted in an improvement of ADWG by 4.8, 5.8 and 5.4% compared to NC.

The results of FCR showed a statistically significant effect (p<0.05) of treatment in the pre-starter, starter and overall periods.

Over the starter period, from day 15 to day 42, piglets, which received VevoVital (PC) or the GH24 lysozyme (SEQ ID NO: 257) included at 50 mg/kg showed an improvement to FCR by 9.5% and 9.1% compared to NC.

As presented in Table 13, during the overall period (day 0 to day 42) piglets receiving feed added with VevoVital (PC) at 5000 mg/kg, the GH24 lysozyme (SEQ ID NO: 4) included at 50 mg/kg showed an improvement on FCR by 9.6% and 7.1% respectively compared to the negative control.

TABLE 13
Growth performance data of piglets fed graded inclusion levels of microbial lysozyme

| | | day 0-14 | | | day 14-42 | | | day 0-42 | | |
|---|---|---|---|---|---|---|---|---|---|---|
| | | ADFI | ADWG | | ADFI | ADWG | | ADFI | ADWG | FCR |
| Treatment | | (g/d) (2) | (g/d) (1) | FCR (2) | (g/d) (2) | (g/d) (1) | FCR (2) | (g/d) (2) | (g/d) (1) | (2) |
| Negative | Mean | 360 | 290 | 1.240^{ab} | 930^{bc} | 603 | 1.544^{b} | 734^{b} | 499 | 1.484^{b} |
| control | SD | 20 | 66 | 0.053 | 34 | 79 | 0.096 | 23 | 69 | 0.072 |
| (NC) | % | 100 | 100 | 100 | 100 | 100 | 100 | 100 | 100 | 100 |
| VevoVital | Mean | 355 | 304 | 1.168^{a} | 886^{ab} | 638 | 1.398^{a} | 709^{ab} | 526 | 1.342^{a} |
| (PC) | SD | 26 | 56 | 0.041 | 33 | 62 | 0.103 | 30 | 52 | 0.079 |
| | % relative | 98.6 | 104.8 | 94.2 | 95.3 | 105.8 | 90.5 | 96.6 | 105.4 | 90.4 |
| | NC | | | | | | | | | |
| NC + SEQ | Mean | 333 | 265 | 1.272^{b} | 944^{c} | 674 | 1.404^{a} | 740^{b} | 538 | 1.379^{a} |
| ID NO: 257 | SD | 42 | 53 | 0.049 | 62 | 89 | 0.044 | 49 | 69 | 0.040 |
| (50 ppm) | % relative | 92.5 | 91.4 | 102.6 | 101.5 | 111.8 | 90.9 | 100.8 | 107.8 | 92.9 |
| | NC | | | | | | | | | |
| | P value | 0.317 | 0.346 | 0.014 | 0.013 | 0.068 | 0.009 | 0.034 | 0.341 | 0.004 |

(1) Mean ± mean deviation of 18 determinations;
(2) Mean ± mean deviation of 6 determinations;
^{a,b,c}Different superscripts in the same column indicate a significant difference (p < 0.05);
ADFI: average daily feed intake;
ADWG: average daily weight gain.

### Conclusion

It can be concluded that in the present study and at the tested dosages and conditions, the GH24 lysozyme supplemented at 50 mg per kg feed to soybean meal, maize, wheat and barley based diet had a numerically improvement on growth performance of piglets although this effect was not statistically significant. Improvement of BWG with the GH24 lysozyme (SEQ ID NO: 257) at 50 mg/kg feed was even higher than the positive control. Of importance, nevertheless, was to observe a statistically significant effect of the GH24 lysozyme (SEQ ID NO: 257) included at 50 mg/kg feed treatment on ADFI and FCR in the starter and overall periods.

### Example 19: Expression of the GH24 Lysozyme from Trichophaea minuta

The gene from Trichophaea minuta was codon optimized using Gene designer and synthesized by Geneart. The gene of interest was cut out using BamHI and XhoI from NEB and the gel purified using Sigma Agarose gel purification Kit. An expression vector pDAU222 was also made, digested with BamHI and XhoI from NEB, and the larger fragment gel purified.

Finally, the construct of interest was made using Clontech In-Fusion® HD PCR Cloning Kit. In the construct, the gene of interest was under the influence of NA2TPI promoter of the vector pDAU222 and it had its native signal for secretion. The gene of interest was having its own terminator codon TAA made to it. The positive clone grown on LB-ampicillin plate in E. coli was re-confirmed by colony PCR and then one positive clone was picked to put into Aspergillus oryzae MT3568 strain. The positive clone in Aspergillus was selected through AMDS selection by plating the transformants on sucrose agar with 1 M acetamide. One positive transformant was then grown in 300 ml DAP-4C media in 1 liter baffled flask at 30° C. for 4 days and then purified as described in example 20.

### Example 20: Purification of the GH24 Lysozyme from Trichophaea minuta (SEQ ID NO: 291)

The target molecule was purified using a Phenyl Toyo coloumn. The purification was carried out at pH 8 using HEPES (50 mM)+1.5 M ammonium sulphate for equilibriation, HEPES (50 mM)+1 M ammonium sulphate to wash and HEPES, pH8 to elute. The purity of the purified enzymes was checked by SDS-PAGE and the concentration of the enzyme determined by Abs 280 nm after a buffer exchange. The purified protein was also MS verified.

### Example 21: Genomic DNA Extraction from Strains of Chaetomium sp., Mortierella sp., Metarhizium sp., Geomyces auratus and Ilyonectria rufa

Strain Chaetomium sp. and Mortierella sp. were inoculated onto a PDA plate and incubated for 7 days at 37° C. in the darkness. Several mycelia-PDA plugs were inoculated into 500 ml shake flasks containing 100 ml of YPG medium. The flasks were incubated for 4-5 days at 37° C. with shaking at 160 rpm.

Strain Metarhizium sp. and Geomyces auratus were inoculated onto a PDA plate and incubated for 7 days at 25° C. in the darkness. Several mycelia-PDA plugs were inoculated into 500 ml shake flasks containing 100 ml of YPG medium. The flasks were incubated for 3 days at 25° C. with shaking at 160 rpm.

Strain Ilyonectria rufa were inoculated onto a PDA plate and incubated for 7 days at 25° C. in the darkness. Several mycelia-PDA plugs were inoculated into 500 ml shake flasks containing 100 ml of YPG medium. The flasks were incubated for 11 days at 25° C. with shaking at 160 rpm.

The mycelia were collected by filtration through MIRACLOTH® (Calbiochem, La Jolla, CA, USA) and frozen under liquid nitrogen. Frozen mycelia were ground, by a mortar and a pestle, to a fine powder, and genomic DNA was isolated using DNeasy® Plant Maxi Kit (24) (QIAGEN GmbH, Hilden, Germany) following the manufacturer's instruction.

### Example 22: Genome Sequencing, Assembly and Annotation

The extracted genomic DNA samples were genome sequenced using an ILLUMINA® HiSeq 2000 System (Illumina, Inc., San Diego, CA, USA).

The raw reads of Mortierella sp. and Ilyonectria rufa were assembled using program Idba (Peng Yu et al., 2010, Research in Computational Molecular Biology 6044:426-440. Springer Berlin Heidelberg.). The raw reads of Chaetomium sp., Metarhizium sp. and Geomyces auratus were assembled using program Spades (Bankevich et al., 2012, Journal of Computational Biology 19 (5): 455-477). The assembled sequences were analyzed using standard bioinformatics methods for gene identification and function prediction. GeneMark-ES fungal version (Ter-Hovhannisyan et al., 2008, Genome Research 18 (12): 1979-1990) was used for gene prediction. Blastall version 2.2.10 (Altschul et al., 1990, Journal of Molecular Biology 215 (3): 403-410, ncbi.nlm.nih.gov/blast/executables/release/2.2.10/) and HMMER version 2.1.1 (National Center for Biotechnology Information (NCBI), Bethesda, MD, USA) were used to predict function based on structural homology. The GH24 family lysozyme polypeptides were identified directly by analysis of the Blast results. The Agene program (Munch and Krogh, 2006, BMC Bioinformatics 7:263) and SignalP program (Nielsen et al., 1997, Protein Engineering 10:1-6) were used to identify start codons. SignalP program was further used to predict signal peptides. Pepstats (Rice et al., 2000, Trends in Genetics 16 (6): 276-277) was used to predict isoelectric points and molecular weights.

### Example 23: Cloning of GH24 Lysozymes (SEQ ID NO: 292, 295, 298, 301, 304)

Five fungal GH24 lysozyme wild type sequences were cloned from Chaetomium sp. (SEQ ID NO: 292), Mortierella sp. (SEQ ID NO: 295), Metarhizium sp. (SEQ ID NO: 298), Geomyces auratus (SEQ ID NO: 301) and Ilyonectria rufa (SEQ ID NO: 304).

The fungal GH24 lysozymes were cloned into an Aspergillus oryzae expression vector pCaHj505 as described in WO 2013/029496. The transcription of the GH24 lysozyme coding sequence with the native secretion signal was under the control of an Aspergillus oryzae alpha-amylase gene promoter.

The final expression plasmids, p505-GH24_Chaet287, p505-GH24_Mort, p505-GH24_Metar, p505-GH24_Geau, and p505-GH24_Ilyru (SEQ ID NO: 292, 295, 298, 301, 304), were individually transformed into an Aspergillus oryzae expression host. The GH24 lysozyme genes were integrated by homologous recombination into the Aspergillus oryzae host genome upon transformation. Four transformants of each transformation were selected from the selective media agar plate and inoculated to 3 ml of YPM medium in 24-well plate and incubated at 30° C., 150 rpm. After 3 days incubation, 20 μl of supernatant from each transformant were analyzed on NuPAGE Novex 4-12% Bis-Tris Gel w/MES according to the manufacturer's instructions. The resulting gel was stained with Instant Blue. SDS-PAGE profiles of the cultures showed that all 5 genes were expressed with 1 or 2 protein bands each detected at 30 KD, 28 KD & 30 KD, 28 KD, 25 KD & 28 KD and 28 KD respectively. The recombinant Aspergillus oryzae strain with the strongest protein band were selected for shaking flask culturing and were inoculated on slant made of slant medium and incubated at 37° C. for 6-7 days. When strains were well grown to fully sporulated, they were inoculated to 2 L shaking flasks each containing 400 ml of YPM and 4-8 flasks for each strain. Flasks were shaking at 80 rpm, 30° C. Cultures were harvested on day 3 and filtered using a 0.45 μm DURAPORE Membrane and were purified as described in example 24 to 28.

### Example 24: Purification of the GH24 Lysozyme from Chaetomium sp. ZY287 (SEQ ID NO: 294)

The culture supernatant was firstly precipitated with ammonium sulfate (80% saturation), then dialyzed with 20 mM PBS at pH 6.5. The solution was filtered with 0.45 um filter and then loaded into SP Fast Flow column (GE Healthcare) equilibrated with 20 mM PBS at pH 6.5. A gradient of NaCl concentration was applied as elution buffer from zero to 1 M, and then elution fractions and flow-through fraction were collected to detect lysozyme activity. The fractions with lysozyme activity were pooled and analyzed by SDS-PAGE, and then concentrated for further evaluation. The protein concentration was determined by Qubit® Protein Assay Kit (Invitrogen, cat Q33212).

### Example 25: Purification of the GH24 Lysozyme from Mortierella sp. ZY002 (SEQ ID NO: 297)

The culture supernatant was firstly precipitated with ammonium sulfate (80% saturation), then dialyzed with 20 mM PBS at pH 7.0. The solution was filtered with 0.45 um filter and then loaded into SP Fast Flow column (GE Healthcare) equilibrated with 20 mM PBS at pH 7.0. A gradient of NaCl concentration was applied as elution buffer from zero to 1 M, and then elution fractions and flow-through fraction were collected to detect lysozyme activity. The fractions with lysozyme activity were pooled and analyzed by SDS-PAGE, and then concentrated for further evaluation. The protein concentration was determined by Qubit® Protein Assay Kit (Invitrogen, cat Q33212).

### Example 26: Purification of the GH24 Lysozyme from Metarhizium sp. XZ2431 (SEQ ID NO: 300)

The culture supernatant was firstly precipitated with ammonium sulfate (80% saturation), then dialyzed with 20 mM PBS at pH 7.0. The solution was filtered with 0.45 um filter and then loaded into SP Fast Flow column (GE Healthcare) equilibrated with 20 mM PBS at pH 7.0. A gradient of NaCl concentration was applied as elution buffer from zero to 1 M, and then elution fractions and flow-through fraction were collected to detect lysozyme activity. The fractions with lysozyme activity were pooled and analyzed by SDS-PAGE, and then concentrated for further evaluation. The protein concentration was determined by Qubit® Protein Assay Kit (Invitrogen, cat Q33212).

### Example 27: Purification of the GH24 Lysozyme from Geomyces auratus (SEQ ID NO: 303)

The culture supernatant was firstly precipitated with ammonium sulfate (80% saturation), then dialyzed with 20 mM PBS at pH 7.0. The solution was filtered with 0.45 um filter and then loaded into SP Fast Flow column (GE Healthcare) equilibrated with 20 mM PBS at pH 7.0. A gradient of NaCl concentration was applied as elution buffer from zero to 1 M, and then elution fractions and flow-through fraction were collected to detect lysozyme activity. The fractions with lysozyme activity were pooled and analyzed by SDS-PAGE, and then concentrated for further evaluation.

Since the purified sample has two bands, the collected sample was added ammonium sulfate with a final conductivity with 180 mS/cm, and then loaded into a Phenyl Sepharose 6 Fast Flow column (GE Healthcare) equilibrated with 20 mM PBS at pH 7.0 with 1.8 M (NH₄)₂SO₄. A concentration gradient of (NH₄)₂SO₄ was applied as elution buffer from 1.8 M to zero. The fractions with lysozyme activity were collected and carried out for SDS-PAGE. The sample also contains two bands, but MS data showed both bands are target proteins. The protein concentration was determined by Qubit® Protein Assay Kit (Invitrogen, cat Q33212).

### Example 28: Purification of the GH24 Lysozyme from Ilyonectria rufa (SEQ ID NO: 306)

The culture supernatant was firstly precipitated with ammonium sulfate (80% saturation), then dialyzed with 20 mM PBS at pH 7.5. The solution was filtered with 0.45 um filter and then loaded into SP Fast Flow column (GE Healthcare) equilibrated with 20 mM PBS at pH 7.5. A gradient of NaCl concentration was applied as elution buffer from zero to 1 M, and then elution fractions and flow-through fraction were collected to detect lysozyme activity. The fractions with lysozyme activity were pooled and analyzed by SDS-PAGE, and then concentrated for further evaluation. The protein concentration was determined by Qubit® Protein Assay Kit (Invitrogen, cat Q33212).

### Example 29: Determination of Lysozyme Activity

Lysozyme activity was determined as described in example 11 herein and is presented in table 14.

TABLE 14
Lysozyme Activity against Micrococcus lysodeikticus and
Exiguobacterium undea as measured by Optical Density Drop

| | Micrococcus | | Exiguobacterium | |
|---|---|---|---|---|
| GH24 Lysozyme | lysodeikticus¹ | | undae¹ | |
| GH24 lysozyme from | +++ | (pH 6) | ++++ | (pH 6) |
| Trichoderma harzianum | | | | |
| (SEQ ID NO: 267) | | | | |
| GH24 lysozyme from | ++++ | (pH 6) | ++++ | (pH 6) |
| Trichophaea minuta | | | | |
| (SEQ ID NO: 291) | | | | |
| GH24 lysozyme from | +++ | (pH 7) | +++ | (pH 7) |
| Chaetomium sp. | | | | |
| ZY287 (SEQ ID NO: 294) | | | | |
| GH24 lysozyme from | ++++ | (pH 6) | +++ | (pH 6) |
| Mortierella sp. | | | | |
| ZY002 (SEQ ID NO: 297) | | | | |
| GH24 lysozyme from | +++ | (pH 6) | +++ | (pH 6) |
| Metarhizium sp. | | | | |
| XZ2431 (SEQ ID NO: 300) | | | | |
| GH24 lysozyme from | +++ | (pH 6) | ++++ | (pH 6) |
| Geomyces auratus | | | | |
| (SEQ ID NO: 303) | | | | |
| GH24 lysozyme from | ++ | (pH 6) | ++++ | (pH 6) |
| Ilyonectria rufa | | | | |
| (SEQ ID NO: 306) | | | | |

¹Means no significant effect; + means small effect; ++ means medium effect; +++ means large effect; ++++ means very large effect.
The pH value in the brackets lists the assay pH based on lysozyme-substrate combination.

The data shows that all of the GH24 lysozymes that naturally comprise the lysozyme enhancing domain (LED) (SEQ ID NO: 267, 291, 294, 297, 300, 303 and 306) showed good activity against both substrates.

### Example 30: Determination of DomT Scores

The DomT scores for the GH24 domain and the LED domain of the lysozymes of the invention were determined using the Lysozyme Enhancing Domain HMM from example 15 and the GH24 catalytic domain HMM from example 16 as described herein. The DomT scores for other prior art lysozyme sequences were also calculated and are presented in table 15 below.

TABLE 15
DomT scores for GH24 catalytic
and Lysozyme Enhancing Domains

| | | | DomT |
|---|---|---|---|
| | | DomT | score |
| | | score | for GH24 |
| Sequence | Domains | of LED¹ | domain |
| SEQ ID NO: 257 | LED + GH24 | 103.0 | 265.9 |
| SEQ ID NO: 264 | LED + GH24 | 127.0 | 257.2 |
| SEQ ID NO: 267 | LED + GH24 | 125.0 | 249.6 |
| SEQ ID NO: 279 | GH24 | — | 251.9 |
| SEQ ID NO: 280 | GH24 | — | 229.8 |
| SEQ ID NO: 291 | LED + GH24 | 103.1 | 263.2 |
| SEQ ID NO: 294 | LED + GH24 | 113.1 | 254.0 |
| SEQ ID NO: 297 | LED + GH24 | 103.1 | 246.3 |
| SEQ ID NO: 300 | LED + GH24 | 117.8 | 242.2 |
| SEQ ID NO: 303 | LED + GH24 | 119.8 | 246.9 |
| SEQ ID NO: 306 | LED + GH24 | 116.7 | 257.5 |
| Lactococcus c2 phage | GH24 phage | — | 74.5 |
| from WO 95/31562 | | | |
| (GENESEQP: AAR85295) | | | |
| Enterobacteria phage | GH24 phage | — | 34.9 |
| T4 lysozyme, SEQ ID | | | |
| NO: 24 of WO 2013/ | | | |
| 021206 (BAK49017) | | | |

¹No score means that there was no alignment to the HMM and thus no score could be generated

No DomT scores could be generated for the lysozyme enhancing domain or the GH24 domain (i.e., there was no sequence alignment) for the other lysozymes tested, including GH25 lysozymes disclosed in WO 2005/080559, WO 2009/102755, WO 2013/076253, WO 2013/076259, GH22 lysozyme such as hen egg white lysozyme and a GH23 lysozyme disclosed in WO 2013/076259 demonstrating that the HMMs are selective for the GH24 catalytic domain and the lysozyme enhancing domain respectively.

Unsurprisingly the prior art GH24 phage lysozymes did not align very well with the HMM of the GH24 catalytic domain as disclosed herein. These phage lysozymes are structurally and taxonomically very different from the fungal GH24 lysozymes of the invention and furthermore do not comprise a LED. In comparison, all of the GH24 lysozymes of the invention gave a domT score of at least 200 for the GH24 catalytic domain and a domT score of at least 100 for the lysozyme enhancing domain indicating good alignment to the HMMs.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

That which is claimed:

1. A recombinant host cell comprising a nucleic acid construct or expression vector comprising a polynucleotide encoding a polypeptide having lysozyme activity, wherein the polynucleotide is operably linked to one or more control sequences that direct the production of the polypeptide in a recombinant host cell and wherein the polypeptide has at least 80% sequence identity to SEQ ID NO: 264.

2. A method comprising cultivating the recombinant host cell of claim 1 under conditions conducive for production of said polypeptide.

3. An animal feed additive comprising a polypeptide having lysozyme activity and at least 80% sequence identity to SEQ ID NO: 264.

4. The animal feed additive of claim 3, wherein the polypeptide has at least 85% sequence identity to SEQ ID NO: 264.

5. The animal feed additive of claim 3, wherein the polypeptide has at least 90% sequence identity to SEQ ID NO: 264.

6. The animal feed additive of claim 3, wherein the polypeptide has at least 95% sequence identity to SEQ ID NO: 264.

7. The animal feed additive of claim 3, wherein the polypeptide has at least 97% sequence identity to SEQ ID NO: 264.

8. The animal feed additive of claim 3, wherein the polypeptide has at least 99% sequence identity to SEQ ID NO: 264.

9. The animal feed additive of claim 3, wherein the polypeptide comprises SEQ ID NO: 264.

10. The animal feed additive of claim 3, wherein the polypeptide comprises a Glycoside Hydrolase Family 24 (GH24) catalytic domain and a lysozyme enhancing domain.

11. The animal feed additive of claim 3, wherein the polypeptide comprises a GH24 catalytic domain comprising motif I: [TAS][VIL][GAC][YFI]GHX[CAYIV] (SEQ ID NO: 252)

12. The animal feed additive of claim 3, wherein the polypeptide comprises a GH24 catalytic domain comprising motif II: LNXN[QE][YFW][GA]ALXS[WFL]X[YF]N (SEQ ID NO: 283).

13. The animal feed additive of claim 3, wherein the polypeptide comprises a lysozyme enhancing domain comprising motif III: [CGY][YF][VIL][ASTP][DG]X[YF][VIT]X[TS][GAN] (SEQ ID NO: 254).

14. The animal feed additive of claim 3, wherein the polypeptide comprises:
(a) GH24 catalytic domain comprising motif I: [TAS][VIL][GAC][YFI]GHX[CAYIV] (SEQ ID NO: 252) and/or a GH24 catalytic domain comprising motif II: LNXN[QE][YFW][GA]ALXS[WFL]X[YF]N (SEQ ID NO: 283); and
(b) a lysozyme enhancing domain comprising motif III:
(SEQ ID NO: 254)
[CGY][YF][VIL][ASTP][DG]X[YF][VIT]X[TS][GAN].

15. The animal feed additive of claim 3, further comprising one or more components selected from additional enzymes, amino acids, antibiotics, forages, microbes, minerals, and vitamins.

16. A method comprising administering the animal feed additive of claim 3 to an animal.

17. The method of claim 3, wherein the animal is a ruminant.

18. The method of claim 3, wherein the animal is monogastric.

19. The method of claim 3, wherein the animal is a poultry.

20. The method of claim 3, wherein the animal is a swine.
